(19) United States
(12) Patent Application Publication
(10) Pub. No.: US 2023/0355722 A1
(43) Pub. Date: Nov. 9, 2023

(54) TREATMENT OF SJOGREN’S SYNDROME WITH NUCLEASE FUSION PROTEINS

(71) Applicant: Resolve Therapeutics, LLC, St. Petersburg, FL (US)

(72) Inventors: James Arthur POSADA, St. Petersburg, FL (US); Daniel BURGE, Sammamish, WA (US)

(21) Appl. No.: 18/012,459

(22) PCT Filed: Jun. 29, 2021
(86) PCT No.: PCT/US2021/039683

§ 371 Date: Dec. 22, 2022

Related U.S. Application Data
(60) Provisional application No. 63/045,705, filed on Jun. 29, 2020.

(51) Int. Cl. A61P 37/06; A61K 38/46; C12Q 1/6869

(52) U.S. Cl. CPC A61K 38/465; A61P 37/06; C12Q 1/6869; C12Q 2600/106; C12Q 2600/158

(57) ABSTRACT

The present disclosure provides methods for treating Sjogren’s syndrome by administering nuclease fusion proteins. The methods of the disclosure are useful to treat symptoms associated with Sjogren’s syndrome, including fatigue.

### CROSS-REFERENCE TO RELATED APPLICATIONS

This application claims the benefit of U.S. Provisional Pat. Application Serial No. 63/045,705 filed on Jun. 29, 2020, the content of which is herein incorporated by reference in its entirety.

### BACKGROUND

Primary Sjogren’s Syndrome (pSS) is an autoimmune disorder that is estimated to afflict between 0.5% to 1% of the general population, of whom nine out of ten patients are women (Ramos-Casals, 2005, Ann Rheum Dis. 64, 347-354; Skopouli, 2000, Semin. Arthritis Rheum. 29:296-304). The majority of women with pSS are characterized by mild to moderate disease which manifests as fatigue, joint pain, and ocular and/or oral dryness. The disease is characterized by the lymphocytic infiltration of salivary and lacrimal glands with subsequent inflammation, damage and loss of function of the glands causing dry eyes and dry mouth. Involvement of major organ systems including lung, kidney, and liver are common systemic manifestations of pSS (Malladi, 2012, Arthritis Care Res. 64:911-918). At a biochemical level, pSS is associated with increased immunoglobulin levels and the production of anti-nuclear antibodies against ribonucleoprotein complexes such as SSA/Ro and SSB/La (Bave, 2005, Arth. & Rheum. 52:1185-1195; Hall 2015, Arth. & Rheum. 67, 2437-2446).

Once formed, RNA-containing immune complexes are readily internalized into immune system cells such as dendritic cells, where the RNA bound to the immune complexes is able to interact with Toll-Like Receptors (TLRs), such as the RNA sensor, TLR7. Although TLRs are thought to operate as key elements of the innate immune system by recognizing pathogen-associated molecular components, it is now clear that host nucleic acids also can activate specific family members including TLR7, TLR8, and TLR9 (Theofilopoulos, 2010, Nat. Rev. Rheum., 6:146-156). Cells expressing these receptors do so without distributing them to the cell surface; rather, TLRs 7/8/9 are sequestered within endosomes (Theofilopoulos, 2010, Nat. Rev. Rheum., 6:146-156). This positioning is postulated to minimize interaction with host nucleic acids. However, when present within an immune complex, nucleic acid antigens are actively internalized into cells via receptor-mediated endocytosis. Effector Fc, complement, and B-cell receptors all may facilitate entry of nucleic acid containing ICs into endosomes (Means, 2005, J. Clin. Invest. 115:407-417; Lau, 2005, J. Exp. Med 202:1171-1177; Brkic 2013, Ann. Rheum. Dis. 72(5):728-735). Once internalized, the nucleic acid is positioned to bind to and activate resident endosomal TLRs. Activated TLRs, in turn, promote type 1 IFN production from pDCs, activate PMNs, and promote B-cell proliferation and autoantibody production. Thus, the nucleic acid-containing antigens contribute to multiple aspects of pSS disease pathophysiology.

Fatigue is one of the most common extraglandular symptoms of Sjogren’s syndrome and is defined by enduring generalized tiredness. An estimated 70% of pSS patients suffer from profound fatigue, which is reported to have a negative impact on quality of life. The biochemical basis of profound fatigue associated with pSS has been studied (Bodewes et al., Front Immunol. 2019, 10:312). Chronic inflammation accompanied by activation of interferon-inducible genes, mimicking the immune response to viral infection has been found in pSS patients (Nezos et al. J. Autoimmun. 2015, 63:47-58; Hall et al., Arthritis & Rheumatology, 2015, 67:2437-2446). One of the roles of the immune system is to detect and respond to infection by retroviruses. Toll-like receptors and other pattern recognition receptors are exquisitely sensitive to the presence of circulating RNA and respond with a robust activation of the innate immune system (Driedonks et al., Front Immunol. 2019: 9; Katze et al., Nat. Rev. Immunol. 2008; 8:644-654). In the case of autoimmune disease such as pSS and systemic lupus erythematosus (SLE) the presence of circulating autoantibodies which present RNA autoantigens to the immune system mimics retroviral infection and drives chronic inflammation.

Approximately 80% of pSS patients have anti-Ro/SSA autoantibodies which bind to autoantigens containing small non-coding RNA molecules (Moutsopoulos et al., Clin. Rheumatol. 1990; 9:123-130; Kattah et al., Immunol. Rev. 2010; 233:126-145; Noll et al., PLoS ONE, 2017; 12:e0190002). These immune complexes can trigger inflammatory cytokine production in vitro (Eloranta et al., Arthritis Rheum. 2009;60:2418-2427). Furthermore, a large observational study in SLE patients demonstrated a correlation between the presence of RNA-containing immune complexes, chronic interferon pathway activation and disease activity (Doedens et al., J. Immunol. 2016; 197:2854-2863). In addition to RNA autoantigens, many non-coding RNAs are found in circulation and many possess inflammatory gene regulatory function (Hur et al, Immune Netw 2019;19:34).

Fatigue associated with Sjogren’s syndrome can be characterized in terms of intensity, duration, and effects on daily function. Notably, in the primary care setting fatigue is strongly associated with depression. (Segal et al. Arthritis Rhem. 2008 December 15; 59(12): 1780-1778). Thus, there exists a need for a means to improve fatigue in patients with autoimmune diseases such as Sjogren’s syndrome.

### SUMMARY OF THE INVENTION

The disclosure relates, at least in part, to RNase-containing nuclease fusion proteins, including RNase-Fc fusion proteins, which are useful to treat Sjogren’s syndrome in human patients in need thereof. In some aspects, the disclosure relates to RNase-containing nuclease fusion proteins, including RNase-Fc fusion proteins, that are useful for treating, reducing or ameliorating fatigue in patients with Sjogren’s syndrome. In some aspects, the disclosure relates to RNase-containing nuclease fusion proteins, including RNase-Fc fusion proteins, that are useful for improving, enhancing or increasing cognitive ability, or reducing, decreasing or ameliorating cognitive deficits in patients with Sjogren’s syndrome. In some aspects, the disclosure relates to RNase-containing nuclease fusion proteins, including RNase-Fc fusion proteins, that are useful for reducing, decreasing or ameliorating depression in patients with Sjogren’s syndrome, including depression associated with fatigue. In some aspects, the disclosure relates to compositions comprising an RNase-Fc fusion protein and one or more pharmaceutically acceptable carriers and/or diluents that are useful in methods for treating or preventing Sjogren’s syndrome, methods for treating, preventing or reducing fatigue in patients with Sjogren’s syndrome, and methods for improving cognitive ability in patients with Sjogren’s syndrome. In some embodiments, the RNase-Fc fusion protein is administered to human patients by injection (e.g., intravenous injection) at a dose of about 5-10 mg/kg, about 2-8 mg/kg, about 3-6 mg/kg, about 3 mg/kg, about 5 mg/kg, or about 10 mg/kg.

In some embodiments, the RNase-Fc fusion protein is RSLV-132. RSLV-132 is a nuclease fusion protein comprising a homodimer of two polypeptides each having the amino acid sequence set forth as SEQ ID NO: 50. Each polypeptide of the homodimer has the configuration shown in FIG. 1 from N- to C- terminus of RNase-Fc, wherein a wild-type human RNase 1 domain (SEQ ID NO: 2) is operably coupled without a linker to the N-terminus of a human IgG1 Fc domain comprising mutations in one of three hinge region cysteine residues to serine (residue 220 or C220S, also referred to herein as “SCC hinge”) and two mutations in the CH2 domain, P238S and P331S. The sequence of the human IgG1 Fc domain with these mutations is set forth in SEQ ID NO: 22.

In some aspects, the disclosure provides a method for treating Sjogren’s disease by reducing fatigue in a human patient in need thereof, the method comprising administering an effective amount of an RNase-containing nuclease fusion protein, such as an RNase-Fc fusion protein, e.g., RSLV-132, to the patient, thereby treating Sjogren’s disease by reducing fatigue in the patient.

In some aspects, the disclosure provides a method for treating Sjogren’s disease by reducing fatigue in a human patient in need thereof, the method comprising administering by intravenous injection a dose of an RNase-containing nuclease fusion protein, such as an RNase-Fc fusion protein, e.g., RSLV-132, of about 5-10 mg/kg to the patient, thereby treating Sjogren’s disease by reducing fatigue in the patient.

In some aspects, the disclosure provides a method for treating Sjogren’s disease by improving cognitive effects in a human patient in need thereof, the method comprising administering an effective amount of an RNase-containing nuclease fusion protein, such as an RNase-Fc fusion protein, e.g., RSLV-132, to the patient, thereby treating Sjogren’s disease by improving cognitive effects in the patient. In some aspects, the cognitive effects in the patient are improved by at least one point in a mental component of ProF relative to a mental component of ProF prior to treatment. In some aspects, the cognitive effects in the patient are improved by greater than 1 point, greater than 2 points, or greater than 3 points in a mental component of ProF relative to a mental component of ProF prior to treatment.

In some aspects, the disclosure provides a method for treating Sjogren’s disease by reducing fatigue in a human patient in need thereof, the method comprising administering an effective amount of an RNase-Fc fusion protein comprising the amino acid sequence as set forth in SEQ ID NO: 50 to the patient, thereby treating Sjogren’s disease by reducing fatigue in the patient. In some aspects, the effective amount of the RNase-Fc fusion protein comprising the amino acid sequence as set forth in SEQ ID NO: 50 is a dose of about 5 mg/kg to about 10 mg/kg. In some aspects, the RNase-Fc fusion protein is administered in the form of a composition with an pharmaceutically acceptable carrier. In some aspects, the composition comprising the RNase-Fc fusion protein comprising the amino acid sequence as set forth in SEQ ID NO: 50 is formulated for intravenous injection (e.g., in a solution).

In some aspects, the disclosure provides a method for treating Sjogren’s disease by reducing fatigue in a human patient in need thereof, comprising administering an effective amount of a pharmaceutical composition to the patient, wherein the composition comprises: an RNase-Fc fusion protein comprising the amino acid sequence as set forth in SEQ ID NO: 50; and one or more pharmaceutically acceptable carriers and/or diluents, thereby treating Sjogren’s disease by reducing fatigue in the patient. In some aspects, the composition comprising the RNase-Fc fusion protein comprising the amino acid sequence as set forth in SEQ ID NO: 50 is formulated for intravenous injection (e.g., in a solution).

In any of the foregoing or related embodiments of the methods of the disclosure, the RNase-Fc fusion protein for use herein comprises a human pancreatic RNase 1. In some aspects, the human pancreatic RNase 1 comprises the amino acid sequence as set forth in SEQ ID NO: 2. In some aspects, the RNase-Fc fusion protein of the disclosure comprises a wild-type human IgG1 Fc domain or a human IgG1 Fc domain comprising one or more mutations. In some aspects, the human IgG1 Fc domain comprises one or more mutations which decrease binding to Fcγ receptors on human cells. In some aspects, the RNase-Fc fusion protein of the disclosure has a reduced effector function optionally selected from the group consisting of opsonization, phagocytosis, complement dependent cytotoxicity, and antibody-dependent cellular cytotoxicity.

In some aspects, the human IgG1 Fc domain comprises a hinge domain, a CH2 domain and a CH3 domain. In some aspects, the human IgG1 Fc domain comprises a substitution of one or more of three hinge region cysteine residues with serine. In some aspects, the Fc domain comprises an SCC mutation (residues 220, 226, and 229), numbering according to the EU index. In some aspects, the human IgG1 Fc domain comprises the amino acid sequence as set forth in SEQ ID NO: 22.

In any of the foregoing or related embodiments of the methods of the disclosure, the RNase-Fc fusion protein for use herein comprises a human pancreatic RNase 1 coupled with or without a linker to a human IgG1 Fc domain comprising a C220S mutation, a P238S mutation and a P331S mutation according to EU numbering.

In any of the foregoing or related embodiments of the methods of the disclosure, the RNase-Fc fusion protein for use herein comprises the amino acid sequence as set forth in SEQ ID NO: 50.

In any of the foregoing or related embodiments of the methods of the disclosure, the RNase-Fc fusion protein, e.g., RSLV-132, or pharmaceutical composition comprising the RNase-Fc fusion protein, for use herein is administered to the patient by intravenous injection. In some aspects, the patient is administered an effective dose of the RNase-Fc fusion protein, e.g., RSLV-132, or pharmaceutical composition comprising the RNase-Fc fusion protein, every two weeks.

In some aspects, the RNase-Fc fusion protein of the disclosure, e.g., RSLV-132, or pharmaceutical composition comprising the RNase-Fc fusion protein, is administered to the patient at a dose of about 5-10 mg/kg. In some aspects, the RNase-Fc fusion protein, e.g., RSLV-132, or pharmaceutical composition comprising the RNase-Fc fusion protein, is administered to the patient at a dose of about 10 mg/kg. In some aspects, the RNase-Fc fusion protein, e.g., RSLV-132, or pharmaceutical composition comprising the RNase-Fc fusion protein, is administered to the patient at a dose of about 5 mg/kg. In some aspects, the RNase-Fc fusion protein of the disclosure, e.g., RSLV-132, or pharmaceutical composition comprising the RNase-Fc fusion protein, is administered to the patient at a dose of about 5-10 mg/kg every two weeks. In some aspects, the RNase-Fc fusion protein, e.g., RSLV-132, or pharmaceutical composition comprising the RNase-Fc fusion protein, is administered to the patient at a dose of about 5-10 mg/kg every two weeks for three months. In some aspects, the RNase-Fc fusion protein, e.g., RSLV-132, or pharmaceutical composition comprising the RNase-Fc fusion protein, is administered to the patient in six biweekly infusions over three months. In some aspects, the RNase-Fc fusion protein, e.g., RSLV-132, or pharmaceutical composition comprising the RNase-Fc fusion protein, is administered to the patient every week for three weeks, and then one administration every two weeks to achieve or maintain a therapeutic effect.

In some aspects, the disclosure provides a method for treating Sjogren’s disease by reducing fatigue in a human patient in need thereof, the method comprising administering a dosing regimen of at least three doses of an RNase-Fc fusion protein, e.g., RSLV-132, or pharmaceutical composition comprising the RNase-Fc fusion protein, wherein each dose is administered to the patient (e.g., by injection, e.g., intravenous injection) at a dose of about 5-10 mg/kg, about 2-8 mg/kg, about 3-6 mg/kg, about 3 mg/kg, about 5 mg/kg, or about 10 mg/kg. In some aspects, the patient is administered at least four doses of the RNase-Fc fusion protein. In some aspects, the patient is administered at least five doses of the RNase-Fc fusion protein. In some aspects, the patient is administered at least six doses of the RNase-Fc fusion protein. In some aspects, the patient is administered at least seven doses of the RNase-Fc fusion protein. In some aspects, the patient is administered at least eight doses of the RNase-Fc fusion protein.

In some aspects, the disclosure provides a method for treating Sjogren’s disease by reducing fatigue in a human patient in need thereof, the method comprising administering a dosing regimen of a dose of an RNase-Fc fusion protein, e.g., RSLV-132, or pharmaceutical composition comprising the RNase-Fc fusion protein, once weekly for at least two weeks, wherein each dose is administered to the patient (e.g., by injection, e.g., intravenous injection) at a dose of about 5-10 mg/kg, about 2-8 mg/kg, about 3-6 mg/kg, about 3 mg/kg, about 5 mg/kg, or about 10 mg/kg. In some aspects, the patient is administered a dose of the RNase-Fc fusion protein every week for at least three weeks. In some aspects, the patient is administered a dose of the RNase-Fc fusion protein every week for at least four weeks. In some aspects, the patient is administered a dose of the RNase-Fc fusion protein every week for at least five weeks. In some aspects, the patient is administered a dose of the RNase-Fc fusion protein every week for at least six weeks. In some aspects, the patient is administered a dose of the RNase-Fc fusion protein every week for at least seven weeks. In some aspects, the patient is administered a dose of the RNase-Fc fusion protein every week for at least eight weeks.

In some aspects, the patient is administered a dose of the RNase-Fc fusion protein every two weeks for at least two weeks. In some aspects, the patient is administered a dose of the RNase-Fc fusion protein every two weeks for at least four weeks. In some aspects, the patient is administered a dose of the RNase-Fc fusion protein every two weeks for at least six weeks. In some aspects, the patient is administered a dose of the RNase-Fc fusion protein every two weeks for at least eight weeks.

In some aspects, the patient is administered a dose of the RNase-Fc fusion protein every week for three weeks and then one administration every two weeks for at least one month. In some aspects, the patient is administered a dose of the RNase-Fc fusion protein every week for three weeks and then one administration every two weeks for at least two months. In some aspects, the patient is administered a dose of the RNase-Fc fusion protein every week for three weeks and then one administration every two weeks for at least three months.

In some aspects, the disclosure provides a method for treating Sjogren’s disease by reducing fatigue in a human patient in need thereof, the method comprising administering an effective amount of an RNase-Fc fusion protein, e.g., RSLV-132, or pharmaceutical composition comprising the RNase-Fc fusion protein, wherein treatment reduces fatigue in the patient by at least a one point in an EULAR SS Patient Reported Index (ESSPRI) score relative to an ESSPRI score prior to treatment. In some aspects, treatment reduces the ESSPRI score by the patient by at least one point relative to the ESSPRI score prior to treatment. In some aspects, fatigue is reduced by the patient to a score of between 4.5 and 5.5 on an ESSPRI scale of 1 to 10.

In some aspects, the disclosure provides a method for treating Sjogren’s disease by reducing fatigue in a human patient in need thereof, the method comprising administering an effective amount of an RNase-Fc fusion protein, e.g., RSLV-132, or pharmaceutical composition comprising the RNase-Fc fusion protein, wherein treatment improves fatigue in the patient by at least one point in a Functional Assessment of Chronic Illness Therapy (FACIT) fatigue scale relative to a FACIT score prior to treatment. In some aspects, treatment improves fatigue in the patient by at least two points in a FACIT fatigue scale. In some aspects, treatment increases the FACIT fatigue score by the patient by at least one point relative to the FACIT fatigue score prior to treatment. In some aspects, treatment increases the FACIT fatigue score by the patient by at least two points relative to the FACIT fatigue score prior to treatment.

In some aspects, the disclosure provides a method for treating Sjogren’s disease by reducing fatigue in a human patient in need thereof, the method comprising administering an effective amount of an RNase-Fc fusion protein, e.g., RSLV-132, or pharmaceutical composition comprising the RNase-Fc fusion protein, wherein treatment reduces fatigue in the patient by at least one point in a Profile of Fatigue (ProF) score relative to a ProF score prior to treatment. In some aspects, treatment reduces fatigue in the patient by at least one point in a mental component of Profile of Fatigue (ProF) score relative to a mental component ProF score prior to treatment. In some aspects, treatment reduces fatigue in the patient by at least one point in a somatic component of Profile of Fatigue (ProF) score relative to a somatic component ProF score prior to treatment.

In some aspects, the disclosure provides a method for treating Sjogren’s disease by improving cognitive function in a human patient in need thereof, the method comprising administering an effective amount of an RNase-Fc fusion protein, e.g., RSLV-132, or pharmaceutical composition comprising the RNase-Fc fusion protein, wherein treatment improves cognitive function in the patient as measured by the Digit Symbol Substitution Test (DSST) test relative to a DSST test score prior to treatment. In some aspects, treatment increases the number of matches completed in 90 seconds by the patient on a Digit Symbol Substitution Test (DSST) test. In some aspects, treatment reduces the time to complete the DSST test by the patient.

In other aspects, the disclosure provides a kit comprising a container comprising an injectable solution comprising an effective amount of an RNase-Fc fusion protein of the disclosure, e.g., RSLV-132, or the RNase-Fc fusion protein comprising the amino acid sequence as set forth in SEQ ID NO: 50 or pharmaceutical composition comprising the RNase-Fc fusion protein; and one or more pharmaceutically acceptable carriers and/or diluents; and instructions for use in treating Sjogren’s disease by reducing fatigue in a human patient in need thereof, wherein the injectable solution is formulated for intravenous administration.

In some aspects, the disclosure provides an RNase-Fc fusion protein, e.g., RSLV-132, or pharmaceutical composition comprising the RNase-Fc fusion protein, for use in method for treating Sjogren’s disease by reducing fatigue in a human patient in need thereof, the treatment comprising administering an effective amount of the RNase-Fc fusion protein to the patient.

In some aspects, the disclosure provides an RNase-Fc fusion protein, e.g., RSLV-132, or pharmaceutical composition comprising the RNase-Fc fusion protein, for use in the manufacture of a medicament for treating Sjogren’s disease by reducing fatigue in a human patient in need thereof.

In some aspects, the disclosure provides an RNase-Fc fusion protein, e.g., RSLV-132, or pharmaceutical composition comprising the RNase-Fc fusion protein, for use in a method for treating Sjogren’s disease by reducing fatigue in a human patient in need thereof, the treatment comprising administering by intravenous injection a dose of the RNase-Fc fusion protein of about 5-10 mg/kg to the patient.

In some aspects, the disclosure provides an RNase-Fc fusion protein, e.g., RSLV-132, or pharmaceutical composition comprising the RNase-Fc fusion protein, for use in the manufacture of a medicament for treating Sjogren’s disease by reducing fatigue in a human patient in need thereof, the use comprising administering by intravenous injection a dose of the RNase-Fc fusion protein of about 5-10 mg/kg to the patient.

In some aspects, the disclosure provides an RNase-Fc fusion protein, e.g., RSLV-132, or pharmaceutical composition comprising the RNase-Fc fusion protein, for use in a method for treating Sjogren’s disease by improving cognitive effects in a human patient in need thereof, the treatment comprising administering an effective amount of the RNase-Fc fusion protein to the patient.

In some aspects, the disclosure provides an RNase-Fc fusion protein, e.g., RSLV-132, or pharmaceutical composition comprising the RNase-Fc fusion protein, for use in the manufacture of a medicament for treating Sjogren’s disease by improving cognitive effects in a human patient in need thereof, the use comprising administering an effective amount of the RNase-Fc fusion protein to the patient.

In some aspects, the disclosure provides an RNase-Fc fusion protein, e.g., RSLV-132, or pharmaceutical composition comprising the RNase-Fc fusion protein, for use in a method for treating Sjogren’s disease by reducing fatigue in a human patient in need thereof, the treatment comprising administering an effective amount of an RNase-Fc fusion protein comprising the amino acid sequence as set forth in SEQ ID NO: 50 to the patient.

In some aspects, the disclosure provides an RNase-Fc fusion protein, e.g., RSLV-132, or pharmaceutical composition comprising the RNase-Fc fusion protein, for use in the manufacture of a medicament for treating Sjogren’s disease by reducing fatigue in a human patient in need thereof, the use comprising administering an effective amount of an RNase-Fc fusion protein comprising the amino acid sequence as set forth in SEQ ID NO: 50 to the patient.

In some aspects, the disclosure provides an RNase-Fc fusion protein, e.g., RSLV-132, or pharmaceutical composition comprising the RNase-Fc fusion protein, for use in a method for treating Sjogren’s disease by reducing fatigue in a human patient in need thereof, the treatment comprising administering an effective amount of a pharmaceutical composition to the patient, wherein the composition comprises: an RNase-Fc fusion protein comprising the amino acid sequence as set forth in SEQ ID NO: 50; and one or more pharmaceutically acceptable carriers and/or diluents.

In some aspects, the disclosure provides an RNase-Fc fusion protein, e.g., RSLV-132, or pharmaceutical composition comprising the RNase-Fc fusion protein, for use in the manufacture of a medicament for treating Sjogren’s disease by reducing fatigue in a human patient in need thereof, the use comprising administering an effective amount of a pharmaceutical composition to the patient, wherein the composition comprises: an RNase-Fc fusion protein comprising the amino acid sequence as set forth in SEQ ID NO: 50; and one or more pharmaceutically acceptable carriers and/or diluents.

In some aspects, the disclosure provides a method of treating Sjogren’s syndrome in a patient in need thereof, the method comprising administering an effective amount of an RNA nuclease agent to the patient, wherein treatment results in an increase in one or more interferon-inducible genes.

In some aspects, the disclosure provides a method of treating Sjogren’s syndrome in a patient in need thereof, the method comprising administering an effective amount of an RNA nuclease agent to the patient, wherein treatment results in an increase in one or more interferon-inducible genes and an improvement in fatigue.

In some aspects, the disclosure provide a method of treating Sjogren’s syndrome in a patient in need thereof, the method comprising administering an effective amount of an RNA nuclease agent to the patient, wherein treatment results in an increase in one or more interferon-inducible genes, wherein the one or more interferon-inducible genes is selected from the group consisting of LY6E, RTP4, EPSTI1, OAS1, OAS2, OASL, BATF2, SERPING1, OTOF, IFITM3, ISG15, HES4, XAF1, OAS3, IFIT3, RSAD2, LAMP3, IFIT1, IFI44L, HERC5, MX1, and IFI44.

In some embodiments, the interferon-inducible gene is OAS1. In some embodiments, the interferon-inducible gene is ISG15. In some embodiments, the interferon-inducible gene is HES4. In some embodiments, the interferon-inducible gene is IFIT1. In some embodiments, the interferon-inducible gene is IFI44L. In some embodiments, the interferon-inducible gene is IFI44.

In some embodiments, treatment results in an improvement in fatigue.

In some embodiments, the RNA nuclease agent is an RNase-Fc. In some embodiments, the RNA nuclease agent is RSLV-132.

In some aspects, the disclosure provides, a method of treating Sjogren’s syndrome in a patient in need thereof, the method comprising administering an effective amount of an RNA nuclease agent to the patient, wherein treatment results in an increase in one or more interferon-inducible genes, wherein the one or more interferon-inducible genes is selected from the group consisting of HERC6, ATF3, GBP3. GBP4, IFITM1, MT2A, TNFAIP6, LAP3, OAS2, PLSCR1, TNFSF10, TIMM10, UBE2L6, IFI35, AIM2, GALM, EIF2AK2, ZBP1, APOL6, STAT1, GBP5, LGALS3BP, SEPT4, FBXO6, SCO2, MOV10, CEACAM1, PML, WARS, CCL8, SOCS1, ZNF684, SAMD9L, PARP10, GBP1, IRF7, IFIT5, IFIH1, DHX58, IFIT2, PARP12, TRIM22, DDX58, STAT2, PARP9, and PARP14.

In some embodiments, the interferon-inducible gene is HERC6. In some embodiments, the interferon-inducible gene is ATF3. In some embodiments, the interferon-inducible gene is GBP3. In some embodiments, the interferon-inducible gene is TNFSF10. In some embodiments, the interferon-inducible gene is TIMM10. In some embodiments, the interferon-inducible gene is CCL8. In some embodiments, the interferon-inducible gene is SOCS1. In some embodiments, the interferon-inducible gene is SAMD9L. In some embodiments, the interferon-inducible gene is DHX58.

In some embodiments, treatment results in an improvement in fatigue.

In some embodiments, the RNA nuclease agent is an RNase-Fc. In some embodiments, the RNA nuclease agent is RSLV-132.

In some aspects, the disclosure provides a method of treating Sjogren’s syndrome in a patient in need thereof, the method comprising administering an effective amount of an RNA nuclease agent to the patient, wherein treatment results in an increase in one or more interferon-inducible genes, wherein the one or more interferon-inducible genes is selected from the group consisting of MDK, CHMP5, SAMD9, C1QA, TCN2, ACTA2, NCOA7, PSMB9, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, SP140, CASP1, TRIM5, NMI, DYNLT1, TRAFD1, TDRD7, BTN3A1, ZC3HAV1, DHRS9, GBP2, TRIM56, HSH2D, and UNC93B1.

In some embodiments, the interferon-inducible gene is MDK. In some embodiments, the interferon-inducible gene is CHMP5. In some embodiments, the interferon-inducible gene is SAMD9. In some embodiments, the interferon inducible gene is BST2. In some embodiments, the interferon-inducible gene is ISG20. In some embodiments, the interferon-inducible gene is DRAP1. In some embodiments, the interferon-inducible gene is PHF11.

In some embodiments, treatment results in an improvement in fatigue.

In some embodiments, the RNA nuclease agent is an RNase-Fc. In some embodiments, the RNA nuclease agent is RSLV-132.

In some aspects, the disclosure provides for the use of an RNA nuclease agent in the manufacture of a medicament for the treatment of Sjogren’s syndrome in a patient, the use comprising administering an effective amount of an RNA nuclease agent to the patient, wherein treatment results in an increase in one or more interferon-inducible genes, wherein the one or more interferon-inducible genes is selected from the group consisting of LY6E, RTP4, EPSTI1, OAS1, OAS2, OASL, BATF2, SERPING1, OTOF, IFITM3, CXCL10, ISG15, HES4, XAF1, OAS3, IFIT3, RSAD2, LAMP3, IFIT1, IFI44L, HERC5, MX1, and IFI44.

In some aspects, the disclosure provides for the use of an RNA nuclease agent in the manufacture of a medicament for the treatment of Sjogren’s syndrome in a patient, the use comprising administering an effective amount of an RNA nuclease agent to the patient, wherein treatment results in an increase in one or more interferon-inducible genes, wherein the one or more interferon-inducible genes is selected from the group consisting of HERC6, ATF3, GBP3. GBP4. IFITM1, MT2A, TNFAIP6, LAP3, OAS2, PLSCR1, TNFSF10, TIMM10, UBE2L6, IFI35, AIM2, GALM, EIF2AK2, ZBP1, APOL6, STAT1, GBP5, LGALS3BP, SEPT4, FBXO6, SCO2, MOV10, CEACAM1, PML, WARS, CCL8, SOCS1, ZNF684, SAMD9L, PARP10, GBP1, IRF7, IFIT5, IFIH1, DHX58, IFIT2, PARP12, TRIM22, DDX58, STAT2, PARP9, and PARP14.

In some aspects, the disclosure provides for the use of an RNA nuclease agent in the manufacture of a medicament for the treatment of Sjogren’s syndrome in a patient, the use comprising administering an effective amount of an RNA nuclease agent to the patient, wherein treatment results in an increase in one or more interferon-inducible genes, wherein the one or more interferon-inducible genes is selected from the group consisting of MDK, CHMP5, SAMD9, C1QA, TCN2, ACTA2, NCOA7, PSMB9, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, SP140, CASP1, TRIM5, NMI, DYNLT1, TRAFD1, TDRD7, BTN3A1, ZC3HAV1, DHRS9, GBP2, TRIM56, HSH2D, and UNC93B1.

In some embodiments, treatment results in an improvement in fatigue.

In some embodiments, the RNA nuclease agent is an RNase-Fc. In some embodiments, the RNA nuclease agent is RSLV-132.

In some aspects, the disclosure provides for an RNA nuclease agent for use in a method of treating Sjogren’s syndrome in a patient, the use comprising administering an effective amount of an RNA nuclease agent to the patient, wherein treatment results in an increase in one or more interferon-inducible genes, wherein the one or more interferon-inducible genes is selected from the group consisting of LY6E, RTP4, EPSTI1, OAS1, OAS2, OASL, BATF2, SERPING1, OTOF, IFITM3, CXCL10, ISG15, HES4, XAF1, OAS3, IFIT3, RSAD2, LAMP3, IFIT1, IFI44L, HERC5, MX1, and IFI44.

In some aspects, the disclosure provides for an RNA nuclease agent for use in a method of treating Sjogren’s syndrome in a patient, the use comprising administering an effective amount of an RNA nuclease agent to the patient, wherein treatment results in an increase in one or more interferon-inducible genes, wherein the one or more interferon-inducible genes is selected from the group consisting of HERC6, ATF3, GBP3. GBP4. IFITM1, MT2A, TNFAIP6, LAP3, OAS2, PLSCR1, TNFSF10, TIMM10, UBE2L6, IFI35, AIM2, GALM, EIF2AK2, ZBP1, APOL6, STAT1, GBP5, LGALS3BP, SEPT4, FBXO6, SCO2, MOV10, CEACAM1, PML, WARS, CCL8, SOCS1, ZNF684, SAMD9L, PARP10, GBP1, IRF7, IFIT5, IFIH1, DHX58, IFIT2, PARP12, TRIM22, DDX58, STAT2, PARP9, and PARP14.

In some aspects, the disclosure provides for an RNA nuclease agent for use in a method of treating Sjogren’s syndrome in a patient, the use comprising administering an effective amount of an RNA nuclease agent to the patient, wherein treatment results in an increase in one or more interferon-inducible genes, wherein the one or more interferon-inducible genes is selected from the group consisting of MDK, CHMP5, SAMD9, C1QA, TCN2, ACTA2, NCOA7, PSMB9, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, SP140, CASP1, TRIM5, NMI, DYNLT1, TRAFD1, TDRD7, BTN3A1, ZC3HAV1, DHRS9, GBP2, TRIM56, HSH2D, and UNC93B1.

In some embodiments, treatment results in an improvement in fatigue.

In some embodiments, the RNA nuclease agent is an RNase-Fc. In some embodiments, the RNA nuclease agent is RSLV-132.

In some aspects, the disclosure provides for an RNA nuclease agent for use in a method of treating Sjogren’s syndrome in a patient, the use comprising administering an effective amount of an RNA nuclease agent to the patient, wherein treatment results in an increase in one or more interferon-inducible genes, wherein the one or more interferon-inducible genes is selected from the group consisting of MDK, CHMP5, SAMD9, C1QA, TCN2, ACTA2, NCOA7, PSMB9, GADD45B, BST2, ISG20, DRAP1, PHF11, TRIM38, ETV7, RBCK1, SP100, ABCA1, IL-18, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, SP140, CASP1, ISG15, TRIM5, NMI, DYNLT1, TRAFD1, TDRD7, BTN3A1, ZC3HAV1, DHRS9, GBP2, CXCL10, TRIM56, HSH2D, and UNC93B1.

In some embodiments, treatment results in an improvement in fatigue.

In some aspects, the disclosure provides a method of identifying a patient having Sjogren’s syndrome as a candidate for treatment with an RNA nuclease agent, comprising (a) determining an interferon-inducible gene expression profile in a sample obtained from the patient; and (b) comparing the interferon-inducible gene expression profile determined in step (a) with an interferon-inducible gene expression profile in a sample obtained from a suitable control subject, wherein the interferon-inducible genes include one or more of the following interferon-inducible genes: MDK, CHMP5, SAMD9, C1QA, TCN2, ACTA2, NCOA7, PSMB9, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, SP140, CASP1, TRIM5, NMI, DYNLT1, TRAFD1, TDRD7, BTN3A1, ZC3HAV1, DHRS9, GBP2, TRIM56, HSH2D, and UNC93B1; and wherein the interferon-inducible gene expression profile indicates the patient is a candidate for treatment with an RNA nuclease agent.

In some aspects, the disclosure provides a method of identifying a patient having Sjogren’s syndrome as a candidate for treatment with an RNA nuclease agent, comprising (a) determining an interferon-inducible gene expression profile in a sample obtained from the patient; and (b) comparing the interferon-inducible gene expression profile determined in step (a) with an interferon-inducible gene expression profile in a sample obtained from a suitable control subject, wherein the interferon-inducible genes include one or more of the following interferon-inducible genes: MDK, CHMP5, SAMD9, C1QA, TCN2, ACTA2, NCOA7, PSMB9, GADD45B, BST2, ISG20, DRAP1, PHF11, TRIM38, ETV7, RBCK1, SP100, ABCA1, IL-18, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, SP140, CASP1, ISG15, TRIM5, NMI, DYNLT1, TRAFD1, TDRD7, BTN3A1, ZC3HAV1, DHRS9, GBP2, CXCL10, TRIM56, HSH2D, and UNC93B1; and wherein the interferon-inducible gene expression profile indicates the patient is a candidate for treatment with an RNA nuclease agent.

In some embodiments, the RNA nuclease agent is an RNase-Fc. In some embodiments, the RNA nuclease agent is RSLV-132.

In some aspects, the disclosure provides a method of treating Sjogren’s syndrome in a patient in need thereof, the method comprising administering an effective amount of an RNA nuclease agent to the patient, wherein treatment results in an increase in one or more interferon-inducible genes, wherein the one or more interferon-inducible genes is selected from one or more module M1.2 interferon inducible genes.

In some aspects, the disclosure provides a method of treating Sjogren’s syndrome in a patient in need thereof, the method comprising administering an effective amount of an RNA nuclease agent to the patient, wherein treatment results in an increase in one or more interferon-inducible genes, wherein the one or more interferon-inducible genes is selected from one or more module M3.4 interferon inducible genes.

In some aspects, the disclosure provides a method of treating Sjogren’s syndrome in a patient in need thereof, the method comprising administering an effective amount of an RNA nuclease agent to the patient, wherein treatment results in an increase in one or more interferon-inducible genes, wherein the one or more interferon-inducible genes is selected from one or more module M5.12 interferon inducible genes.

In some embodiments, treatment results in an improvement in fatigue.

In some embodiments, the RNA nuclease agent is an RNase-Fc. In some embodiments, the RNA nuclease agent is RSLV-132.

In some aspects, the disclosure provides for the use of an RNA nuclease agent in the manufacture of a medicament for the treatment of Sjogren’s syndrome in a patient, the use comprising administering an effective amount of an RNA nuclease agent to the patient, wherein treatment results in an increase in one or more interferon-inducible genes, wherein the one or more interferon-inducible genes is selected from one or more module M1.2 interferon inducible genes.

In some aspects, the disclosure provides for the use of an RNA nuclease agent in the manufacture of a medicament for the treatment of Sjogren’s syndrome in a patient, the use comprising administering an effective amount of an RNA nuclease agent to the patient, wherein treatment results in an increase in one or more interferon-inducible genes, wherein the one or more interferon-inducible genes is selected from one or more module M3.4 interferon inducible genes.

In some aspects, the disclosure provides for the use of an RNA nuclease agent in the manufacture of a medicament for the treatment of Sjogren’s syndrome in a patient, the use comprising administering an effective amount of an RNA nuclease agent to the patient, wherein treatment results in an increase in one or more interferon-inducible genes, wherein the one or more interferon-inducible genes is selected from one or more module M5.12 interferon inducible genes.

In some embodiments, treatment results in an improvement in fatigue.

In some embodiments, the RNA nuclease agent is an RNase-Fc. In some embodiments, the RNA nuclease agent is RSLV-132.

In some aspects, the disclosure provides an RNA nuclease agent for use in a method of treating Sjogren’s syndrome in a patient, the use comprising administering an effective amount of an RNA nuclease agent to the patient, wherein treatment results in an increase in one or more interferon-inducible genes, wherein the one or more interferon-inducible genes is selected from one or more module M1.2 interferon inducible genes.

In some aspects, the disclosure provides an RNA nuclease agent for use in a method of treating Sjogren’s syndrome in a patient, the use comprising administering an effective amount of an RNA nuclease agent to the patient, wherein treatment results in an increase in one or more interferon-inducible genes, wherein the one or more interferon-inducible genes is selected from one or more module M3.4 interferon inducible genes.

In some aspects, the disclosure provides an RNA nuclease agent for use in a method of treating Sjogren’s syndrome in a patient, the use comprising administering an effective amount of an RNA nuclease agent to the patient, wherein treatment results in an increase in one or more interferon-inducible genes, wherein the one or more interferon-inducible genes is selected from one or more module M5.12 interferon inducible genes.

In some embodiments, treatment results in an improvement in fatigue.

In some embodiments, the RNA nuclease agent is an RNase-Fc. In some embodiments, the RNA nuclease agent is RSLV-132.

In some aspects, the disclosure provides a method of treating Sjogren’s syndrome in a patient in need thereof, the method comprising administering an effective amount of an RNA nuclease agent to the patient, wherein treatment results in a decrease in one or more long non-coding RNA (lncRNA) molecules. In some embodiments, the one or more lncRNA molecules is one or more lncRNA molecules that mediates the expression of one or more pro-inflammatory genes. In some embodiments, the one or more pro-inflammatory genes is a pro-inflammatory gene related to autoimmune disease and/or immune system function.

In some embodiments, the one or more lncRNA molecules is selected from the group consisting of PXN-AS1, AP002852.1, AL009176.1, MKX-AS1, AC109464.1, AL590560.2, AC020908.3, AL731533.3, AL353743.2, AC007405.1, DPRXP2, ST20-AS1, AC020916.1, AC027601.1, AL358334.2, AL358394.2, AL022238.2, AC116366.2, AP003031.1, CYP4F30P, AL390071.1, AC008532.1, CPHL1P, AC008870.4, AC008763.1, AL355388.2, AC110926.2, AL133415.1, AL591503.1, DLGAP1-AS1, CHL1-AS2, AC010326.4, AC051649.1, HCG27, AL034417.2, AP001160.1, AC009435.1, LINC01499, AL161621.1, AP001469.1, AC021491.2, AC010168.1, SLC44A3-AS1, AC011445.2, ZMIZ1-AS1, AC027018.1, AL591379.1, AC061992.1, AL513348.1, AC092617.1, AC015853.3, AC138655.1, AC108134.3, LINC01515, AC243829.2, AC020911.1, AC016168.4, AL590705.1, FAM3D-AS1, AC078881.1, AC004466.2, AC018866.1, AC010531.3, AJ011931.2, TUSC8, PTOV1-AS1, RPS4XP2, AC021148.2, AC009041.2, AC093525.6, LINC01516, AL670729.3, C18ORF65, AC098818.2, FAM230C, LINC01988, AC068831.1, CYP51A1-AS1, LINC01227, LINC00895, LINC01519, AC114730.3, MIPEPP3, ITPRIP-AS1, AC138207.5, LINC01091, AC012645.3, AC082651.1, DLGAP1-AS3, AC080011.1, AC010148.1, AC078962.1, AL132822.1, LINC02552, AC098484.2, AC090587.1, LINC00173, PIK3CD-AS1, AL021393.1, AL450322.2, AL591368.1, AC010894.4, AC067930.8, AC024267.3, AC104984.1, AC109495.1, AC129926.1, KLF3-AS1, AL512306.3, LINC01448, AC080013.6, and DACT3-AS1.

In some embodiments, the one or more lncRNA molecules is selected from the group consisting of PXN-AS1, AC116366.2, HCG27, AL034417.2, ZMIZ1-AS1, AL513348.1, AC243829.2, LINC01227, LINC01091, AC080011.1, AL591368.1, AC109495.1, KLF3-AS1, and LINC01448.

In some embodiments, treatment results in an improvement in fatigue.

In some embodiments, the RNA nuclease agent is an RNase-Fc. In some embodiments, the RNA nuclease agent is RSLV-132.

In some aspects, the disclosure provides for the use of an RNA nuclease agent in the manufacture of a medicament for the treatment of Sjogren’s syndrome in a patient, the use comprising administering an effective amount of an RNA nuclease agent to the patient, wherein treatment results in a decrease in one or more lncRNA molecules. In some embodiments, the one or more lncRNA molecules is an lncRNA molecule that regulates the transcription of a gene involved in inflammation. In some embodiments, the one or more lncRNA molecules is an lncRNA molecule that regulates the transcription of a pro-inflammatory gene.

In some embodiments, the one or more lncRNA molecules is selected from the group consisting of PXN-AS1, AP002852.1, AL009176.1, MKX-AS1, AC109464.1, AL590560.2, AC020908.3, AL731533.3, AL353743.2, AC007405.1, DPRXP2, ST20-AS1, AC020916.1, AC027601.1, AL358334.2, AL358394.2, AL022238.2, AC116366.2, AP003031.1, CYP4F30P, AL390071.1, AC008532.1, CPHL1P, AC008870.4, AC008763.1, AL355388.2, AC110926.2, AL133415.1, AL591503.1, DLGAP1-AS1, CHL1-AS2, AC010326.4, AC051649.1, HCG27, AL034417.2, AP001160.1, AC009435.1, LINC01499, AL161621.1, AP001469.1, AC021491.2, AC010168.1, SLC44A3-AS1, AC011445.2, ZMIZ1-AS1, AC027018.1, AL591379.1, AC061992.1, AL513348.1, AC092617.1, AC015853.3, AC138655.1, AC108134.3, LINC01515, AC243829.2, AC020911.1, AC016168.4, AL590705.1, FAM3D-AS1, AC078881.1, AC004466.2, AC018866.1, AC010531.3, AJ011931.2, TUSC8, PTOV1-AS1, RPS4XP2, AC021148.2, AC009041.2, AC093525.6, LINC01516, AL670729.3, C18ORF65, AC098818.2, FAM230C, LINC01988, AC068831.1, CYP51A1-AS1, LINC01227, LINC00895, LINC01519, AC114730.3, MIPEPP3, ITPRIP-AS1, AC138207.5, LINC01091, AC012645.3, AC082651.1, DLGAP1-AS3, AC080011.1, AC010148.1, AC078962.1, AL132822.1, LINC02552, AC098484.2, AC090587.1, LINC00173, PIK3CD-AS1, AL021393.1, AL450322.2, AL591368.1, AC010894.4, AC067930.8, AC024267.3, AC104984.1, AC109495.1, AC129926.1, KLF3-AS1, AL512306.3, LINC01448, AC080013.6, and DACT3-AS1.

In some embodiments, the one or more lncRNA molecules is selected from the group consisting of PXN-AS1, AC116366.2, HCG27, AL034417.2, ZMIZ1-AS1, AL513348.1, AC243829.2, LINC01227, LINC01091, AC080011.1, AL591368.1, AC109495.1, KLF3-AS1, and LINC01448.

In some embodiments, treatment results in an improvement in fatigue.

In some embodiments, the RNA nuclease agent is an RNase-Fc. In some embodiments, the RNA nuclease agent is RSLV-132.

In some aspects, the disclosure provides an RNA nuclease agent for use in a method of treating Sjogren’s syndrome in a patient, the use comprising administering an effective amount of an RNA nuclease agent to the patient, wherein treatment results in a decrease in one or more lncRNA molecules. In some embodiments, the one or more lncRNA molecules is an lncRNA molecule that regulates the transcription of a gene involved in inflammation. In some embodiments, the one or more lncRNA molecules is an lncRNA molecule that regulates the transcription of a pro-inflammatory gene.

In some embodiments, the one or more lncRNA molecules is selected from the group consisting of PXN-AS1, AP002852.1, AL009176.1, MKX-AS1, AC109464.1, AL590560.2, AC020908.3, AL731533.3, AL353743.2, AC007405.1, DPRXP2, ST20-AS1, AC020916.1, AC027601.1, AL358334.2, AL358394.2, AL022238.2, AC116366.2, AP003031.1, CYP4F30P, AL390071.1, AC008532.1, CPHL1P, AC008870.4, AC008763.1, AL355388.2, AC110926.2, AL133415.1, AL591503.1, DLGAP1-AS1, CHL1-AS2, AC010326.4, AC051649.1, HCG27, AL034417.2, AP001160.1, AC009435.1, LINC01499, AL161621.1, AP001469.1, AC021491.2, AC010168.1, SLC44A3-AS1, AC011445.2, ZMIZ1-AS1, AC027018.1, AL591379.1, AC061992.1, AL513348.1, AC092617.1, AC015853.3, AC138655.1, AC108134.3, LINC01515, AC243829.2, AC020911.1, AC016168.4, AL590705.1, FAM3D-AS1, AC078881.1, AC004466.2, AC018866.1, AC010531.3, AJ011931.2, TUSC8, PTOV1-AS1, RPS4XP2, AC021148.2, AC009041.2, AC093525.6, LINC01516, AL670729.3, C18ORF65, AC098818.2, FAM230C, LINC01988, AC068831.1, CYP51A1-AS1, LINC01227, LINC00895, LINC01519, AC114730.3, MIPEPP3, ITPRIP-AS1, AC138207.5, LINC01091, AC012645.3, AC082651.1, DLGAP1-AS3, AC080011.1, AC010148.1, AC078962.1, AL132822.1, LINC02552, AC098484.2, AC090587.1, LINC00173, PIK3CD-AS1, AL021393.1, AL450322.2, AL591368.1, AC010894.4, AC067930.8, AC024267.3, AC104984.1, AC109495.1, AC129926.1, KLF3-AS1, AL512306.3, LINC01448, AC080013.6, and DACT3-AS1.

In some embodiments, the one or more lncRNA molecules is selected from the group consisting of PXN-AS1, AC116366.2, HCG27, AL034417.2, ZMIZ1-AS1, AL513348.1, AC243829.2, LINC01227, LINC01091, AC080011.1, AL591368.1, AC109495.1, KLF3-AS1, and LINC01448.

In some embodiments, treatment results in an improvement in fatigue.

In some embodiments, the RNA nuclease agent is an RNase-Fc. In some embodiments, the RNA nuclease agent is RSLV-132.

In some aspects, the disclosure provides a method of treating Sjogren’s syndrome in a patient in need thereof, the method comprising administering an effective amount of an RNA nuclease agent to the patient, wherein treatment results in a change in expression of one or more inflammatory-related genes, wherein the one or more inflammatory-related gene(s) is selected from the group consisting of CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, PLA2G7, NUPR1, ZC3H12A, TPST1, CDK19, CCN3, IL5, IL1RAP, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and CXCL1..

In some aspects, the disclosure provides a method of treating Sjogren’s syndrome in a patient in need thereof, the method comprising administering an effective amount of an RNA nuclease agent to the patient, wherein treatment results in a decrease in expression of one or more inflammatory-related genes. In some embodiments, the one or more inflammatory-related gene(s) is selected from the group consisting of NUPR1, ZC3H12A, TPST1, CDK19, CCN3, IL5, IL1RAP, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and/or CXCL1.

In some aspects, the disclosure provides a method of treating Sjogren’s syndrome in a patient in need thereof, the method comprising administering an effective amount of an RNA nuclease agent to the patient, wherein treatment results in an increase in expression of one or more inflammatory-related genes. In some embodiments, the one or more inflammatory-related gene(s) is selected from the group consisting of CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, and/or PLA2G7.

In some embodiments, treatment results in an improvement in fatigue.

In some embodiments, the RNA nuclease agent is an RNase-Fc. In some embodiments, the RNA nuclease agent is RSLV-132.

In some aspects, the disclosure provides for the use of an RNA nuclease agent in the manufacture of a medicament for the treatment of Sjogren’s syndrome in a patient, the use comprising administering an effective amount of an RNA nuclease agent to the patient, wherein treatment results in a change in expression of one or more inflammatory-related genes. In some embodiments, the one or more inflammatory-related gene(s) is selected from the group consisting of CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, PLA2G7, NUPR1, ZC3H12A, TPST1, CDK19, CCN3, IL5, IL1RAP, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and CXCL1.

In some aspects, the disclosure provides for the use of an RNA nuclease agent in the manufacture of a medicament for the treatment of Sjogren’s syndrome in a patient, the use comprising administering an effective amount of an RNA nuclease agent to the patient, wherein treatment results in a decrease in expression of one or more inflammatory-related genes.

### BRIEF DESCRIPTION OF THE DRAWINGS

These and other features, aspects, and advantages of the present invention will become better understood with regard to the following description, and accompanying drawing, where:

FIG. 1 depicts the configuration of RSLV-132, a homodimeric RNase-Fc fusion protein comprising two polypeptides. Each polypeptide of the homodimer has the configuration RNase-Fc, wherein a wild-type human RNase 1 domain is operably coupled without a linker to the N-terminus of a human IgG1 Fc domain comprising an SCC hinge and CH2 mutations P238S and P331S.

FIG. 2 graphically depicts an improvement in ESSPRI score in pSS patients treated with RSLV-132 as compared with patients treated with placebo. The ESSPRI score was assessed in pSS patients treated with RSLV-132 and placebo on study days 1 (baseline), 29, 57, 85, and 99/end of treatment. A decrease in ESSPRI score of at least one point is clinically meaningful.

FIG. 3 graphically depicts an improvement in ESSPRI score in patients treated with RSLV-132 as compared with patients treated with placebo. The change in the ESSPRI score over time from baseline is provided on the y-axis. The ESSPRI score was assessed in patients treated with RSLV-132 and placebo on study days 1 (baseline), 29, 57, 85, and 99/end of treatment. A decrease in ESSPRI score of at least one point is clinically meaningful.

FIG. 4 graphically depicts the mean change from baseline in the fatigue component of the ESSPRI for the RSLV-132 and placebo groups (p=0.136). An improvement in the fatigue component of the ESSPRI score in patients treated with RSLV-132 as compared with patients treated with placebo is shown. The change in the fatigue component of the ESSPRI score over time from baseline is provided on the y-axis. The ESSPRI score was assessed in patients treated with RSLV-132 and placebo on study days 1 (baseline), 29, 57, 85, and 99/end of treatment. Results were analyzed using separate 1- way Analysis of Variance (ANOVA) models for each visit, each testing the null hypothesis (H₀) that the true mean difference between treatment groups was zero. Unadjusted alpha = 0.05. All standard error bars use Day 99 standard errors.

FIG. 5A graphically depicts an improvement in the fatigue component of the ESSPRI score in patients treated with RSLV-132 as compared with patients treated with placebo. The fatigue component of the ESSPRI score is provided on the y-axis. The ESSPRI score was assessed in patients treated with RSLV-132 and placebo on study days 1 (baseline) and 99.

FIG. 5B graphically depicts the pain component of the ESSPRI score in patients treated with RSLV-132 as compared with patients treated with placebo. The pain component of the ESSPRI score is provided on the y-axis. The ESSPRI score was assessed in patients treated with RSLV-132 and placebo on study days 1 (baseline) and 99.

FIG. 5C graphically depicts the dryness component of the ESSPRI score in patients treated with RSLV-132 as compared with patients treated with placebo. The dryness component of the ESSPRI score is provided on the y-axis. The ESSPRI score was assessed in patients treated with RSLV-132 and placebo on study days 1 (baseline) and 99.

FIG. 6 graphically depicts the mean change from baseline in the FACIT for RSLV-132 and placebo groups (p=0.92). An improvement in FACIT fatigue score in patients treated with RSLV-132 as compared with patients treated with placebo is shown. The FACIT fatigue score was assessed in patients treated with RSLV-132 and placebo on study days 1 (baseline), 29, 57, 85, and 99/end of treatment. An increase in the FACIT fatigue score indicates an improvement in fatigue. Results were analyzed using separate 1- way Analysis of Variance (ANOVA) models for each visit, each testing the null hypothesis (H₀) that the true mean difference between treatment groups was zero. Unadjusted alpha = 0.05. All standard error bars use Day 99 standard errors.

FIG. 7 graphically depicts an improvement in ProF in patients treated with RSLV-132 as compared with patients treated with placebo. The change in the ProF score over time from baseline is provided on the y-axis. The ProF score was assessed in patients treated with RSLV-132 and placebo on study days 1 (baseline), 29, 57, 85, and 99/end of treatment. A reduction in the ProF score indicates an improvement in fatigue.

FIG. 8 graphically depicts the mean change in the mental fatigue component of the ProF for the RSLV-132 and placebo groups (p=0.046). An improvement in the mental component of ProF in patients treated with RSLV-132 as compared with patients treated with placebo is shown. The change in the mental component of the ProF score over time from baseline is provided on the y-axis. The mental component of the ProF score was assessed in patients treated with RSLV-132 and placebo on study days 1 (baseline), 29, 57, 85, and 99/end of treatment. A reduction in the ProF score indicates an improvement in fatigue. Results were analyzed using separate 1- way Analysis of Variance (ANOVA) models for each visit, each testing the null hypothesis (H₀) that the true mean difference between treatment groups was zero. Unadjusted alpha = 0.05. All standard error bars use Day 99 standard errors.

FIG. 9 graphically depicts an improvement in the somatic component of ProF in patients treated with RSLV-132 as compared with patients treated with placebo. The change in the somatic component of the ProF score over time from baseline is provided on the y-axis. The somatic component of the ProF score was assessed in patients treated with RSLV-132 and placebo on study days 1 (baseline), 29, 57, 85, and 99/end of treatment. A reduction in the ProF score indicates an improvement in fatigue.

FIGS. 10A and 10B provide the results of the Digit Symbol Substitution Test (DSST) in patients treated with RSLV-132 and placebo. The DSST test was administered to patients at baseline (day 1) and at day 99 of the study. As shown in FIG. 10A, “Total 90s” refers to the total number of symbols matched to numbers in 90 seconds. “Completion” refers to the time to complete the test in seconds. From the initial baseline test (day 1) to follow-up (day 99) a statistically significant improvement in the time to complete the DSST test was observed in patients treated with RSLV-132. FIG. 10B graphically depicts the increase in time-to-complete for placebo and the decrease in time-to-completion for RSLV-132 treated patients.

FIGS. 11A-C depict heat maps showing changes in gene expression of interferon-inducible genes from day 1 (baseline) to day 99. The change in gene expression of three modules (M1.2, M3.4, and M5.12) of interferon inducible genes among the placebo group (n=7) and the RSLV-132 treated group (n=20) is shown. Genes with increased expression were represented on a red scale and those with decreased expression were represented on a blue scale. FIG. 11A depicts changes in gene expression in module M1.2. FIG. 11B depicts changes in gene expression in module M3.4. FIG. 11C depicts changes in gene expression in module 5.12.

FIGS. 12A-C depict heat maps showing changes in gene expression of interferon-inducible genes from day 1 (baseline) to day 99. The change in gene expression of the three modules (M1.2, M3.4, and M5.12) of interferon-inducible genes among the subgroup of RSLV-132 treated subjects who experienced a clinical response (R) compared to RSLV-132 treated patients who did not experience a clinical response (NR) and placebo treated patients is shown. Genes with increased expression were represented on a red scale and those with decreased expression were represented on a blue scale. FIG. 12A depicts changes in gene expression in module M1.2. FIG. 12B depicts changes in gene expression in module M3.4. FIG. 12C depicts changes in gene expression in module 5.12.

FIG. 13 depicts a heat map showing mean values for changes in expression of inflammation-related genes, comparing days 1 and 99 for the placebo group, non-responder (NR) and responder (R) subjects within the RSLV-132 group. Response is defined as subjects who experienced a MCII in 2 of 3 of the PRO instruments (FACIT-F, ProF, and ESSPRI).

FIG. 14 depicts a heat map showing mean values for changes in expression in lncRNA molecules that showed a strong negative correlation with an improvement in FACIT-F score, using log2 fold changes comparing days 1 and 99 for the placebo group, non-responder (NR), and responder (R) subjects within the RSLV-132 group. The asterisks indicate lncRNA molecules which have been identified in GWAS studies as mediating the expression of pro-inflammatory genes related to autoimmune diseases and/or immune system function.

### DETAILED DESCRIPTION

The present disclosure provides RNase-containing nuclease fusion proteins, including RNase-Fc fusion proteins that digest circulating RNA and RNA complexed with autoantibodies and immune complexes to thereby treat patients with primary Sjogren’s syndrome (pSS). The disclosure also provides methods for treating diseases characterized by elevated levels of circulating RNA and/or RNA-containing autoantibodies such as Sjogren’s syndrome, and methods for treating symptoms of diseases characterized by elevated levels of circulating RNA and RNA-containing autoantibodies, such as Sjogren’s syndrome associated fatigue in human patients in need thereof. The present disclosure also provides effective treatment and dosing regimens for administering RNase-containing nuclease fusion proteins, including RNase-Fc fusion proteins to human patients with Sjogren’s syndrome, including patients with pSS, in need thereof.

The present disclosure is based, at least in part, on the evaluation of the clinical impact of nuclease therapy by increasing RNA digestion activity in the blood of pSS patients with an RNase-containing nuclease fusion protein. In some embodiments, the RNase-containing nuclease fusion protein is RSLV-132. RSLV-132 is a biologic drug comprised of a full-length, catalytically active, human RNase moiety fused to the amino terminus of an engineered human IgG1 domain. The drug is engineered to remain in circulation and not enter cells bearing Fc receptors. The RNase portion of RSLV-132 maintained full enzymatic activity compared to wild-type human RNase and has a serum half-life of approximately 19 days.

The disclosure is based, at least in part, on the discovery that treatment of patients with pSS by administration of a RNase-containing nuclease fusion protein, such as RSLV-132, reduces fatigue associated with pSS in the patients. Without being bound by theory, it is believed that the RNase-containing nuclease fusion proteins of the disclosure are capable of digesting circulating RNA, and RNA complexed with autoantibodies and immune complexes in patients with an autoimmune disease to thereby reduce symptoms of the autoimmune disease, such as Sjogren’s syndrome associated fatigue.

It is also believed, without being bound by theory, that treatment of patients with Sjogren’s syndrome by administration of a RNase-containing nuclease fusion protein, such as RSLV-132, reduces circulating RNA whether it is associated with autoantibodies or free in the circulation.

It was discovered that treatment of pSS patients, by administration of a RNase-containing nuclease fusion protein, such as RSLV-132, resulted in an improvement of fatigue in patients by three separate tests which consistently showed an improvement in fatigue following treatment. In particular, when pSS patients were administered the EULAR Sjogren’s Syndrome Patient Reported Index (ESSPRI) following treatment with RSLV-132, patients experienced a clinically meaningful decrease in the ESSPRI score of one point or greater in the fatigue component of the ESSPRI score. A decrease in the ESSPRI score is associated with improved fatigue. Administration of the Functional Assessment of Chronic Illness Test (FACIT) to these patients following treatment with RSLV-132 resulted in an increase in the FACIT fatigue score, which is associated with reduced fatigue. Likewise, administration of the Profile of Fatigue (ProF) test following treatment with RSLV-132 resulted in a decrease in the ProF score which is associated with reduced fatigue.

Furthermore, it was discovered that following treatment of pSS patients with RLSV-132, patients demonstrated improved cognitive ability as measured by the Digit Symbol Substitution Test (DSST). Accordingly, the present disclosure provides RNase-containing nuclease fusion proteins, including RNase-Fc fusion proteins that are useful in methods of treating Sjogren’s syndrome, methods of reducing Sjogren’s syndrome associated fatigue, and methods of improving cognitive ability patients with Sjogren’s syndrome. The present disclosure also provides compositions comprising an RNase-Fc fusion protein and one or more pharmaceutically acceptable carriers and/or diluents that are useful in methods of treating Sjogren’s syndrome, methods of reducing Sjogren’s syndrome associated fatigue, and methods of improving cognitive ability patients with Sjogren’s syndrome. In some embodiments, the RNase-Fc fusion protein is RSLV-132. In some embodiments, the RNase-Fc fusion protein is administered to human patients at a dose of about 5-10 mg/kg, about 2-8 mg/kg, about 3-6 mg/kg, about 3 mg/kg, about 5 mg/kg, or about 10 mg/kg.

Furthermore, increased expression of interferon-inducible genes has typically been thought to be associated with higher disease activity. In contrast, it was surprisingly discovered that, at the biochemical level, an improvement in fatigue in patients treated with RSLV-132 correlated with an unexpected increase in the expression of selected interferon-inducible genes. For example, the mean expression of the interferon-inducible genes in the M1.2, M3.4, and M5.12 modules increased in RSLV-132 treated patients as compared to placebo treated patients. In addition, the subgroup of RSLV-132 treated patients who experienced a clinical response (defined as subjected who achieved a minimal clinically important improvement (MCII) in two of the three instruments, ESSPRI, FACIT-F, or ProF) revealed a larger difference in RSLV-132 stimulated interferon-inducible gene upregulation as compared to RSLV-132 treated patients who did not experience a clinical response. Without being bound by theory, it is believed that the expression of interferon-inducible genes may have multiple, possibly opposing inputs depending on specific disease pathology.

It was surprisingly discovered that an improvement in fatigue in patients treated with RSLV-132 was correlated with a decrease in expression in select lncRNA molecules. Several lncRNA molecules that were shown to have reduced expression in patients that respond to RSLV-132 treatment regulate the transcription of genes involved in inflammation, regulate the transcription of pro-inflammatory genes, and/or have been associated with autoimmune diseases and autoimmune function. The data in the application also demonstrate a correlation between an improvement in fatigue in patients treated with RSLV-132 and a general decrease in expression of inflammatory related genes. Without being bound by theory, it is believed that there is a change in the inflammatory state of patients that respond to treatment with RSLV-132. For example, patients that responded to treatment with RSLV-132 (reduced fatigue) exhibited a general decrease in expression of inflammatory related genes as compared to patients that did not respond to treatment. The data provided herein provide evidence that treatment with RSLV-132 resulted in clinical benefit as evidenced by this pharmacodynamic effect in patients with Sjogren’s syndrome.

The present disclosure provides evidence that fatigue experienced by pSS patients can be improved by pharmacological intervention with nuclease therapy, such as an RNase-containing nuclease fusion protein.

### Definitions

Terms used in the claims and specification are defined as set forth below unless otherwise specified.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

An “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, larger “peptide insertions” can be made, e.g. insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

As used herein, the term “auto-antibody” refers to an antibody produced by a subject’s immune system that is directed against one or more of the subject’s own proteins or nucleic acids. In some embodiments, autoantibodies are anti-Ro/SSA autoantibodies. In some embodiments, autoantibodies are anti-Ro52/60 autoantibodies. In some embodiments, the terms anti-Ro/SSA autoantibodies and anti-Ro52/60 autoantibodies are used interchangeably.

“Polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

“Nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res 1991;19:5081; Ohtsuka et al., JBC 1985;260:2605-8); Rossolini et al., Mol Cell Probes 1994;8:91-8). For arginine and leucine, modifications at the second base can also be conservative. The term nucleic acid is used interchangeably with gene, cDNA, and mRNA encoded by a gene.

Polynucleotides of the present invention can be composed of any polyribonucleotide or polydeoxribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

As used herein, the term “operably linked” or “operably coupled” refers to a juxtaposition wherein the components described are in a relationship permitting them to function in their intended manner.

As used herein, the term “glycosylation” or “glycosylated” refers to a process or result of adding sugar moieties to a molecule.

As used herein, the term “altered glycosylation” refers to a molecule that is aglycosylated, deglycosylated, or underglycosylated.

As used herein, “glycosylation site(s)” refers to both sites that potentially could accept a carbohydrate moiety, as well as sites within the protein on which a carbohydrate moiety has actually been attached and includes any amino acid sequence that could act as an acceptor for an oligosaccharide and/or carbohydrate.

As used herein, the term “aglycosylation” or “aglycosylated” refers to the production of a molecule in an unglycosylated form (e.g., by engineering a protein or polypeptide to lack amino acid residues that serve as acceptors of glycosylation). Alternatively, the protein or polypeptide can be expressed in, e.g., E. coli, to produce an aglycosylated protein or polypeptide.

As used herein, the term “deglycosylation” or “deglycosylated” refers to the process or result of enzymatic removal of sugar moieties on a molecule.

As used herein, the term “underglycosylation” or “underglycosylated” refers to a molecule in which one or more carbohydrate structures that would normally be present if produced in a mammalian cell has been omitted, removed, modified, or masked.

As used herein, the term “Fc region” and “Fc domain” is the portion of a native immunoglobulin formed by the respective Fc domains (or Fc moieties) of its two heavy chains without the variable regions which bind antigen. In some embodiments, an Fc domain begins in the hinge region just upstream of the papain cleavage site and ending at the C-terminus of the antibody. Accordingly, a complete Fc domain comprises at least a hinge domain, a CH2 domain, and a CH3 domain. In certain embodiments, an Fc domain comprises at least one of: a hinge (e.g., upper, middle, and/or lower hinge region) domain, a CH2 domain, a CH3 domain, a CH4 domain, or a variant, portion, or fragment thereof. In other embodiments, an Fc domain comprises a complete Fc domain (i.e., a hinge domain, a CH2 domain, and a CH3 domain). In one embodiment, an Fc domain comprises a hinge domain (or portion thereof) fused to a CH3 domain (or portion thereof). In another embodiment, an Fc domain comprises a CH2 domain (or portion thereof) fused to a CH3 domain (or portion thereof). In another embodiment, an Fc domain consists of a CH3 domain or portion thereof. In another embodiment, an Fc domain consists of a hinge domain (or portion thereof) and a CH3 domain (or portion thereof). In another embodiment, an Fc domain consists of a CH2 domain (or portion thereof) and a CH3 domain. In another embodiment, an Fc domain consists of a hinge domain (or portion thereof) and a CH2 domain (or portion thereof). In one embodiment, an Fc domain lacks at least a portion of a CH2 domain (e.g., all or part of a CH2 domain). In one embodiment, an Fc domain of the invention comprises at least the portion of an Fc molecule known in the art to be required for FcRn binding. In one embodiment, an Fc domain of the invention comprises at least the portion of an Fc molecule known in the art to be required for Protein A binding. In one embodiment, an Fc domain of the invention comprises at least the portion of an Fc molecule known in the art to be required for protein G binding. An Fc domain herein generally refers to a polypeptide comprising all or part of the Fc domain of an immunoglobulin heavy-chain. This includes, but is not limited to, polypeptides comprising the entire CHI, hinge, CH2, and/or CH3 domains as well as fragments of such peptides comprising only, e.g., the hinge, CH2, and CH3 domain. The Fc domain may be derived from an immunoglobulin of any species and/or any subtype, including, but not limited to, a human IgG1, IgG2, IgG3, IgG4, IgD, IgA, IgE, or IgM antibody. The Fc domain encompasses native Fc and Fc variant molecules. As with Fc variants and native Fc’s, the term Fc domain includes molecules in monomeric or multimeric form, whether digested from whole antibody or produced by other means.

As set forth herein, it will be understood by one of ordinary skill in the art that any Fc domain may be modified such that it varies in amino acid sequence from the native Fc domain of a naturally occurring immunoglobulin molecule.

The Fc domains of an RNase-Fc fusion protein of the disclosure may be derived from different immunoglobulin molecules. For example, an Fc domain of an RNase-Fc fusion protein may comprise a CH2 and/or CH3 domain derived from an IgG1 molecule and a hinge region derived from an IgG3 molecule. In another example, an Fc domain can comprise a chimeric hinge region derived, in part, from an IgG1 molecule and, in part, from an IgG3 molecule. In another example, an Fc domain can comprise a chimeric hinge derived, in part, from an IgG1 molecule and, in part, from an IgG4 molecule. The wild type human IgG1 Fc domain has the amino acid sequence set forth in SEQ ID NO: 20.

As used herein, the term “serum half-life” refers to the time required for the in vivo serum RNase-Fc fusion protein concentration to decline by 50%. The shorter the serum half-life of the RNase-Fc fusion protein, the shorter time it will have to exert a therapeutic effect.

As used herein, the term “RNA nuclease agent” refers to an agent comprising an RNase domain. In some embodiments, the RNA nuclease agent is an RNase-containing nuclease fusion protein. In some embodiments, the RNA nuclease agent is an RNase-Fc fusion protein. In some embodiments, the RNase domain of the RNA nuclease agent is human pancreatic RNase 1. In some embodiments, the RNA nuclease agent is a polypeptide. In some embodiments, the RNA nuclease agent is RSLV-132.

As used herein, the term “RNase-containing nuclease fusion protein” refers to polypeptides that comprise at least one nuclease domain operably linked, with or without a linker, to a PK moiety (pharmacokinetic moiety), such as an Fc domain, or a variant or fragment thereof, and nucleic acids encoding such polypeptides. In some embodiments, the RNase-containing nuclease fusion protein is an “RNase-Fc fusion protein” which refers to polypeptides that comprise at least one nuclease domain operably linked, with or without a linker, to an Fc domain, or a variant or fragment thereof, and nucleic acids encoding such polypeptides. In some embodiments, the RNase-Fc fusion protein is a polypeptide that comprises at least two nuclease domains operably linked, with or without a linker, to an Fc domain, or a variant or fragment thereof, and nucleic acids encoding such polypeptides. In some embodiments, the nuclease domain is a human RNase 1. In some embodiments, the RNase-Fc fusion protein comprises one or more RNase domains and one or more Fc domains. In some embodiments, the RNase-Fc fusion protein comprises one or more RNase-domains, one or more Fc domains, and one or more DNase domains. In some embodiments, an RNase-Fc fusion protein comprises an RNase 1 domain operably linked to the N- or C- terminus of an Fc domain and a DNase domain operably linked to the N- or C- terminus of the Fc domain. In some embodiments, an RNase-Fc fusion protein is a tandem RNase-Fc fusion protein, e.g., a one or more RNase 1 domains and/or one or more DNase domains linked in tandem to either the N- or C- terminus of one or more Fc domains. In some embodiments, an RNase-Fc fusion protein is a homodimeric RNase-Fc fusion protein (two of the same polypeptides). In some embodiments, an RNase-Fc fusion protein is a heterodimeric RNase-Fc fusion protein (two different polypeptides). In some embodiments, the domains of the RNase-Fc fusion protein are operably linked with a linker domain. In some embodiments, the domains of the RNase-Fc fusion protein are operably linked without a linker domain.

As used herein, the term “tandem RNase-Fc fusion protein” refers to a polypeptide that comprises at least two nuclease domains linked in tandem (from N- to C- terminus) and an Fc domain, or a variant or fragment thereof, and nucleic acids encoding such polypeptides. In some embodiments, a tandem RNase-Fc fusion protein is a polypeptide comprising at least two RNase 1 domains operably linked in tandem to at least one Fc domain. In some embodiments, a tandem RNase-Fc fusion protein is a polypeptide comprising at least one DNase1 domain and at least one RNase1 domain operably linked in tandem to at least one Fc domain. In some embodiments, a tandem RNase-Fc fusion protein includes from N- to C- terminus a DNase1 domain, a first linker, an RNase1 domain, a second linker, and an Fc domain, or a variant or fragment thereof.

As used herein, the term “heterodimeric RNase-Fc fusion protein” refers to a heterodimer comprising a first and a second polypeptide, which together comprise at least two nuclease domains and two Fc domains, variants or fragment thereof, and nucleic acids encoding such polypeptides. In some embodiments, the heterodimer comprises a first RNase 1 domain operably linked with or without a linker to the N- or C- terminus of a first Fc domain, and a second RNase 1 domain operably linked with or without a linker to the N- or C- terminus of a second Fc domain, such that the first RNase 1 and second RNase 1 domains are located at the same end (N- or C- terminus) of the heterodimer. In some embodiments, the heterodimer comprises a first RNase 1 domain operably linked with or without a linker to the N- terminus of a first Fc domain, and a second RNase 1 operably linked with or without a linker to the C-terminus of the second Fc domain. In some embodiments, the first RNase1 domain is operably linked with or without a linker to the C- terminus of the first Fc domain, and the second RNase1 domain is operably linked with or without a linker to the N- terminus of the second Fc domain. In some embodiments, the first and second RNase 1 domains of the heterodimer are different. In some embodiments, a heterodimeric RNase-Fc fusion protein is a heterodimer comprising at least one DNasel domain and at least one RNasel domain operably linked to at least one Fc domain, wherein the DNase 1 domain is operably linked with or without a linker to the N- or C-terminus of a first Fc domain and an RNasel domain is operably linked with or without a linker the N- or C- terminus of a same (first Fc domain) or a different Fc domain (second Fc domain), such that the DNase 1 domain and the RNase 1 domain are located on opposite ends (N- or C-terminus) of either the same (first Fc domain) or different Fc domain (second Fc domain). In some embodiments, the heterodimer comprises a DNase.

As used herein, the term “homodimeric RNase-Fc fusion protein” refers to a homodimer comprising a first and a second polypeptide, which together comprise at least two of the same nuclease domains and two Fc domains, variants or fragments thereof, and nucleic acids encoding such polypeptides. In some embodiments, the homodimer comprises a first RNase 1 domain operably linked with or without a linker to the N- or C- terminus of a first Fc domain, and a second RNase 1 domain operably linked with or without a linker to the N- or C- terminus of a second Fc domain, such that the first RNase 1 and second RNase 1 domains are located at the same end (N- or C- terminus) of the homodimer. In some embodiments, the first and second RNase 1 domains of the homodimer are identical. In some embodiments, the homodimer comprises an RNase 1 domain operably linked with or without a linker to the N- or C- terminus of an Fc domain, and a DNase domain operably linked with or without a linker to the N- or C-terminus of the Fc domain. In some embodiments, the RNase 1 and DNase domains are located at the same end (N- or C- terminus) of the Fc domain. In some embodiments, the RNase 1 and DNase domains are located on opposite ends of the Fc domain.

As used herein, the term “dimer” refers to a macromolecular complex formed by two macromolecules (e.g., polypeptides). A “homodimer” refers to a dimer that is formed by two identical macromolecules (e.g., polypeptides). A “heterodimer” refers to a dimer that is formed by two different macromolecules (e.g., polypeptides).

As used herein, the term “variant” refers to a polypeptide derived from a wild-type nuclease (e.g., RNase) or Fc domain and differs from the wild-type by one or more alteration(s), i.e., a substitution, insertion, and/or deletion, at one or more positions. A substitution means a replacement of an amino acid occupying a position with a different amino acid. A deletion means removal of an amino acid occupying a position. An insertion means adding 1 or more, such as 1-3 amino acids, immediately adjacent to an amino acid occupying a position. Variant polypeptides necessarily have less than 100% sequence identity or similarity with the wild-type polypeptide. In some embodiments, the variant polypeptide will have an amino acid sequence from about 75% to less than 100% amino acid sequence identity or similarity with the amino acid sequence of wild-type polypeptide, or from about 80% to less than 100%, or from about 85% to less than 100%, or from about 90% to less than 100% (e.g., 91%, 92%, 93%, 94%, 95%, 96%, 97%, 98%o, 99%) or from about 95% to less than 100%, e.g., over the length of the variant polypeptide.

In certain aspects, the RNase-Fc fusion proteins employ one or more “linker domains,” such as polypeptide linkers. As used herein, the term “linker domain” refers to one or more amino acids which connect two or more peptide domains in a linear polypeptide sequence. As used herein, the term “polypeptide linker” refers to a peptide or polypeptide sequence (e.g., a synthetic peptide or polypeptide sequence) which connects two or more polypeptide domains in a linear amino acid sequence of a protein. For example, polypeptide linkers may be used to operably link a nuclease domain (e.g., RNase) to an Fc domain. Such polypeptide linkers in some embodiments provide flexibility to the polypeptide molecule. In some embodiments the polypeptide linker is used to connect (e.g., genetically fuse) an RNase domain to an Fc domain. An RNase-Fc fusion protein may include more than one linker domain or peptide linker. Various peptide linkers are known in the art.

As used herein, the term “gly-ser polypeptide linker” refers to a peptide that consists of glycine and serine residues. An exemplary gly/ser polypeptide linker comprises the amino acid sequence (Gly₄Ser)n. In some embodiments, n is 1 or more, such as 2 or more, 3 or more, 4 or more, 5 or more, 6 or more, 7 or more, 8 or more, 9 or more, or 10 or more (e.g., (Gly₄Ser)10). Another exemplary gly/ser polypeptide linker comprises the amino acid sequence Ser(Gly₄Ser)n. In some embodiments, n is 1 or more, such as 2 or more, 3 or more, 4 or more, 5 or more, 6 or more, 7 or more, 8 or more, 9 or more, or 10 or more (e.g., Ser(Gly₄Ser)10).

As used herein, the terms “coupled,” “conjugated,” “linked,” “fused,” or “fusion,” are used interchangeably. These terms refer to the joining together of two more elements or components or domains, by whatever means including chemical conjugation or recombinant means. Methods of chemical conjugation (e.g., using heterobifunctional crosslinking agents) are known in the art.

A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence. Polypeptides derived from another polypeptide may have one or more mutations relative to the starting polypeptide, e.g., one or more amino acid residues which have been substituted with another amino acid residue or which has one or more amino acid residue insertions or deletions.

In one embodiment, there is one amino acid difference between a starting polypeptide sequence and the sequence derived therefrom. Identity or similarity with respect to this sequence is defined herein as the percentage of amino acid residues in the candidate sequence that are identical (i.e., same residue) with the starting amino acid residues, after aligning the sequences and introducing gaps, if necessary, to achieve the maximum percent sequence identity.

In one embodiment, a polypeptide of the disclosure consists of, consists essentially of, or comprises an amino acid sequence as set forth in the Sequence Listing or Sequence Table disclosed herein and functionally active variants thereof. In an embodiment, a polypeptide includes an amino acid sequence at least 80%, such as at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to an amino acid sequence set forth in the Sequence Listing or Sequence Table disclosed herein. In some embodiments, a polypeptide includes a contiguous amino acid sequence at least 80%, such as at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to a contiguous amino acid sequence set forth in the Sequence Listing or Sequence Table disclosed herein. In some embodiments, a polypeptide includes an amino acid sequence having at least 10, such as at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 200, at least 300, at least 400, or at least 500 (or any integer within these numbers) contiguous amino acids of an amino acid sequence set forth in Sequence Listing or Sequence Table disclosed herein.

In some embodiments, the RNase-Fc fusion proteins of the disclosure are encoded by a nucleotide sequence. Nucleotide sequences of the disclosure can be useful for a number of applications, including: cloning, gene therapy, protein expression and purification, mutation introduction, DNA vaccination of a host in need thereof, antibody generation for, e.g., passive immunization, PCR, primer and probe generation, siRNA design and generation (see, e.g., the Dharmacon siDesign website), and the like. In some embodiments, the nucleotide sequence of the disclosure comprises, consists of, or consists essentially of, a nucleotide sequence that encodes the amino acid sequence of the RNase-Fc fusion proteins selected from the Sequence Table or Sequence Listing. In some embodiments, a nucleotide sequence includes a nucleotide sequence at least 80%, such as at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to a nucleotide sequence encoding an amino acid sequence of the Sequence Listing or Sequence Table disclosed herein. In some embodiments, a nucleotide sequence includes a contiguous nucleotide sequence at least 80%, such as at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to a contiguous nucleotide sequence encoding an amino acid sequence set forth in the Sequence Listing or Sequence Table disclosed herein. In some embodiments, a nucleotide sequence includes a nucleotide sequence having at least 10, such as at least 15, such as at least 20, at least 25, at least 30, at least 35, at least 40, at least 45, at least 50, at least 55, at least 60, at least 65, at least 70, at least 75, at least 80, at least 85, at least 90, at least 95, at least 100, at least 200, at least 300, at least 400, or at least 500 (or any integer within these numbers) contiguous nucleotides of a nucleotide sequence encoding an amino acid sequence set forth in the Sequence Listing or Sequence Table disclosed herein.

It will also be understood by one of ordinary skill in the art that the RNase-Fc fusion proteins may be altered such that they vary in sequence from the naturally occurring or native sequences from which their components (e.g., nuclease domains, linker domains, and Fc domains) are derived, while retaining the desirable activity of the native sequences. For example, nucleotide or amino acid substitutions leading to conservative substitutions or changes at “non-essential” amino acid residues may be made. An isolated nucleic acid molecule encoding a non-natural variant can be created by introducing one or more nucleotide substitutions, additions or deletions into the nucleotide sequence of the RNase-Fc fusion protein such that one or more amino acid substitutions, additions or deletions are introduced into the encoded protein. Mutations may be introduced by standard techniques, such as site-directed mutagenesis and PCR-mediated mutagenesis.

The RNase-Fc fusion proteins may comprise conservative amino acid substitutions at one or more amino acid residues, e.g., at essential or non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). Thus, a nonessential amino acid residue in an RNase-Fc fusion protein is preferably replaced with another amino acid residue from the same side chain family. In another embodiment, a string of amino acids can be replaced with a structurally similar string that differs in order and/or composition of side chain family members. Alternatively, in another embodiment, mutations may be introduced randomly along all or part of a coding sequence, such as by saturation mutagenesis, and the resultant mutants can be incorporated into the RNase-Fc fusion proteins and screened for their ability to bind to the desired target.

As used herein, the term “gene expression profile” refers to a technique that identifies genes being expressed in a sample and/or determines the degree of their expression at a specified time. The term “interferon-inducible gene expression profile” refers to a gene expression profile that identifies interferon-inducible genes being expressed and/or determines the degree of their expression at a specified time. In some embodiments, an interferon-inducible gene expression profile includes one or more of the genes in module M1.2, M3.4, and/or M5.12. In some embodiments, an interferon-inducible gene expression profile includes one or more of the genes in module M1.2. In some embodiments, an interferon-inducible gene expression profile includes one or more of the genes in module M3.4. In some embodiments, an interferon-inducible gene expression profile includes one or more of the genes in module M5.12. In some embodiments, an interferon-inducible gene expression profile includes modules M1.2, M3.4, and/or M5.12. In some embodiments, an interferon-inducible gene expression profile is module M1.2. In some embodiments, an interferon-inducible gene expression profile is module M3.4. In some embodiments, an interferon-inducible gene expression profile is module M5.12.

In some embodiments, a gene expression profile comprises a profile of inflammation-related genes.

In some embodiments, an expression profile includes lncRNA molecules. In some embodiments, an expression profile includes lncRNA molecules that regulate the transcription of genes involved in inflammation and/or regulate the transcription of proinflammatory genes. In some embodiments, the pro-inflammatory genes are pro-inflammatory genes related to autoimmune disease. In some embodiments, the pro-inflammatory genes are pro-inflammatory genes related immune system function.

The term “ameliorating” refers to any therapeutically beneficial result in the treatment of a disease state, e.g., an autoimmune disease state (e.g., SLE, Sjogren’s syndrome), including prophylaxis, lessening in the severity or progression, remission, or cure thereof.

As used herein, the terms “primary Sjogren’s syndrome (pSS),” “Sjogren’s syndrome,” “Sjogren’s disease,” and “Sjogren’s” can be used interchangeably.

The term “in situ” refers to processes that occur in a living cell growing separate from a living organism, e.g., growing in tissue culture.

The term “in vivo” refers to processes that occur in a living organism.

The term “mammal” or “subject” or “patient” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv Appl Math 1981;2:482, by the homology alignment algorithm of Needleman & Wunsch, J Mol Biol 1970;48:443, by the search for similarity method of Pearson & Lipman, PNAS 1988;85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al, infra).

One example of an algorithm that is suitable for determining percent sequence identity and sequence similarity is the BLAST algorithm, which is described in Altschul et al., J Mol Biol 1990;215:403-10. Software for performing BLAST analyses is publicly available through the National Center for Biotechnology Information website.

The term “sufficient amount” means an amount sufficient to produce a desired effect.

The term “therapeutically effective amount” is an amount that is effective to ameliorate a symptom of a disease. A therapeutically effective amount can be a “prophylactically effective amount” as prophylaxis can be considered therapy.

The term “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

It must be noted that, as used in the specification and the appended claims, the singular forms “a,” “an” and “the” include plural referents unless the context clearly dictates otherwise.

### RNase-Containing Nuclease Fusion Proteins

The RNase-containing nuclease fusion proteins of the disclosure, including RNase-Fc fusion proteins, include at least one enzymatically active RNase domain, or fragment or variant thereof, such as a human RNase 1, or fragment or variant thereof, operably linked to a PK moiety which provides a scaffold and/or extends the in vivo half-life of the RNase domain as compared to a nuclease domain without such a PK moiety. In some aspects, the RNase-containing nuclease fusion protein is a RNase-Fc fusion protein which includes at least one enzymatically active RNase domain, or fragment or variant thereof, such as a human RNase 1, or fragment or variant thereof, operably linked to an Fc domain, such as a human IgG1 Fc domain, or a variant or fragment thereof, that alters the serum half-life of the nuclease molecule to which it is fused compared to nuclease molecules that are not fused to the Fc domain, or a variant or fragment thereof.

In some embodiments, the RNase-containing nuclease fusion proteins of the disclosure, including RNase-Fc fusion proteins are operably coupled to the Fc domain, or a variant or fragment thereof, via a linker domain. In some embodiments, the linker domain is a linker peptide. In some embodiments, the linker domain is a linker nucleotide.

In some embodiments, the RNase-containing nuclease fusion protein of the disclosure, including RNase-Fc fusion proteins include a leader sequence, e.g., a leader peptide. In some embodiments, the leader molecule is a leader peptide positioned at the N-terminus of the nuclease domain. In some embodiments, an RNase-Fc fusion protein comprises a leader peptide at the N-terminus of the molecule, wherein the leader peptide is later cleaved from the RNase-Fc fusion protein. Methods for generating nucleic acid sequences encoding a leader peptide fused to a recombinant protein are well known in the art. In some embodiments, the RNase-Fc fusion proteins are expressed either with or without a leader fused to their N-terminus. The protein sequence of an RNase-Fc fusion protein of the present disclosure following cleavage of a fused leader peptide can be predicted and/or deduced by one of skill in the art.

In some embodiments the leader is a VK3 leader peptide (VK3LP), wherein the leader peptide is fused to the N-terminus of the RNase-Fc fusion protein. Such leader sequences can improve the level of synthesis and secretion of the RNase-Fc fusion protein in mammalian cells. In some embodiments, the leader is cleaved, yielding RNase-Fc fusion proteins. In some embodiments, an RNase-Fc fusion protein of the present disclosure is expressed without a leader peptide fused to its N-terminus, and the resulting RNase-Fc fusion protein has an N-terminal methionine.

In some embodiments, the RNase-Fc fusion proteins of the disclosure include a VK3 leader peptide fused to the N-terminus of the RNase-Fc fusion protein, e.g., SEQ ID NO: 49 (RSLV-132). In some embodiments, the RNase-Fc fusion proteins of the disclosure do not include a leader sequence, e.g., SEQ ID NO: 50 (RSLV-132).

In some embodiments, the RNase-Fc fusion protein includes an RNase domain operably coupled to the N- or C-terminus of an Fc domain, or a variant or fragment thereof. In some embodiments, the RNase-Fc fusion protein comprises both an RNase domain and a DNase domain,

In some embodiments, the RNase-Fc fusion protein includes two nuclease domains (e.g., two RNase domains) operably coupled to each other in tandem and further operably coupled to the N- or C-terminus of the same or different Fc domains, or a variant or fragment thereof.

The Sequence Table provides the sequences of exemplary RNase-Fc fusion proteins of various configurations.

In some embodiments, an RNase-Fc fusion protein is a multi-nuclease protein (e.g., two RNA nucleases, or an RNase and a DNase) fused to the same or different Fc domains, or a variant or fragment thereof, that specifically binds to extracellular immune complexes.

In one embodiment, the nuclease domain is operably coupled (e.g., chemically conjugated or genetically fused (e.g., either directly or via a polypeptide linker)) to the N-terminus of a Fc domain, or a variant or fragment thereof. In another embodiment, the nuclease domain is operably coupled (e.g., chemically conjugated or genetically fused (e.g., either directly or via a polypeptide linker)) to the C-terminus of a Fc domain, or a variant or fragment thereof. In other embodiments, a nuclease domain is operably coupled (e.g., chemically conjugated or genetically fused (e.g., either directly or via a polypeptide linker)) via an amino acid side chain of a Fc domain, or a variant or fragment thereof.

In certain embodiments, the RNase-Fc fusion proteins of the disclosure comprise two or more nuclease domains and at least one Fc domain, or a variant or fragment thereof. For example, nuclease domains may be operably coupled to both the N-terminus and C-terminus of the same or different Fc domains, or variants or fragments thereof, with optional linkers between the nuclease domains and the Fc domain(s), variant(s) or fragment(s) thereof. In some embodiments, the nuclease domains are identical, e.g., RNase and RNase. In other embodiments, the nuclease domains are different, e.g., two different RNA nucleases or RNase and DNase.

In some embodiments, two or more nuclease domains are operably coupled to each other (e.g., via a polypeptide linker) in series, and the tandem array of nuclease domains is operably coupled (e.g., chemically conjugated or genetically fused (e.g., either directly or via a polypeptide linker)) to either the C-terminus or the N-terminus of the same or different Fc domains, or variants or fragments thereof. In other embodiments, the tandem array of nuclease domains is operably coupled to both the N-terminus and the C-terminus of the same Fc domain, or a variant or fragment thereof. In some embodiments, the nuclease domains are operably linked in tandem (e.g., N- RNase- RNase -C, N-RNase-DNase-C, or N-DNase-RNase-C) with or without a linker to the N-or C- terminus of the same or different Fc domains. In some embodiments, the tandem RNase-Fc fusion proteins form a homodimer or a heterodimer.

In other embodiments, one or more nuclease domains are inserted between two Fc domains, or variants or fragments thereof. For example, one or more nuclease domains may form all or part of a polypeptide linker of an RNase-Fc fusion protein of the disclosure.

In some embodiments, the RNase-Fc fusion proteins comprise at least two nuclease domains (e.g., RNase and RNase or RNase and DNase), at least one linker domain, and at least one Fc domain, or a variant or fragment thereof.

In some embodiments, the RNase-Fc fusion proteins of the disclosure comprise a Fc domain, or a variant or fragment thereof, as described herein, thereby increasing serum half-life and bioavailability of the RNase-Fc fusion proteins. In some embodiments, an RNase-Fc fusion protein comprises one or more polypeptides such as a polypeptide comprising an amino acid sequence as shown in any of SEQ ID NOs: 44-58.

It will be understood by the skilled artisan that other configurations of the nuclease domains and Fc domains are possible, with the inclusion of optional linkers between the nuclease domains and/or between the nuclease domains and Fc domain. It will also be understood that domain orientation can be altered, so long as the nuclease domains are active in the particular configuration tested.

In certain embodiments, the RNase-Fc fusion proteins of the disclosure have at least one nuclease domain specific for a target molecule which mediates a biological effect. In another embodiment, binding of the RNase-Fc fusion proteins of the disclosure to a target molecule (e.g. RNA or DNA) results in the reduction or elimination of the target molecule, e.g., from a cell, a tissue, or from circulation.

In other embodiments, the RNase-Fc fusion proteins of the disclosure may be assembled together or with other polypeptides to form binding proteins having two or more polypeptides (“multimers”), wherein at least one polypeptide of the multimer is an RNase-Fc fusion protein of the disclosure. Exemplary multimeric forms include dimeric, trimeric, tetrameric, and hexameric altered binding proteins and the like. In one embodiment, the polypeptides of the multimer are the same (i.e., homomeric altered binding proteins, e.g., homodimers, homotetramers). In another embodiment, the polypeptides of the multimer are different (e.g., heteromeric). In one embodiment, the RNase-Fc fusion proteins of the disclosure are assembled together to form a dimer. In one embodiment, the dimer is a homodimer. In one embodiment, the dimer is a heterodimer.

In some embodiments, an RNase-Fc fusion protein has a serum half-life that is increased at least about 1.5-fold, such as at least 3-fold, at least 5-fold, at least 10-fold, at least about 20-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1000-fold, or 1000-fold or greater relative to the corresponding nuclease molecules not fused to the Fc domain, or a variant or fragment thereof. In other embodiments, an RNase-Fc fusion protein has a serum half-life that is decreased at least about 1.5-fold, such as at least 3-fold, at least 5-fold, at least 10-fold, at least about 20-fold, at least about 50-fold, at least about 100-fold, at least about 200-fold, at least about 300-fold, at least about 400-fold, at least about 500-fold, or 500-fold or lower relative to the corresponding nuclease molecules not fused to the Fc domain, or a variant or fragment thereof. Routine art-recognized methods can be used to determine the serum half-life of RNase-Fc fusion proteins of the disclosure.

In some embodiments, the activity of the RNase in the RNase-Fc fusion protein is not less than about 10-fold less, such as 9-fold less, 8-fold less, 7-fold less, 6-fold less, 5-fold less, 4-fold less, 3-fold less, or 2-fold less than the activity of a control RNase molecule. In some embodiments, the activity of the RNase in the RNase-Fc fusion protein is about equal to the activity of a control RNase molecule.

In some embodiments, the RNase-Fc fusion proteins can be active towards extracellular immune complexes containing DNA and/or RNA, e.g., either in soluble form or deposited as insoluble complexes.

In some embodiments, the activity of the RNase-Fc fusion protein is detectable in vitro and/or in vivo.

In another aspect, a multifunctional RNase molecule is provided that is attached to another enzyme or antibody having binding specificity, such as an scFv targeted to RNA or DNA or a second nuclease domain with the same or different specificities as the first domain.

In some embodiments, linker domains include (gly4ser) 3, 4 or 5 variants that alter the length of the linker by 5 amino acid progressions. In another embodiment, a linker domain is approximately 18 amino acids in length and includes an N-linked glycosylation site, which can be sensitive to protease cleavage in vivo. In some embodiments, an N-linked glycosylation site can protect the RNase-Fc fusion proteins from cleavage in the linker domain. In some embodiments, an N-linked glycosylation site can assist in separating the folding of independent functional domains separated by the linker domain.

In some embodiments, the linker domain is an NLG linker (VDGASSPVNVSSPSVQDI) (SEQ ID NO: 37).

In some embodiments, the RNase-Fc fusion proteins includes substantially all or at least an enzymatically active fragment of a DNase. In some embodiments, the DNase is a Type I secreted DNase, preferably a human DNase such as mature human pancreatic DNase 1 (UniProtKB entry P24855, SEQ ID NO: 6). In some embodiments, a naturally occurring variant allele, A114F (SEQ ID NO: 8), which shows reduced sensitivity to actin is included in a DNase1 of an RNase-Fc fusion protein (see Pan et al., JBC 1998;273:18374-81; Zhen et al., BBRC 1997;231:499-504; Rodriguez et al., Genomics 1997;42:507-13). In other embodiments, a naturally occurring variant allele, G105R (SEQ ID NO: 9), which exhibits high DNase activity relative to wild type DNase1, is included in a DNase1 of an RNase-Fc fusion protein (see Yasuda et al., Int J Biochem Cell Biol 2010;42:1216-25). In some embodiments, this mutation is introduced into an RNase-FC fusion protein to generate a more stable derivative of human DNase1. In some embodiments, the DNase is human, wild type DNase1 or human, DNase1 A114F mutated to remove all potential N-linked glycosylation sites, i.e., asparagine residues at positions 18 and 106 of the DNase1 domain set forth in SEQ ID NO: 6 (i.e., human DNase1 N18S/N106S/A114F, SEQ ID NO: 11), which correspond to asparagine residues at positions 40 and 128, respectively, of full length pancreatic DNasel with the native leader (SEQ ID NO: 5).

In some embodiments, the DNase is a human DNasel comprising one or more basic (i.e., positively charged) amino acid substitutions to increase DNase functionality and chromatin cleavage. In some embodiments, basic amino acids are introduced into human DNase1 at the DNA binding interface to enhance binding with negatively charged phosphates on DNA substrates (see US 7407785; US 6391607). This hyperactive DNase1 may be referred to as “chromatin cutter.”

In some embodiments, 1, 2, 3, 4, 5 or 6 basic amino acid substitutions are introduced into DNasel. For example, one or more of the following residues is mutated to enhance DNA binding: Gln9, Glu13, Thr14, His44, Asn74, Asn110, Thr205. In some embodiments one or more of the foregoing amino acids are substituted with basic amino acids such as, arginine, lysine and/or histidine. For example, a human DNase can include one or more of the following substitutions: Q9R, E13R, T14K, H44K, N74K, N110R, T205K. In some embodiments, the human DNasel also includes an A114F substitution, which reduces sensitivity to actin (see US 6348343). In one embodiment, the human DNasel includes the following substitutions: E13R, N74K, A114F and T205K.

In some embodiments, the human DNasel further includes mutations to remove potential glycosylation sites, e.g., asparagine residues at positions 18 and 106 of the DNasel domain set forth in SEQ ID NO: 6, which correspond to asparagines residues at positions 40 and 128, respectively of full length pancreatic DNase1 with the native leader. In one embodiment, the human DNase1 includes the following substitutions: E13R/N74K/A114F/T205K/N18S/N106S.

In some embodiments, the DNase is DNase 1-like (DNaseL) enzyme, 1-3 (UniProtKB entry Q13609; SEQ ID NO: 15). In some embodiments, the DNase is three prime repair exonuclease 1 (TREX1; UniProtKB entry Q9NSU2; SEQ ID NO: 16). In some embodiments, the DNase is DNase2. In some embodiments, the DNase2 is DNAse2 alpha (i.e., DNase2; UnitProtKB entry 000115 SEQ ID NO: 18) or DNase2 beta (i.e., DNase2-like acid DNase; UnitProtKB entry Q8WZ79; SEQ ID NO: 19). In some embodiments, the N-linked glycosylation sites of DNase 1L3, TREX1, DNase2 alpha, or DNase2 beta are mutated such as to remove potential N-linked glycosylation sites. In some embodiments, a DNase-linker-Fc domain containing a 20 or 25 aa linker domain is made.

In some embodiments, the activity of the DNase in the RNase-Fc fusion protein is not less than about 10-fold less, such as 9-fold less, 8-fold less, 7-fold less, 6-fold less, 5-fold less, 4-fold less, 3-fold less, or 2-fold less than the activity of a control DNase molecule. In some embodiments, the activity of the DNase in the RNase-Fc fusion protein is about equal to the activity of a control DNase molecule.

In some embodiments, the RNase-Fc fusion protein of the disclosure includes a human RNase 1. In some embodiments, the RNase-Fc fusion protein includes a wild-type human RNase 1 domain. In some embodiments, the RNase-Fc fusion protein includes human pancreatic RNasel (UniProtKB entry P07998; SEQ ID NO: 1) of the RNase A family. In some embodiments, the RNase-Fc fusion protein includes the mature form of human pancreatic RNasel as set forth in SEQ ID NO: 2. In some embodiments, the RNase-Fc domain includes a human RNase 1 domain having one or more mutations. In some embodiments, the human RNasel is mutated to remove all potential N-linked glycosylation sites, i.e., asparagine residues at positions 34, 76, and 88 of the RNasel domain set forth in SEQ ID NO: 2 (human RNasel N34S/N76S/N88S, SEQ ID NO: 4), which correspond to asparagine residues at positions 62, 104, and 116, respectively, of full length pancreatic RNase1 with the native leader (SEQ ID NO: 1). In some embodiments, a RNase1-linker-Fc containing a 20 or 25 aa linker domain is made.

In some embodiments, the RNase-Fc fusion protein includes a mammalian RNase 1. In some embodiments, the RNase-Fc fusion protein includes a primate RNase 1. In some embodiments, the RNase-Fc fusion protein includes a rodent RNase 1. In some embodiments, the RNase-Fc fusion protein includes a mouse RNase 1. In some embodiments, the RNase-Fc fusion protein includes a rat RNase 1. In some embodiments, the RNase-Fc fusion protein includes a monkey RNase 1. In some embodiments, the RNase-Fc fusion protein includes a goat RNase 1. In some embodiments, the RNase-Fc fusion protein includes a rabbit RNase 1. In some embodiments, the RNase-Fc fusion protein includes a horse RNase 1. In some embodiments, the RNase-Fc fusion protein includes a canine RNase 1. In some embodiments, the RNase 1 domain is a mutant RNase 1 domain.

In some embodiments, an RNase-Fc fusion protein includes an RNase molecule attached to an Fc domain that specifically binds to extracellular immune complexes. In some embodiments, the Fc domain does not effectively bind Fcγ receptors. In one aspect, the RNase-Fc fusion protein does not effectively bind C1q. In other aspects, the RNase-Fc fusion protein comprises an in frame Fc domain from IgG1. In other aspects, the RNase-Fc fusion protein further comprises mutations in the hinge, CH2, and/or CH3 domains. In other aspects, the mutations are P238S, P331S or N297S, and may include mutations in one or more of the three hinge cysteines. In some such aspects, the mutations are in one or more of three hinge cysteines at residues 220, 226, and 229, numbering according to the EU index, such as substitution of one or more cysteine residues with serine, for example, C220S, C226S and/or C229S. In some embodiments, one of three hinge region cysteines are replaced by serine, for example, C220S also referred to herein as “SCC hinge”. In some embodiments, all three hinge region cysteines are replaced by serine, C220S, C226S and C229S, also referred to herein as “SSS hinge”. In other aspects, the RNase-Fc fusion proteins contain the SCC hinge, but are otherwise wild type for human IgG1 Fc CH2 and CH3 domains, and bind efficiently to Fc receptors, facilitating uptake of the RNase-Fc fusion protein into the endocytic compartment of cells to which they are bound. In other aspects, the RNase-Fc fusion protein has activity against single and/or double-stranded RNA substrates.

In some aspects, an RNase-Fc fusion protein includes a mutant Fc domain. In some aspects, an RNase-Fc fusion protein includes a mutant, IgG1 Fc domain. In some aspects, a mutant Fc domain comprises one or more mutations in the hinge, CH2, and/or CH3 domains. In some aspects, a mutant Fc domain includes a P238S mutation. In some aspects, a mutant Fc domain includes a P331S mutation. In some aspects, a mutant Fc domain includes a P238S mutation and a P331S mutation. In some aspects, a mutant Fc domain comprises P238S and/or P331S, and may include mutations in one or more of the three hinge cysteines. In some aspects, a mutant Fc domain comprises P238S and/or P331S, and/or one or more mutations in the three hinge cysteines. In some aspects, a mutant Fc domain comprises P238S and/or P331S, and/or mutations in the three hinge cysteines to SSS or in one hinge cysteine to SCC. In some aspects, a mutant Fc domain comprises P238S and P331S and mutations in the three hinge cysteines. In some aspects, a mutant Fc domain comprises P238S and P331S and either SCC or SSS. In some aspects, a mutant Fc domain comprises P238S and P331S and SCC. In some aspects, a mutant Fc domain includes P238S SSS. In some aspects, a mutant Fc domain includes P331S and either SCC or SSS. In some aspects, a mutant Fc domain includes mutations in one or more of the three hinge cysteines. In some aspects, a mutant Fc domain includes mutations in the three hinge cysteines. In some aspects, a mutant Fc domain includes mutations in the three hinge cysteines to SSS. In some aspects, a mutant Fc domain includes mutations in one of the three hinge cysteines to SCC. In some aspects, a mutant Fc domain includes SCC or SSS. In some aspects, a mutant Fc domain is as shown in any of SEQ ID NOs: 21-28. In some aspects, an RNase-Fc fusion protein is as shown in any of SEQ ID NOs 44-58. In some aspects, an RNase-Fc fusion protein comprises a wild-type, human RNasel domain linked to a mutant, human IgG1 Fc domain comprising SCC, P238S, and P331S, or a mutant, human IgG1 Fc domain comprising SSS, P238S, and P331S. In some embodiments, an RNase-Fc fusion protein is shown in SEQ ID NOs: 45-46. In some embodiments, an RNase-Fc fusion protein is shown in SEQ ID NO: 50.

In some aspects, an RNase-Fc fusion protein comprises a wild-type, human RNase1 domain linked via a (Gly₄Ser)4 linker domain to a mutant, human IgG1 Fc domain comprising SCC, P238S, and P331S or a mutant, human IgG1 Fc domain comprising SSS, P238S, and P331S. In some aspects, an RNase-Fc fusion protein is shown in SEQ ID NOs: 47-48.

In some embodiments, an RNase-Fc fusion protein comprises a human DNasel G105R A114F domain linked via a (Gly₄Ser)4 linker domain to a mutant, human IgG1 Fc domain comprising SCC, P238S, and P331S linked via a NLG linker domain to a wild-type, human RNasel domain. In some embodiments, an RNase-Fc fusion protein comprises a human DNasel G105R A114F domain linked via a (Gly₄Ser)4 linker domain to a mutant, human IgG1 Fc domain comprising SSS, P238S, and P331S linked via a NLG linker domain to a wild-type, human RNasel domain. In some aspects, an RNase-Fc fusion protein is shown in SEQ ID NOs: 51-52.

In some aspects, an RNase-Fc fusion protein comprises a wild-type, human RNase1 domain linked via a (Gly₄Ser)4 linker domain to a mutant, human IgG1 Fc domain comprising SCC, P238S, and P331S linked via a NLG linker domain to a human DNase1 G105R A114F domain. In some embodiments, an RNase-Fc fusion protein comprises a wild-type, human RNasel domain linked via a (Gly₄Ser)4 linker domain to a mutant, human IgG1 Fc domain comprising SSS, P238S, and P331S linked via a NLG linker domain to a human DNase1 G105R A114F domain. In some aspects, a RNase-Fc fusion protein is shown in SEQ ID NOs: 53-54.

In some aspects, a RNase-Fc fusion protein comprises a wild-type, human RNase1 domain linked to a mutant, human IgG1 Fc domain comprising SCC, P238S, and P331S linked via a NLG linker domain to a human DNasel G105R A114F domain. In some aspects, a RNase-Fc fusion protein comprises a wild-type, human RNasel domain linked to a mutant, human IgG1 Fc domain comprising SSS, P238S, and P331S linked via a NLG linker domain to a human DNase1 G105R A114F domain. In some aspects, a RNase-Fc fusion protein is shown in SEQ ID NOs: 55-58.

In some aspects, the activity of the RNase-Fc fusion protein is detectable in vitro and/or in vivo.

In some embodiments, RNase-Fc fusion proteins include an RNase domain and an Fc domain, wherein the RNase1 domain is located at the COOH side of the Fc. In other embodiments, RNase-Fc fusion proteins include an RNase domain and an Fc domain, wherein the RNase1 domain is located at the NH2 side of the Fc. In some embodiments, RNase-Fc fusion proteins include: RNase-Fc; Fc-RNase; Fc-linker-RNase; RNase-linker-Fc, RNase-Fc-DNase; DNase-Fc-RNase; RNase-linker-Fc-linker-DNase; DNase-linker-Fc-linker-RNase; RNase-Fc-linker-DNase; DNase-Fc-linker-RNase; RNase-linker-Fc-DNase; DNase-linker-Fc-RNase.

In some embodiments, fusion junctions between enzyme domains and the other domains of the RNase-Fc fusion protein are optimized.

In some embodiments, the targets of the RNase enzyme activity of RNase-Fc fusion proteins are primarily extracellular, consisting of, e.g., RNA contained in immune complexes with anti-RNP autoantibody and RNA expressed on the surface of cells undergoing apoptosis. In some embodiments, the RNase-Fc fusion protein is active in the acidic environment of the endocytic vesicles. In some embodiments, an RNase-Fc fusion protein includes a wild-type (wt) Fc domain in order to, e.g, allow the molecule to bind FcR and enter the endocytic compartment through the entry pathway used by immune complexes. In some embodiments, an RNase-Fc fusion protein including a Fc domain, or a variant or fragment thereof, is adapted to be active both extracellularly and in the endocytic environment (where TLR7 can be expressed). In some aspects, this allows an RNase-Fc fusion protein including a wild-type Fc domain, or a variant or fragment thereof, to stop TLR7 signaling through previously engulfed immune complexes or by RNAs that activate TLR7 after viral infection. In some embodiments, the wild type RNase of an RNase-Fc fusion protein is not resistant to inhibition by an RNase cytoplasmic inhibitor. In some embodiments, the wild type RNase of an RNase-Fc fusion protein is not active in the cytoplasm of a cell.

In some embodiments, RNase-Fc fusion proteins include RNase. In some embodiments, RNase-Fc fusion proteins include both DNase and RNase. In some embodiments, these RNase-Fc fusion proteins improve therapy of Sjogren’s syndrome because they digest or degrade immune complexes containing RNA, DNA, or a combination of both RNA and DNA, and are active extracellularly. In some embodiments, the RNase-Fc fusion proteins of the disclosure reduce fatigue in patients in Sjogren’s syndrome.

In some embodiments, the disclosure provides nucleic acids encoding one or more RNase-Fc fusion proteins for use in gene therapy methods for treating or preventing disorders, diseases, and conditions. The gene therapy methods relate to the introduction of RNase-Fc fusion protein nucleic acid (DNA, RNA and antisense DNA or RNA) sequences into an animal in need thereof to achieve expression of the polypeptide or polypeptides of the present disclosure. This method can include introduction of one or more polynucleotides encoding an RNase-Fc fusion protein of the present disclosure operably coupled to a promoter and any other genetic elements necessary for the expression of the RNase-Fc fusion protein by the target tissue.

In gene therapy applications, RNase-Fc fusion protein genes are introduced into cells in order to achieve in vivo synthesis of a therapeutically effective genetic product. “Gene therapy” includes both conventional gene therapies where a lasting effect is achieved by a single treatment, and the administration of gene therapeutic agents, which involves the one time or repeated administration of a therapeutically effective DNA or mRNA. The oligonucleotides can be modified to enhance their uptake, e.g., by substituting their negatively charged phosphodiester groups by uncharged groups.

### Fc Domains

In some embodiments, the polypeptide comprising one or more nuclease domains, or variant or fragment thereof is operably coupled, with or without a linker domain, to a Fc domain, which serves as a scaffold as well as a means to increase the serum half-life of the polypeptide. In some embodiments, the one or more nuclease domains and/or the Fc domain is aglycosylated, deglycosylated, or underglycosylated. In some embodiments, the Fc domain is a mutant or variant Fc domain, or a fragment of an Fc domain.

Suitable Fc domains are well-known in the art and include, but are not limited to, Fc and Fc variants, such as those disclosed in WO2011/053982, WO 02/060955, WO 02/096948, WO05/047327, WO05/018572, and US 2007/0111281 (the contents of the foregoing are incorporated herein by reference). It is within the abilities of the skilled artisan to use routine methods to introduce Fc domains (e.g., cloning, conjugation) into the RNase-Fc fusion proteins disclosed herein (with or without altered glycosylation).

In some embodiments, the Fc domain is a wild type human IgG1 Fc, such as is shown in SEQ ID NO: 20. In some embodiments, the Fc domain is a human IgG1 Fc domain having one or more mutations.

In some embodiments, the Fc domain is a wild type human IgG4 Fc, such as is shown in SEQ ID NOs: 30-31. In some embodiments, the Fc domain is a human IgG4 Fc domain having one or more mutations.

In some embodiments, an Fc domain is altered or modified, e.g., by mutation which results in an amino acid addition, deletion, or substitution. As used herein, the term “Fc domain variant” refers to an Fc domain having at least one amino acid modification, such as an amino acid substitution, as compared to the wild-type Fc from which the Fc domain is derived. For example, wherein the Fc domain is derived from a human IgG1 antibody, a variant comprises at least one amino acid mutation (e.g., substitution) as compared to a wild type amino acid at the corresponding position of the human IgG1 Fc region. The amino acid substitution(s) of an Fc variant may be located at a position within the Fc domain referred to as corresponding to the position number that that residue would be given in an Fc region in an antibody (numbering according to EU index).

In one embodiment, the Fc variant comprises one or more amino acid substitutions at an amino acid position(s) located in a hinge region or portion thereof. In another embodiment, the Fc variant comprises one or more amino acid substitutions at an amino acid position(s) located in a CH2 domain or portion thereof. In another embodiment, the Fc variant comprises one or more amino acid substitutions at an amino acid position(s) located in a CH3 domain or portion thereof. In another embodiment, the Fc variant comprises one or more amino acid substitutions at an amino acid position(s) located in a CH4 domain or portion thereof.

In some embodiments, the Fc domain comprises one or more of the following amino acid substitutions: T350V, L351Y, F405A, and Y407V. In some embodiments, the Fc domain comprises one or more of the following amino acid substitutions: T350V, T366L, K392L, and T394W.

In some embodiments, the human IgG1 Fc region has a mutation at N83 (i.e., N297 by Kabat numbering), yielding an aglycosylated Fc region (e.g., Fc N83S; SEQ ID NO: 21). In some embodiments, the human IgG1 Fc domain includes mutations in one or more of the three hinge region cysteines (residues 220, 226, and 229, numbering according to the EU index). In some embodiments, one or more of the three hinge cysteines in the Fc domain can be mutated to SCC (SEQ ID NO: 24) or SSS (SEQ ID NO: 25), where in “S” represents an amino acid substitution of cysteine with serine (wherein CCC refers to the three cysteines present in the wild type hinge domain). Accordingly “SCC” indicates an amino acid substitution to serine of only the first cysteine of the three hinge region cysteines (residues 220, 226, and 229, numbering according to the EU index), whereas “SSS” indicates that all three cysteines in the hinge region are substituted with serine (residues 220, 226, and 229, numbering according to the EU index).

In some aspects, the Fc domain is a human IgG1 Fc domain having one or more mutations.

In some aspects, a mutant Fc domain comprises one or more mutations in the hinge, CH2, and/or CH3 domains.

In some aspects, the Fc domain is a human IgG4 Fc domain having one or more mutations. In some embodiments, mutations in the IgG4 Fc domain include one or more mutations selected from the following group of mutations: F296Y, E356K, R409K, and H345R. In some embodiments, mutations in the IgG4 Fc domain includes one or more mutation selected from the following group of mutations: F296Y, R409K, and K439E. In some embodiments, the RNase-Fc fusion proteins disclosed herein include a first polypeptide comprising a mutant IgG4 Fc domain, wherein the Fc domain includes mutations F296Y, E356K, R409K, and H345R, and a second polypeptide comprising a mutant IgG4 Fc domain, wherein the CH3 domain includes mutations F296Y, R409K, and K439E. In some embodiments, a mutant IgG4 Fc domain comprises one or more mutations in the hinge, CH2, and/or CH3 domains.

### CH2 Substitutions

In some aspects, a mutant Fc domain includes a P238S mutation. In some aspects, a mutant Fc domain includes a P331S mutation. In some aspects, a mutant Fc domain includes a P238S mutation and a P331S mutation. In some aspects, a mutant Fc domain comprises P238S and/or P331S, and may include mutations in one or more of the three hinge cysteines (residues 220, 226, and 229), numbering according to the EU index. In some aspects, a mutant Fc domain comprises P238S and/or P331S, and/or one or more mutations in the three hinge cysteines (residues 220, 226, and 229), numbering according to the EU index. In some aspects, a mutant Fc domain comprises P238S and/or P331S, and/or mutations in a hinge cysteine to SCC or in the three hinge cysteines to SSS. In some aspects, a mutant Fc domain comprises P238S and P331S and mutations in at least one of the three hinge cysteines. In some aspects, a mutant Fc domain comprises P238S and P331S and SCC. In some aspects, a mutant Fc domain comprises P238S and P331S and SSS. In some aspects, a mutant Fc domain includes P238S and SCC or SSS. In some aspects, a mutant Fc domain includes P331S and SCC or SSS. (All numbering according to the EU index).

In some aspects, a mutant Fc domain includes a mutation at a site of N-linked glycosylation, such as N297, e.g., a substitution of asparagine for another amino acid such as serine, e.g., N297S. In some aspects, a mutant Fc domain includes a mutation at a site of N-linked glycosylation, such as N297, e.g., a substitution of asparagine for another amino acid such as serine, e.g., N297S and a mutation in one or more of the three hinge cysteines. In some aspects, a mutant Fc domain includes a mutation at a site of N-linked glycosylation, such as N297, e.g., a substitution of asparagine for another amino acid such as serine, e.g., N297S and mutations in one of the three hinge cysteines to SCC or all three cysteines to SSS. In some aspects, a mutant Fc domain includes a mutation at a site of N-linked glycosylation, such as N297, e.g., a substitution of asparagine for another amino acid such as serine, e.g., N297 and one or more mutations in the CH2 domain which decrease FcγR binding and/or complement activation, such as mutations at P238 or P331 or both, e.g., P238S or P331S or both P238S and P331S. In some aspects, such mutant Fc domains can further include a mutation in the hinge region, e.g., SCC or SSS. (All numbering according to the EU index.) In some aspects, the mutant Fc domain is as shown in the Sequence Table or Sequence Listing herein.

### CH3 Substitutions

In some embodiments, heterodimers are formed by mutations in the CH3 domain of the Fc domain on the heterodimeric RNase-Fc fusion proteins disclosed herein. Heavy chains were first engineered for heterodimerization using a “knobs-into-holes” strategy (Rigway B, et al., Protein Eng., 9 (1996) pp. 617-621, incorporated herein by reference). The term “knob-into-hole” refers to the technology directing the pairing of two polypeptides together in vitro or in vivo by introducing a pertuberance (knob) into one polypeptide and a cavity (hole) into the other polypeptide at an interface in which they interact. See e.g., WO 96/027011, WO 98/050431, US 5,731,168, US2007/0178552, WO2009089004, US 20090182127. In particular, a combination of mutations in the CH3 domain can be used to form heterodimers, for example, S354C, T366W in the “knob” heavy chain, and Y349C, T366S, L368A, Y407V in the “hole” heavy chain. In another example, T366Y in the “knob” heavy chain, and Y407T in the “hole” heavy chain. In some embodiments, the heterodimeric RNase-Fc fusion protein disclosed herein includes a first CH3 domain having the knob mutation T366W and a second CH3 domain having the hole mutations T366S, L368A, and Y407V. (Numbering according to the EU index.) In some embodiments, the RNase-Fc fusion proteins disclosed herein includes a first CH3 domain having the knob mutation T366Y and a second CH3 domain having the hole mutation Y407T.

In some embodiments, the CH3 mutations are those described in US 2012/0149876 A1, US 2017/0158779, US 9574010, and US 9562109, each of which is incorporated herein by reference; and Von Kreudenstein, T.S. et al. mABs, 5 (2013), pp. 646-654, incorporated herein by reference) and include the following mutations: T350V, L351Y, F405A, and Y407V (first CH3 domain); and T350V, T366L, K392L, T394W (second CH3 domain). In some embodiments, the heterodimeric RNase-Fc fusion proteins disclosed herein include a first CH3 domain having T350V, L351Y, F405A, and Y407V mutations and a second CH3 domain having T350V, T366L, K392L, T394W mutations. (Numbering according to the EU index.)

In some embodiments, heterodimers are formed by mutations in the CH3 domain of the Fc domain on the RNase-Fc fusion protein disclosed herein. In particular, a combination of mutations in the CH3 domain can be used to form heterodimers with high heterodimeric stability and purity; for example, See e.g., Von Kreudenstein et al., mAbs 5:5, 646-654; September-October 2013, and US 2012/0149876 A1, US 2017/0158779, US 9574010, and US 9562109, each of which is incorporated herein by reference in its entirety. In some embodiments, mutations in the Fc domain include one or more mutations selected from the following group of mutations: T350V, L351Y, F405A, and Y407V. In some embodiments, mutations in the Fc domain include one or more mutation selected from the following group of mutations: T350V, T366L, K392L, and T394W. In some embodiments, the RNase-Fc fusion protein disclosed herein include a CH3 domain having mutations T350V, L351Y, F405A, and Y407V. In some embodiments, the RNase-Fc fusion prootein disclosed herein include a CH3 domain having mutations T350V, T366L, K392L, and T394W. In some embodiments, the RNase-Fc fusion proteins disclosed herein include a first polypeptide comprising a mutant Fc domain, wherein the CH3 domain includes mutations T350V, L351Y, F405A, and Y407V, and a second polypeptide comprising a mutant Fc domain, wherein the CH3 domain includes mutations T350V, T366L, K392L, and T394W.

Other mutations in the CH3 domain of the Fc domain are contemplated to preferentially form heterodimers. For example, See e.g., Von Kreudenstein et al., mAbs 5:5, 646-654; September-October 2013, incorporated herein by reference). In some embodiments, mutations in the Fc domain of the first polypeptide include one or more mutations selected from, the following group of mutations: T350V, L351Y, F405A, and Y407V, and mutations in the Fc domain of the second polypeptide include one or more mutations selected from the following group of mutations: T350V, T366L, K392M, and T394W. In some embodiments, mutations in the Fc domain of the first polypeptide include one or more mutations selected from the following group of mutations: L351Y, F405A, and Y407V, mutations in the Fc domain of the second polypeptide include one or more mutations selected from the following group of mutations: T366L, K392M, and T394W.

In some embodiments, the CH3 mutations are those described by Moore, G.L. et al. (mABs, 3 (2011), pp. 546-557) and include the following mutations: S364H and F405A (first CH3 domain); and Y349T and T394F (second CH3 domain). In some embodiments, the heterodimeric RNase-Fc fusion protein disclosed herein includes a first CH3 domain having S364H and F405A mutations and a second CH3 domain having Y349T and T394F mutations. (Numbering according to the EU index.)

In some embodiments, the CH3 mutations are those described by Gunasekaran, K. et al. (J. Biol. Chem., 285 (2010), pp. 19637-19646) and include the following mutations: K409D and K392D (first CH3 domain); and D399K and E365K (second CH3 domain). In some embodiments, the heterodimeric RNase-Fc fusion protein disclosed herein includes a first CH3 domain having K409D and K392D mutations and a second CH3 domain having D399K and E365K mutations. (Numbering according to the EU index.)

The RNase-Fc fusion proteins of the disclosure may employ art-recognized Fc variants which are known to impart an alteration in effector function and/or FcR binding. For example, a change (e.g., a substitution) at one or more of the amino acid positions disclosed in International PCT Publications WO88/07089A1, WO96/14339A1, WO98/05787A1, WO98/23289A1, WO99/51642A1, WO99/58572A1, WO00/09560A2, WO00/32767A1, WO00/42072A2, WO02/44215A2, WO02/060919A2, WO03/074569A2, WO04/016750A2, WO04/029207A2, WO04/035752A2, WO04/063351 A2, WO04/074455A2, WO04/099249A2, WO05/040217A2, WO04/044859, WO05/070963A1, WO05/077981A2, WO05/092925A2, WO05/123780A2, WO06/019447A1, WO06/047350A2, and WO06/085967A2; U.S. Pat. Publication Nos. US2007/0231329, US2007/0231329, US2007/0237765, US2007/0237766, US2007/0237767, US2007/0243188, US20070248603, US20070286859, US20080057056; or U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; 7,083,784; and 7,317,091, each of which is incorporated by reference herein. In one embodiment, the specific change (e.g., the specific substitution of one or more amino acids disclosed in the art) may be made at one or more of the disclosed amino acid positions. In another embodiment, a different change at one or more of the disclosed amino acid positions (e.g., the different substitution of one or more amino acid position disclosed in the art) may be made.

Other amino acid mutations in the Fc domain are contemplated to reduce binding to the Fc gamma receptor and Fc gamma receptor subtypes. The assignment of amino acids residue numbers to an Fc domain is in accordance with the definitions of Kabat. See, e.g., Sequences of Proteins of Immunological Interest (Table of Contents, Introduction and Constant Region Sequences sections), 5th edition, Bethesda, MD:NIH vol. 1:647-723 (1991); Kabat et al., “Introduction” Sequences of Proteins of Immunological Interest, US Dept of Health and Human Services, NIH, 5th edition, Bethesda, MD vol. 1:xiii-xcvi (1991); Chothia & Lesk, J. Mol. Biol. 196:901-917 (1987); Chothia et al., Nature 342:878-883 (1989), each of which is herein incorporated by reference for all purposes.”

For example, mutations at positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 322, 324, 327, 329, 330, 331, 333, 334, 335, 337, 338, 340, 356, 360, 373, 376, 378, 379, 382, 388, 389, 398, 414, 416, 419, 430, 434, 435, 437, 438 or 439 of the Fc region can alter binding as described in U.S. Pat. No. 6,737,056, issued May 18, 2004, incorporated herein by reference in its entirety. This patent reported that changing Pro331 in IgG3 to Ser resulted in six fold lower affinity as compared to unmutated IgG3, indicating the involvement of Pro331 in Fc gamma RI binding. In addition, amino acid modifications at positions 234, 235, 236, and 237, 297, 318, 320 and 322 are disclosed as potentially altering receptor binding affinity in U.S. 5,624,821, issued Apr. 29, 1997 and incorporated herein by reference in its entirety. (Numbering according to the EU index.)

Further mutations contemplated for use include, e.g., those described in U.S. Pat. App. Pub. No. 2006/0235208, published Oct. 19, 2006 and incorporated herein by reference in its entirety. This publications describe Fc variants that exhibit reduced binding to Fc gamma receptors, reduced antibody dependent cell-mediated cytotoxicity, or reduced complement dependent cytotoxicity, that comprise at least one amino acid modification in the Fc region, including 232G, 234G, 234H, 235D, 235G, 235H, 236I, 236N, 236P, 236R, 237K, 237L, 237N, 237P, 238K, 239R, 265G, 267R, 269R, 270H, 297S, 299A, 299I, 299V, 325A, 325L, 327R, 328R, 329K, 330I, 330L, 330N, 330P, 330R, and 331L (numbering is according to the EU index), as well as double mutants 236R/237K, 236R/325L, 236R/328R, 237K/325L, 237K/328R, 325L/328R, 235G/236R, 267R/269R, 234G/235G, 236R/237K/325L, 236R/325L/328R, 235G/236R/237K, and 237K/325L/328R. Other mutations contemplated for use as described in this publication include 227G, 234D, 234E, 234G, 234I, 234Y, 235D, 235I, 235S, 236S, 239D, 246H, 255Y, 258H, 260H, 2641, 267D, 267E, 268D, 268E, 272H, 272I, 272R, 281D, 282G, 283H, 284E, 293R, 295E, 304T, 324G, 324I, 327D, 327A, 328A, 328D, 328E, 328F, 328I, 328M, 328N, 328Q, 328T, 328V, 328Y, 330I, 330L, 330Y, 332D, 332E, 335D, an insertion of G between positions 235 and 236, an insertion of A between positions 235 and 236, an insertion of S between positions 235 and 236, an insertion of T between positions 235 and 236, an insertion of N between positions 235 and 236, an insertion of D between positions 235 and 236, an insertion of V between positions 235 and 236, an insertion of L between positions 235 and 236, an insertion of G between positions 235 and 236, an insertion of A between positions 235 and 236, an insertion of S between positions 235 and 236, an insertion of T between positions 235 and 236, an insertion of N between positions 235 and 236, an insertion of D between positions 235 and 236, an insertion of V between positions 235 and 236, an insertion of L between positions 235 and 236, an insertion of G between positions 297 and 298, an insertion of A between positions 297 and 298, an insertion of S between positions 297 and 298, an insertion of D between positions 297 and 298, an insertion of G between positions 326 and 327, an insertion of A between positions 326 and 327, an insertion of T between positions 326 and 327, an insertion of D between positions 326 and 327, and an insertion of E between positions 326 and 327 (numbering is according to the EU index). Additionally, mutations described in U.S. Pat. App. Pub. No. 2006/0235208 include 227G/332E, 234D/332E, 234E/332E, 234Y/332E, 234I/332E, 234G/332E, 235I/332E, 235S/332E, 235D/332E, 235E/332E, 236S/332E, 236A/332E, 236S/332D, 236A/332D, 239D/268E, 246H/332E, 255Y/332E, 258H/332E, 260H/332E, 264I/332E, 267E/332E, 267D/332E, 268D/332D, 268E/332D, 268E/332E, 268D/332E, 268E/330Y, 268D/330Y, 272R/332E, 272H/332E, 283H/332E, 284E/332E, 293R/332E, 295E/332E, 304T/332E, 324I/332E, 324G/332E, 324I/332D, 324G/332D, 327D/332E, 328A/332E, 328T/332E, 328V/332E, 328I/332E, 328F/332E, 328Y/332E, 328M/332E, 328D/332E, 328E/332E, 328N/332E, 328Q/332E, 328A/332D, 328T/332D, 328V/332D, 328I/332D, 328F/332D, 328Y/332D, 328M/332D, 328D/332D, 328E/332D, 328N/332D, 328Q/332D, 330L/332E, 330Y/332E, 330I/332E, 332D/330Y, 335D/332E, 239D/332E, 239D/332E/330Y, 239D/332E/330L, 239D/332E/330I, 239D/332E/268E, 239D/332E/268D, 239D/332E/327D, 239D/332E/284E, 239D/268E/330Y, 239D/332E/268E/330Y, 239D/332E/327A, 239D/332E/268E/327A, 239D/332E/330Y/327A, 332E/330Y/268 E/327A, 239D/332E/268E/330Y/327A, Insert G>297-298/332E, Insert A>297-298/332E, Insert S>297-298/332E, Insert D>297-298/332E, Insert G>326-327/332E, Insert A>326-327/332E, Insert T>326-327/332E, Insert D>326-327/332E, Insert E>326-327/332E, Insert G>235-236/332E, Insert A>235-236/332E, Insert S>235-236/332E, Insert T>235-236/332E, Insert N>235-236/332E, Insert D>235-236/332E, Insert V>235-236/332E, Insert L>235-236/332E, Insert G>235-236/332D, Insert A>235-236/332D, Insert S>235-236/332D, Insert T>235-236/332D, Insert N>235-236/332D, Insert D>235-236/332D, Insert V>235-236/332D, and Insert L>235-236/332D (numbering according to the EU index) are contemplated for use. The mutant L234A/L235A is described, e.g., in U.S. Pat. App. Pub. No. 2003/0108548, published Jun. 12, 2003 and incorporated herein by reference in its entirety. In embodiments, the described modifications are included either individually or in combination. (Numbering according to the EU index.)

### PK Moieties

In some embodiments, the RNase is operably coupled to a PK moiety, which serves as a scaffold as well as a means to increase the serum half-life of the RNase.

Suitable PK moieties are well-known in the art and include, but are not limited to, albumin, transferrin, Fc, and their variants, and polyethylene glycol (PEG) and its derivatives. Suitable PK moieties include, but are not limited to, HSA, or variants or fragments thereof, such as those disclosed in US 5,876,969, WO 2011/124718, and WO 2011/0514789; Fc and Fc variants, such as those disclosed in WO2011/053982, WO 02/060955, WO 02/096948, WO05/047327, WO05/018572, and US 2007/0111281; transferrin, or variants or fragments thereof, as disclosed in US 7,176,278 and US 8,158,579; and PEG or derivatives, such as those disclosed in Zalipsky et al. (“Use of Functionalized Poly(Ethylene Glycols) for Modification of Polypeptides” in Polyethylene Glycol Chemistry: Biotechnical and Biomedical Applications, J. M. Harris, Plenus Press, New York (1992)), and in Zalipsky et al. Advanced Drug Reviews 1995:16: 157-182), and U.S. Pat. Nos. 4,640,835, 4,496,689, 4,301,144, 4,670,417, 4,791,192, 4,179,337, and 5,932,462 (the contents of the foregoing are incorporated herein by reference). It is within the abilities of the skilled artisan to use routine methods to operably couple PK moieties (e.g., cloning, conjugation) to the RNase of the invention.

In some embodiments, the PK moiety is HSA, which is naturally aglycosylated.

In some embodiments, the PK moiety is a wild type Fc (SEQ ID NO: 20).

In certain embodiments, an Fc domain is altered or modified, e.g., by amino acid mutation (e.g., addition, deletion, or substitution). As used herein, the term “Fc domain variant” refers to an Fc domain having at least one amino acid modification, such as an amino acid substitution, as compared to the wild-type Fc from which the Fc domain is derived. For example, wherein the Fc domain is derived from a human IgG1 antibody, a variant comprises at least one amino acid mutation (e.g., substitution) as compared to a wild type amino acid at the corresponding position of the human IgG1 Fc region. For example, wherein the Fc domain is derived from a human IgG4 antibody, a variant comprises at least one amino acid mutation (e.g., substitution) as compared to a wild type amino acid at the corresponding position of the human IgG4 Fc region.

In some embodiments, the PK moiety is any of the Fc variants described herein.

In some embodiments, the PK moiety is a wild type HST. In other embodiments, the PK moiety is a HST with a mutations at N413 and/or N611 and/or S12 (S12 is a potential O-linked glycosylation site), yielding a HST with altered glycosylation (i.e., HST N413 S, HST N611S, HST N413 S/N611S and HST S12A/N413S/N611S).

### Linker Domains

In some embodiments, an RNase-Fc fusion protein includes a linker domain. In some embodiments, an RNase-Fc fusion protein includes a plurality of linker domains. In some embodiments, the linker domain is a polypeptide linker. In certain aspects, it is desirable to employ a polypeptide linker to fuse Fc, or a variant or fragment thereof, with one or more nuclease domains to form an RNase-Fc fusion protein.

In one embodiment, the polypeptide linker is synthetic. As used herein, the term “synthetic” with respect to a polypeptide linker includes peptides (or polypeptides) which comprise an amino acid sequence (which may or may not be naturally occurring) that is linked in a linear sequence of amino acids to a sequence (which may or may not be naturally occurring) (e.g., a Fc sequence) to which it is not naturally linked in nature. For example, the polypeptide linker may comprise non-naturally occurring polypeptides which are modified forms of naturally occurring polypeptides (e.g., comprising a mutation such as an addition, substitution or deletion) or which comprise a first amino acid sequence (which may or may not be naturally occurring). The polypeptide linkers of the invention may be employed, for instance, to ensure that Fc, or a variant or fragment thereof, is juxtaposed to ensure proper folding and formation of a functional Fc, or a variant or fragment thereof. Preferably, a polypeptide linker compatible with the instant invention will be relatively non-immunogenic and not inhibit any non-covalent association among monomer subunits of a binding protein.

In certain embodiments, the RNase-Fc fusion protein employs an NLG linker as set forth in SEQ ID NO: 37.

In certain embodiments, the RNase-Fc fusion proteins of the disclosure employ a polypeptide linker to join any two or more domains in frame in a single polypeptide chain. In one embodiment, the two or more domains may be independently selected from any of the Fc domains, or variants or fragments thereof, or nuclease domains discussed herein. In some embodiments, the RNase domain of the RNase-Fc fusion protein is operably coupled to the Fc domain via a linker domain. In some embodiments, a polypeptide linker can be used to fuse identical Fc fragments, thereby forming a homodimeric Fc region. In other embodiments, a polypeptide linker can be used to fuse different Fc fragments, thereby forming a heterodimeric Fc region. In other embodiments, a polypeptide linker of the invention can be used to genetically fuse the C-terminus of a first Fc fragment to the N-terminus of a second Fc fragment to form a complete Fc domain.

In one embodiment, a polypeptide linker comprises a portion of a Fc domain, or a variant or fragment thereof. For example, in one embodiment, a polypeptide linker can comprise a Fc fragment (e.g., C or N domain), or a different portion of a Fc domain or variant thereof.

In another embodiment, a polypeptide linker comprises or consists of a gly-ser linker. As used herein, the term “gly-ser linker” refers to a peptide that consists of glycine and serine residues. An exemplary gly/ser linker comprises an amino acid sequence of the formula (Gly₄Ser)n, wherein n is a positive integer (e.g., 1, 2, 3, 4, or 5). A preferred gly/ser linker is (Gly₄Ser)4. Another preferred gly/ser linker is (Gly₄Ser)3. Another preferred gly/ser linker is (Gly₄Ser)5. In certain embodiments, the gly-ser linker may be inserted between two other sequences of the polypeptide linker (e.g., any of the polypeptide linker sequences described herein). In other embodiments, a gly-ser linker is attached at one or both ends of another sequence of the polypeptide linker (e.g., any of the polypeptide linker sequences described herein). In yet other embodiments, two or more gly-ser linker are incorporated in series in a polypeptide linker.

In other embodiments, a polypeptide linker of the invention comprises a biologically relevant peptide sequence or a sequence portion thereof. For example, a biologically relevant peptide sequence may include, but is not limited to, sequences derived from an anti-rejection or anti-inflammatory peptide. Said anti-rejection or anti-inflammatory peptides may be selected from the group consisting of a cytokine inhibitory peptide, a cell adhesion inhibitory peptide, a thrombin inhibitory peptide, and a platelet inhibitory peptide. In a preferred embodiment, a polypeptide linker comprises a peptide sequence selected from the group consisting of an IL-1 inhibitory or antagonist peptide sequence, an erythropoietin (EPO)-mimetic peptide sequence, a thrombopoietin (TPO)-mimetic peptide sequence, G-CSF mimetic peptide sequence, a TNF-antagonist peptide sequence, an integrin-binding peptide sequence, a selectin antagonist peptide sequence, an anti-pathogenic peptide sequence, a vasoactive intestinal peptide (VIP) mimetic peptide sequence, a calmodulin antagonist peptide sequence, a mast cell antagonist, a SH3 antagonist peptide sequence, an urokinase receptor (UKR) antagonist peptide sequence, a somatostatin or cortistatin mimetic peptide sequence, and a macrophage and/or T-cell inhibiting peptide sequence. Exemplary peptide sequences, any one of which may be employed as a polypeptide linker, are disclosed in U.S. Pat. No. 6,660,843, which is incorporated by reference herein.

Other linkers that are suitable for use in RNase-Fc fusion proteins are known in the art, for example, the serine-rich linkers disclosed in US 5,525,491, the helix forming peptide linkers (e.g., A(EAAAK)nA (n=2-5)) disclosed in Arai et al., Protein Eng 2001;14:529-32, and the stable linkers disclosed in Chen et al., Mol Pharm 2011;8:457-65, i.e., the dipeptide linker LE, a thrombin-sensitive disulfide cyclopeptide linker, and the alpha-helix forming linker LEA(EAAAK)₄ALEA(EAAAK)₄ALE (SEQ ID NO: 39).

Other exemplary linkers include GS linkers (i.e., (GS)n), GGSG (SEQ ID NO: 40) linkers (i.e., (GGSG)n), GSAT linkers (SEQ ID NO: 41), SEG linkers, and GGS linkers (i.e., (GGSGGS)n), wherein n is a positive integer (e.g., 1, 2, 3, 4, or 5). Other suitable linkers for use in the RNase-Fc fusion proteins can be found using publicly available databases, such as the Linker Database (ibi.vu.nl/programs/linkerdbwww). The Linker Database is a database of inter-domain linkers in multi-functional enzymes which serve as potential linkers in novel fusion proteins (see, e.g., George et al., Protein Engineering 2002;15:871-9).

It will be understood that variant forms of these exemplary polypeptide linkers can be created by introducing one or more nucleotide substitutions, additions or deletions into the nucleotide sequence encoding a polypeptide linker such that one or more amino acid substitutions, additions or deletions are introduced into the polypeptide linker. Mutations may be introduced by standard techniques, such as site-directed mutagenesis and PCR-mediated mutagenesis.

Polypeptide linkers of the disclosure are at least one amino acid in length and can be of varying lengths. In one embodiment, a polypeptide linker of the invention is from about 1 to about 50 amino acids in length. As used in this context, the term “about” indicates +/- two amino acid residues. Since linker length must be a positive integer, the length of from about 1 to about 50 amino acids in length, means a length of from 1 to 48-52 amino acids in length. In another embodiment, a polypeptide linker of the disclosure is from about 10-20 amino acids in length. In another embodiment, a polypeptide linker of the disclosure is from about 15 to about 50 amino acids in length.

In another embodiment, a polypeptide linker of the disclosure is from about 20 to about 45 amino acids in length. In another embodiment, a polypeptide linker of the disclosure is from about 15 to about 25 amino acids in length. In another embodiment, a polypeptide linker of the disclosure is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56,57, 58, 59, 60, or 61 or more amino acids in length.

Polypeptide linkers can be introduced into polypeptide sequences using techniques known in the art. Modifications can be confirmed by DNA sequence analysis. Plasmid DNA can be used to transform host cells for stable production of the polypeptides produced.

### RNase-Containing Nuclease Fusion Proteins With Altered Glycosylation

Glycosylation (e.g., O-lined or N-linked glycosylation) can impact the serum half-life of the RNase-containing nuclease fusion protein of the disclosure by, e.g., minimizing their removal from circulation by mannose and asialoglycoprotein receptors and other lectin-like receptors. Accordingly, in some embodiments, the RNase-containing nuclease fusion proteins of the disclosure, including RNase-Fc proteins are prepared in aglycosylated, deglycosylated, or underglycosylated form. Preferably, N-linked glycosylation is altered and the RNase-Fc fusion protein is aglycosyated.

In some embodiments, all asparagine residues in a RNase-Fc fusion protein that conform to the Asn-X-Ser/Thr (X can be any other naturally occurring amino acid except Pro) consensus are mutated to residues that do not serve as acceptors of N-linked glycosylation (e.g., serine, glutamine), thereby eliminating glycosylation of the RNase-Fc fusion protein when synthesized in a cell that glycosylates proteins.

In some embodiments, RNase-Fc fusion proteins lacking N-linked glycosylation sites are produced in mammalian cells. In one embodiment, the mammalian cell is a CHO cell. Accordingly, in a specific embodiment, an aglycosylated RNase-Fc fusion protein is produced in a CHO cell.

In other embodiments, a reduction or lack of N-glycosylation is achieved by, e.g., producing RNase-Fc fusion proteins in a host (e.g., bacteria such as E. coli), mammalian cells engineered to lack one or more enzymes important for glycosylation, or mammalian cells treated with agents that prevent glycosylation, such as tunicamycin (an inhibitor of Dol-PP-GlcNAc formation).

In some embodiments, the RNase-Fc fusion proteins are produced in lower eukaryotes engineered to produce glycoproteins with complex N-glycans, rather than high mannose type sugars (see, e.g., US2007/0105127).

In some embodiments, glycosylated RNase-Fc fusion proteins (e.g., those produced in mammalian cells such as CHO cells) are treated chemically or enzymatically to remove one or more carbohydrate residues (e.g., one or more mannose, fucose, and/or N-acetylglucosamine residues) or to modify or mask one or more carbohydrate residues. Such modifications or masking may reduce binding of the RNase-Fc fusion proteins to mannose receptors, and/or asialoglycoprotein receptors, and/or other lectin-like receptors. Chemical deglycosylation can be achieved by treating a RNase-Fc fusion protein with trifluoromethane sulfonic acid (TFMS), as disclosed in, e.g., Sojar et al., JBC 1989;264:2552-9 and Sojar et al., Methods Enzymol 1987;138:341-50, or by treating with hydrogen fluoride, as disclosed in Sojar et al. (1987, supra). Enzymatic removal of N-linked carbohydrates from RNase-Fc fusion proteins can be achieved by treating a RNase-Fc fusion protein with protein N-glycosidase (PNGase) A or F, as disclosed in Thotakura et al. (Methods Enzymol 1987;138:350-9). Other art-recognized commercially available deglycosylating enzymes that are suitable for use include endo-alpha-N-acetyl-galactosaminidase, endoglycosidase F1, endoglycosidase F2, endoglycosidase F3, and endoglycosidase H. In some embodiments, one or more of these enzymes can be used to deglycosylate the RNase-Fc fusion proteins of the disclosure. Alternative methods for deglycosylation are disclosed in, e.g., US 8,198,063.

In some embodiments, the RNase-Fc fusion proteins are partially deglycosylated. Partial deglycosylation can be achieved by treating the RNase-Fc fusion protein with an endoglycosidase (e.g., endoglycosidase H), which cleaves N-linked high mannose carbohydrate but not complex type carbohydrates, leaving a single GlcNAc residue linked to the asparagine. RNase-Fc fusion proteins treated with endoglycosidase H will lack high mannose carbohydrates, resulting in a reduced interaction with the hepatic mannose receptor. Although this receptor recognizes terminal GlcNAc, the probability of a productive interaction with the single GlcNAc on the protein surface is not as great as with an intact high mannose structure.

In other embodiments, glycosylation of a RNase-Fc fusion protein is modified, e.g., by oxidation, reduction, dehydration, substitution, esterification, alkylation, sialylation, carbon-carbon bond cleavage, or the like, to reduce clearance of the RNase-Fc fusion protein from blood. In some embodiments, the RNase-Fc fusion proteins are treated with periodate and sodium borohydride to modify the carbohydrate structure. Periodate treatment oxidizes vicinal diols, cleaving the carbon-carbon bond and replacing the hydroxyl groups with aldehyde groups; borohydride reduces the aldehydes to hydroxyls. Many sugar residues include vicinal diols and, therefore, are cleaved by this treatment. Prolonged serum half-life with periodate and sodium borohydride is exemplified by the sequential treatment of the lysosomal enzyme β-glucuronidase with these agents (see, e.g., Houba et al. (1996) Bioconjug Chem 1996:7:606-11; Stahl et al. PNAS 1976;73:4045-9; Achord et al. Pediat. Res 1977;11:816-22; Achord et al. Cell 1978;15:269-78). A method for treatment with periodate and sodium borohydride is disclosed in Hickman et al., BBRC 1974;57:55-61. A method for treatment with periodate and cyanoborohydride, which increases the serum half-life and tissue distribution of ricin, is disclosed in Thorpe et al. Eur J Biochem 1985;147:197-206.

In one embodiment, the carbohydrate structures of a RNase-Fc fusion protein can be masked by addition of one or more additional moieties (e.g., carbohydrate groups, phosphate groups, alkyl groups, etc.) that interfere with recognition of the structure by a mannose or asialoglycoprotein receptor or other lectin-like receptors.

In some embodiments, one or more potential glycosylation sites are removed by mutation of the nucleic acid encoding the RNase-Fc fusion protein, thereby reducing glycosylation (underglycosylation) of the RNase-Fc fusion protein when synthesized in a cell that glycosylates proteins, e.g., a mammalian cell such as a CHO cell. In some embodiments, it may be desirable to selectively underglycosylate the RNase-Fc fusion protein by mutating the potential N-linked glycosylation sites therein if, e.g., the underglycosylated RNase-Fc fusion protein exhibits increased activity or contributes to increased serum half-life. In other embodiments, it may be desirable to underglycosylate portions of the RNase-Fc fusion protein such that certain domains lack N-glycosylation if, for example, such a modification improves the serum half-life of the RNase-Fc fusion protein. Alternatively, other amino acids in the vicinity of glycosylation acceptors can be modified, disrupting a recognition motif for glycosylation enzymes without necessarily changing the amino acid that would normally be glycosylated.

In some embodiments, glycosylation of a RNase-Fc fusion protein can be altered by introducing glycosylation sites. For example, the amino acid sequence of the RNase-Fc fusion protein can be modified to introduce the consensus sequence for N-linked glycosylation of Asn-X-Ser/Thr (X is any amino acid other than proline). Additional N-linked glycosylation sites can be added anywhere throughout the amino acid sequence of the RNase-Fc fusion protein. Preferably, the glycosylation sites are introduced in position in the amino acid sequence that does not substantially reduce the activity of the RNase-Fc fusion protein.

The addition of O-linked glycosylation sites has been reported to alter serum half-life of proteins, such as growth hormone, follicle-stimulating hormone, IGFBP-6, Factor IX, and many others (e.g., as disclosed in Okada et al., Endocr Rev 2011;32:2-342; Weenen et al., J Clin Endocrinol Metab 2004;89:5204-12; Marinaro et al., European Journal of Endocrinology 2000;142:512-6; US 2011/0154516). Accordingly, in some embodiments, O-linked glycosylation (on serine/threonine residues) of the RNase-Fc fusion protein is altered. Methods for altering O-linked glycosylation are routine in the art and can be achieved, e.g., by beta-elimination (see, e.g., Huang et al., Rapid Communications in Mass Spectrometry 2002;16:1199-204; Conrad, Curr Protoc MolBiol 2001; Chapter 17:Unit17.15A; Fukuda, Curr Protoc Mol Biol 2001;Chapter 17;Unit 17.15B; Zachara et al., Curr Protoc Mol Biol 2011; Unit 17.6;); by using commercially available kits (e.g., GlycoProfile™ Beta-Elimination Kit, Sigma); or by subjecting RNase-Fc fusion proteins to treatment with a series of exoglycosidases such as, but not limited to, β1-4 galactosidase and β -N-acetylglucosaminidase, until only Gal β1-3GalNAc and/or GlcNAc β1-3GalNAc remains, followed by treatment with, e.g., endo-α-N-acetylgalactosaminidase (i.e., O-glycosidase). Such enzymes are commercially available from, e.g., New England Biolabs. In yet other embodiments, the RNase-Fc fusion proteins are altered to introduce O-linked glycosylation in the RNase-Fc fusion protein as disclosed in, e.g., Okada et al. (supra), Weenen et al. (supra), US2008/0274958; and US2011/0171218. In some embodiments, one or more O-linked glycosylation consensus sites are introduced into the RNase-Fc fusion protein, such as CXXGGT/S-C (SEQ ID NO: 59) (van den Steen et al., In Critical Reviews in Biochemistry and Molecular Biology, Michael Cox, ed., 1998;33:151-208), NST-E/D-A (SEQ ID NO: 60), NITQS (SEQ ID NO: 61), QSTQS (SEQ ID NO: 62), D/E-FT-R/K-V (SEQ ID NO: 63), C-E/D-SN (SEQ ID NO: 64), and GGSC-K/R (SEQ ID NO: 65). Additional O-linked glycosylation sites can be added anywhere throughout the amino acid sequence of the RNase-Fc fusion protein. Preferably, the glycosylation sites are introduced in position in the amino acid sequence that does not substantially reduce the activity of the RNase-Fc fusion protein. Alternatively, O-linked sugar moieties are introduced by chemically modifying an amino acid in the RNase-Fc fusion protein as described in, e.g., WO 87/05330 and Aplin et al., CRC Crit Rev Biochem 1981;259-306).

In some embodiments, both N-linked and O-linked glycosylation sites are introduced into the RNase-Fc fusion protein, preferably in positions in the amino acid sequence that do not substantially reduce the activity of the RNase-Fc fusion protein.

It is well within the abilities of the skilled artisan to introduce, reduce, or eliminate glycosylation (e.g., N-linked or O-linked glycosylation) in a RNase-Fc fusion protein and determine using routine methods in the art whether such modifications in glycosylation status increases or decreases the activity or serum half-life of the RNase-Fc fusion protein.

In some embodiments, the RNase-Fc fusion protein may comprise an altered glycoform (e.g., an underfucosylated or fucose-free glycan).

In some embodiments, a RNase-Fc fusion protein with altered glycosylation has a serum half-life that is increased at least about 1.5-fold, such as at least 3-fold, at least 5-fold, at least 10-fold, at least about 20-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1000-fold, or 1000-fold or greater relative to the corresponding glycosylated RNase-Fc fusion proteins (e.g., a RNase-Fc fusion protein in which potential N-linked glycosylation sites are not mutated). Routine art-recognized methods can be used to determine the serum half-life of RNase-Fc fusion proteins with altered glycosylation status.

In some embodiments, a RNase-Fc fusion protein with altered glycosylation (e.g., a aglycosylated, deglycosylated, or underglycosylated RNase-Fc fusion protein) retains at least 50%, such as at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or 100% of the activity of the corresponding glycosylated RNase-Fc fusion protein (e.g., a RNase-Fc fusion protein in which potential N-linked glycosylation sites are not mutated).

In some embodiments, altering the glycosylation status of the RNase-Fc fusion protein may increase activity, either by directly increasing activity, or by increasing bioavailability (e.g., serum half-life). Accordingly, in some embodiments, the activity of a RNase-Fc fusion protein with altered glycosylation is increased by at least 1.3-fold, such as at least 1.5-fold, at least 2-fold, at least 2.5-fold, at least 3-fold, at least 3.5-fold, at least 4-fold, at least 4.5-fold, at least 5-fold, at least 5.5-fold, at least 6-fold, at least 6.5-fold, at least 7-fold, at least 7.5-fold, at least 8-fold, at least 8.5-fold, at least 9-fold, at least 9.5 fold, or 10-fold or greater, relative to the corresponding glycosylated RNase-Fc fusion protein (e.g., a RNase-Fc fusion protein in which potential N-linked glycosylation sites are not mutated).

The skilled artisan can readily determine the glycosylation status of RNase-Fc fusion protein using art-recognized methods. In a preferred embodiment, the glycosylation status is determined using mass spectrometry. In other embodiments, interactions with Concanavalin A (Con A) can be assessed to determine whether a RNase-Fc fusion protein is underglycosylated. An underglycosylated RNase-Fc fusion protein is expected to exhibit reduced binding to Con A-Sepharose when compared to the corresponding glycosylated RNase-Fc fusion protein. SDS-PAGE analysis can also be used to compare the mobility of an underglycosylated protein and corresponding glycosylated protein. The underglycosylated protein is expected to have a greater mobility in SDS-PAGE compared to the glycosylated protein. Other suitable art-recognized methods for analyzing protein glycosylation status are disclosed in, e.g., Roth et al., International Journal of Carbohydrate Chemistry 2012;1-10.

Pharmacokinetics, such as serum half-life, of RNase-Fc fusion proteins with different glycosylation status can be assayed using routine methods, e.g., by introducing the RNase-Fc fusion proteins in mice, e.g., intravenously, taking blood samples at pre-determined time points, and assaying and comparing levels and/or activity of the RNase-Fc fusion proteins in the samples.

### Methods of Making RNase-Fc Fusion Proteins

The RNase-containing nuclease fusion proteins of the disclosure, including RNase-Fc fusion proteins are made in transformed or transfected host cells using recombinant DNA techniques. To do so, a recombinant DNA molecule coding for the RNase-Fc fusion protein is prepared. Methods of preparing such DNA molecules are well known in the art. For instance, sequences coding for the RNase-Fc fusion proteins could be excised from DNA using suitable restriction enzymes. Alternatively, the DNA molecule could be synthesized using chemical synthesis techniques, such as the phosphoramidate method. Also, a combination of these techniques could be used.

The invention also includes a vector capable of expressing the RNase-Fc fusion proteins in an appropriate host. The vector comprises the DNA molecule that codes for the RNase-Fc fusion proteins operably coupled to appropriate expression control sequences. Methods of affecting this operative linking, either before or after the DNA molecule is inserted into the vector, are well known. Expression control sequences include promoters, activators, enhancers, operators, ribosomal nuclease domains, start signals, stop signals, cap signals, polyadenylation signals, and other signals involved with the control of transcription or translation.

The resulting vector having the DNA molecule thereon is used to transform or transfect an appropriate host. In some embodiments, the RNase-Fc fusion proteins of the disclosure may be made by co-transfecting or co-transforming two or more expression vectors comprising DNA that codes for an RNase-Fc fusion protein into an appropriate host. This transformation or transfection may be performed using methods well known in the art.

Any of a large number of available and well-known host cells may be used in the practice of this invention. The selection of a particular host is dependent upon a number of factors recognized by the art. These include, for example, compatibility with the chosen expression vector, toxicity of the RNase-Fc fusion proteins encoded by the DNA molecule, rate of transformation or transfection, ease of recovery of the RNase-Fc fusion proteins, expression characteristics, bio-safety and costs. A balance of these factors must be struck with the understanding that not all hosts may be equally effective for the expression of a particular DNA sequence. Within these general guidelines, useful microbial hosts include bacteria (such as E. coli), yeast (such as Saccharomyces) and other fungi, insects, plants, mammalian (including human) cells in culture, or other hosts known in the art. In some embodiments, the RNase-Fc fusion proteins are produced in CHO cells.

Next, the transformed or transfected host is cultured and purified. Host cells may be cultured under conventional fermentation or culture conditions so that the desired compounds are expressed. Such fermentation and culture conditions are well known in the art. Finally, the RNase-Fc fusion proteins are purified from culture by methods well known in the art.

The compounds may also be made by synthetic methods. For example, solid phase synthesis techniques may be used. Suitable techniques are well known in the art, and include those described in Merrifield (1973), Chem. Polypeptides, pp. 335-61 (Katsoyannis and Panayotis eds.); Merrifield (1963), J. Am. Chem. Soc. 85: 2149; Davis et al., Biochem Intl 1985;10: 394-414; Stewart and Young (1969), Solid Phase Peptide Synthesis; U.S. Pat. No. 3,941,763; Finn et al. (1976), The Proteins (3rd ed.) 2: 105-253; and Erickson et al. (1976), The Proteins (3rd ed.) 2: 257-527. In some embodiments, compounds that contain derivatized peptides or which contain non-peptide groups may be synthesized by well-known organic chemistry techniques.

Other methods are of molecule expression/synthesis are generally known in the art to one of ordinary skill.

### Pharmaceutical Compositions

In certain embodiments, RNase-containing nuclease fusion proteins of the disclosure, including RNase-Fc fusion proteins are administered alone. In certain embodiments, an RNase-Fc fusion protein is administered prior to the administration of at least one other therapeutic agent. In certain embodiments, an RNase-Fc fusion protein is administered concurrent with the administration of at least one other therapeutic agent. In certain embodiments, an RNase-Fc fusion protein is administered subsequent to the administration of at least one other therapeutic agent. In other embodiments, an RNase-Fc fusion protein is administered prior to the administration of at least one other therapeutic agent. As will be appreciated by one of skill in the art, in some embodiments, the RNase-Fc fusion protein is combined with the other agent/compound. In some embodiments, the RNase-Fc fusion protein and other agent are administered concurrently. In some embodiments, the RNase-Fc fusion protein and other agent are not administered simultaneously, with the RNase-Fc fusion protein being administered before or after the agent is administered. In some embodiments, the subject receives both the RNase-Fc fusion protein and the other agent during a same period of prevention, occurrence of a disorder, and/or period of treatment.

Pharmaceutical compositions of the disclosure can be administered in combination therapy, i.e., combined with other agents. In certain embodiments, the combination therapy comprises the RNase-Fc fusion protein, in combination with at least one other agent. Agents include, but are not limited to, in vitro synthetically prepared chemical compositions, antibodies, antigen binding regions, and combinations and conjugates thereof. In certain embodiments, an agent can act as an agonist, antagonist, allosteric modulator, or toxin.

In certain embodiments, the disclosure provides for pharmaceutical compositions comprising a RNase-Fc fusion protein together with a pharmaceutically acceptable diluent, carrier, solubilizer, emulsifier, preservative and/or adjuvant.

In certain embodiments, the disclosure provides for pharmaceutical compositions comprising a RNase-Fc fusion protein and a therapeutically effective amount of at least one additional therapeutic agent, together with a pharmaceutically acceptable diluent, carrier, solubilizer, emulsifier, preservative and/or adjuvant.

In certain embodiments, acceptable formulation materials preferably are nontoxic to recipients at the dosages and concentrations employed. In some embodiments, the formulation material(s) are for s.c. and/or I.V. administration. In certain embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as gelatin); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington’s Pharmaceutical Sciences, 18th Edition, A. R. Gennaro, ed., Mack Publishing Company (1995). In some embodiments, the formulation comprises PBS; 20 mM NaOAC, pH 5.2, 50 mM NaCl; and/or 10 mM NAOAC, pH 5.2, 9% Sucrose.

In certain embodiments, a RNase-Fc fusion protein and/or a therapeutic molecule is linked to a half-life extending vehicle known in the art. Such vehicles include, but are not limited to, polyethylene glycol, glycogen (e.g., glycosylation of the RNase-Fc fusion protein), and dextran. Such vehicles are described, e.g., in U.S. Application Ser. No. 09/428,082, now U.S. Pat. No. 6,660,843 and published PCT Application No. WO 99/25044.

In certain embodiments, the optimal pharmaceutical composition will be determined by one skilled in the art depending upon, for example, the intended route of administration, delivery format and desired dosage. See, for example, Remington’s Pharmaceutical Sciences, supra. In certain embodiments, such compositions may influence the physical state, stability, rate of in vivo release and rate of in vivo clearance of the fusion proteins of the disclosure.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In some embodiments, the saline comprises isotonic phosphate-buffered saline. In certain embodiments, pharmaceutical compositions comprise Tris buffer of about pH 7.0-8.5, or acetate buffer of about H 4.0-5.5, which can further include sorbitol or a suitable substitute therefore. In certain embodiments, a composition comprising an RNase-Fc fusion protein, with or without at least one additional therapeutic agents, can be prepared for storage by mixing the selected composition having the desired degree of purity with optional formulation agents (Remington’s Pharmaceutical Sciences, supra) in the form of a lyophilized cake or an aqueous solution. Further, in certain embodiments, a composition comprising an RNase-Fc fusion protein, with or without at least one additional therapeutic agent, can be formulated as a lyophilizate using appropriate excipients such as sucrose.

In certain embodiments, the pharmaceutical composition can be selected for parenteral delivery. In certain embodiments, the compositions can be selected for inhalation or for delivery through the digestive tract, such as orally. The preparation of such pharmaceutically acceptable compositions is within the ability of one skilled in the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments, when parenteral administration is contemplated, a therapeutic composition can be in the form of a pyrogen-free, parenterally acceptable aqueous solution comprising a desired RNase-Fc fusion protein, with or without additional therapeutic agents, in a pharmaceutically acceptable vehicle. In certain embodiments, a vehicle for parenteral injection is sterile distilled water in which an RNase-Fc fusion protein, with or without at least one additional therapeutic agent, is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

In certain embodiments, a pharmaceutical composition can be formulated for inhalation. In certain embodiments, an RNase-Fc fusion protein, with or without at least one additional therapeutic agent, can be formulated as a dry powder for inhalation. In certain embodiments, an inhalation solution comprising an RNase-Fc fusion protein, with or without at least one additional therapeutic agent, can be formulated with a propellant for aerosol delivery. In certain embodiments, solutions can be nebulized. Pulmonary administration is further described in PCT application no. PCT/US94/001875, which describes pulmonary delivery of chemically modified proteins.

In certain embodiments, it is contemplated that formulations can be administered orally. In certain embodiments, an RNase-Fc fusion protein, with or without at least one additional therapeutic agents, that is administered in this fashion can be formulated with or without those carriers customarily used in the compounding of solid dosage forms such as tablets and capsules. In certain embodiments, a capsule can be designed to release the active portion of the formulation at the point in the gastrointestinal tract when bioavailability is maximized and pre-systemic degradation is minimized. In certain embodiments, at least one additional agent can be included to facilitate absorption of an RNase-Fc fusion protein and/or any additional therapeutic agents. In certain embodiments, diluents, flavorings, low melting point waxes, vegetable oils, lubricants, suspending agents, tablet disintegrating agents, and binders can also be employed.

In certain embodiments, a pharmaceutical composition can involve an effective quantity of an RNase-Fc fusion protein, with or without at least one additional therapeutic agents, in a mixture with non-toxic excipients which are suitable for the manufacture of tablets. In certain embodiments, by dissolving the tablets in sterile water, or another appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving an RNase-Fc fusion protein, with or without at least one additional therapeutic agent(s), in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In certain embodiments, sustained-release preparations can include semipermeable polymer matrices in the form of shaped articles, e.g. films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al, Biopolymers, 22:547-556 (1983)), poly (2-hydroxyethyl-methacrylate) (Langer et al., J Biomed Mater Res, 15: 167-277 (1981) and Langer, Chem Tech, 12:98-105 (1982)), ethylene vinyl acetate (Langer et al, supra) or poly-D(-)-3-hydroxybutyric acid (EP 133,988). In certain embodiments, sustained release compositions can also include liposomes, which can be prepared by any of several methods known in the art. See, e.g., Eppstein et al, PNAS, 82:3688-3692 (1985); EP 036,676; EP 088,046 and EP 143,949.

The pharmaceutical composition to be used for in vivo administration typically is sterile. In certain embodiments, this can be accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In certain embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included. In certain embodiments, the effective amount of a pharmaceutical composition comprising an RNase-Fc fusion protein, with or without at least one additional therapeutic agent, to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which an RNase-Fc fusion protein, with or without at least one additional therapeutic agent, is being used, the route of administration, and the size (body weight, body surface or organ size) and/or condition (the age and general health) of the patient. In certain embodiments, the clinician can titer the dosage and modify the route of administration to obtain the optimal therapeutic effect. In certain embodiments, a typical dosage can range from about 0.5 mg/kg to up to about 50 mg/kg or more, depending on the factors mentioned above. In certain embodiments, the dosage can range from about 5-10 mg/kg, about 2-8 mg/kg, about 3-6 mg/kg, about 3 mg/kg, about 5 mg/kg, or about 10 mg/kg.

In certain embodiments, the frequency of dosing will take into account the pharmacokinetic parameters of an RNase-Fc fusion protein and/or any additional therapeutic agents in the formulation used. In certain embodiments, a clinician will administer the composition until a dosage is reached that achieves the desired effect. In certain embodiments, the composition can therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In certain embodiments, the route of administration of the pharmaceutical composition is in accord with known methods, e.g. orally, through injection by intravenous, intraperitoneal, intracerebral (intra-parenchymal), intracerebroventricular, intramuscular, subcutaneously, intra-ocular, intraarterial, intraportal, or intralesional routes; by sustained release systems or by implantation devices. In certain embodiments, the compositions can be administered by bolus injection or continuously by infusion, or by implantation device.

In certain embodiments, the composition can be administered locally via implantation of a membrane, sponge or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration.

In certain embodiments, it can be desirable to use a pharmaceutical composition comprising an RNase-Fc fusion protein, with or without at least one additional therapeutic agent, in an ex vivo manner. In such instances, cells, tissues and/or organs that have been removed from the patient are exposed to a pharmaceutical composition comprising an RNase-Fc fusion protein, with or without at least one additional therapeutic agent, after which the cells, tissues and/or organs are subsequently implanted back into the patient.

In certain embodiments, an RNase-Fc fusion protein and/or any additional therapeutic agents can be delivered by implanting certain cells that have been genetically engineered, using methods such as those described herein, to express and secrete the polypeptides. In certain embodiments, such cells can be animal or human cells, and can be autologous, heterologous, or xenogeneic. In certain embodiments, the cells can be immortalized. In certain embodiments, in order to decrease the chance of an immunological response, the cells can be encapsulated to avoid infiltration of surrounding tissues. In certain embodiments, the encapsulation materials are typically biocompatible, semi-permeable polymeric enclosures or membranes that allow the release of the protein product(s) but prevent the destruction of the cells by the patient’s immune system or by other detrimental factors from the surrounding tissues.

### In Vitro Assays

Various in vitro assays known in the art can be used to assess the efficacy of the RNase-containing nuclease fusion proteins of the disclosure, including RNase-Fc fusion proteins.

For example, cultured human PBMCs from normal subjects, lupus patient PBMCs, or Sjogren’s PBMCs are isolated, cultured, and treated with various stimuli (e.g., TLR ligands, costimulatory antibodies, immune complexes, and normal or autoimmune sera), in the presence or absence of the RNase-Fc fusion proteins. Cytokine production by the stimulated cells can be measured using commercially available reagents, such as the antibody pair kits from Biolegend (San Diego, CA) for various cytokines (e.g., IL-6, IL-8, IL-10, IL-4, IFN-gamma, and TNF-alpha). Culture supernatants are harvested at various time points as appropriate for the assay (e.g., 24, 48 hours, or later time points) to determine the effects that the RNase-Fc fusion proteins have on cytokine production. IFN-alpha production is measured using, e.g., anti-human IFN-alpha antibodies and standard curve reagents available from PBL interferon source (Piscataway, NJ). Similar assays are performed using human lymphocyte subpopulations (isolated monocytes, B cells, pDCs, T cells, etc.); purified using, e.g., commercially available magnetic bead based isolation kits available from Miltenyi Biotech (Auburn, CA).

Multi-color flow cytometry can be used to assess the effects of the RNase-Fc fusion proteins on immune cell activation by measuring the expression of lymphocyte activation receptors such as CD5, CD23, CD69, CD80, CD86, and CD25 in PBMCs or isolated cell subpopulations at various time points after stimulation using routine art-recognized methods.

The efficacy of RNase-Fc fusion proteins can also be tested by incubating SLE or Sjogren’s patient serum with normal human pDCs to activate IFN output, as described in, e.g., Ahlin et al., Lupus 2012:21:586-95; Mathsson et al., Clin Expt Immunol 2007;147:513-20; and Chiang et al., J Immunol 2011;186:1279-1288. Without being bound by theory, circulating nucleic acid-containing immune complexes in SLE or Sjogren’s patient sera facilitate nucleic acid antigen entry into pDC endosomes via Fc receptor-mediated endocytosis, followed by binding of nucleic acids to and activation of endosomal TLRs 7, 8, and 9. To assess the impact of the RNase-Fc fusion proteins, SLE or Sjogren’s patient sera or plasma is pretreated with the RNase-Fc fusion proteins, followed by addition to cultures of pDC cells isolated from healthy volunteers. Levels of IFN-α produced are then determined at multiple time points. By degrading nucleic-acid containing immune complexes, effective RNase-Fc fusion proteins are expected to reduce the quantity of IFN-α produced.

The effectiveness of RNase-Fc fusion proteins is demonstrated by comparing the results of an assay from cells treated with an RNase-Fc fusion protein disclosed herein to the results of the assay from cells treated with control formulations. After treatment, the levels of the various markers (e.g., cytokines, cell-surface receptors, proliferation) described above are generally improved in an effective RNase-Fc fusion protein treated group relative to the marker levels existing prior to the treatment, or relative to the levels measured in a control group.

### Methods of Treatment

The RNase-containing nuclease fusion proteins of the disclosure, including RNase-Fc fusion proteins of the disclosure are particularly effective in the treatment of autoimmune disorders or abnormal immune responses. In this regard, it will be appreciated that the RNase-containing nuclease fusion proteins of the disclosure, including RNase-Fc fusion proteins of the present disclosure are used to control, suppress, modulate, treat, or eliminate unwanted immune responses to both external and autoantigens. In some embodiments, the RNase-containing nuclease fusion proteins of the disclosure, including RNase-Fc fusion protein of the disclosure are used to treat or reduce fatigue in a patient with an autoimmune disorder. In some embodiments, the fatigue is Sjogren’s syndrome associated fatigue.

In some aspects, the RNase-containing nuclease fusion proteins of the disclosure, including RNase-Fc fusion proteins are useful to treat an autoimmune disease, in a human patient by administering an RNase-containing nuclease fusion protein of the disclosure, such as RNase-Fc fusion protein in an effective amount or a sufficient amount to the human patient in need thereof, thereby treating the disease. Any route of administration suitable for achieving the desired effect is contemplated by the disclosure (e.g., intravenous, intramuscular, subcutaneous). Treatment of the disease condition may result in a decrease in the symptoms associated with the condition, which may be long-term or short-term, or even a transient beneficial effect. In some embodiments, treatment of Sjogren’s syndrome results in a decrease in fatigue associated with the disease or condition.

### Biochemical Assays

In some embodiments, an RNase-containing nuclease fusion protein of the disclosure, including an RNase-Fc fusion protein is administered to human patients in need thereof to treat Sjogren’s syndrome. In some aspects, the effectiveness of an RNase-Fc fusion protein is demonstrated by comparing the IFN-alpha levels, IFN-alpha response gene levels, autoantibody titers, kidney function and pathology, and/or circulating immune complex levels in human patients treated with an RNase-Fc fusion protein disclosed herein compared to placebo.

For example, a human subject in need of treatment is selected or identified (e.g., a patient who fulfills the American-European Consensus Sjogren’s Classification Criteria). The subject can be in need of, e.g., reducing a cause or symptom of Sjogren’s syndrome, e.g., pSS. In some embodiments, the patient has Sjogren’s syndrome and is in need of reducing fatigue. The identification of the subject can occur in a clinical setting, or elsewhere, e.g., in the subject’s home through the subject’s own use of a self-testing kit.

At baseline (day 1), a suitable first dose of an RNase-containing nuclease fusion protein of the disclosure, including an RNase-Fc fusion protein is administered to the patient in need thereof. The RNase-Fc fusion protein is formulated as described herein. The patient’s condition is evaluated at baseline (day 1) and after a period of time following the first dose, e.g., day 8, day 15, day 29, day 43, day 57, day 71, day 85, day 99 or at the end of the study, e.g., by measuring IFN-alpha levels, IFN-alpha response gene levels, autoantibody titers, kidney function and pathology, and/or circulating immune complex levels. Other relevant criteria can also be measured. The number and strength of doses are adjusted according to the subject’s needs. After treatment, the subject’s IFN-alpha levels, IFN-alpha response gene levels, autoantibody titers, kidney function and pathology, and/or circulating immune complex levels are lowered and/or improved relative to the levels existing prior to the treatment, or relative to the levels measured in a similarly afflicted but untreated/control subject.

### Fatigue Assays

Various patient reported outcome (PRO) instruments have been used and validated in the measurement of fatigue in subjects with chronic diseases. Such PROs are known in the art and can be used to assess the efficacy of RNase-containing nuclease fusion proteins of the disclosure, including RNase-Fc fusion proteins.

### EULAR Sjogren’s Syndrome Disease Activity Index (ESSDAI)

The European League Against Rheumatism (EULAR) Sjogren’s Syndrome Disease Activity Index (ESSDAI) was developed to measure disease activity in patients with primary Sjogren’s syndrome (SS). ESSDAI includes 12 domains (ie, organ systems: cutaneous, respiratory, renal, articular, muscular, peripheral nervous system (PNS), central nervous system (CNS), hematological, glandular, constitutional, lymphadenopathic, biological). Each domain is divided into 3-4 levels of activity. The definition of each activity level is provided by a detailed description of what should be considered in that domain. Possible scores range between 0 -123 and about eighty percent of patients have a score ≤13. Moderately active disease is an ESSDAI of ≥5 and a minimal clinically important improvement (MCII) in ESSDAI is a decrease of at least three points.

In some embodiments, the effectiveness of RNase-containing nuclease fusion proteins of the disclosure, including the RNase-Fc fusion proteins or pharmaceutical compositions thereof is demonstrated by assessing an improvement in fatigue in patients following treatment with an RNase-containing nuclease fusion protein of the disclosure, including an RNase-Fc fusion protein or pharmaceutical composition thereof. In some embodiments, fatigue is reduced in the patient as measured by the ESSDAI when compared to the level of fatigue in the patient prior to treatment, and/or when compared to a patient treated with a control formulation. In some embodiments, fatigue is generally reduced in the patient as measured by the ESSDAI when compared to the level of fatigue in a healthy subject.

In some embodiments, the effectiveness of RNase-containing nuclease fusion proteins of the disclosure, including the RNase-Fc fusion proteins or pharmaceutical compositions thereof is demonstrated by assessing an improvement in disease activity as measured by ESSDAI in patients following treatment with an RNase-containing nuclease fusion protein of the disclosure, including an RNase-Fc fusion protein or pharmaceutical composition thereof. In some embodiments, disease activity is reduced as measured by ESSDAI when compared to the level of disease activity as measured by ESSDAI in the patient prior to treatment, and/or when compared to a patient treated with a control formulation. In some embodiments, fatigue is generally reduced in the patient as measured by the ESSDAI when compared to the level of fatigue in a healthy subject.

### EULAR Sjogren’s Syndrome Patient Reported Index (ESSPRI)

The European League Against Rheumatism (EULAR) Sjogren’s Syndrome (SS) Patient Reported Index (ESSPRI) was developed to assess the symptoms of patients with primary Sjogren’s syndrome (Seror et al., Ann. Rheum. Dis. 2011;70:968-972). ESSPRI was developed as a global score to measure all important and disabling symptoms of primary Sjogren’s syndrome: dryness, limb pain, and fatigue. ESSPRI has been shown to be sufficient to measure each of the symptoms without loss of content validity and the score is easy to calculate.

The ESSPRI is a patient-administered questionnaire that assess the symptoms of patients with primary Sjogren’s syndrome. The questionnaire includes three scales, one for each of the following symptoms: (1) dryness, (2) limb pain, and (3) fatigue. Each component of the ESSPRI is measured with a single 0-10 numerical scale and the global ESSPRI score is the mean of the three scales: (dryness + limb pain + fatigue)/3. A decrease of at least one point in the ESSPRI score is clinically meaningful.

In some embodiments, the effectiveness of RNase-containing nuclease fusion proteins of the disclosure, including the RNase-Fc fusion proteins or pharmaceutical compositions thereof is demonstrated by assessing an improvement in fatigue in patients following treatment with an RNase-containing nuclease fusion protein of the disclosure, including an RNase-Fc fusion protein or pharmaceutical composition thereof. After treatment, fatigue is generally reduced in the patient as measured by the ESSPRI when compared to the level of fatigue in the patient prior to treatment, and/or when compared to a patient treated with a control formulation. In some embodiments, fatigue is generally reduced in the patient as measured by the ESSPRI when compared to the level of fatigue in a healthy subject.

In some embodiments, the effectiveness of RNase-containing nuclease fusion proteins of the disclosure, including the RNase-Fc fusion proteins or pharmaceutical compositions thereof is demonstrated by assessing an improvement in disease activity as measured by ESSPRI in patients following treatment with an RNase-containing nuclease fusion protein of the disclosure, including an RNase-Fc fusion protein or pharmaceutical composition thereof. After treatment, disease activity is generally reduced in the patient as measured by the ESSPRI when compared to the level of disease activity as measured by ESSPRI in the patient prior to treatment, and/or when compared to a patient treated with a control formulation. In some embodiments, fatigue is generally reduced in the patient as measured by the ESSPRI when compared to the level of fatigue in a healthy subject. In some embodiments, disease activity is measured by the ESSPRI scale, which assesses the symptoms of patients with primary Sjogren’s syndrome. In some embodiments, the ESSPRI questionnaire includes the assessment of the following symptoms: (1) dryness, (2) pain, and (3) fatigue.

### FACIT-Fatigue

The Functional Assessment of Chronic Illness Therapy Fatigue scale (FACIT-Fatigue) is used to assess an individual’s level of fatigue during their usual daily activities over the past week. The FACIT-Fatigue questionnaire and Scoring & Interpretation Materials are available from FACIT.org (Elmhurst, Ill., USA). The FACIT-Fatigue questionnaire provides an array of generic and targeted measures. The FACIT fatigue scale has many benefits including high internal validity, high test-retest reliability, reliability and sensitivity to change in patients with a variety of chronic health conditions, ease of use, and use in a variety of settings. (K.F. Tennant, Try This: best Practices in Nursing Care to Older Adults, Issue 30, 2012; Chandran et al., Ann. Rheum. Dis. 2007; 66: 936-939).

The FACIT-Fatigue is a 13-item questionnaire originally developed to measure fatigue in patients with cancer and is now use in patients with Sjogren’s syndrome to measure fatigue. The patient is asked to answer 13 questions scored from 0 to 4 (0=not at all, 1=a little bit, 2=somewhat, 3=quite a bit, 4=very much). The fatigue scale has 13 items, with 52 as the highest possible score. A higher score in the fatigue scale corresponds to a lower level of fatigue and indicates better quality of life.

To calculate the FACIT-fatigue score, the response scores on negatively phrased questions are reversed and then the 13 item responses are added. Eleven items with responses have their scores reversed (item score=4-response, if the response is not missing), and two items (items 7-8) have their responses unchanged. All items are added so that higher scores correspond to less fatigue. In cases where individual questions are skipped, scores are prorated using the average of other answers in the scale.

$$\begin{matrix}\text{FACIT-Fatigue=} \\ \text{13*} \left[\text{sum} \left(\text{reversed items}\right) + \text{sum} \left(\text{items 7-8}\right)\right] / \text{number of answered items}\end{matrix}$$

In some embodiments, the effectiveness of RNase-containing nuclease fusion proteins of the disclosure, including RNase-Fc fusion proteins or pharmaceutical compositions thereof is demonstrated by assessing an improvement in fatigue in patients treated with an RNase-containing nuclease fusion protein of the disclosure, including an RNase-Fc fusion protein or pharmaceutical composition thereof. After treatment, fatigue is generally reduced in the patient as measured by the FACIT fatigue scale when compared to the level of fatigue in the patient prior to treatment, and/or when compared to a patient treated with a control formulation.

### Profile of Fatigue

The Profile of Fatigue (ProF) was developed to establish an assessment tool that was effective in characterizing fatigue associated with primary Sjogren’s syndrome. Therefore, the words patients with primary Sjogren’s syndrome use to express their complaints of fatigue, discomfort, and pain were used in the ProF questionnaire. The ProF has been shown to be a reliable and valid instrument for measuring the severity of fatigue and general discomfort in patients with primary Sjogren’s syndrome.

The ProF is a 16 item self-administered questionnaire divided into two domains, one for somatic fatigue and one for mental fatigue. The somatic fatigue domain includes 12 items divided into four facets: (a) need rest (four items), (b) poor starting (three items), (c) low stamina (three items), and (d) weak muscles (two items). The mental fatigue domain includes 4 items is divided into two facets: (a) poor concentration (two items) and (b) poor memory (two items). Patient’s score each item on a scale of 0-7 (0 = ‘no problem at all’ and 7 = “as bad as imaginable’) based on how the patient felt at their worst over the past two weeks. The score for each facet can be obtained by adding up the item scores within each facet and dividing the sum by the number of items in each facet. The score for each domain (e.g., somatic, mental) can be obtained by adding up the facet scores within each domain and dividing the sum by the number of facets within each domain. Higher scores indicates greater fatigue. (Bowman et al., Rheumatology, 2004; 43: 758-764; Strombeck et al., Scand. J. Rheumatol. 2005;34:455-459; Segal et al., Arthritis Rheum. 2008 Dec. 15; 59(12):1780-1787).

In some embodiments, the effectiveness of RNase-containing nuclease fusion proteins of the disclosure, including RNase-Fc fusion proteins or pharmaceutical compositions thereof is demonstrated by assessing an improvement in fatigue in patients treated with an RNase-containing nuclease fusion protein of the disclosure, including an RNase-Fc fusion protein or pharmaceutical composition thereof. After treatment, fatigue is generally reduced in the patient as measured by PROF when compared to the level of fatigue in the patient prior to treatment, and/or when compared to a patient treated with a control formulation.

### Assessing a Reduction in Sjogren’s Syndrome Associate Fatigue

In some embodiments, the effectiveness of an RNase-containing nuclease fusion protein of the disclosure, including an RNase-Fc fusion protein is demonstrated by assessing a reduction of fatigue in patients treated with the RNase-containing nuclease fusion protein, including the RNase-Fc fusion protein. In some embodiments, a patient treated with an RNase-Fc fusion protein will demonstrate a reduction in fatigue when compared to the level of fatigue in the patient prior to treatment, and/or when compared to a patient treated with a control formulation. In some embodiments, the fatigue is Sjogren’s syndrome associated fatigue.

In some embodiments, the patient’s condition is evaluated by measuring fatigue in the patient by one or more patient reported indices (e.g., ESSPRI, PROF, FACIT) as compared to the level of fatigue in the patient prior to treatment or relative to the levels of fatigue in a similarly afflicted untreated or control patient. In some embodiments, the effectiveness of a RNase-Fc fusion protein is demonstrated by assessing the EULAR SS Patient Reported Index (ESSPRI), the Profile or Fatigue (PROF) and/or the Functional Assessment of Chronic Illness Therapy (FACIT) fatigue scale in patients treated with a RNase-Fc fusion protein disclosed herein when compared to patients treated with control formulations. In some embodiments, a patient treated with a RNase-Fc fusion protein will demonstrate an improvement in the ESSPRI index, PROF, and/or the FACIT fatigue scale when compared to the patient’s ESSPRI index, PROF, and/or the FACIT fatigue scale prior to the treatment, or when compared to a patient treated with a control formulation.

For example, a human subject in need of treatment is selected or identified (e.g., a patient who fulfills the American College of Rheumatology criteria for SLE, or a patient who fulfills the American-European Consensus Sjogren’s Classification Criteria). The subject can be in need of, e.g., reducing a cause or symptom of SLE or Sjogren’s syndrome, such as fatigue. The identification of the subject can occur in a clinical setting, or elsewhere, e.g., in the subject’s home through the subject’s own use of a self-testing kit.

At baseline (day 1), a suitable first dose of a RNase-Fc fusion is administered to the subject. The RNase-Fc fusion protein is formulated as described herein. The patient’s condition is evaluated at baseline (day 1) and after a period of time following the first dose, e.g., day 8, day 15, day 29, day 43, day 57, day 71, day 85, day 99 or at the end of the study, e.g., by ESSPRI index, PROF, and/or the FACIT fatigue scale. Other relevant criteria can also be measured. The number and strength of doses are adjusted according to the subject’s needs. After treatment, an improvement in one or more of the following outcomes can be noted: (1) an improvement in the ESSPRI index relative to the ESSPRI index prior to treatment, or relative to a similarly afflicted but untreated/control subject, (2) an improvement in the PROF relative to the PROF prior to treatment, or relative to a similarly afflicted but untreated/control subject, (3) an improvement can be noted in the FACIT fatigue scale relative to the FACIT fatigue scale prior to treatment, or relative to a similarly afflicted but untreated/control subject. In some embodiments, an improvement in the ESSPRI index is a clinically meaningful improvement. A clinically meaningful improvement in the ESSPRI index is a decrease of at least one point in the ESSPRI score.

### Neuropsychological Analysis of Fatigue Assays

Various neuropsychological assays known in the art can be used to assess the efficacy of RNase-containing nuclease fusion proteins of the disclosure, including RNase-Fc fusion proteins.

### Digit Symbol Substitution Test

The Digit Symbol Substitution Test (DSST) provides a valid and sensitive test to measure cognitive dysfunction that is impacted by many domains. The DSST is sensitive to both the presence of cognitive dysfunction as well as a change in cognitive function across a range of clinical populations, including patients with Sjogren’s Syndrome. This neuropsychological test is widely used, highly validated, and extremely sensitive test reading out on executive function related inputs.

DSST is a time limited paper-and-pencil cognitive test that is given on a single sheet of paper. The test requires a patient to match symbols to numbers according to a key at the top of the paper. The patient copies the symbol into spaces below a row of numbers and the number of correct symbols within the allowed time (e.g., 90 or 120 seconds) is calculated. The test provides data on the accuracy and rate of performing the task. A patients performance on the DSST correlates with real-world functional outcomes, such as the ability to accomplish everyday tasks, and recovery from functional disability in a range of psychiatric conditions. The DSST test can be used to assess attention and/or focus in the patient.

DSST is a polyfactorial test that measures a range of cognitive operations and provides a practical and effective method to monitor cognitive function over time. To perform well on the DSST the patient must have intact motor speed, attention and visuoperceptual functions, including scanning and the ability to write or draw (i.e., basic mental dexterity). DSST offers high sensitivity to detect cognitive impairment and has many benefits including brevity, reliability, sensitivity to change, and minimal impact on language, culture and education on test performance. (Jaeger, J., Journal of Clinical Psycopharmacology, 38(5), 513-518, October 2018).

Furthermore, DSST is used in clinical development to define pharmacokinetic/pharmacodynamic (PK/PD) relationship. This test is also used as a PD biomarker in CNS studies and can discriminate between two doses of selective serotonin reuptake inhibitors (SSRI).

In some embodiments, the effectiveness of an RNase-containing nuclease fusion protein of the disclosure, including an RNase-Fc fusion proteins or pharmaceutical composition thereof is demonstrated by assessing an improvement in cognitive function in patients treated with an RNase-containing nuclease fusion protein of the disclosure, including an RNase-Fc fusion protein or pharmaceutical composition thereof. After treatment, cognitive function is generally improved in the patient as measured by the DSST test when compared to the level of cognitive function in the patient prior to treatment, and/or when compared to a patient treated with a control formulation.

### Assessing Improvement in Sjogren’s Syndrome Associated Cognitive Function

In some embodiments, the effectiveness of RNase-containing nuclease fusion proteins of the disclosure, including RNase-Fc fusion proteins is demonstrated by assessing an improvement in cognitive function in patients treated with an RNase-containing nuclease fusion proteins of the disclosure, including an RNase-Fc fusion protein. In some embodiments, a patient treated with an RNase-Fc fusion protein demonstrates an improvement in cognitive function when compared to the level of cognitive function in the patient prior to treatment, and/or when compared to a patient treated with a control formulation. In some embodiments, the patient has Sjogren’s syndrome.

In some embodiments, the patient’s condition is evaluated by measuring cognitive function in the patient by one or more neuropsychological assays (e.g., DSST) as compared to the level of cognitive function in the patient prior to treatment or relative to the level of cognitive function in a similarly afflicted untreated or control patient. In some embodiments, the effectiveness of a RNase-Fc fusion protein is demonstrated by assessing the results of a DSST test in patients treated with a RNase-Fc fusion protein disclosed herein when compared to patients treated with control formulations. In some embodiments, a patient treated with a RNase-Fc fusion protein will demonstrate an improvement in the DSST test when compared to the patient’s DSST test score prior to the treatment, or when compared to a patient treated with a control formulation.

For example, a human subject in need of treatment is selected or identified (e.g., a patient who fulfills the American-European Consensus Sjogren’s Classification Criteria). The subject can be in need of, e.g., reducing a cause or symptom of Sjogren’s syndrome, such as cognitive dysfunction. The identification of the subject can occur in a clinical setting, or elsewhere, e.g., in the subject’s home through the subject’s own use of a self-testing kit.

At baseline (day 1), a suitable first dose of a RNase-Fc fusion is administered to the subject. The RNase-Fc fusion protein is formulated as described herein. The patient’s condition is evaluated at baseline (day 1) and after a period of time following the first dose, e.g., day 8, day 15, day 29, day 43, day 57, day 71, day 85, day 99 or at the end of the study, e.g., by DSST. Other relevant criteria can also be measured. The number and strength of doses are adjusted according to the subject’s needs. After treatment, an improvement in the DSST test score relative to the DSST test score prior to treatment, or relative to a similarly afflicted but untreated/control subject.

### Patient Criteria

In some embodiments, a patient is a patient with primary Sjogren’s syndrome. In some embodiments, a patient is a patient with Sjogren’s syndrome associated fatigue. In some embodiments, the patient is a patient with fatigue. In some embodiments, the patient is a human patient. In some embodiments, the patient has elevated levels of anti-Ro52/60 autoantibodies. In some embodiments, the patient has a positive IFN signature. In some embodiments, the patient has elevated levels of anti-Ro52/60 antibodies and a positive IFN signature.

In some embodiments, a positive IFN signature is elevated expression levels of HERC5, CMPK2, and EPSTI1. In some embodiments, a positive IFN signature is elevated expression levels of HERC5, CMPK2, and EPSTI1, wherein the expression levels are 2 standard deviations above the values in healthy subjects. In some embodiments, a positive IFN signature is elevated expression levels of BERC5. In some embodiments, a positive IFN signature is elevated expression levels of HERC5, wherein the expression levels are 2 standard deviations above the values in healthy subjects. In some embodiments, a positive IFN signature is elevated expression levels of CMPK2. In some embodiments, a positive IFN signature is elevated expression levels of CMPK2, wherein the expression levels are 2 standard deviations above the values in healthy subjects. In some embodiments, a positive IFN signature is elevated expression levels of EPSTI1. In some embodiments, a positive IFN signature is elevated expression levels of EPSTI1, wherein the expression levels are 2 standard deviations above the values in healthy subjects. In some embodiments, a positive IFN signature is elevated expression levels of CMPK2, and EPSTI1. In some embodiments, a positive IFN signature is elevated expression levels of CMPK2, and EPSTI1, wherein the expression levels are 2 standard deviations above the values in healthy subjects. In some embodiments, a positive IFN signature is elevated expression levels of BERC5 and EPSTI1. In some embodiments, a positive IFN signature is elevated expression levels of BERC5 and EPSTI1, wherein the expression levels are 2 standard deviations above the values in healthy subjects. In some embodiments, a positive IFN signature is elevated expression levels of BERC5 and CMPK2. In some embodiments, a positive IFN signature is elevated expression levels of BERC5 and CMPK, wherein the expression levels are 2 standard deviations above the values in healthy subjects.

### Biomarkers

In some embodiments a patient with Sjogren’s syndrome has elevated levels of one or more biomarkers as compared to a healthy subject. In some embodiments a patient with Sjogren’s syndrome associated fatigue has elevated levels of one or more biomarkers as compared to a healthy subject. In some embodiments a patient with fatigue has elevated levels of one or more biomarkers as compared to a healthy subject. In some embodiments a patient with Sjogren’s syndrome has reduced levels of one or more biomarkers as compared to a healthy subject. In some embodiments a patient with Sjogren’s syndrome associated fatigue has reduced levels of one or more biomarkers as compared to a healthy subject. In some embodiments a patient with fatigue has reduced levels of one or more biomarkers as compared to a healthy subject. In some embodiments a patient with Sjogren’s syndrome has elevated levels of one or more biomarkers and reduced levels of one or more biomarkers as compared to a healthy subject. In some embodiments a patient with Sjogren’s syndrome associated fatigue has elevated levels of one or more biomarkers and reduced levels of one or more biomarkers as compared to a healthy subject. In some embodiments a patient with fatigue has elevated levels of one or more biomarkers and reduced levels of one or more biomarkers as compared to a healthy subject.

In some embodiments, the biomarker is anti-Ro52/60 autoantibodies.

In some embodiments, the biomarker is complement C3 (C3).

In some embodiments, the biomarker is complement C4 (C4).

In some embodiments, the biomarker is complement C3 (C3) and complement C4 (C4).

In some embodiments, the biomarker is immunoglobulin G (IgG) levels (mg/dL).

In some embodiments, the biomarker is erythrocyte sedimentation rate (ESR).

In some embodiments, the biomarkers are anti-Ro52/60 autoantibodies and C3.

In some embodiments, the biomarkers are anti-Ro52/60 autoantibodies and C4.

In some embodiments, the biomarkers are anti-Ro52/60 autoantibodies, C3 and C4.

In some embodiments, the biomarkers are anti-Ro52/60 autoantibodies and IgG.

In some embodiments, the biomarkers are anti-Ro52/60 autoantibodies and ESR.

In some embodiments, the biomarkers are C3 and IgG.

In some embodiments, the biomarkers are C3 and ESR.

In some embodiments, the biomarkers are C4 and IgG.

In some embodiments, the biomarkers are C4 and ESR.

In some embodiments, the biomarkers are C3, C4 and IgG.

In some embodiments, the biomarkers are C3, C4 and ESR.

In some embodiments, the biomarkers are IgG and ESR.

In some embodiments, the biomarkers are anti-Ro52/60 autoantibodies, C3 and IgG.

In some embodiments, the biomarkers are anti-Ro52/60 autoantibodies, C3 and ESR.

In some embodiments, the biomarkers are anti-Ro52/60 autoantibodies, C4 and IgG.

In some embodiments, the biomarkers are anti-Ro52/60 autoantibodies, C4 and ESR.

In some embodiments, the biomarkers are anti-Ro52/60 autoantibodies, IgG and ESR.

In some embodiments, the biomarkers are anti-Ro52/60 autoantibodies, C3, C4, and IgG.

In some embodiments, the biomarkers are anti-Ro52/60 autoantibodies, C3, C4, and ESR.

In some embodiments, the biomarkers are anti-Ro52/60 autoantibodies, C3, IgG, and ESR.

In some embodiments, the biomarkers are anti-Ro52/60 autoantibodies, C4, IgG, and ESR.

In some embodiments, the biomarkers are C3, C4, IgG, and ESR.

In some embodiments, the biomarkers are anti-Ro52/60 autoantibodies, C3, C4, IgG, and ESR.

In some embodiments a patient with Sjogren’s syndrome has increased levels of anti-Ro52/60 autoantibodies as compared to a healthy subject. In some embodiments a patient with Sjogren’s syndrome has reduced levels of C3 as compared to a healthy subject. In some embodiments a patient with Sjogren’s syndrome has reduced levels of C4 as compared to a healthy subject. In some embodiments a patient with Sjogren’s syndrome has reduced levels of C3 and C4 as compared to a healthy subject. In some embodiments a patient with Sjogren’s syndrome has increased ESR as compared to a healthy subject. In some embodiments a patient with Sjogren’s syndrome has reduced levels of IgG as compared to a healthy subject. In some embodiments a patient with Sjogren’s syndrome has increased levels of IgG as compared to a healthy subject. In some embodiments a patient with Sjogren’s syndrome has no change in IgG levels as compared to a healthy subject.

In some embodiments a patient with Sjogren’s syndrome has increased levels of anti-Ro52/60 autoantibodies and reduced levels of C3 as compared to a healthy subject. In some embodiments a patient with Sjogren’s syndrome has increased levels of anti-Ro52/60 autoantibodies and reduced levels of C4 as compared to a healthy subject. In some embodiments a patient with Sjogren’s syndrome has increased levels of anti-Ro52/60 autoantibodies and reduced levels of C3 and C4 as compared to a healthy subject. In some embodiments a patient with Sjogren’s syndrome has increased levels of anti-Ro52/60 autoantibodies and increased ESR as compared to a healthy subject.

In some embodiments a patient with Sjogren’s syndrome has reduced levels of C3 and increased ESR as compared to a healthy subject. In some embodiments a patient with Sjogren’s syndrome has reduced levels of C4 and increased ESR as compared to a healthy subject. In some embodiments a patient with Sjogren’s syndrome has reduced levels of C3 and C4 and increased ESR as compared to a healthy subject.

In some embodiments a patient with Sjogren’s syndrome has increased levels of anti-Ro52/60 autoantibodies, reduced levels of C3 and C4, and increased ESR as compared to a healthy subject.

In some embodiments a patient with Sjogren’s syndrome has increased levels of anti-Ro52/60 autoantibodies and reduced levels of IgG as compared to a healthy subject. In some embodiments a patient with Sjogren’s syndrome has increased levels of anti-Ro52/60 autoantibodies and increased levels of IgG as compared to a healthy subject.

In some embodiments a patient with Sjogren’s syndrome has reduced levels of C3 and reduced levels of IgG as compared to a healthy subject. In some embodiments a patient with Sjogren’s syndrome has reduced levels of C3 and increased levels of IgG as compared to a healthy subject.

In some embodiments a patient with Sjogren’s syndrome has reduced levels of C4 and reduced levels of IgG as compared to a healthy subject. In some embodiments a patient with Sjogren’s syndrome has reduced levels of C4 and increased levels of IgG as compared to a healthy subject.

In some embodiments a patient with Sjogren’s syndrome has reduced levels of C3 and C4 and reduced levels of IgG as compared to a healthy subject. In some embodiments a patient with Sjogren’s syndrome has reduced levels of C3 and C4 and increased levels of IgG as compared to a healthy subject.

In some embodiments a patient with Sjogren’s syndrome has increased levels of ESR and reduced levels of IgG as compared to a healthy subject. In some embodiments a patient with Sjogren’s syndrome has increased levels of ESR and increased levels of IgG as compared to a healthy subject.

In some embodiments a patient with Sjogren’s syndrome has increased levels of anti-Ro52/60 autoantibodies, reduced levels of C3 and reduced levels of IgG as compared to a healthy subject. In some embodiments a patient with Sjogren’s syndrome has increased levels of anti-Ro52/60 autoantibodies, reduced levels of C3 and increased levels of IgG as compared to a healthy subject.

In some embodiments a patient with Sjogren’s syndrome has increased levels of anti-Ro52/60 autoantibodies, reduced levels of C4 and reduced levels of IgG as compared to a healthy subject. In some embodiments a patient with Sjogren’s syndrome has increased levels of anti-Ro52/60 autoantibodies, reduced levels of C4 and increased levels of IgG as compared to a healthy subject.

In some embodiments a patient with Sjogren’s syndrome has increased levels of anti-Ro52/60 autoantibodies, reduced levels of C3 and C4 and reduced levels of IgG as compared to a healthy subject. In some embodiments a patient with Sjogren’s syndrome has increased levels of anti-Ro52/60 autoantibodies, reduced levels of C3 and C4 and increased levels of IgG as compared to a healthy subject.

In some embodiments a patient with Sjogren’s syndrome has increased levels of anti-Ro52/60 autoantibodies, increased levels ESR and reduced levels of IgG as compared to a healthy subject. In some embodiments a patient with Sjogren’s syndrome has increased levels of anti-Ro52/60 autoantibodies, increased levels ESR and increased levels of IgG as compared to a healthy subject.

In some embodiments a patient with Sjogren’s syndrome has reduced levels of C3, increased levels of ESR, and reduced levels of IgG as compared to a healthy subject. In some embodiments a patient with Sjogren’s syndrome has reduced levels of C3, increase levels of ESR, and increased levels of IgG as compared to a healthy subject.

In some embodiments a patient with Sjogren’s syndrome has reduced levels of C4, increased levels of ESR, and reduced levels of IgG as compared to a healthy subject. In some embodiments a patient with Sjogren’s syndrome has reduced levels of C4, increase levels of ESR, and increased levels of IgG as compared to a healthy subject.

In some embodiments a patient with Sjogren’s syndrome has reduced levels of C3 and C4, increased levels of ESR, and reduced levels of IgG as compared to a healthy subject. In some embodiments a patient with Sjogren’s syndrome has reduced levels of C3 and C4, increase levels of ESR, and increased levels of IgG as compared to a healthy subject.

In some embodiments a patient with Sjogren’s syndrome has increased levels of anti-Ro52/60 autoantibodies, increased levels ESR, reduced levels of C3, and reduced levels of IgG as compared to a healthy subject. In some embodiments a patient with Sjogren’s syndrome has increased levels of anti-Ro52/60 autoantibodies, increased levels ESR, reduced levels of C3 and increased levels of IgG as compared to a healthy subject.

In some embodiments a patient with Sjogren’s syndrome has increased levels of anti-Ro52/60 autoantibodies, increased levels ESR, reduced levels of C4, and reduced levels of IgG as compared to a healthy subject. In some embodiments a patient with Sjogren’s syndrome has increased levels of anti-Ro52/60 autoantibodies, increased levels ESR, reduced levels of C4 and increased levels of IgG as compared to a healthy subject.

In some embodiments a patient with Sjogren’s syndrome has increased levels of anti-Ro52/60 autoantibodies, increased levels ESR, reduced levels of C3 and C4, and reduced levels of IgG as compared to a healthy subject. In some embodiments a patient with Sjogren’s syndrome has increased levels of anti-Ro52/60 autoantibodies, increased levels ESR, reduced levels of C3 and C4 and increased levels of IgG as compared to a healthy subject.

In some embodiments, treatment with an RNase-containing nuclease fusion protein (for example, an RNase-Fc) increases expression of one or more biomarkers and decreases expression of one or more biomarkers.

In some embodiments, treatment with an RNase-containing nuclease fusion protein (for example, an RNase-Fc) decreases expression of one or more biomarkers. In some embodiments, treatment with an RNase-containing nuclease fusion protein (for example, an RNase-Fc) decreases anti-Ro52/60 autoantibodies. In some embodiments, treatment with an RNase-containing nuclease fusion protein (for example, an RNase-Fc) decreases complement C3 (C3). In some embodiments, treatment with an RNase-containing nuclease fusion protein (for example, an RNase-Fc) decreases complement C4 (C4). In some embodiments, treatment with an RNase-containing nuclease fusion protein (for example, an RNase-Fc) decreases complement C3 (C3) and complement C4 (C4). In some embodiments, treatment with an RNase-containing nuclease fusion protein (for example, an RNase-Fc) decreases immunoglobulin G (IgG) levels (mg/dL). In some embodiments, treatment with an RNase-containing nuclease fusion protein (for example, an RNase-Fc) decreases erythrocyte sedimentation rate (ESR). In some embodiments, treatment with an RNase-containing nuclease fusion protein (for example, an RNase-Fc) decreases anti-Ro52/60 autoantibodies and C3. In some embodiments, treatment with an RNase-containing nuclease fusion protein (for example, an RNase-Fc) decreases anti-Ro52/60 autoantibodies and C4. In some embodiments, treatment with an RNase-containing nuclease fusion protein (for example, an RNase-Fc) decreases anti-Ro52/60 autoantibodies, C3 and C4. In some embodiments, treatment with an RNase-containing nuclease fusion protein (for example, an RNase-Fc) decreases anti-Ro52/60 autoantibodies and IgG. In some embodiments, treatment with an RNase-containing nuclease fusion protein (for example, an RNase-Fc) decreases anti-Ro52/60 autoantibodies and ESR. In some embodiments, treatment with an RNase-containing nuclease fusion protein (for example, an RNase-Fc) decreases C3 and IgG. In some embodiments, treatment with an RNase-containing nuclease fusion protein (for example, an RNase-Fc) decreases C3 and ESR. In some embodiments, treatment with an RNase-containing nuclease fusion protein (for example, an RNase-Fc) decreases C4 and IgG. In some embodiments, treatment with an RNase-containing nuclease fusion protein (for example, an RNase-Fc) decreases C4 and ESR. In some embodiments, treatment with an RNase-containing nuclease fusion protein (for example, an RNase-Fc) decreases C3, C4 and IgG. In some embodiments, treatment with an RNase-containing nuclease fusion protein (for example, an RNase-Fc) decreases C3, C4 and ESR. In some embodiments, treatment with an RNase-containing nuclease fusion protein (for example, an RNase-Fc) decreases IgG and ESR. In some embodiments, treatment with an RNase-containing nuclease fusion protein (for example, an RNase-Fc) decreases anti-Ro52/60 autoantibodies, C3 and IgG. In some embodiments, treatment with an RNase-containing nuclease fusion protein (for example, an RNase-Fc) decreases anti-Ro52/60 autoantibodies, C3 and ESR. In some embodiments, treatment with an RNase-containing nuclease fusion protein (for example, an RNase-Fc) decreases anti-Ro52/60 autoantibodies, C4 and IgG. In some embodiments, treatment with an RNase-containing nuclease fusion protein (for example, an RNase-Fc) decreases anti-Ro52/60 autoantibodies, C4 and ESR. In some embodiments, treatment with an RNase-containing nuclease fusion protein (for example, an RNase-Fc) decreases anti-Ro52/60 autoantibodies, IgG and ESR. In some embodiments, treatment with an RNase-containing nuclease fusion protein (for example, an RNase-Fc) decreases anti-Ro52/60 autoantibodies, C3, C4, and IgG. In some embodiments, treatment with an RNase-containing nuclease fusion protein (for example, an RNase-Fc) decreases anti-Ro52/60 autoantibodies, C3, C4, and ESR. In some embodiments, treatment with an RNase-containing nuclease fusion protein (for example, an RNase-Fc) decreases anti-Ro52/60 autoantibodies, C3, IgG, and ESR. In some embodiments, treatment with an RNase-containing nuclease fusion protein (for example, an RNase-Fc) decreases anti-Ro52/60 autoantibodies, C4, IgG, and ESR. In some embodiments, treatment with an RNase-containing nuclease fusion protein (for example, an RNase-Fc) decreases C3, C4, IgG, and ESR. In some embodiments, treatment with an RNase-containing nuclease fusion protein (for example, an RNase-Fc) decreases anti-Ro52/60 autoantibodies, C3, C4, IgG, and ESR.

In some embodiments, treatment with an RNase-containing nuclease fusion protein (for example, an RNase-Fc) increases expression of one or more biomarkers. In some embodiments, treatment with an RNase-containing nuclease fusion protein (for example, an RNase-Fc) increases anti-Ro52/60 autoantibodies. In some embodiments, treatment with an RNase-containing nuclease fusion protein (for example, an RNase-Fc) increases complement C3 (C3). In some embodiments, treatment with an RNase-containing nuclease fusion protein (for example, an RNase-Fc) increases complement C4 (C4). In some embodiments, treatment with an RNase-containing nuclease fusion protein (for example, an RNase-Fc) increases complement C3 (C3) and complement C4 (C4). In some embodiments, treatment with an RNase-containing nuclease fusion protein (for example, an RNase-Fc) increases immunoglobulin G (IgG) levels (mg/dL). In some embodiments, treatment with an RNase-containing nuclease fusion protein (for example, an RNase-Fc) increases erythrocyte sedimentation rate (ESR). In some embodiments, treatment with an RNase-containing nuclease fusion protein (for example, an RNase-Fc) increases anti-Ro52/60 autoantibodies and C3. In some embodiments, treatment with an RNase-containing nuclease fusion protein (for example, an RNase-Fc) increases anti-Ro52/60 autoantibodies and C4. In some embodiments, treatment with an RNase-containing nuclease fusion protein (for example, an RNase-Fc) increases anti-Ro52/60 autoantibodies, C3 and C4. In some embodiments, treatment with an RNase-containing nuclease fusion protein (for example, an RNase-Fc) increases anti-Ro52/60 autoantibodies and IgG. In some embodiments, treatment with an RNase-containing nuclease fusion protein (for example, an RNase-Fc) increases anti-Ro52/60 autoantibodies and ESR. In some embodiments, treatment with an RNase-containing nuclease fusion protein (for example, an RNase-Fc) increases C3 and IgG. In some embodiments, treatment with an RNase-containing nuclease fusion protein (for example, an RNase-Fc) increases C3 and ESR. In some embodiments, treatment with an RNase-containing nuclease fusion protein (for example, an RNase-Fc) increases C4 and IgG. In some embodiments, treatment with an RNase-containing nuclease fusion protein (for example, an RNase-Fc) increases C4 and ESR. In some embodiments, treatment with an RNase-containing nuclease fusion protein (for example, an RNase-Fc) increases C3, C4 and IgG. In some embodiments, treatment with an RNase-containing nuclease fusion protein (for example, an RNase-Fc) increases C3, C4 and ESR. In some embodiments, treatment with an RNase-containing nuclease fusion protein (for example, an RNase-Fc) increases IgG and ESR. In some embodiments, treatment with an RNase-containing nuclease fusion protein (for example, an RNase-Fc) increases anti-Ro52/60 autoantibodies, C3 and IgG. In some embodiments, treatment with an RNase-containing nuclease fusion protein (for example, an RNase-Fc) increases anti-Ro52/60 autoantibodies, C3 and ESR. In some embodiments, treatment with an RNase-containing nuclease fusion protein (for example, an RNase-Fc) increases anti-Ro52/60 autoantibodies, C4 and IgG. In some embodiments, treatment with an RNase-containing nuclease fusion protein (for example, an RNase-Fc) increases anti-Ro52/60 autoantibodies, C4 and ESR. In some embodiments, treatment with an RNase-containing nuclease fusion protein (for example, an RNase-Fc) increases anti-Ro52/60 autoantibodies, IgG and ESR. In some embodiments, treatment with an RNase-containing nuclease fusion protein (for example, an RNase-Fc) increases anti-Ro52/60 autoantibodies, C3, C4, and IgG. In some embodiments, treatment with an RNase-containing nuclease fusion protein (for example, an RNase-Fc) increases anti-Ro52/60 autoantibodies, C3, C4, and ESR. In some embodiments, treatment with an RNase-containing nuclease fusion protein (for example, an RNase-Fc) increases anti-Ro52/60 autoantibodies, C3, IgG, and ESR. In some embodiments, treatment with an RNase-containing nuclease fusion protein (for example, an RNase-Fc) increases anti-Ro52/60 autoantibodies, C4, IgG, and ESR. In some embodiments, treatment with an RNase-containing nuclease fusion protein (for example, an RNase-Fc) increases C3, C4, IgG, and ESR. In some embodiments, treatment with an RNase-containing nuclease fusion protein (for example, an RNase-Fc) increases anti-Ro52/60 autoantibodies, C3, C4, IgG, and ESR.

In some embodiments, treatment with an RNase-containing nuclease fusion protein (for example, an RNase-Fc) increases levels of C3 and decreases ESR in a subject. In some embodiments, treatment with an RNase-containing nuclease fusion protein (for example, an RNase-Fc) increases levels of C4 and decreases ESR in a subject. In some embodiments, treatment with an RNase-containing nuclease fusion protein (for example, an RNase-Fc) increases levels of C3 and C4 and decreases ESR in a subject. In some embodiments, treatment with an RNase-containing nuclease fusion protein (for example, an RNase-Fc) increases levels of IgG and decreases ESR in a subject. In some embodiments, treatment with an RNase-containing nuclease fusion protein (for example, an RNase-Fc) decreases levels of IgG and decreases ESR in a subject.

In some embodiments, treatment with an RNase-containing nuclease fusion protein (for example, an RNase-Fc) decreases levels of anti-Ro52/60 autoantibodies and increases levels of C3 in a subject. In some embodiments, treatment with an RNase-containing nuclease fusion protein (for example, an RNase-Fc) decreases levels of anti-Ro52/60 autoantibodies and increases levels of C4 in a subject. In some embodiments, treatment with an RNase-containing nuclease fusion protein (for example, an RNase-Fc) decreases levels of anti-Ro52/60 autoantibodies and increases levels of C3 and C4 in a subject. In some embodiments, treatment with an RNase-containing nuclease fusion protein (for example, an RNase-Fc) decreases levels of anti-Ro52/60 autoantibodies and decreases ESR in a subject. In some embodiments, treatment with an RNase-containing nuclease fusion protein (for example, an RNase-Fc) decreases levels of anti-Ro52/60 autoantibodies and increases levels of IgG in a subject. In some embodiments, treatment with an RNase-containing nuclease fusion protein (for example, an RNase-Fc) decreases levels of anti-Ro52/60 autoantibodies and decreases levels of IgG in a subject.

In some embodiments, treatment with an RNase-containing nuclease fusion protein (for example, an RNase-Fc) increases levels of IgG and increases levels of C3 in a subject. In some embodiments, treatment with an RNase-containing nuclease fusion protein (for example, an RNase-Fc) decreases levels of IgG and increases levels of C3 in a subject. In some embodiments, treatment with an RNase-containing nuclease fusion protein (for example, an RNase-Fc) increases levels of IgG and increases levels of C4 in a subject. In some embodiments, treatment with an RNase-containing nuclease fusion protein (for example, an RNase-Fc) decreases levels of IgG and increases levels of C4 in a subject. In some embodiments, treatment with an RNase-containing nuclease fusion protein (for example, an RNase-Fc) decreases levels of IgG and increases levels of C3 and C4 in a subject. In some embodiments, treatment with an RNase-containing nuclease fusion protein (for example, an RNase-Fc) increases levels of IgG and increases levels of C3 and C4 in a subject.

In some embodiments, treatment with an RNase-containing nuclease fusion protein (for example, an RNase-Fc) decreases levels of anti-Ro52/60 autoantibodies, increases levels of C3, and decreases levels of ESR in a subject. In some embodiments, treatment with an RNase-containing nuclease fusion protein (for example, an RNase-Fc) decreases levels of anti-Ro52/60 autoantibodies, increases levels of C4 and decreases levels of ESR in a subject. In some embodiments, treatment with an RNase-containing nuclease fusion protein (for example, an RNase-Fc) decreases levels of anti-Ro52/60 autoantibodies, decreases levels of ESR and increases levels of C3 and C4 in a subject. In some embodiments, treatment with an RNase-containing nuclease fusion protein (for example, an RNase-Fc) decreases levels of anti-Ro52/60 autoantibodies, decreases levels of ESR and increases levels of IgG in a subject. In some embodiments, treatment with an RNase-containing nuclease fusion protein (for example, an RNase-Fc) decreases levels of anti-Ro52/60 autoantibodies, decreases levels of ESR, and decreases levels of IgG in a subject. In some embodiments, treatment with an RNase-containing nuclease fusion protein (for example, an RNase-Fc) decreases levels of anti-Ro52/60 autoantibodies, increases levels of C3 and increases levels of IgG in a subject. In some embodiments, treatment with an RNase-containing nuclease fusion protein (for example, an RNase-Fc) decreases levels of anti-Ro52/60 autoantibodies, increases levels of C3, and decreases levels of IgG in a subject. In some embodiments, treatment with an RNase-containing nuclease fusion protein (for example, an RNase-Fc) decreases levels of anti-Ro52/60 autoantibodies, increases levels of C4 and increases levels of IgG in a subject. In some embodiments, treatment with an RNase-containing nuclease fusion protein (for example, an RNase-Fc) decreases levels of anti-Ro52/60 autoantibodies, increases levels of C4, and decreases levels of IgG in a subject. In some embodiments, treatment with an RNase-containing nuclease fusion protein (for example, an RNase-Fc) decreases levels of anti-Ro52/60 autoantibodies, increases levels of C3 and C4 and increases levels of IgG in a subject. In some embodiments, treatment with an RNase-containing nuclease fusion protein (for example, an RNase-Fc) decreases levels of anti-Ro52/60 autoantibodies, increases levels of C3 and C4, and decreases levels of IgG in a subject.

In some embodiments, treatment with an RNase-containing nuclease fusion protein (for example, an RNase-Fc) decreases levels of anti-Ro52/60 autoantibodies, increases levels of C3, decreases levels of ESR and increases levels of IgG in a subject. In some embodiments, treatment with an RNase-containing nuclease fusion protein (for example, an RNase-Fc) decreases levels of anti-Ro52/60 autoantibodies, increases levels of C3, decreases levels of ESR, and decreases levels of IgG in a subject. In some embodiments, treatment with an RNase-containing nuclease fusion protein (for example, an RNase-Fc) decreases levels of anti-Ro52/60 autoantibodies, increases levels of C4, decreases levels of ESR and increases levels of IgG in a subject. In some embodiments, treatment with an RNase-containing nuclease fusion protein (for example, an RNase-Fc) decreases levels of anti-Ro52/60 autoantibodies, increases levels of C4, decreases levels of ESR, and decreases levels of IgG in a subject. In some embodiments, treatment with an RNase-containing nuclease fusion protein (for example, an RNase-Fc) decreases levels of anti-Ro52/60 autoantibodies, increases levels of C3 and C4, decreases levels of ESR and increases levels of IgG in a subject. In some embodiments, treatment with an RNase-containing nuclease fusion protein (for example, an RNase-Fc) decreases levels of anti-Ro52/60 autoantibodies, increases levels of C3 and C4, decreases levels of ESR, and decreases levels of IgG in a subject.

### Dosing Regimens

The RNase-containing nuclease fusion proteins of the disclosure, including RNase-Fc fusion proteins of the present disclosure and/or the pharmaceutical compositions of the present disclosure, are formulated into pharmaceutically-acceptable dosage forms for human subjects by conventional methods known to those of skill in the art. In some embodiments, actual dosage levels of the active ingredient (i.e., RNase-Fc fusion) in the pharmaceutical compositions of this disclosure are varied so as to obtain an amount of the active ingredient which is effective to achieve the desired therapeutic response for a particular human patient, composition, and/or mode of administration, without being unacceptably toxic to the patient.

The selected dosage level will depend upon a variety of factors including the activity of the particular RNase-Fc fusion protein, the route of administration, the time of administration, the rate of excretion or metabolism of the particular RNase-Fc fusion protein being administered, the rate and extent of absorption, the duration of the treatment, other drugs, compounds and/or materials used in combination with the particular RNase-Fc fusion protein administered, the age, sex, weight, condition, general health and prior medical history of the patient being treated, and like factors well known in the medical arts.

In general, a suitable dose of an RNase-Fc fusion protein or composition of the disclosure is an amount of the active ingredient which is the lowest dose effective to produce a therapeutic effect in the human subject. Such an effective dose will generally depend upon the factors described above. Generally, intravenous, oral, and subcutaneous doses of the RNase-Fc fusion protein or composition of this disclosure for a human patient, when used for the indicated effects, ranges from about 0.5 mg to about 50 mg per kilogram of body weight per week. In some embodiments, the RNase-Fc fusion protein or pharmaceutical composition of the disclosure is administered by injection (e.g., by intravenous injection, e.g., by infusion) to a human patient in need thereof in a dose of about 0.5 mg to about 50 mg per kilogram of body weight per week.

In some embodiments, the RNase-Fc fusion proteins or compositions of the present disclosure are administered in doses to human patients generally from about 1-20 mg/kg per week, 2-10 mg/kg per week, 5-15 mg/kg per week, 5-10 mg/kg per week, or 2-5 mg/kg per week. In some embodiments, doses of greater than 10 mg/kg, or greater than 15 mg/kg or greater than 20 mg/kg per week may be necessary. In some embodiments, doses of less than 20 mg/kg, or less than 15 mg/kg or less than 10 mg/kg per week may be necessary. In some embodiments, parenteral doses such as, for example, i.v. administration to a human patient are from about 5-10 mg/kg per week. In some embodiments, the RNase-Fc fusion protein is administered to human patients at a weekly, biweekly, monthly or semi-monthly (e.g., every 2 months, every 3 months) dose of about 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg 10 mg/kg, 11 mg/kg, 12 mg/kg, 14 mg/kg, 15 mg/kg, 16 mg/kg, 17 mg/kg, 18 mg/kg, 19 mg/kg 20 mg/kg, 21 mg/kg, 22 mg/kg, 23 mg/kg, 24 mg/kg 25 mg/kg. In some embodiments, the RNase-Fc fusion protein is administered to human patients at a weekly, biweekly, monthly or semi-monthly at a dose of 1 mg/kg. In some embodiments, the RNase-Fc fusion protein is administered to human patients at a weekly, biweekly, monthly or semi-monthly at a dose of 2 mg/kg. In some embodiments, the RNase-Fc fusion protein is administered to human patients at a weekly, biweekly, monthly or semi-monthly at a dose of 3 mg/kg. In some embodiments, the RNase-Fc fusion protein is administered to human patients at a weekly, biweekly, monthly or semi-monthly at a dose of 4 mg/kg. In some embodiments, the RNase-Fc fusion protein is administered to human patients at a weekly, biweekly, monthly or semi-monthly at a dose of 5 mg/kg. In some embodiments, the RNase-Fc fusion protein is administered to human patients at a weekly, biweekly, monthly or semi-monthly at a dose of 6 mg/kg. In some embodiments, the RNase-Fc fusion protein is administered to human patients at a weekly, biweekly, monthly or semi-monthly at a dose of 7 mg/kg. In some embodiments, the RNase-Fc fusion protein is administered to human patients at a weekly, biweekly, monthly or semi-monthly at a dose of 8 mg/kg. In some embodiments, the RNase-Fc fusion protein is administered to human patients at a weekly, biweekly, monthly or semi-monthly at a dose of 9 mg/kg. In some embodiments, the RNase-Fc fusion protein is administered to human patients at a weekly, biweekly, monthly or semi-monthly at a dose of 10 mg/kg. In some embodiments, the RNase-Fc fusion protein is administered to human patients at a weekly, biweekly, monthly or semi-monthly at a dose of 12 mg/kg. In some embodiments, the RNase-Fc fusion protein is administered to human patients at a weekly, biweekly, monthly or semi-monthly at a dose of 15 mg/kg. In some embodiments, the foregoing dose is formulated for intravenous injection.

If desired, the effective weekly, biweekly, monthly or semi-monthly dose of the RNase-Fc fusion protein or composition of the disclosure is administered to a human patient as two, three, four, five, six or more sub-doses administered separately at appropriate intervals throughout the day (e.g., as an intravenous injection or infusion), optionally, in unit dosage forms. In some embodiments, dosing is one administration per day. In some embodiments, dosing is one or more administrations per every 2, 3, 4, 5, 6, 7, 8, 9, 10, 11, 12, 13, 14 days, or every 1, 2, 3, 4, 5, 6, 7, 8, 9, 10, 11, 12 weeks, or every 1, 2, 3, 4, 5, 6, 7, 8, 9, 10, 11, 12 months, as needed, to obtain a therapeutic effect (e.g., sufficient to digest circulating RNA complexed with autoantibodies and/or RNA containing immune complexes). In some embodiments, dosing is one administration (e.g., intravenous injection or infusion) once every week. In some embodiments, dosing is one or more administrations once every two weeks. In some embodiments, dosing is one administration once every two weeks. In some embodiments, dosing is one or more administrations every month. In some embodiments, dosing is one administration every month. In some embodiments, dosing is one or more administrations semi-monthly (e.g., every 2 months, every 3 months). In some embodiments, dosing is one administration every 2 months or every 3 months. In some embodiments, the foregoing dose is formulated for intravenous injection.

In some embodiments, dosing is one administration (e.g., intravenous injection or infusion) once every week for two weeks, and then one administration every two weeks thereafter to achieve or maintain a therapeutic effect. In some embodiments, dosing is one administration once every week for three weeks, and then one administration every two weeks thereafter to achieve or maintain a therapeutic effect. In some embodiments, dosing is one administration once every week for four weeks, and then one administration every two weeks thereafter to achieve or maintain a therapeutic effect. In some embodiments, dosing is one administration once every week for two weeks, and then one administration every month thereafter to achieve or maintain a therapeutic effect. In some embodiments, dosing is one administration once every week for three weeks, and then one administration every month thereafter to achieve or maintain a therapeutic effect. In some embodiments, dosing is one administration once every week for four weeks, and then one administration every month thereafter to achieve or maintain a therapeutic effect. In some embodiments, the foregoing dose is formulated for intravenous injection. As used herein, an initial weekly administration followed by biweekly, monthly or semi-monthly dosing is referred to as a “loading dose.”

In some embodiments, the RNase-Fc fusion protein or composition of the disclosure is administered (e.g., by intravenous injection or infusion) weekly at a dose of about 1 mg/kg. In some embodiments, the RNase-Fc fusion protein or composition of the disclosure is administered weekly at a dose of about 2 mg/kg. In some embodiments, the RNase-Fc fusion protein or composition of the disclosure is administered weekly at a dose of about 3 mg/kg. In some embodiments, the RNase-Fc fusion protein or composition of the disclosure is administered weekly at a dose of about 4 mg/kg. In some embodiments, the RNase-Fc fusion protein or composition of the disclosure is administered weekly at a dose of about 5 mg/kg. In some embodiments, the RNase-Fc fusion protein or composition of the disclosure is administered weekly at a dose of about 6 mg/kg. In some embodiments, the RNase-Fc fusion protein or composition of the disclosure is administered weekly at a dose of about 7 mg/kg. In some embodiments, the RNase-Fc fusion protein or composition of the disclosure is administered weekly at a dose of about 8 mg/kg. In some embodiments, the RNase-Fc fusion protein or composition of the disclosure is administered weekly at a dose of about 9 mg/kg. In some embodiments, the RNase-Fc fusion protein or composition of the disclosure is administered weekly at a dose of about 10 mg/kg. In some embodiments, the RNase-Fc fusion protein or composition of the disclosure is administered weekly at a dose of about 12 mg/kg. In some embodiments, the RNase-Fc fusion protein or composition of the disclosure is administered weekly at a dose of about 15 mg/kg. In some embodiments, the foregoing dose is formulated for intravenous injection. As used herein, weekly is understood to have the art-accepted meaning of every week.

In some embodiments, the RNase-Fc fusion protein or composition of the disclosure is administered (e.g., by intravenous injection or infusion) biweekly at a dose of about 1 mg/kg. In some embodiments, the RNase-Fc fusion protein or composition of the disclosure is administered biweekly at a dose of about 2 mg/kg. In some embodiments, the RNase-Fc fusion protein or composition of the disclosure is administered biweekly at a dose of about 3 mg/kg. In some embodiments, the RNase-Fc fusion protein or composition of the disclosure is administered biweekly at a dose of about 4 mg/kg. In some embodiments, the RNase-Fc fusion protein or composition of the disclosure is administered biweekly at a dose of about 5 mg/kg. In some embodiments, the RNase-Fc fusion protein or composition of the disclosure is administered biweekly at a dose of about 6 mg/kg. In some embodiments, the RNase-Fc fusion protein or composition of the disclosure is administered biweekly at a dose of about 7 mg/kg. In some embodiments, the RNase-Fc fusion protein or composition of the disclosure is administered biweekly at a dose of about 8 mg/kg. In some embodiments, the RNase-Fc fusion protein or composition of the disclosure is administered biweekly at a dose of about 9 mg/kg. In some embodiments, the RNase-Fc fusion protein or composition of the disclosure is administered biweekly at a dose of about 10 mg/kg. In some embodiments, the RNase-Fc fusion protein or composition of the disclosure is administered biweekly at a dose of about 12 mg/kg. In some embodiments, the RNase-Fc fusion protein or composition of the disclosure is administered biweekly at a dose of about 15 mg/kg. In some embodiments, the foregoing dose is formulated for intravenous injection. As used herein, biweekly is understood to have the art-accepted meaning of every two weeks.

In some embodiments, the RNase-Fc fusion protein or composition of the disclosure is administered (e.g., by intravenous injection or infusion) every third week at a dose of about 1 mg/kg. In some embodiments, the RNase-Fc fusion protein or composition of the disclosure is administered every third week at a dose of about 2 mg/kg. In some embodiments, the RNase-Fc fusion protein or composition of the disclosure is administered every third week at a dose of about 3 mg/kg. In some embodiments, the RNase-Fc fusion protein or composition of the disclosure is administered every third week at a dose of about 4 mg/kg. In some embodiments, the RNase-Fc fusion protein or composition of the disclosure is administered every third week at a dose of about 5 mg/kg. In some embodiments, the RNase-Fc fusion protein or composition of the disclosure is administered every third week at a dose of about 6 mg/kg. In some embodiments, the RNase-Fc fusion protein or composition of the disclosure is administered every third week at a dose of about 7 mg/kg. In some embodiments, the RNase-Fc fusion protein or composition of the disclosure is administered every third week at a dose of about 8 mg/kg. In some embodiments, the RNase-Fc fusion protein or composition of the disclosure is administered every third week at a dose of about 9 mg/kg. In some embodiments, the RNase-Fc fusion proteins are administered every third week at a dose of about 10 mg/kg. In some embodiments, the RNase-Fc fusion protein or composition of the disclosure is administered every third week at a dose of about 12 mg/kg. In some embodiments, the RNase-Fc fusion protein or composition of the disclosure is administered every third week at a dose of about 15 mg/kg. In some embodiments, the foregoing dose is formulated for intravenous injection. As used herein, every third week is understood to have the art-accepted meaning of once every three weeks.

In some embodiments, the RNase-Fc fusion protein or composition of the disclosure is administered (e.g., by intravenous injection or infusion) monthly at a dose of about 1 mg/kg. In some embodiments, the RNase-Fc fusion protein or composition of the disclosure is administered monthly at a dose of about 2 mg/kg. In some embodiments, the RNase-Fc fusion protein or composition of the disclosure is administered monthly at a dose of about 3 mg/kg. In some embodiments, the RNase-Fc fusion protein or composition of the disclosure is administered monthly at a dose of about 4 mg/kg. In some embodiments, the RNase-Fc fusion protein or composition of the disclosure is administered monthly at a dose of about 5 mg/kg. In some embodiments, the RNase-Fc fusion protein or composition of the disclosure is administered monthly at a dose of about 6 mg/kg. In some embodiments, the RNase-Fc fusion protein or composition of the disclosure is administered monthly at a dose of about 7 mg/kg. In some embodiments, the RNase-Fc fusion protein or composition of the disclosure is administered monthly at a dose of about 8 mg/kg. In some embodiments, the RNase-Fc fusion protein or composition of the disclosure is administered monthly at a dose of about 9 mg/kg. In some embodiments, the RNase-Fc fusion protein or composition of the disclosure is administered monthly at a dose of about 10 mg/kg. In some embodiments, the RNase-Fc fusion protein or composition of the disclosure is administered monthly at a dose of about 12 mg/kg. In some embodiments, the RNase-Fc fusion protein or composition of the disclosure is administered monthly at a dose of about 15 mg/kg. In some embodiments, the foregoing dose is formulated for intravenous injection. As used herein, monthly is understood to have the art-accepted meaning of every month.

In some embodiments, the RNase-Fc fusion protein or composition of the disclosure is administered (e.g., by intravenous injection or infusion) weekly at a dose of about 2 mg/kg for two weeks, and then biweekly at a dose of about 2 mg/kg. In some embodiments, the RNase-Fc fusion protein or composition of the disclosure is administered weekly at a dose of about 2 mg/kg for three weeks, and then biweekly at a dose of about 2 mg/kg. In some embodiments, the RNase-Fc fusion protein or composition of the disclosure is administered weekly at a dose of about 2 mg/kg for four weeks, and then biweekly at a dose of about 2 mg/kg. In some embodiments, the RNase-Fc fusion protein or composition of the disclosure is administered weekly at a dose of about 2 mg/kg for two weeks, and then monthly at a dose of about 2 mg/kg. In some embodiments, the RNase-Fc fusion protein or composition of the disclosure is administered weekly at a dose of about 2 mg/kg for three weeks, and then monthly at a dose of about 2 mg/kg. In some embodiments, the RNase-Fc fusion protein or composition of the disclosure is administered weekly at a dose of about 2 mg/kg for four weeks, and then monthly at a dose of about 2 mg/kg. In some embodiments, the foregoing dose is formulated for intravenous injection.

In some embodiments, the RNase-Fc fusion protein or composition of the disclosure is administered (e.g., by intravenous injection or infusion) weekly at a dose of about 5 mg/kg for two weeks, and then biweekly at a dose of about 5 mg/kg. In some embodiments, the RNase-Fc fusion protein or composition of the disclosure is administered weekly at a dose of about 5 mg/kg for three weeks, and then biweekly at a dose of about 5 mg/kg. In some embodiments, the RNase-Fc fusion protein or composition of the disclosure is administered weekly at a dose of about 5 mg/kg for four weeks, and then biweekly at a dose of about 5 mg/kg. In some embodiments, the RNase-Fc fusion protein or composition of the disclosure is administered weekly at a dose of about 5 mg/kg for two weeks, and then monthly at a dose of about 5 mg/kg. In some embodiments, the RNase-Fc fusion protein or composition of the disclosure is administered weekly at a dose of about 5 mg/kg for three weeks, and then monthly at a dose of about 5 mg/kg. In some embodiments, the RNase-Fc fusion protein or composition of the disclosure is administered weekly at a dose of about 5 mg/kg for four weeks, and then monthly at a dose of about 5 mg/kg. In some embodiments, the foregoing dose is formulated for intravenous injection.

In some embodiments, the RNase-Fc fusion protein or composition of the disclosure is administered (e.g., by intravenous injection or infusion) weekly at a dose of about 10 mg/kg for two weeks, and then biweekly at a dose of about 10 mg/kg. In some embodiments, the RNase-Fc fusion protein or composition of the disclosure is administered weekly at a dose of about 10 mg/kg for three weeks, and then biweekly at a dose of about 10 mg/kg. In some embodiments, the RNase-Fc fusion protein or composition of the disclosure is administered weekly at a dose of about 10 mg/kg for four weeks, and then biweekly at a dose of about 10 mg/kg. In some embodiments, the RNase-Fc fusion protein or composition of the disclosure is administered weekly at a dose of about 10 mg/kg for two weeks, and then monthly at a dose of about 10 mg/kg. In some embodiments, the RNase-Fc fusion protein or composition of the disclosure is administered weekly at a dose of about 10 mg/kg for three weeks, and then monthly at a dose of about 10 mg/kg. In some embodiments, the RNase-Fc fusion protein or composition of the disclosure is administered weekly at a dose of about 10 mg/kg for four weeks, and then monthly at a dose of about 10 mg/kg. In some embodiments, the foregoing dose is formulated for intravenous injection.

As would be understood in the art, weekly, biweekly, every third week, or monthly administrations may be in one or more administrations or sub-doses as discussed above.

In one embodiment, an effective amount of an RNase-Fc fusion protein is about 2 mg/kg per human subject per week. In one embodiment, an effective amount of an RNase-Fc fusion protein is about 3 mg/kg per human subject per week. In one embodiment, an effective amount of an RNase-Fc fusion protein is about 4 mg/kg per human subject per week. In one embodiment, an effective amount of an RNase-Fc fusion protein is about 5 mg/kg per human subject per week. In one embodiment, an effective amount of an RNase-Fc fusion protein is about 6 mg/kg per human subject per week. In one embodiment, an effective amount of an RNase-Fc fusion protein is about 7 mg/kg per human subject per week. In one embodiment, an effective amount of an RNase-Fc fusion protein is about 8 mg/kg per human subject per week. In one embodiment, an effective amount of an RNase-Fc fusion protein is about 9 mg/kg per human subject per week. In one embodiment, an effective amount of an RNase-Fc fusion protein is about 10 mg/kg per human subject per week. In some embodiments, the foregoing dose is formulated for intravenous injection.

In one embodiment, an effective amount of an RNase-Fc fusion protein is about 2 mg/kg per human subject every 2 weeks. In one embodiment, an effective amount of an RNase-Fc fusion protein is about 3 mg/kg per human subject every 2 weeks. In one embodiment, an effective amount of an RNase-Fc fusion protein is about 4 mg/kg per human subject every 2 weeks. In one embodiment, an effective amount of an RNase-Fc fusion protein is about 5 mg/kg per human subject every 2 weeks. In one embodiment, an effective amount of an RNase-Fc fusion protein is about 6 mg/kg per human subject every 2 weeks. In one embodiment, an effective amount of an RNase-Fc fusion protein is about 7 mg/kg per human subject every 2 weeks. In one embodiment, an effective amount of an RNase-Fc fusion protein is about 8 mg/kg per human subject every 2 weeks. In one embodiment, an effective amount of an RNase-Fc fusion protein is about 9 mg/kg per human subject every 2 weeks. In one embodiment, an effective amount of an RNase-Fc fusion protein is about 10 mg/kg per human subject every 2 weeks. In some embodiments, the foregoing dose is formulated for intravenous injection.

### Methods and Uses of Interferon-Inducible Molecules

Provided herein are diagnostic and therapeutic methods and uses of the interferon-inducible molecules (for example, interferon-inducible genes, interferon-inducible proteins) described herein. Further provided herein are methods of identifying subjects having Sjogren’s syndrome that have achieved a response to treatment with an RNA nuclease agent as described herein by detecting the presence of or determining the amount or expression level of one or more interferon-inducible molecules (e.g., amount or expression level) in a sample obtained from the subject, wherein the presence of or the amount or expression level of the one or more interferon-inducible molecules indicates the subject achieved a response to the RNA nuclease agent.

### Interferon-Inducible Molecules

In some aspects, the disclosure provides methods for detecting the presence of or determining an amount or expression level of an interferon-inducible molecule (for example, an interferon-inducible gene) in a sample from a subject (e.g., a sample from a subject with Sjogren’s syndrome). In some aspects, the disclosure provides methods for treating a subject having Sjogren’s syndrome a with an RNase nuclease agent (e.g., RSLV-132), wherein treatment results in an increase in one or more interferon-inducible molecules (e.g., interferon-inducible genes).

As used herein, the term “interferon-inducible molecule” refers to a molecule that is regulated by interferon. In some embodiments, the interferon-inducible molecule is regulated by interferon-α. In some embodiments, the interferon-inducible molecule is regulated by interferon-β. In some embodiments, the interferon-inducible molecule is regulated by interferon-γ. In some embodiments, the interferon-inducible molecule is regulated by interferon-α and interferon-β. In some embodiments, an interferon-inducible molecule is an interferon-inducible gene. In some embodiments, an interferon-inducible molecule is an interferon-inducible protein. In some embodiments, the amount of an interferon-inducible molecule, such as an interferon-inducible protein or an interferon-inducible gene, is increased by interferon. In some embodiments, the activity of an interferon-inducible protein or an interferon-inducible gene, is increased by interferon. In some embodiments, interferon-inducible molecules (e.g., interferon inducible-proteins or interferon inducible genes) are used as surrogates for the interferon pathway. In some embodiments, expression of the interferon-inducible molecules (e.g., interferon inducible-proteins or interferon inducible genes) provide a readout on interferon-activity. In some embodiments, administration of an RNA nuclease agent to a subject with Sjogren’s syndrome results in an increase in expression of one or more interferon-inducible genes. In some embodiments, administration of an RNA nuclease agent to a subject with Sjogren’s syndrome results in an improvement in fatigue. In some embodiments, an RNA nuclease agent is RSLV-132.

In some embodiments, the expression level of one or more interferon-inducible molecules (e.g., interferon inducible-proteins or interferon inducible genes) in a sample from a patient with Sjogren’s syndrome is compared to the expression level of one or more interferon-inducible molecules (e.g., interferon inducible-proteins or interferon inducible genes) in a control. In some embodiments, a control is a sample from a healthy subject. In some embodiments, a control is a sample taken from the patient prior to treatment. In some embodiments, a control is a sample take from the patient prior to disease onset. In some embodiments, a control is a sample taken from a subject that did not respond to treatment.

In some embodiments, an interferon-inducible molecule is an interferon-inducible gene contained in at least one of the M1.2, M3.4, and M5.12 modules. The modules have been described by Chiche et al., Arthritis and Rheumatology, Vol 66, No. 6, 2014, 1583-1595. In some embodiments, an interferon-inducible gene is a gene contained in the M1.2 module. In some embodiments, an interferon-inducible gene is a gene contained in the M3.4 module. In some embodiments, an interferon-inducible gene is a gene contained in the M5.12 module. In some embodiments, an interferon-inducible gene is a gene regulated by interferon-α. In some embodiments, the interferon-inducible molecule is regulated by interferon-β. In some embodiments, the interferon-inducible molecule is regulated by interferonf-γ. In some embodiments, the interferon-inducible molecule is regulated by interferon-α and interferon-β. In some embodiments, an interferon-inducible gene is a surrogate for the interferon pathway. In some embodiments, expression of an interferon-inducible gene provides a readout on interferon activity. In some embodiments, an increase in expression of an interferon-inducible gene correlates with an increase in interferon activity.

In some embodiments, an interferon-inducible molecule is an interferon-inducible gene.

In some embodiments, one or more interferon-inducible genes are selected from LY6E, RTP4, EPSTI1, OAS1, OAS2, OASL, BATF2, SERPING1, OTOF, IFITM3, CXCL10, ISG15, HES4, XAF1, OAS3, IFIT3, RSAD2, LAMP3, IFIT1, IFI44L, HERC5, MX1, and IFI44.

In some embodiments, one or more interferon-inducible genes are selected from LY6E, RTP4, EPSTI1, OAS1, OAS2, OASL, BATF2, SERPING1, OTOF, IFITM3, ISG15, HES4, XAF1, OAS3, IFIT3, RSAD2, LAMP3, IFIT1, IFI44L, HERC5, MX1, and IFI44.

In some embodiments, one or more interferon-inducible genes are selected from LY6E, RTP4, OAS1, OAS2, OASL, BATF2, SERPING1, OTOF, IFITM3, CXCL10, ISG15, HES4, XAF1, OAS3, IFIT3, RSAD2, LAMP3, IFIT1, IFI44L, HERC5, MX1, and IFI44.

In some embodiments, one or more interferon-inducible genes are selected from LY6E, RTP4, OAS1, OAS2, OASL, BATF2, SERPING1, OTOF, IFITM3, ISG15, HES4, XAF1, OAS3, IFIT3, RSAD2, LAMP3, IFIT1, IFI44L, HERC5, MX1, and IFI44.

In some embodiments, one or more interferon-inducible genes are selected from LY6E, RTP4, EPSTI1, OAS1, OAS2, OASL, OTOF, CXCL10, ISG15, HES4, XAF1, OAS3, IFIT3, RSAD2, LAMP3, IFIT1, IFI44L, HERC5, MX1, and IFI44.

In some embodiments, one or more interferon-inducible genes are selected from LY6E, RTP4, OAS1, OAS2, OASL, OTOF, CXCL10, ISG15, HES4, XAF1, OAS3, IFIT3, RSAD2, LAMP3, IFIT1, IFI44L, HERC5, MX1, and IFI44.

In some embodiments, one or more interferon-inducible genes are selected from LY6E, RTP4, EPSTI1, OAS1, OAS2, OASL, OTOF, ISG15, HES4, XAF1, OAS3, IFIT3, RSAD2, LAMP3, IFIT1, IFI44L, HERC5, MX1, and IFI44.

In some embodiments, one or more interferon-inducible genes are selected from LY6E, RTP4, OAS1, OAS2, OASL, OTOF, ISG15, HES4, XAF1, OAS3, IFIT3, RSAD2, LAMP3, IFIT1, IFI44L, HERC5, MX1, and IFI44.

In some embodiments, one or more interferon-inducible genes are selected from LY6E, RTP4, EPSTI1, OAS1, OAS2, OASL, BATF2, OTOF, CXCL10, ISG15, HES4, XAF1, OAS3, IFIT3, RSAD2, LAMP3, IFIT1, IFI44L, HERC5, MX1, and IFI44.

In some embodiments, one or more interferon-inducible genes are selected from LY6E, RTP4, OAS1, OAS2, OASL, BATF2, OTOF, CXCL10, ISG15, HES4, XAF1, OAS3, IFIT3, RSAD2, LAMP3, IFIT1, IFI44L, HERC5, MX1, and IFI44.

In some embodiments, one or more interferon-inducible genes are selected from LY6E, RTP4, EPSTI1, OAS1, OAS2, OASL, BATF2, OTOF, ISG15, HES4, XAF1, OAS3, IFIT3, RSAD2, LAMP3, IFIT1, IFI44L, HERC5, MX1, and IFI44.

In some embodiments, one or more interferon-inducible genes are selected from LY6E, RTP4, OAS1, OAS2, OASL, BATF2, OTOF, ISG15, HES4, XAF1, OAS3, IFIT3, RSAD2, LAMP3, IFIT1, IFI44L, HERC5, MX1, and IFI44.

In some embodiments, one or more interferon-inducible genes are selected from LY6E, RTP4, EPSTI1, OAS1, OAS2, OASL, CXCL10, ISG15, HES4, XAF1, OAS3, IFIT3, RSAD2, LAMP3, IFIT1, IFI44L, HERC5, MX1, and IFI44.

In some embodiments, one or more interferon-inducible genes are selected from LY6E, RTP4, EPSTI1, OAS1, OAS2, OASL, ISG15, HES4, XAF1, OAS3, IFIT3, RSAD2, LAMP3, IFIT1, IFI44L, HERC5, MX1, and IFI44.

In some embodiments, one or more interferon-inducible genes are selected from LY6E, RTP4, OAS1, OAS2, OASL, CXCL10, ISG15, HES4, XAF1, OAS3, IFIT3, RSAD2, LAMP3, IFIT1, IFI44L, HERC5, MX1, and IFI44.

In some embodiments, one or more interferon-inducible genes are selected from LY6E, RTP4, OAS1, OAS2, OASL, ISG15, HES4, XAF1, OAS3, IFIT3, RSAD2, LAMP3, IFIT1, IFI44L, HERC5, MX1, and IFI44.

In some embodiments, an interferon-inducible gene is LY6E. In some embodiments, an interferon-inducible gene is RTP4. In some embodiments, an interferon-inducible gene is EPSTI1. In some embodiments, an interferon-inducible gene is OAS1. In some embodiments, an interferon-inducible gene is OAS2. In some embodiments, an interferon-inducible gene is OASL. In some embodiments, an interferon-inducible gene is BATF2. In some embodiments, an interferon-inducible gene is SERPING1. In some embodiments, an interferon-inducible gene is OTOF. In some embodiments, an interferon-inducible gene is IFITM3. In some embodiments, an interferon-inducible gene is CXCL10. In some embodiments, an interferon-inducible gene is ISG15. In some embodiments, an interferon-inducible gene is HES4. In some embodiments, an interferon-inducible gene is XAF1. In some embodiments, an interferon-inducible gene is OAS3. In some embodiments, an interferon-inducible gene is IFIT3. In some embodiments, an interferon-inducible gene is RSAD2. In some embodiments, an interferon-inducible gene is LAMP3. In some embodiments, an interferon-inducible gene is IFIT1. In some embodiments, an interferon-inducible gene is IFI44L. In some embodiments, an interferon-inducible gene is HERC5. In some embodiments, an interferon-inducible gene is MX1. In some embodiments, an interferon-inducible gene is IFI44.

In some embodiments, one or more interferon-inducible genes are selected from HERC6, ATF3, GBP3, GBP4, IFITM1, MT2A, TNFAIP6, LAP3, OAS2, PLSCR1, TNFSF10, TIMM10, UBE2L6, IFI35, AIM2, GALM, EIF2AK2, ZBP1, APOL6, STAT1, GBP5, LGALS3BP, SEPT4, FBXO6, SCO2, MOV10, CEACAM1, PML, WARS, CCL8, SOCS1, ZNF684, SAMD9L, PARP10, GBP1, IRF7, IFIT5, IFIH1, DHX58, IFIT2, PARP12, TRIM22, DDX58, STAT2, PARP9, and PARP14.

In some embodiments, one or more interferon-inducible genes are selected from HERC6, ATF3, GBP3, IFITM1, MT2A, TNFAIP6, LAP3, OAS2, PLSCR1, TNFSF10, TIMM10, UBE2L6, IFI35, AIM2, GALM, EIF2AK2, ZBP1, APOL6, GBP5, LGALS3BP, SEPT4, FBXO6, SCO2, MOV10, CEACAM1, PML, WARS, CCL8, SOCS1, ZNF684, SAMD9L, PARP10, GBP1, IRF7, IFIT5, IFIH1, DHX58, IFIT2, PARP12, DDX58, and PARP14.

In some embodiments, one or more interferon-inducible genes are selected from HERC6, ATF3, GBP3, IFITM1, TNFAIP6, LAP3, OAS2, PLSCR1, TNFSF10, TIMM10, UBE2L6, IFI35, AIM2, GALM, EIF2AK2, ZBP1, APOL6, STAT1, GBP5, CCL8, SOCS1, ZNF684, SAMD9L, PARP10, GBP1, IRF7, IFIT5, IFIH1, DHX58, IFIT2, PARP12, TRIM22, DDX58, STAT2, PARP9, and PARP14.

In some embodiments, one or more interferon-inducible genes are selected from HERC6, ATF3, GBP3, IFITM1, TNFAIP6, LAP3, OAS2, PLSCR1, TNFSF10, TIMM10, UBE2L6, IFI35, AIM2, GALM, EIF2AK2, ZBP1, APOL6, GBP5, CCL8, SOCS1, ZNF684, SAMD9L, PARP10, GBP1, IRF7, IFIT5, IFIH1, DHX58, IFIT2, PARP12, DDX58, and PARP14.

In some embodiments, one or more interferon-inducible genes are selected from HERC6, ATF3, GBP3, GBP4, IFITM1, MT2A, TNFAIP6, LAP3, OAS2, PLSCR1, TNFSF10, TIMM10, UBE2L6, IFI35, AIM2, GALM, EIF2AK2, ZBP1, APOL6, STAT1, GBP5, LGALS3BP, FBXO6, SCO2, CCL8, SOCS1, ZNF684, SAMD9L, PARP10, GBP1, IRF7, IFIT5, IFIH1, DHX58, IFIT2, PARP12, TRIM22, DDX58, STAT2, PARP9, and PARP14.

In some embodiments, one or more interferon-inducible genes are selected from HERC6, ATF3, GBP3, IFITM1, MT2A, TNFAIP6, LAP3, OAS2, PLSCR1, TNFSF10, TIMM10, UBE2L6, IFI35, AIM2, GALM, EIF2AK2, ZBP1, APOL6, GBP5, LGALS3BP, FBXO6, SCO2, CCL8, SOCS1, ZNF684, SAMD9L, PARP10, GBP1, IRF7, IFIT5, IFIH1, DHX58, IFIT2, PARP12, DDX58, and PARP14.

In some embodiments, one or more interferon-inducible genes are selected from HERC6, ATF3, GBP3, IFITM1, MT2A, TNFAIP6, LAP3, OAS2, PLSCR1, TNFSF10, TIMM10, IFI35, AIM2, GALM, EIF2AK2, ZBP1, STAT1, GBP5, CCL8, SOCS1, SAMD9L, PARP10, GBP1, IRF7, IFIT5, IFIH1, DHX58, IFIT2, PARP12, TRIM22, DDX58, STAT2, PARP9, and PARP14.

In some embodiments, one or more interferon-inducible genes are selected from HERC6, ATF3, GBP3, IFITM1, MT2A, TNFAIP6, LAP3, OAS2, PLSCR1, TNFSF10, TIMM10, IFI35, AIM2, GALM, EIF2AK2, ZBP1, GBP5, CCL8, SOCS1, SAMD9L, PARP10, GBP1, IRF7, IFIT5, IFIH1, DHX58, IFIT2, PARP12, DDX58, and PARP14.

In some embodiments, one or more interferon-inducible genes are selected from HERC6, ATF3, GBP3, GBP4, IFITM1, MT2A, TNFAIP6, LAP3, OAS2, PLSCR1, TNFSF10, TIMM10, UBE2L6, IFI35, AIM2, EIF2AK2, ZBP1, APOL6, STAT1, SEPT4, SCO2, CCL8, SOCS1, ZNF684, SAMD9L, PARP10, GBP1, IRF7, IFIT5, IFIH1, DHX58, IFIT2, PARP12, TRIM22, DDX58, STAT2, PARP9, and PARP14.

In some embodiments, one or more interferon-inducible genes are selected from HERC6, ATF3, GBP3, IFITM1, MT2A, TNFAIP6, LAP3, OAS2, PLSCR1, TNFSF10, TIMM10, UBE2L6, IFI35, AIM2, EIF2AK2, ZBP1, APOL6, SEPT4, SCO2, CCL8, SOCS1, ZNF684, SAMD9L, PARP10, GBP1, IRF7, IFIT5, IFIH1, DHX58, IFIT2, PARP12, DDX58, and PARP14.

In some embodiments, one or more interferon-inducible genes are selected from HERC6, ATF3, IFITM1, MT2A, TNFAIP6, LAP3, OAS2, PLSCR1, TNFSF10, IFI35, EIF2AK2, ZBP1, APOL6, STAT1, SEPT4, SCO2, CCL8, SOCS1, ZNF684, SAMD9L, PARP10, IRF7, IFIT5, IFIH1, DHX58, IFIT2, PARP12, TRIM22, DDX58, PARP9, and PARP14.

In some embodiments, one or more interferon-inducible genes are selected from HERC6, ATF3, IFITM1, MT2A, TNFAIP6, LAP3, OAS2, PLSCR1, TNFSF10, IFI35, EIF2AK2, ZBP1, APOL6, SEPT4, SCO2, CCL8, SOCS1, ZNF684, SAMD9L, PARP10, IRF7, IFIT5, IFIH1, DHX58, IFIT2, PARP12, DDX58, and PARP14.

In some embodiments, one or more interferon-inducible genes are selected from HERC6, ATF3, GBP3, IFITM1, MT2A, TNFAIP6, LAP3, OAS2, PLSCR1, TNFSF10, IFI35, EIF2AK2, ZBP1, STAT1, SEPT4, SCO2, CCL8, SOCS1, ZNF684, SAMD9L, PARP10, IRF7, IFIT5, IFIH1, DHX58, IFIT2, PARP12, TRIM22, DDX58, PARP9, and PARP14.

In some embodiments, one or more interferon-inducible genes are selected from HERC6, ATF3, GBP3, IFITM1, MT2A, TNFAIP6, LAP3, OAS2, PLSCR1, TNFSF10, IFI35, EIF2AK2, ZBP1, SEPT4, SCO2, CCL8, SOCS1, ZNF684, SAMD9L, PARP10, IRF7, IFIT5, IFIH1, DHX58, IFIT2, PARP12, DDX58, and PARP14.

In some embodiments, an interferon-inducible gene is HERC6. In some embodiments, an interferon-inducible gene is ATF3. In some embodiments, an interferon-inducible gene is GBP3. In some embodiments, an interferon-inducible gene is GBP4. In some embodiments, an interferon-inducible gene is IFITM1. In some embodiments, an interferon-inducible gene is MT2A. In some embodiments, an interferon-inducible gene is TNFAIP6. In some embodiments, an interferon-inducible gene is LAP3. In some embodiments, an interferon-inducible gene is OAS2. In some embodiments, an interferon-inducible gene is PLSCR1. In some embodiments, an interferon-inducible gene is TNFSF10. In some embodiments, an interferon-inducible gene is TIMM10. In some embodiments, an interferon-inducible gene is UBE2L6. In some embodiments, an interferon-inducible gene is IFI35. In some embodiments, an interferon-inducible gene is AIM2. In some embodiments, an interferon-inducible gene is GALM. In some embodiments, an interferon-inducible gene is EIF2AK2. In some embodiments, an interferon-inducible gene is ZBP1. In some embodiments, an interferon-inducible gene is APOL6. In some embodiments, an interferon-inducible gene is STAT1. In some embodiments, an interferon-inducible gene is GBP5. In some embodiments, an interferon-inducible gene is LGALS3BP. In some embodiments, an interferon-inducible gene is SEPT4. In some embodiments, an interferon-inducible gene is FBXO6. In some embodiments, an interferon-inducible gene is SCO2. In some embodiments, an interferon-inducible gene is MOV10. In some embodiments, an interferon-inducible gene is CEACAM1. In some embodiments, an interferon-inducible gene is PML. In some embodiments, an interferon-inducible gene is WARS. In some embodiments, an interferon-inducible gene is CCL8. In some embodiments, an interferon-inducible gene is SOCS1. In some embodiments, an interferon-inducible gene is ZNF684. In some embodiments, an interferon-inducible gene is SAMD9L. In some embodiments, an interferon-inducible gene is PARP10. In some embodiments, an interferon-inducible gene is GBP1. In some embodiments, an interferon-inducible gene is IRF7. In some embodiments, an interferon-inducible gene is IFIT5. In some embodiments, an interferon-inducible gene is IFIH1. In some embodiments, an interferon-inducible gene is DHX58. In some embodiments, an interferon-inducible gene is IFIT2. In some embodiments, an interferon-inducible gene is PARP12. In some embodiments, an interferon-inducible gene is TRIM22. In some embodiments, an interferon-inducible gene is DDX58. In some embodiments, an interferon-inducible gene is STAT2. In some embodiments, an interferon-inducible gene is PARP9. In some embodiments, an interferon-inducible gene is PARP14.

In some embodiments, one or more interferon-inducible genes are selected from MDK, CHMP5, SAMD9, C1QA, TCN2, ACTA2, NCOA7, PSMB9, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, SP140, CASP1, TRIM5, NMI, DYNLT1, TRAFD1, TDRD7, BTN3A1, ZC3HAV1, DHRS9, GBP2, TRIM56, HSH2D, and UNC93B1.

In some embodiments, one or more interferon-inducible genes are selected from MDK, CHMP5, SAMD9, C1QA, TCN2, ACTA2, NCOA7, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, CASP1, NMI, DYNLT1, TRAFD1, TDRD7, BTN3A1, ZC3HAV1, DHRS9, GBP2, TRIM56, HSH2D, and UNC93B1.

In some embodiments, one or more interferon-inducible genes are selected from MDK, CHMP5, SAMD9, ACTA2, NCOA7, PSMB9, GADD45B, BST2, ISG20, DRAP1, PHF11, TRIM38, ETV7, SP100, SP110, PARP10, SP140, CASP1, TRIM5, NMI, DYNLT1, TRAFD1, TDRD7, BTN3A1, ZC3HAV1, DHRS9, TRIM56, HSH2D, and UNC93B1.

In some embodiments, one or more interferon-inducible genes are selected from MDK, CHMP5, SAMD9, ACTA2, NCOA7, GADD45B, BST2, ISG20, DRAP1, PHF11, TRIM38, ETV7, SP100, SP110, PARP10, CASP1, NMI, DYNLT1, TRAFD1, TDRD7, BTN3A1, ZC3HAV1, DHRS9, TRIM56, HSH2D, and UNC93B1.

In some embodiments, one or more interferon-inducible genes are selected from MDK, CHMP5, SAMD9, ACTA2, NCOA7, PSMB9, BST2, ISG20, DRAP1, PHF11, SP100, TMEM140, NBN, LGALS9, IFI16, TRIM21, SRBD1, PARP10, SP140, CASP1, TRIM5, NMI, DYNLT1, TRAFD1, TDRD7, BTN3A1, ZC3HAV1, TRIM56, HSH2D, and UNC93B1.

In some embodiments, one or more interferon-inducible genes are selected from MDK, CHMP5, SAMD9, ACTA2, NCOA7, BST2, ISG20, DRAP1, PHF11, SP100, TMEM140, NBN, LGALS9, IFI16, TRIM21, SRBD1, PARP10, CASP1, NMI, DYNLT1, TRAFD1, TDRD7, BTN3A1, ZC3HAV1, TRIM56, HSH2D, and UNC93B1.

In some embodiments, one or more interferon-inducible genes are selected from MDK, CHMP5, SAMD9, ACTA2, NCOA7, BST2, ISG20, DRAP1, PHF11, SP100, PARP10, SP140, CASP1, NMI, DYNLT1, TRAFD1.

In some embodiments, one or more interferon-inducible genes are selected from MDK, CHMP5, SAMD9, ACTA2, NCOA7, BST2, ISG20, DRAP1, PHF11, SP100, PARP10, CASP1, NMI, DYNLT1, TRAFD1.

In some embodiments, one or more interferon-inducible genes are selected from MDK, CHMP5, SAMD9, C1QA, NCOA7, PSMB9, GADD45B, BST2, ISG20, DRAP1, PHF11, TRIM38, RBCK1, SP100, SP110, NBN, LGALS9, IFI16, PARP10, SP140, CASP1, TRIM5, NMI, DYNLT1, TRAFD1, TDRD7, BTN3A1, ZC3HAV1, and UNC93B1.

In some embodiments, one or more interferon-inducible genes are selected from MDK, CHMP5, SAMD9, C1QA, NCOA7, GADD45B, BST2, ISG20, DRAP1, PHF11, TRIM38, RBCK1, SP100, SP110, NBN, LGALS9, IFI16, PARP10, CASP1, NMI, DYNLT1, TRAFD1, TDRD7, BTN3A1, ZC3HAV1, and UNC93B1.

In some embodiments, one or more interferon-inducible genes are selected from CHMP5, SAMD9, NCOA7, PSMB9, GADD45B, BST2, ISG20, PHF11, TRIM38, RBCK1, SP100, SP110, NBN, LGALS9, IFI16, PARP10, SP140, CASP1, TRIM5, NMI, DYNLT1, TDRD7, ZC3HAV1, DHRS9, and UNC93B1.

In some embodiments, one or more interferon-inducible genes are selected from CHMP5, SAMD9, NCOA7, GADD45B, BST2, ISG20, PHF11, TRIM38, RBCK1, SP100, SP110, NBN, LGALS9, IFI16, PARP10, CASP1, NMI, DYNLT1, TDRD7, ZC3HAV1, DHRS9, and UNC93B1.

In some embodiments, one or more interferon-inducible genes are selected from MDK, CHMP5, SAMD9, NCOA7, BST2, ISG20, PHF11, TRIM38, SP100, SP110, NBN, IFI16, PARP10, SP140, TRIM5, NMI, DYNLT1, TDRD7, and UNC93B1.

In some embodiments, one or more interferon-inducible genes are selected from MDK, CHMP5, SAMD9, NCOA7, BST2, ISG20, PHF11, TRIM38, SP100, SP110, NBN, IFI16, PARP10, NMI, DYNLT1, TDRD7, and UNC93B1.

In some embodiments, an interferon-inducible gene is MDK. In some embodiments, an interferon-inducible gene is CHMP5. In some embodiments, an interferon-inducible gene is SAMD9. In some embodiments, an interferon-inducible gene is C1QA. In some embodiments, an interferon-inducible gene is TCN2. In some embodiments, an interferon-inducible gene is ACTA2. In some embodiments, an interferon-inducible gene is NCOA7. In some embodiments, an interferon-inducible gene is PSMB9. In some embodiments, an interferon-inducible gene is GADD45B. In some embodiments, an interferon-inducible gene is BST2. In some embodiments, an interferon-inducible gene is ISG20. In some embodiments, an interferon-inducible gene is DRAP1. In some embodiments, an interferon-inducible gene is PHF11. In some embodiments, an interferon-inducible gene is TRIM38. In some embodiments, an interferon-inducible gene is ETV7. In some embodiments, an interferon-inducible gene is RBCK1. In some embodiments, an interferon-inducible gene is SP100. In some embodiments, an interferon-inducible gene is ABCA1. In some embodiments, an interferon-inducible gene is RHBDF2. In some embodiments, an interferon-inducible gene is CPT1B. In some embodiments, an interferon-inducible gene is TAP1. In some embodiments, an interferon-inducible gene is NTNG2. In some embodiments, an interferon-inducible gene is TAP2. In some embodiments, an interferon-inducible gene is REC8. In some embodiments, an interferon-inducible gene is LHFPL2. In some embodiments, an interferon-inducible gene is TRIM25. In some embodiments, an interferon-inducible gene is ADAR. In some embodiments, an interferon-inducible gene is ZNFX1. In some embodiments, an interferon-inducible gene is SP110. In some embodiments, an interferon-inducible gene is TMEM140. In some embodiments, an interferon-inducible gene is NBN. In some embodiments, an interferon-inducible gene is LGALS9. In some embodiments, an interferon-inducible gene is IFI16. In some embodiments, an interferon-inducible gene is TRIM21. In some embodiments, an interferon-inducible gene is SRBD1. In some embodiments, an interferon-inducible gene is PARP10. In some embodiments, an interferon-inducible gene is SP140. In some embodiments, an interferon-inducible gene is CASP1. In some embodiments, an interferon-inducible gene is TRIM5. In some embodiments, an interferon-inducible gene is NMI. In some embodiments, an interferon-inducible gene is DYNLT1. In some embodiments, an interferon-inducible gene is TRAFD1. In some embodiments, an interferon-inducible gene is TDRD7. In some embodiments, an interferon-inducible gene is BTN3A1. In some embodiments, an interferon-inducible gene is ZC3HAV1. In some embodiments, an interferon-inducible gene is DHRS9. In some embodiments, an interferon-inducible gene is GBP2. In some embodiments, an interferon-inducible gene is TRIM56. In some embodiments, an interferon-inducible gene is HSH2D. In some embodiments, an interferon-inducible gene is UNC93B1.

In some embodiments, one or more interferon-inducible genes are selected from LY6E, RTP4, EPSTI1, OAS1, OAS2, OASL, BATF2, SERPING1, OTOF, IFITM3, CXCL10, ISG15, HES4, XAF1, OAS3, IFIT3, RSAD2, LAMP3, IFIT1, IFI44L, HERC5, MX1, IFI44, HERC6, ATF3, GBP3, GBP4, IFITM1, MT2A, TNFAIP6, LAP3, OAS2, PLSCR1, TNFSF10, TIMM10, UBE2L6, IFI35, AIM2, GALM, EIF2AK2, ZBP1, APOL6, STAT1, GBP5, LGALS3BP, SEPT4, FBXO6, SCO2, MOV10, CEACAM1, PML, WARS, CCL8, SOCS1, ZNF684, SAMD9L, PARP10, GBP1, IRF7, IFIT5, IFIH1, DHX58, IFIT2, PARP12, TRIM22, DDX58, STAT2, PARP9, PARP14, MDK, CHMP5, SAMD9, C1QA, TCN2, ACTA2, NCOA7, PSMB9, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, SP140, CASP1, TRIM5, NMI, DYNLT1, TRAFD1, TDRD7, BTN3A1, ZC3HAV1, DHRS9, GBP2, TRIM56, HSH2D, and UNC93B1.

In some embodiments, one or more interferon-inducible genes are selected from LY6E, RTP4, OAS1, OAS2, OASL, BATF2, SERPING1, OTOF, IFITM3, ISG15, HES4, XAF1, OAS3, IFIT3, RSAD2, LAMP3, IFIT1, IFI44L, HERC5, MX1, IFI44, HERC6, ATF3, GBP3, IFITM1, MT2A, TNFAIP6, LAP3, OAS2, PLSCR1, TNFSF10, TIMM10, UBE2L6, IFI35, AIM2, GALM, EIF2AK2, ZBP1, APOL6, GBP5, LGALS3BP, SEPT4, FBXO6, SCO2, MOV10, CEACAM1, PML, WARS, CCL8, SOCS1, ZNF684, SAMD9L, PARP10, GBP1, IRF7, IFIT5, IFIH1, DHX58, IFIT2, PARP12, DDX58, PARP14, MDK, CHMP5, SAMD9, C1QA, TCN2, ACTA2, NCOA7, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, CASP1, NMI, DYNLT1, TRAFD1, TDRD7, BTN3A1, ZC3HAV1, DHRS9, GBP2, TRIM56, HSH2D, and UNC93B1.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in one or more interferon-inducible genes. In some embodiments, one or more interferon-inducible genes are selected from LY6E, RTP4, EPSTI1, OAS1, OAS2, OASL, BATF2, SERPING1, OTOF, IFITM3, CXCL10, ISG15, HES4, XAF1, OAS3, IFIT3, RSAD2, LAMP3, IFIT1, IFI44L, HERC5, MX1, and IFI44.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in one or more interferon-inducible genes. In some embodiments, one or more interferon-inducible genes are selected from HERC6, ATF3, GBP3. GBP4, IFITM1, MT2A, TNFAIP6, LAP3, OAS2, PLSCR1, TNFSF10, TIMM10, UBE2L6, IFI35, AIM2, GALM, EIF2AK2, ZBP1, APOL6, STAT1, GBP5, LGALS3BP, SEPT4, FBXO6, SCO2, MOV10, CEACAM1, PML, WARS, CCL8, SOCS1, ZNF684, SAMD9L, PARP10, GBP1, IRF7, IFIT5, IFIH1, DHX58, IFIT2, PARP12, TRIM22, DDX58, STAT2, PARP9, and PARP14.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in one or more interferon-inducible genes. In some embodiments, one or more interferon-inducible genes are selected from MDK, CHMP5, SAMD9, C1QA, TCN2, ACTA2, NCOA7, PSMB9, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, SP140, CASP1, TRIM5, NMI, DYNLT1, TRAFD1, TDRD7, BTN3A1, ZC3HAV1, DHRS9, GBP2, TRIM56, HSH2D, and UNC93B1.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in one or more interferon-inducible genes. In some embodiments, one or more interferon-inducible genes are selected from LY6E, RTP4, OAS1, OAS2, OASL, BATF2, SERPING1, OTOF, IFITM3, CXCL10, ISG15, HES4, XAF1, OAS3, IFIT3, RSAD2, LAMP3, IFIT1, IFI44L, HERC5, MX1, and IFI44.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in one or more interferon-inducible genes. In some embodiments, one or more interferon-inducible genes are selected from LY6E, RTP4, EPSTI1, OAS1, OAS2, OASL, BATF2, SERPING1, OTOF, IFITM3, ISG15, HES4, XAF1, OAS3, IFIT3, RSAD2, LAMP3, IFIT1, IFI44L, HERC5, MX1, and IFI44.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in one or more interferon-inducible genes. In some embodiments, one or more interferon-inducible genes are selected from LY6E, RTP4, OAS1, OAS2, OASL, BATF2, SERPING1, OTOF, IFITM3, ISG15, HES4, XAF1, OAS3, IFIT3, RSAD2, LAMP3, IFIT1, IFI44L, HERC5, MX1, and IFI44.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in one or more interferon-inducible genes. In some embodiments, one or more interferon-inducible genes are selected from HERC6, ATF3, GBP3. IFITM1, MT2A, TNFAIP6, LAP3, OAS2, PLSCR1, TNFSF10, TIMM10, UBE2L6, IFI35, AIM2, GALM, EIF2AK2, ZBP1, APOL6, GBP5, LGALS3BP, SEPT4, FBXO6, SCO2, MOV10, CEACAM1, PML, WARS, CCL8, SOCS1, ZNF684, SAMD9L, PARP10, GBP1, IRF7, IFIT5, IFIH1, DHX58, IFIT2, PARP12, DDX58, and PARP14.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in one or more interferon-inducible genes. In some embodiments, one or more interferon-inducible genes are selected from MDK, CHMP5, SAMD9, C1QA, TCN2, ACTA2, NCOA7, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, CASP1, NMI, DYNLT1, TRAFD1, TDRD7, BTN3A1, ZC3HAV1, DHRS9, GBP2, TRIM56, HSH2D, and UNC93B1.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in one or more interferon-inducible genes and an improvement in fatigue. In some embodiments, one or more interferon-inducible genes are selected from LY6E, RTP4, EPSTI1, OAS1, OAS2, OASL, BATF2, SERPING1, OTOF, IFITM3, CXCL10, ISG15, HES4, XAF1, OAS3, IFIT3, RSAD2, LAMP3, IFIT1, IFI44L, HERC5, MX1, and IFI44.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in one or more interferon-inducible genes and an improvement in fatigue. In some embodiments, one or more interferon-inducible genes are selected from HERC6, ATF3, GBP3. GBP4, IFITM1, MT2A, TNFAIP6, LAP3, OAS2, PLSCR1, TNFSF10, TIMM10, UBE2L6, IFI35, AIM2, GALM, EIF2AK2, ZBP1, APOL6, STAT1, GBP5, LGALS3BP, SEPT4, FBXO6, SCO2, MOV10, CEACAM1, PML, WARS, CCL8, SOCS1, ZNF684, SAMD9L, PARP10, GBP1, IRF7, IFIT5, IFIH1, DHX58, IFIT2, PARP12, TRIM22, DDX58, STAT2, PARP9, and PARP14.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in one or more interferon-inducible genes and an improvement in fatigue. In some embodiments, one or more interferon-inducible genes are selected from MDK, CHMP5, SAMD9, C1QA, TCN2, ACTA2, NCOA7, PSMB9, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, SP140, CASP1, TRIM5, NMI, DYNLT1, TRAFD1, TDRD7, BTN3A1, ZC3HAV1, DHRS9, GBP2, TRIM56, HSH2D, and UNC93B1.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in one or more interferon-inducible genes and an improvement in fatigue. In some embodiments, one or more interferon-inducible genes are selected from LY6E, RTP4, OAS1, OAS2, OASL, BATF2, SERPING1, OTOF, IFITM3, CXCL10, ISG15, HES4, XAF1, OAS3, IFIT3, RSAD2, LAMP3, IFIT1, IFI44L, HERC5, MX1, and IFI44.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in one or more interferon-inducible genes and an improvement in fatigue. In some embodiments, one or more interferon-inducible genes are selected from LY6E, RTP4, EPSTI1, OAS1, OAS2, OASL, BATF2, SERPING1, OTOF, IFITM3, ISG15, HES4, XAF1, OAS3, IFIT3, RSAD2, LAMP3, IFIT1, IFI44L, HERC5, MX1, and IFI44.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in one or more interferon-inducible genes and an improvement in fatigue. In some embodiments, one or more interferon-inducible genes are selected from LY6E, RTP4, OAS1, OAS2, OASL, BATF2, SERPING1, OTOF, IFITM3, ISG15, HES4, XAF1, OAS3, IFIT3, RSAD2, LAMP3, IFIT1, IFI44L, HERC5, MX1, and IFI44.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in one or more interferon-inducible genes and an improvement in fatigue. In some embodiments, one or more interferon-inducible genes are selected from HERC6, ATF3, GBP3. IFITM1, MT2A, TNFAIP6, LAP3, OAS2, PLSCR1, TNFSF10, TIMM10, UBE2L6, IFI35, AIM2, GALM, EIF2AK2, ZBP1, APOL6, GBP5, LGALS3BP, SEPT4, FBXO6, SCO2, MOV10, CEACAM1, PML, WARS, CCL8, SOCS1, ZNF684, SAMD9L, PARP10, GBP1, IRF7, IFIT5, IFIH1, DHX58, IFIT2, PARP12, DDX58, and PARP14.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in one or more interferon-inducible genes and an improvement in fatigue. In some embodiments, one or more interferon-inducible genes are selected from MDK, CHMP5, SAMD9, C1QA, TCN2, ACTA2, NCOA7, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, CASP1, NMI, DYNLT1, TRAFD1, TDRD7, BTN3A1, ZC3HAV1, DHRS9, GBP2, TRIM56, HSH2D, and UNC93B1.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in one or more interferon-inducible genes selected from modules M1.2, M3.4, and M5.12. In some embodiments, treatment results in an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in one or more interferon-inducible genes from module M1.2. In some embodiments, treatment results in an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in one or more interferon-inducible genes from module M3.4. In some embodiments, treatment results in an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in one or more interferon-inducible genes from module M5.12. In some embodiments, treatment results in an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in at least 10%, 20%, 30%, 40%, 50%, 60%, 70%, 80%, 90%, or 100% of the genes in module M1.2. In some embodiments, treatment results in an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in at least 10%, 20%, 30%, 40%, 50%, 60%, 70%, 80%, 90%, or 100% of the genes in module M3.4. In some embodiments, treatment results in an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in at least 10%, 20%, 30%, 40%, 50%, 60%, 70%, 80%, 90%, or 100% of the genes in module M5.4. In some embodiments, treatment results in an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in at least 10%, 20%, 30%, 40%, 50%, 60%, 70%, 80%, 90%, or 100% of one or more of the genes in module M1.2. In some embodiments, treatment results in an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in at least 10%, 20%, 30%, 40%, 50%, 60%, 70%, 80%, 90%, or 100% of one or more of the genes in module M3.4. In some embodiments, treatment results in an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in at least 10%, 20%, 30%, 40%, 50%, 60%, 70%, 80%, 90%, or 100% of one or more of the genes in module M5.4. In some embodiments, treatment results in an improvement in fatigue.

In some embodiments, one or more M1.2 module interferon-inducible genes are selected from LY6E, RTP4, EPSTI1, OAS1, OAS2, OASL, BATF2, SERPING1, OTOF, IFITM3, CXCL10, ISG15, HES4, XAF1, OAS3, IFIT3, RSAD2, LAMP3, IFIT1, IFI44L, HERC5, MX1, and IFI44.

In some embodiments, one or more M3.4 module interferon-inducible genes are selected from HERC6, ATF3, GBP3, GBP4, IFITM1, MT2A, TNFAIP6, LAP3, OAS2, PLSCR1, TNFSF10, TIMM10, UBE2L6, IFI35, AIM2, GALM, EIF2AK2, ZBP1, APOL6, STAT1, GBP5, LGALS3BP, SEPT4, FBXO6, SCO2, MOV10, CEACAM1, PML, WARS, CCL8, SOCS1, ZNF684, SAMD9L, PARP10, GBP1, IRF7, IFIT5, IFIH1, DHX58, IFIT2, PARP12, TRIM22, DDX58, STAT2, PARP9, and PARP14.

In some embodiments, one or more M5.12 module interferon-inducible genes are selected from MDK, CHMP5, SAMD9, C1QA, TCN2, ACTA2, NCOA7, PSMB9, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, SP140, CASP1, TRIM5, NMI, DYNLT1, TRAFD1, TDRD7, BTN3A1, ZC3HAV1, DHRS9, GBP2, TRIM56, HSH2D, and UNC93B1.

### Diagnostic Methods

The disclosure provides methods related to detecting and/or quantifying one or more interferon-inducible molecules (e.g., interferon-inducible genes) described herein, in one or more samples, wherein the detection and/or quantification of the one or more interferon-inducible molecules individually or in combination, will indicate a likelihood that a patient with Sjogren’s syndrome has achieved a response to an RNA nuclease agent (e.g., RSLV-132).

Provided herein are methods for monitoring a response of a patient with Sjogren’s syndrome treated with an RNA nuclease agent (e.g., RSLV-132), the method comprising: determining the expression level and/or activity of one or more interferon-inducible molecules (e.g., an interferon-inducible gene), in a first sample from the patient obtained prior to treatment with the RNA nuclease agent; determining the expression level and/or activity of the one or more interferon-inducible molecules (e.g., an interferon-inducible gene) in a second sample from the patient obtained after treatment with the RNA nuclease agent; comparing the expression level and/or activity of the one or more interferon-inducible molecules (e.g., an interferon-inducible gene) in the second sample with the expression level and/or activity of the one or more interferon-inducible molecules (e.g., an interferon-inducible gene) in the first sample, wherein a change of the expression level and/or activity of the one or more interferon-inducible molecules (e.g., an interferon-inducible gene) in the second sample relative to the expression level and/or activity of the one or more interferon-inducible molecules (e.g., an interferon-inducible gene) in the first sample indicates a response of the patient treated with the RNA nuclease agent. In some aspects, the one or more interferon-inducible molecules is an interferon-inducible gene. In some embodiments, one or more interferon-inducible genes are selected from LY6E, RTP4, EPSTI1, OAS1, OAS2, OASL, BATF2, SERPING1, OTOF, IFITM3, CXCL10, ISG15, HES4, XAF1, OAS3, IFIT3, RSAD2, LAMP3, IFIT1, IFI44L, HERC5, MX1, and IFI44. In some embodiments, one or more interferon-inducible genes are selected from LY6E, RTP4, EPSTI1, OAS1, OAS2, OASL, BATF2, SERPING1, OTOF, IFITM3, ISG15, HES4, XAF1, OAS3, IFIT3, RSAD2, LAMP3, IFIT1, IFI44L, HERC5, MX1, and IFI44. In some embodiments, one or more interferon-inducible genes are selected from LY6E, RTP4, OAS1, OAS2, OASL, BATF2, SERPING1, OTOF, IFITM3, CXCL10, ISG15, HES4, XAF1, OAS3, IFIT3, RSAD2, LAMP3, IFIT1, IFI44L, HERC5, MX1, and IFI44. In some embodiments, one or more interferon-inducible genes are selected from LY6E, RTP4, OAS1, OAS2, OASL, BATF2, SERPING1, OTOF, IFITM3, ISG15, HES4, XAF1, OAS3, IFIT3, RSAD2, LAMP3, IFIT1, IFI44L, HERC5, MX1, and IFI44. In some embodiments, one or more interferon-inducible genes are selected from HERC6, ATF3, GBP3, GBP4, IFITM1, MT2A, TNFAIP6, LAP3, OAS2, PLSCR1, TNFSF10, TIMM10, UBE2L6, IFI35, AIM2, GALM, EIF2AK2, ZBP1, APOL6, STAT1, GBP5, LGALS3BP, SEPT4, FBXO6, SCO2, MOV10, CEACAM1, PML, WARS, CCL8, SOCS1, ZNF684, SAMD9L, PARP10, GBP1, IRF7, IFIT5, IFIH1, DHX58, IFIT2, PARP12, TRIM22, DDX58, STAT2, PARP9, and PARP14. In some embodiments, one or more interferon-inducible genes are selected from HERC6, ATF3, GBP3, IFITM1, MT2A, TNFAIP6, LAP3, OAS2, PLSCR1, TNFSF10, TIMM10, UBE2L6, IFI35, AIM2, GALM, EIF2AK2, ZBP1, APOL6, GBP5, LGALS3BP, SEPT4, FBXO6, SCO2, MOV10, CEACAM1, PML, WARS, CCL8, SOCS1, ZNF684, SAMD9L, PARP10, GBP1, IRF7, IFIT5, IFIH1, DHX58, IFIT2, PARP12, DDX58, and PARP14. In some embodiments, one or more interferon-inducible genes are selected from MDK, CHMP5, SAMD9, C1QA, TCN2, ACTA2, NCOA7, PSMB9, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, SP140, CASP1, TRIM5, NMI, DYNLT1, TRAFD1, TDRD7, BTN3A1, ZC3HAV1, DHRS9, GBP2, TRIM56, HSH2D, and UNC93B1. In some embodiments, one or more interferon-inducible genes are from MDK, CHMP5, SAMD9, C1QA, TCN2, ACTA2, NCOA7, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, CASP1, NMI, DYNLT1, TRAFD1, TDRD7, BTN3A1, ZC3HAV1, DHRS9, GBP2, TRIM56, HSH2D, and UNC93B1.

Further provided herein are methods for monitoring a response of a patient with Sjogren’s syndrome treated with an RNA nuclease agent (e.g., RSLV-132), the method comprising: determining the expression level and/or activity of one or more interferon-inducible molecules (e.g., an interferon-inducible gene), in a first sample from the patient obtained prior to treatment with the RNA nuclease agent; determining the expression level and/or activity of the one or more interferon-inducible molecules (e.g., an interferon-inducible gene) in a second sample from the patient obtained after treatment with the RNA nuclease agent; comparing the expression level and/or activity of the one or more interferon-inducible molecules (e.g., an interferon-inducible gene) in the second sample with the expression level and/or activity of the one or more interferon-inducible molecules (e.g., an interferon-inducible gene) in the first sample, wherein an increase in the expression level and/or activity of the one or more interferon-inducible molecules (e.g., an interferon-inducible gene) in the second sample relative to the expression level and/or activity of the one or more interferon-inducible molecules (e.g., an interferon-inducible gene) in the first sample indicates a response of the patient treated with the RNA nuclease agent. In some aspects, one or more interferon-inducible molecules is an interferon-inducible gene. In some embodiments, the one or more interferon-inducible genes are selected from LY6E, RTP4, EPSTI1, OAS1, OAS2, OASL, BATF2, SERPING1, OTOF, IFITM3, CXCL10, ISG15, HES4, XAF1, OAS3, IFIT3, RSAD2, LAMP3, IFIT1, IFI44L, HERC5, MX1, and IFI44. In some embodiments, one or more interferon-inducible genes are selected from LY6E, RTP4, EPSTI1, OAS1, OAS2, OASL, BATF2, SERPING1, OTOF, IFITM3, ISG15, HES4, XAF1, OAS3, IFIT3, RSAD2, LAMP3, IFIT1, IFI44L, HERC5, MX1, and IFI44. In some embodiments, one or more interferon-inducible genes are selected from LY6E, RTP4, OAS1, OAS2, OASL, BATF2, SERPING1, OTOF, IFITM3, CXCL10, ISG15, HES4, XAF1, OAS3, IFIT3, RSAD2, LAMP3, IFIT1, IFI44L, HERC5, MX1, and IFI44. In some embodiments, one or more interferon-inducible genes are selected from LY6E, RTP4, OAS1, OAS2, OASL, BATF2, SERPING1, OTOF, IFITM3, ISG15, HES4, XAF1, OAS3, IFIT3, RSAD2, LAMP3, IFIT1, IFI44L, HERC5, MX1, and IFI44. In some embodiments, one or more interferon-inducible genes are selected from HERC6, ATF3, GBP3, GBP4, IFITM1, MT2A, TNFAIP6, LAP3, OAS2, PLSCR1, TNFSF10, TIMM10, UBE2L6, IFI35, AIM2, GALM, EIF2AK2, ZBP1, APOL6, STAT1, GBP5, LGALS3BP, SEPT4, FBXO6, SCO2, MOV10, CEACAM1, PML, WARS, CCL8, SOCS1, ZNF684, SAMD9L, PARP10, GBP1, IRF7, IFIT5, IFIH1, DHX58, IFIT2, PARP12, TRIM22, DDX58, STAT2, PARP9, and PARP14. In some embodiments, one or more interferon-inducible genes are selected from HERC6, ATF3, GBP3, IFITM1, MT2A, TNFAIP6, LAP3, OAS2, PLSCR1, TNFSF10, TIMM10, UBE2L6, IFI35, AIM2, GALM, EIF2AK2, ZBP1, APOL6, GBP5, LGALS3BP, SEPT4, FBXO6, SCO2, MOV10, CEACAM1, PML, WARS, CCL8, SOCS1, ZNF684, SAMD9L, PARP10, GBP1, IRF7, IFIT5, IFIH1, DHX58, IFIT2, PARP12, DDX58, and PARP14. In some embodiments, one or more interferon-inducible genes are selected from MDK, CHMP5, SAMD9, C1QA, TCN2, ACTA2, NCOA7, PSMB9, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, SP140, CASP1, TRIM5, NMI, DYNLT1, TRAFD1, TDRD7, BTN3A1, ZC3HAV1, DHRS9, GBP2, TRIM56, HSH2D, and UNC93B1. In some embodiments, one or more interferon-inducible genes are from MDK, CHMP5, SAMD9, C1QA, TCN2, ACTA2, NCOA7, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, CASP1, NMI, DYNLT1, TRAFDI1, TDRD7, BTN3A1, ZC3HAV1, DHRS9, GBP2, TRIM56, HSH2D, and UNC93B1.

Further provided herein are methods for monitoring a response of a patient with Sjogren’s syndrome treated with an RNA nuclease agent (e.g., RSLV-132), the method comprising: determining the expression level and/or activity of one or more interferon-inducible molecules (e.g., an interferon-inducible gene), in a first sample from the patient obtained prior to treatment with the RNA nuclease agent; determining the expression level and/or activity of the one or more interferon-inducible molecules (e.g., an interferon-inducible gene) in a second sample from the patient obtained after treatment with the RNA nuclease agent; comparing the expression level and/or activity of the one or more interferon-inducible molecules (e.g., an interferon-inducible gene) in the second sample with the expression level and/or activity of the one or more interferon-inducible molecules (e.g., an interferon-inducible gene) in the first sample, wherein an decrease in the expression level and/or activity of the one or more interferon-inducible molecules (e.g., an interferon-inducible gene) in the second sample relative to the expression level and/or activity of the one or more interferon-inducible molecules (e.g., an interferon-inducible gene) in the first sample indicates a response of the patient treated with the RNA nuclease agent. In some aspects, one or more interferon-inducible molecules is an interferon-inducible gene.

### Determination of Interferon-Inducible Molecules

The presence of or an amount or expression level of one or more interferon-inducible molecules (e.g., interferon-inducible genes, interferon-inducible proteins) described herein in a sample can be detected or determined by a number of methodologies and techniques, which are known in the art and understood by the skilled artisan, including, but not limited to, immunohistochemistry (IHC), immunofluorescence (IF), Western blot analysis, immunoprecipitation, molecular binding assays, enzyme-linked immunosorbent assay (ELISA), enzyme-linked immunofiltration assay (ELIFA), flow cytometry, MassARRAY, proteomics, quantitative blood based assays (e.g., Serum ELISA), biochemical enzymatic activity assays, in situ hybridization, fluorescence in situ hybridization (FISH), Southern analysis, Northern analysis, whole genome sequencing, polymerase chain reaction (PCR) including quantitative real time PCR (qRT-PCR) and other amplification type detection methods, such as, for example, branched DNA, SISBA, TMA and the like, RNA-Seq, microarray analysis, gene expression profiling, and/or serial analysis of gene expression (SAGE), as well as any one of the wide variety of assays that can be performed by protein, gene, and/or tissue array analysis. Typical protocols for evaluating the status of genes and gene products are found, for example in Ausubel et al., eds., 1 995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used. Diagnostic antibodies that bind the interferon-inducible molecules of the disclosure are available from a variety of commercial sources, such as BD Biosciences, ebiosciences, BioLegend, Abcam, and the like.

### Nucleic Acid Techniques

In some embodiments, the expression level of an interferon-inducible molecule (e.g., an interferon-inducible gene), described herein, may be a nucleic acid expression level. In some embodiments, the nucleic acid expression level is determined using qPCR, rtPCR, RNA-Seq, multiplex qPCR or RT-qPCR, microarray analysis, gene expression profiling, SAGE, MassARRAY technique, or in situ hybridization (e.g., FISH). In some embodiments, the expression level of an interferon-inducible molecule (e.g., interferon-inducible gene) is determined in cells from a patient with Sjogren’s syndrome. In some embodiments, the expression level of an interferon-inducible molecule (e.g., interferon-inducible gene) described herein is determined in blood cells from a patient with Sjogren’s syndrome.

Methods for the evaluation of mRNAs in cells are known in the art and include, for example, hybridization assays using complementary DNA probes (such as in situ hybridization using labeled riboprobes specific for the one or more genes, Northern blot and related techniques) and various nucleic acid amplification assays (such as RT-PCR using complementary primers specific for one or more of the genes, and other amplification type detection methods, such as, for example, branched DNA, SISBA, TMA and the like). In addition, such methods can include one or more steps that allow one to determine the levels of target mRNA in a biological sample (e.g., by simultaneously examining the levels a comparative control mRNA sequence of a “housekeeping” gene such as an actin family member).

The nucleic acid sequences of the interferon-inducible molecules (e.g., interferon-inducible genes) of the disclosure are known in the art. In some embodiments, the interferon-inducible molecule of the disclosure is an interferon-inducible gene. In some embodiments, one or more interferon-inducible genes are selected from LY6E, RTP4, EPSTI1, OAS1, OAS2, OASL, BATF2, SERPING1, OTOF, IFITM3, CXCL10, ISG15, HES4, XAF1, OAS3, IFIT3, RSAD2, LAMP3, IFIT1, IFI44L, HERC5, MX1, and IFI44. In some embodiments, one or more interferon-inducible genes are selected from LY6E, RTP4, EPSTI1, OAS1, OAS2, OASL, BATF2, SERPING1, OTOF, IFITM3, ISG15, HES4, XAF1, OAS3, IFIT3, RSAD2, LAMP3, IFIT1, IFI44L, HERC5, MX1, and IFI44. In some embodiments, one or more interferon-inducible genes are selected from LY6E, RTP4, OAS1, OAS2, OASL, BATF2, SERPING1, OTOF, IFITM3, CXCL10, ISG15, HES4, XAF1, OAS3, IFIT3, RSAD2, LAMP3, IFIT1, IFI44L, HERC5, MX1, and IFI44. In some embodiments, one or more interferon-inducible genes are selected from LY6E, RTP4, OAS1, OAS2, OASL, BATF2, SERPING1, OTOF, IFITM3, ISG15, HES4, XAF1, OAS3, IFIT3, RSAD2, LAMP3, IFIT1, IFI44L, HERC5, MX1, and IFI44. In some embodiments, one or more interferon-inducible genes are selected from HERC6, ATF3, GBP3, GBP4, IFITM1, MT2A, TNFAIP6, LAP3, OAS2, PLSCR1, TNFSF10, TIMM10, UBE2L6, IFI35, AIM2, GALM, EIF2AK2, ZBP1, APOL6, STAT1, GBP5, LGALS3BP, SEPT4, FBXO6, SCO2, MOV10, CEACAM1, PML, WARS, CCL8, SOCS1, ZNF684, SAMD9L, PARP10, GBP1, IRF7, IFIT5, IFIH1, DHX58, IFIT2, PARP12, TRIM22, DDX58, STAT2, PARP9, and PARP14. In some embodiments, one or more interferon-inducible genes are selected from HERC6, ATF3, GBP3, IFITM1, MT2A, TNFAIP6, LAP3, OAS2, PLSCR1, TNFSF10, TIMM10, UBE2L6, IFI35, AIM2, GALM, EIF2AK2, ZBP1, APOL6, GBP5, LGALS3BP, SEPT4, FBXO6, SCO2, MOV10, CEACAM1, PML, WARS, CCL8, SOCS1, ZNF684, SAMD9L, PARP10, GBP1, IRF7, IFIT5, IFIH1, DHX58, IFIT2, PARP12, DDX58, and PARP14. In some embodiments, one or more interferon-inducible genes are selected from MDK, CHMP5, SAMD9, C1QA, TCN2, ACTA2, NCOA7, PSMB9, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, SP140, CASP1, TRIM5, NMI, DYNLT1, TRAFD1, TDRD7, BTN3A1, ZC3HAV1, DHRS9, GBP2, TRIM56, HSH2D, and UNC93B1. In some embodiments, one or more interferon-inducible genes are from MDK, CHMP5, SAMD9, C1QA, TCN2, ACTA2, NCOA7, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, CASP1, NMI, DYNLT1, TRAFD1, TDRD7, BTN3A1, ZC3HAV1, DHRS9, GBP2, TRIM56, HSH2D, and UNC93B1.

In some embodiments, the sequence of the amplified target cDNA can be determined. Methods include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes whose expression correlates with increased or reduced clinical benefit of treatment comprising an RNA nuclease agent (e.g., RSLV-132), may be arrayed on a solid support. Hybridization of a labeled probe with a particular array member indicates that the sample from which the probe was derived expresses that gene.

Inclusion of any of the diagnostic methods described herein as part of any method directed to methods for identifying patients likely to benefit from treatment as described herein (e.g., selection of a therapeutic treatment or intervention) or to the development of treatments (e.g., enrollment patients in clinical trials) provides an advantage over those methods that do not include the diagnostic methods, in that a patient population whose members are predicted to need and/or not need, benefit, or respond to treatment may be identified.

Accordingly, in some embodiments, the interferon-inducible molecule (e.g., interferon-inducible gene) suitable for use in the present disclosure is a diagnostic interferon-inducible molecule (e.g., interferon-inducible gene). In some embodiments, the interferon-inducible molecule (e.g., interferon-inducible gene) is a monitoring interferon-inducible molecule (e.g., interferon-inducible gene). In some embodiments, the interferon-inducible molecule (e.g., interferon-inducible gene) is a predictive interferon-inducible molecule (e.g., interferon-inducible gene)

Interferon-inducible molecules (e.g., interferon-inducible genes) can be a substance or a biological event whose detection indicates a particular physiological state (e.g., a diseased state). For example, the presence of an interferon-inducible gene in the serum of a patient may indicate disease (e.g., Sjogren’s syndrome). Interferon-inducible molecules (e.g., interferon-inducible genes) measured in patients before treatment can be used to identify suitable patients for inclusion in a clinical trial. Interferon-inducible molecules (e.g., interferon-inducible genes) changes after treatment may predict or identify safety problems related to a candidate drug, or reveal pharmacologic activity expected to predict an eventual benefit of treatment. Interferon-inducible molecules (e.g., interferon-inducible genes) may reduce uncertainty in drug development and evaluation by providing quantifiable predictions about drug performance, and they can contribute to dose selection. Composite interferon-inducible molecules (e.g., interferon-inducible genes) include several individual interferon-inducible molecules (e.g., interferon-inducible genes) in a stated algorithm which reaches a single interpretive readout when a single interferon-inducible molecule (e.g., interferon-inducible gene) fails to provide all the relevant information required for assessment.

A surrogate end-point is an interferon-inducible molecule (e.g., interferon-inducible gene) that is intended to substitute for a clinical end point and is expected, based on epidemiologic, therapeutic, pathophysiologic, or other scientific evidence, to predict clinical benefit.

### Protein Techniques

In some embodiments, the amount of an interferon-inducible molecule (e.g., an interferon-inducible protein) is measured by determining the protein expression level of the interferon-inducible molecules (e.g., interferon-inducible protein). There are a number of techniques that measure or determine protein expression levels known in the art and described herein that may be used in the methods provided by the disclosure. For example, in some embodiments, a protein expression level of a the interferon-inducible molecule (e.g., interferon-inducible protein) is determined using a method selected from the group consisting of flow cytometry (e.g., fluorescence-activated cell sorting (FACS™)), Western blot, enzyme-linked immunosorbent assay (ELISA), immunoprecipitation, immunohistochemistry (IHC), immunofluorescence, radioimmunoassay, dot blotting, immunodetection methods, HPLC, surface plasmon resonance, optical spectroscopy, mass spectrometry, and HPLC.

In some embodiments, a sample is contacted with an antibody that specifically binds to an interferon-inducible molecule (e.g., interferon-inducible protein) described herein under conditions permissive for binding of the interferon-inducible molecules (e.g., interferon-inducible protein), and the presence of a complex formed by the antibody and the interferon-inducible molecule (e.g., interferon-inducible protein) is detected. In some embodiments, a sample is contacted with a combination of antibodies that specifically bind to a combination of interferon-inducible molecules (e.g., interferon-inducible proteins) described herein. In some embodiments, the protein expression level of the interferon-inducible molecules (e.g., interferon-inducible protein) is determined in cells from a patient with Sjogren’s syndrome. In some embodiments, the protein expression level of the interferon-inducible molecule (e.g., interferon-inducible protein) is determined in blood cells from a patient with Sjogren’s syndrome.

In some embodiments, the amount of an interferon-inducible molecule (e.g., interferon-inducible protein) in a sample is determined using a diagnostic antibody that binds the interferon-inducible molecule (e.g., interferon-inducible protein). In some embodiments, the anti-interferon-inducible molecule diagnostic antibody specifically binds the interferon-inducible molecule (e.g., interferon-inducible protein). In some embodiments, the diagnostic antibody is a non-human antibody. In some embodiments, the diagnostic antibody is a rat, mouse, or rabbit antibody. In some embodiments, the diagnostic antibody is a monoclonal antibody. In some embodiments, the diagnostic antibody is directly labeled. In other embodiments, the diagnostic antibody is indirectly labeled.

### Methods and Uses of Inflammatory-Related Molecules

Provided herein are diagnostic and therapeutic methods and uses of the inflammatory-related molecules (for example, inflammatory-related genes, inflammatory-related proteins, pro-inflammatory molecules) described herein. Further provided herein are methods of identifying subjects having Sjogren’s disease as likely to respond to treatment with an RNA nuclease agent as described herein by detecting the presence of or determining the amount or expression level of one or more inflammatory-related molecules (e.g., amount or expression level) in a sample obtained from the subject, wherein the presence of or the amount or expression level of the one or more inflammatory-related molecules indicates the subject is likely to respond to treatment with the RNA nuclease agent.

Further provided herein are methods of identifying subjects having Sjogren’s syndrome that have achieved a response to treatment with an RNA nuclease agent as described herein by detecting the presence of or determining the amount or expression level of one or more inflammatory-related molecules (e.g., amount or expression level) in a sample obtained from the subject, wherein the presence of or the amount or expression level of the one or more inflammatory-related molecules indicates the subject achieved a response to the RNA nuclease agent. In some embodiments, a change in expression of one or more inflammatory-related molecules of the disclosure indicates the subject has achieved a response to the RNA nuclease agent (e.g., RSLV-132). In some embodiments, a decrease in expression of one or more inflammatory-related molecules of the disclosure indicates the subject has achieved a response to the RNA nuclease agent (e.g., RSLV-132). In some embodiments, an increase in expression of one or more inflammatory-related molecules of the disclosure indicates the subject has achieved a response to the RNA nuclease agent (e.g., RSLV-132).

In some embodiments, the expression level of one or more inflammatory-related molecules in the sample from a patient with Sjogren’s syndrome is compared to the expression level of one or more inflammatory-related molecules in a control. In some embodiments, a control is a sample from a healthy subject. In some embodiments, a control is a sample taken from the patient prior to treatment. In some embodiments, a control is a sample take from the patient prior to disease onset. In some embodiments, a control is a sample taken from a subject that did not respond to treatment.

### Inflammatory-Related Molecules

In some aspects, the disclosure provides methods for detecting the presence of or determining an amount or expression level of an inflammatory-related molecule (for example, an inflammatory-related gene) in a sample from a subject (e.g., a sample from a subject with Sjogren’s syndrome). In some aspects, the disclosure provides methods for treating a subject having Sjogren’s syndrome a with an RNase nuclease agent (e.g., RSLV-132), wherein treatment results in a decrease in one or more inflammatory-related molecules. In some aspects, the disclosure provides methods for treating a subject having Sjogren’s syndrome a with an RNase nuclease agent (e.g., RSLV-132), wherein treatment results in an increase in one or more inflammatory-related molecules. In some aspects, the disclosure provides methods for treating a subject having Sjogren’s syndrome a with an RNase nuclease agent (e.g., RSLV-132), wherein treatment results in a change in expression of one or more inflammatory-related molecules.

In some aspects, the disclosure provides methods for identifying a subject having Sjogren’s disease as a candidate for treatment with an RNase nuclease agent (e.g., RSLV-132) by determining the inflammatory-related gene expression profile in the a sample obtained from the subject, and comparing the inflammatory-related gene expression profile determined in the sample obtained from the subject with an inflammatory-related gene expression profile in a sample obtained from a suitable control, wherein the inflammatory-related gene expression profile indicates that the subject is a candidate for treatment with an RNA nuclease agent (e.g., RSLV-132).

In some embodiments, a control is a sample from a healthy subject. In some embodiments, a control is a sample taken from the patient prior to treatment. In some embodiments, a control is a sample take from the patient prior to disease onset. In some embodiments, a control is a sample taken from a subject that did not respond to treatment.

As used herein, the term “inflammatory-related molecule” refers to a molecule that functions in inflammation or an inflammatory response. In some embodiments, the inflammatory-related molecule is a pro-inflammatory molecule. In some embodiments, the inflammatory-related molecule is an anti-inflammatory molecule. In some embodiments, the inflammatory-related molecule is an inflammatory mediator. In some embodiments, the inflammatory-related molecule is an inflammatory-related protein. In some embodiments, the inflammatory-related molecule is an inflammatory-related cytokine. In some embodiments, the inflammatory-related molecule is an inflammatory-related gene.

In some embodiments, an inflammatory-related gene is CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, PLA2G7, NUPR1, ZC3H12A, TPST1, CDK19, CCN3, IL5, IL1RAP, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and/or CXCL1.

In some embodiments, an inflammatory-related gene is CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, and/or PLA2G7.

In some embodiments, an inflammatory related gene is NUPR1, ZC3H12A, TPST1, CDK19, CCN3, IL5, IL1RAP, TBXA2R, GBA, SLC11A1, NEMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and/or CXCL1.

In some embodiments, an inflammatory related gene is CD36. In some embodiments, an inflammatory related gene is RIPK2. In some embodiments, an inflammatory related gene is PTPN2. In some embodiments, an inflammatory related gene is APOL3. In some embodiments, an inflammatory related gene is HGF. In some embodiments, an inflammatory related gene is BIRC3. In some embodiments, an inflammatory related gene is SETD6. In some embodiments, an inflammatory related gene is CD47. In some embodiments, an inflammatory related gene is IDO1. In some embodiments, an inflammatory related gene is ACER3. In some embodiments, an inflammatory related gene is APPL1. In some embodiments, an inflammatory related gene is SNX4. In some embodiments, an inflammatory related gene is CCR2. In some embodiments, an inflammatory related gene is CD163. In some embodiments, an inflammatory related gene is VAMP7. In some embodiments, an inflammatory related gene is TBC1D23. In some embodiments, an inflammatory related gene is CXCL10. In some embodiments, an inflammatory related gene is AIMP1. In some embodiments, an inflammatory related gene is CYBB. In some embodiments, an inflammatory related gene is PLA2G7. In some embodiments, an inflammatory related gene is NUPR1. In some embodiments, an inflammatory related gene is ZC3H12A. In some embodiments, an inflammatory related gene is TPST1. In some embodiments, an inflammatory related gene is CDK19. In some embodiments, an inflammatory related gene is CCN3. In some embodiments, an inflammatory related gene is IL5. In some embodiments, an inflammatory related gene is IL1RAP. In some embodiments, an inflammatory related gene is TBXA2R. In some embodiments, an inflammatory related gene is GBA. In some embodiments, an inflammatory related gene is SLC11A1. In some embodiments, an inflammatory related gene is MMP25. In some embodiments, an inflammatory related gene is CSF1. In some embodiments, an inflammatory related gene is FOS. In some embodiments, an inflammatory related gene is TYROBP. In some embodiments, an inflammatory related gene is FPR2. In some embodiments, an inflammatory related gene is LTBR4. In some embodiments, an inflammatory related gene is MAPKAPK2. In some embodiments, an inflammatory related gene is STAT5B. In some embodiments, an inflammatory related gene is ORM2. In some embodiments, an inflammatory related gene is BCL6. In some embodiments, an inflammatory related gene is IL1RN. In some embodiments, an inflammatory related gene is SIRPA. In some embodiments, an inflammatory related gene is TREM1. In some embodiments, an inflammatory related gene is GPS2. In some embodiments, an inflammatory related gene is FPR1. In some embodiments, an inflammatory related gene is PGLYRP1. In some embodiments, an inflammatory related gene is PIK3CD. In some embodiments, an inflammatory related gene is NFAM1. In some embodiments, an inflammatory related gene is IL17RA. In some embodiments, an inflammatory related gene is IL6R. In some embodiments, an inflammatory related gene is AKT1. In some embodiments, an inflammatory related gene is TNFRSF1A. In some embodiments, an inflammatory related gene is F11R. In some embodiments, an inflammatory related gene is ADAM8. In some embodiments, an inflammatory related gene is NLRP12. In some embodiments, an inflammatory related gene is NDST1. In some embodiments, an inflammatory related gene is IL1R2. In some embodiments, an inflammatory related gene is MAPK13. In some embodiments, an inflammatory related gene is MMP9. In some embodiments, an inflammatory related gene is NOD2. In some embodiments, an inflammatory related gene is CXCL1.

In some aspects, the disclosure provides methods for treating Sjogren’s disease in a patient in need thereof, the method comprising administering an effective amount of an RNA nuclease agent (e.g., RSLV-132) to the patient, wherein treatment results in a change in expression in one or more inflammatory-related genes. In some embodiments, the inflammatory-related gene is one or more of the following genes CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, PLA2G7, NUPR1, ZC3H12A, TPST1, CDK19, CCN3, IL5, IL1RAP, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and/or CXCL1.

In some aspects, the disclosure provides methods for treating Sjogren’s disease in a patient in need thereof, the method comprising administering an effective amount of an RNA nuclease agent (e.g., RSLV-132) to the patient, wherein treatment results in a change in expression in one or more inflammatory-related genes and am improvement in fatigue. In some embodiments, the inflammatory-related gene is one or more of the following genes CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, PLA2G7, NUPR1, ZC3H12A, TPST1, CDK19, CCN3, IL5, IL1RAP, TBXA2R, GBA, SLC11A1, NEMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and/or CXCL1.

In some aspects, the disclosure provides methods for treating Sjogren’s disease in a patient in need thereof, the method comprising administering an effective amount of an RNA nuclease agent (e.g., RSLV-132) to the patient, wherein treatment results in a decrease in expression of one or more inflammatory-related genes. In some embodiments, the inflammatory-related gene is one or more of the following genes NUPR1, ZC3H12A, TPST1, CDK19, CCN3, IL5, IL1RAP, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and/or CXCL1.

In some aspects, the disclosure provides methods for treating Sjogren’s disease in a patient in need thereof, the method comprising administering an effective amount of an RNA nuclease agent (e.g., RSLV-132) to the patient, wherein treatment results in a decrease in expression of one or more inflammatory-related genes and an improvement in fatigue. In some embodiments, the inflammatory-related gene is one or more of the following genes NUPR1, ZC3H12A, TPST1, CDK19, CCN3, IL5, IL1RAP, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and/or CXCL1.

In some aspects, the disclosure provides methods for treating Sjogren’s disease in a patient in need thereof, the method comprising administering an effective amount of an RNA nuclease agent (e.g., RSLV-132) to the patient, wherein treatment results in an increase in expression of one or more inflammatory-related genes. In some embodiments, the inflammatory-related gene is one or more of the following genes CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, and/or PLA2G7.

In some aspects, the disclosure provides methods for treating Sjogren’s disease in a patient in need thereof, the method comprising administering an effective amount of an RNA nuclease agent (e.g., RSLV-132) to the patient, wherein treatment results in an increase in expression of one or more inflammatory-related genes and an improvement in fatigue. In some embodiments, the inflammatory-related gene is one or more of the following genes CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, and/or PLA2G7.

In some aspects the disclosure provides methods for identifying a subject having Sjogren’s disease as a candidate for treatment with an RNA nuclease agent by determining an inflammatory-related gene expression profile in a sample obtained from the subject; and comparing the inflammatory-related gene expression profile from the subject having Sjogren’s disease with an inflammatory-related gene expression profile in a sample obtained from a suitable control subject, wherein the inflammatory-related gene expression profile indicates that the subject is a candidate for treatment with an RNA nuclease agent. In some aspects, the inflammatory-related genes include one or more of the following genes CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, PLA2G7, NUPR1, ZC3H12A, TPST1, CDK19, CCN3, IL5, IL1RAP, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and/or CXCL1.

In some aspects the disclosure provides methods for identifying a subject having Sjogren’s disease as a candidate for treatment with an RNA nuclease agent by determining an inflammatory-related gene expression profile in a sample obtained from the subject; and comparing the inflammatory-related gene expression profile from the subject having Sjogren’s disease with an inflammatory-related gene expression profile in a sample obtained from a suitable control subject, wherein the inflammatory-related gene expression profile indicates that the subject is a candidate for treatment with an RNA nuclease agent. In some aspects, the inflammatory-related genes include one or more of the following genes NUPR1, ZC3H12A, TPST1, CDK19, CCN3, IL5, IL1RAP, TBXA2R, GBA, SLC11A1, NEMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and/or CXCL1.

In some aspects the disclosure provides methods for identifying a subject having Sjogren’s disease as a candidate for treatment with an RNA nuclease agent by determining an inflammatory-related gene expression profile in a sample obtained from the subject; and comparing the inflammatory-related gene expression profile from the subject having Sjogren’s disease with an inflammatory-related gene expression profile in a sample obtained from a suitable control subject, wherein the inflammatory-related gene expression profile indicates that the subject is a candidate for treatment with an RNA nuclease agent. In some aspects, the inflammatory-related genes include one or more of the following genes CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, and/or PLA2G7.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a decrease in expression of at least 10%, 20%, 30%, 40%, 50%, 60%, 70%, 80%, 90%, or 100% of one or more of the inflammatory-related genes of the disclosure. In some embodiments, treatment results in a decrease in expression of at least 10%, 20%, 30%, 40%, 50%, 60%, 70%, 80%, 90%, or 100% of one or more of the inflammatory-related genes of the disclosure and an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in expression of at least 10%, 20%, 30%, 40%, 50%, 60%, 70%, 80%, 90%, or 100% of one or more of the inflammatory-related genes of the disclosure. In some embodiments, treatment results in an increase in expression of at least 10%, 20%, 30%, 40%, 50%, 60%, 70%, 80%, 90%, or 100% of one or more of the inflammatory-related genes of the disclosure and an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a change in expression of at least 10%, 20%, 30%, 40%, 50%, 60%, 70%, 80%, 90%, or 100% of one or more of the inflammatory-related genes of the disclosure. In some embodiments, treatment results in a change in expression of at least 10%, 20%, 30%, 40%, 50%, 60%, 70%, 80%, 90%, or 100% of one or more of the inflammatory-related genes of the disclosure and an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a change in expression of at least 10%, 20%, 30%, 40%, 50%, 60%, 70%, 80%, 90%, or 100% of one or more inflammatory-related genes selected from CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, PLA2G7, NUPR1, ZC3H12A, TPST1, CDK19, CCN3, IL5, IL1RAP, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and/or CXCL1. In some embodiments, treatment results in a change in expression of at least 10%, 20%, 30%, 40%, 50%, 60%, 70%, 80%, 90%, or 100% of one or more inflammatory related genes of the disclosure and an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a decrease in expression of at least 10%, 20%, 30%, 40%, 50%, 60%, 70%, 80%, 90%, or 100% of one or more inflammatory-related genes selected from NUPR1, ZC3H12A, TPST1, CDK19, CCN3, IL5, IL1RAP, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and/or CXCL1. In some embodiments, treatment results in a decrease in expression of at least 10%, 20%, 30%, 40%, 50%, 60%, 70%, 80%, 90%, or 100% of one or more inflammatory-related genes of the disclosure and an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in an increase in expression of at least 10%, 20%, 30%, 40%, 50%, 60%, 70%, 80%, 90%, or 100% of one or more inflammatory-related genes selected from CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, and/or PLA2G7. In some embodiments, treatment results in an increase in expression of at least 10%, 20%, 30%, 40%, 50%, 60%, 70%, 80%, 90%, or 100% of one or more inflammatory-related genes of the disclosure and an improvement in fatigue.

In some aspects the disclosure provides methods of identifying a subject with Sjogen’s syndrome that is likely to respond to treatment with an RNA nuclease agent as described herein (e.g., RSLV-132) by detecting the presence of an inflammatory-related molecule (e.g., inflammatory-related gene) in a sample obtained from the subject, wherein the presence of the inflammatory-related molecule (e.g., inflammatory-related gene) indicates the subject is likely to respond to treatment with the agent. In some aspects, the amount or expression level of the inflammatory-related molecule (e.g., inflammatory-related gene) in a sample is determined and compared to a reference amount or reference expression level of the inflammatory-related molecule (e.g., inflammatory-related gene). In some aspects, when the amount or expression level of the inflammatory-related molecule (e.g., inflammatory-related gene) in the sample is increased relative to the reference amount or reference expression level of the inflammatory-related molecule (e.g., inflammatory-related gene), then the patient is likely to respond to treatment with an RNA nuclease agent as disclosed herein. In some aspects, when the amount or expression level of the inflammatory-related molecule (e.g., inflammatory-related gene) in the sample is decreased relative to the reference amount or reference expression level of the inflammatory-related molecule (e.g., inflammatory-related gene), then the patient is likely to respond to treatment with an RNA nuclease agent as disclosed herein. In some aspects, when the amount or expression level of the inflammatory-related molecule (e.g., inflammatory-related gene) in the sample is changed relative to the reference amount or reference expression level of the inflammatory-related molecule (e.g., inflammatory-related gene), then the patient is likely to respond to treatment with an RNA nuclease agent as disclosed herein.

In some aspects, the disclosure provides a methods of identifying a patient likely to respond to treatment with an RNA nuclease agent as described herein (e.g., RSLV-132) in which a sample from the patient is contacted with a nucleic acid probe that hybridizes to a complementary target sequence in the DNA or RNA of an inflammatory-related molecule (e.g., inflammatory-related gene), thereby forming a hybridization complex between the nucleic acid probe and the DNA or RNA of the inflammatory-related molecule (e.g., inflammatory-related gene). To detect hybridization of the probe to the target DNA or RNA sequence of the inflammatory-related molecule (e.g., inflammatory-related gene), the probe is labeled with a molecular marker; for example, a radioactive marker, a fluorescent marker, an enzymatic marker, or digoxigenin. In some aspects, the presence of the probe-target complex indicates the patient is likely to respond to treatment. In some aspects, the amount of probe-target complex in the sample is compared to a control. In some aspects, when the amount of probe- target complex in the sample is increased relative to the control then the patient is likely to respond to treatment with the RNA nuclease agent (e.g., RSLV-132). In some aspects, when the amount of probe-target complex in the sample is decreased relative to the control then the patient is likely to respond to treatment with the RNA nuclease agent (e.g., RSLV-132). In some aspects, when the amount of probe- target complex in the sample is changed relative to the control then the patient is likely to respond to treatment with the RNA nuclease agent (e.g., RSLV-132).

In some aspects, the disclosure provides a method of identifying a patient with Sjogren’s syndrome as likely to respond to treatment with an RNA nuclease agent as described herein (e.g., RSLV-132) in which a sample from the patient is contacted with an antibody that specifically binds to an inflammatory-related molecule (e.g., inflammatory-related protein), or antigen-binding fragment thereof, thereby forming a complex with the inflammatory-related molecule, and the presence of the antibody-inflammatory-related molecule complex is detected, wherein the presence of the complex indicates the patient is likely to respond to treatment. In some aspects, the amount of antibody-inflammatory-related molecule complex in the sample is compared to a control. In some aspects, when the amount of antibody- inflammatory-related molecule complex in the sample is increased relative to the control then the patient is likely to respond to treatment with the RNA nuclease agent (e.g., RSLV-132). In some aspects, when the amount of antibody- inflammatory-related molecule complex in the sample is decreased relative to the control then the patient is likely to respond to treatment with the RNA nuclease agent (e.g., RSLV-132). In some aspects, when the amount of antibody-inflammatory-related molecule complex in the sample is changed relative to the control then the patient is likely to respond to treatment with the RNA nuclease agent (e.g., RSLV-132).

### Diagnostic Methods

The disclosure provides methods related to detecting and/or quantifying one or more inflammatory-related molecules (e.g., inflammatory-related genes) described herein (e.g., CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, ISG15, PLA2G7, NUPR1, ZC3H12A, TPST1, CDK19, CCN3, IL5, IL1RAP, CXCL10, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, IL-18, F11R, ADAM8, NLRP12, NDST1, IL1R2, MAPK13, MMP9, NOD2, and/or CXCL1), in one or more samples, wherein the detection and/or quantification of the one or more inflammatory-related molecules individually or in combination, will indicate a likelihood that an RNA nuclease agent (e.g., RSLV-132) will provide a therapeutic effect or benefit to a patient with Sjogren’s disease.

Provided herein are methods for identifying a subject having Sjogren’s disease as a candidate for treatment with an RNA nuclease agent by determining an inflammatory-related gene expression profile in a sample obtained from the subject; and comparing the inflammatory-related gene expression profile from the subject having Sjogren’s disease with an inflammatory-related gene expression profile in a sample obtained from a suitable control subject, wherein the inflammatory-related gene expression profile indicates that the subject is a candidate for treatment with an RNA nuclease agent (e.g., RSLV-132). In some aspects, the inflammatory-related genes in the gene expression profile include CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, PLA2G7, NUPR1, ZC3H12A, TPST1, CDK19, CCN3, IL5, IL1RAP, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and/or CXCL1. In some aspects, the inflammatory-related genes in the gene expression profile include CD36, RIPK2, PTPN2, APOL3, HGF, BIRC3, SETD6, CD47, IDO1, ACER3, APPL1, SNX4, CCR2, CD163, VAMP7, ISG15, TBC1D23, CXCL10, AIMP1, CYBB, PLA2G7, NUPR1, ZC3H12A, TPST1, CDK19, CCN3, IL5, IL1RAP, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, CXCL10 NFAM1, IL17RA, IL6R, AKT1, TNFRSF1A, IL-18, F11R, ADAM8, NLRP12, NDST1, IL1R2, MAPK13, MMP9, NOD2, and/or CXCL1. In some aspects, the inflammatory-related genes in the gene expression profile include NUPR1, ZC3H12A, TPST1, CDK19, CCN3, IL5, IL1RAP, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and/or CXCL1. In some aspects, the inflammatory-related genes in the gene expression profile include CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, and/or PLA2G7.

In some embodiments, the expression level of one or more inflammatory-related molecules (e.g., inflammatory-related genes) of the disclosure in the sample from a patient with Sjogren’s syndrome is compared to the expression level of the one or more one or more inflammatory-related molecules (e.g., inflammatory-related genes) in a control. In some embodiments, a control is a sample from a healthy subject. In some embodiments, a control is a sample taken from the patient prior to treatment. In some embodiments, a control is a sample take from the patient prior to disease onset. In some embodiments, a control is a sample taken from a subject that did not respond to treatment.

Further provided herein are methods for identifying a subject having Sjogren’s disease as a candidate for treatment with an RNA nuclease agent by determining an inflammatory-related gene expression profile in a sample obtained from the subject; and comparing the inflammatory-related gene expression profile from the subject having Sjogren’s disease with an inflammatory-related gene expression profile in a sample obtained from a suitable control, wherein the inflammatory-related gene expression profile indicates that the subject is a candidate for treatment with an RNA nuclease agent (e.g., RSLV-132) when the expression of one or more inflammatory-related genes in the sample from the subject having Sjogren’s disease is elevated as compared to of one or more inflammatory-related genes in the sample obtained from a suitable control. In some embodiments, a control is a sample from a healthy subject. In some embodiments, a control is a sample taken from the patient prior to the onset of disease. In some embodiments, a control is a sample taken from a subject that did not respond to treatment.

Further provided herein are methods for identifying a subject having Sjogren’s disease as a candidate for treatment with an RNA nuclease agent by determining an inflammatory-related gene expression profile in a sample obtained from the subject; and comparing the inflammatory-related gene expression profile from the subject having Sjogren’s disease with an inflammatory-related gene expression profile in a sample obtained from a suitable control, wherein the inflammatory-related gene expression profile indicates that the subject is a candidate for treatment with an RNA nuclease agent when the expression of one or more inflammatory-related genes in the sample from the subject having Sjogren’s disease is less than the amount of one or more inflammatory-related genes in the sample obtained from a suitable control. In some embodiments, a control is a sample from a healthy subject. In some embodiments, a control is a sample taken from the patient prior to the onset of disease. In some embodiments, a control is a sample taken from a subject that did not respond to treatment.

Further provided herein are methods of identifying a patient with Sjogren’s disease as likely to respond to treatment with an RNA nuclease agent (e.g., RSLV-132), the method comprising: determining an expression level of one or more inflammatory-related molecules (e.g., inflammatory-related gene) in a sample obtained from the patient relative to a reference expression level of the inflammatory-related molecule (e.g., inflammatory-related gene), wherein the expression level of the inflammatory-related molecule (e.g., inflammatory-related gene) in the sample relative to the reference expression level of the inflammatory-related molecule (e.g., inflammatory-related gene) indicates a likelihood the patient will respond to treatment.

Further provided herein are methods of identifying a patient with Sjogren’s disease as likely to respond to treatment with an RNA nuclease agent (e.g., RSLV-132), the method comprising: determining an expression level of one or more inflammatory-related genes in a sample obtained from the patient; and comparing the expression level of one or more inflammatory-related genes in the sample to a reference expression level of one or more inflammatory-related genes, wherein the patient is likely to respond to treatment when the expression level of one or more inflammatory-related genes in the sample is elevated as compared to the reference expression level of one or more inflammatory-related genes. In some embodiments, the reference expression level is determined from a sample in a healthy subject. In some embodiments, the reference expression level is determined from a sample taken from the patient prior to disease onset. In some embodiments, the reference expression level is determined in a sample taken from the patient prior to treatment. In some embodiments, the reference expression level is determined in a sample taken from the patient that did not respond to treatment.

Further provided herein are methods of identifying a patient with Sjogren’s disease as likely to respond to treatment with an RNA nuclease agent (e.g., RSLV-132), the method comprising: determining an expression level of one or more inflammatory-related genes in a sample obtained from the patient; and comparing the expression level of one or more inflammatory-related genes in the sample to a reference expression level of one or more inflammatory-related genes, wherein the patient is likely to respond to treatment when the expression level of one or more inflammatory-related genes in the sample is less than the reference expression level of one or more inflammatory-related genes. In some embodiments, the reference expression level is determined from a sample in a healthy subject. In some embodiments, the reference expression level is determined from a sample taken from the patient prior to disease onset. In some embodiments, the reference expression level is determined in a sample taken from the patient prior to treatment. In some embodiments, the reference expression level is determined in a sample taken from the patient that did not respond to treatment.

Further provided herein are methods of identifying a patient with Sjogren’s disease as likely to respond to treatment with an RNA nuclease agent (e.g., RSLV-132), the method comprising: determining the expression levels of a panel of inflammatory-related genes in a sample from the patient; comparing the expression levels of the panel in the sample to the expression levels of the panel in a control, wherein a change in the expression levels of the inflammatory-related genes in the sample relative to the expression levels of the inflammatory-related genes in the control indicates a likelihood the patient will respond to treatment. In some embodiments, the expression levels of inflammatory-related genes in the sample is increased relative to the expression levels of inflammatory-related genes in the control. In some embodiments, the expression levels of inflammatory-related genes in the sample is decreased relative to the expression levels of inflammatory-related genes in the control. In some embodiments, the control is a sample taken from a healthy subject. In some embodiments, the control is a sample taken from the patient prior to disease onset. In some embodiments, the control is a sample taken from the patient prior to treatment. In some embodiments, the reference expression level is determined in a sample taken from the patient that did not respond to treatment.

Further provided herein are methods of identifying a patient with Sjogren’s disease as likely to respond to treatment with an RNA nuclease agent (e.g., RSLV-132), the method comprising: contacting a sample with a nucleic acid probe that hybridizes to a complementary target sequence in the DNA or RNA of an inflammatory-related molecule (e.g., inflammatory-related gene), thereby forming a hybridization complex between the nucleic acid probe and the DNA or RNA of the inflammatory-related molecule (e.g., inflammatory-related gene); determining an amount of complex in the sample relative to an amount of complex in a reference sample, wherein the amount of the complex in the sample relative to the amount of the complex in the reference sample indicates a likelihood the patient is susceptible treatment with the RNA nuclease agent (e.g., RSLV-132). In some embodiments, the amount of the complex in the sample is increased relative to the amount of the complex in the reference sample. In some embodiments, the amount of the complex in the sample is decreased relative to the amount of the complex in the reference sample.

Further provided herein are methods of identifying a patient with Sjogren’s disease as likely to respond to treatment with an RNA nuclease agent (e.g., RSLV-132), the method comprising: contacting a sample with at least one diagnostic antibody, or antigen-binding fragment thereof, that specifically binds to an inflammatory-related molecule (e.g., inflammatory-related gene), thereby forming a diagnostic antibody-inflammatory-related molecule complex; determining an amount of complex in the sample relative to an amount of complex in a reference sample, wherein the amount of the complex in the sample relative to the amount of the complex in the reference sample indicates a likelihood the patient is susceptible treatment with the RNA nuclease agent (e.g., RSLV-132). In some embodiments, the amount of the complex in the sample is increased relative to the amount of the complex in the reference sample. In some embodiments, the amount of the complex in the sample is decreased relative to the amount of the complex in the reference sample.

Further provided herein are methods for monitoring a response of a patient with Sjogren’s disease treated with an RNA nuclease agent (e.g., RSLV-132), the method comprising: determining the expression level and/or activity of one or more inflammatory-related molecules (e.g., an inflammatory-related gene), in a first sample from the patient obtained prior to treatment with the RNA nuclease agent; determining the expression level and/or activity of the one or more inflammatory-related molecules (e.g., an inflammatory-related gene) in a second sample from the patient obtained after treatment with the RNA nuclease agent; comparing the expression level and/or activity of the one or more inflammatory-related molecules (e.g., an inflammatory-related gene) in the second sample with the expression level and/or activity of the one or more inflammatory-related molecules (e.g., an inflammatory-related gene) in the first sample, wherein a change of the expression level and/or activity of the one or more inflammatory-related molecules (e.g., an inflammatory-related gene) in the second sample relative to the expression level and/or activity of the one or more inflammatory-related molecules (e.g., an inflammatory-related gene) in the first sample indicates a response of the patient treated with the RNA nuclease agent. In some aspects, the one or more inflammatory-related molecules is an inflammatory-related gene. In some embodiments, the inflammatory-related gene is one or more of CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, PLA2G7, NUPR1, ZC3H12A, TPST1, CDK19, CCN3, IL5, IL1RAP, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and/or CXCL1.

In some embodiments, the inflammatory-related gene is one or more of CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, ISG15, PLA2G7, NUPR1, ZC3H12A, TPST1, CDK19, CCN3, IL5, IL1RAP, CXCL10, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, IL-18, F11R, ADAM8, NLRP12, NDST1, IL1R2, MAPK13, MMP9, NOD2, and/or CXCL1.

Further provided herein are methods for monitoring a response of a patient with Sjogren’s disease treated with an RNA nuclease agent (e.g., RSLV-132), the method comprising: determining the expression level and/or activity of one or more inflammatory-related molecules (e.g., an inflammatory-related gene), in a first sample from the patient obtained prior to treatment with the RNA nuclease agent; determining the expression level and/or activity of the one or more inflammatory-related molecules (e.g., an inflammatory-related gene) in a second sample from the patient obtained after treatment with the RNA nuclease agent; comparing the expression level and/or activity of the one or more inflammatory-related molecules (e.g., an inflammatory-related gene) in the second sample with the expression level and/or activity of the one or more inflammatory-related molecules (e.g., an inflammatory-related gene) in the first sample, wherein a decrease in the expression level and/or activity of the one or more inflammatory-related molecules (e.g., an inflammatory-related gene) in the second sample relative to the expression level and/or activity of the one or more inflammatory-related molecules (e.g., an inflammatory-related gene) in the first sample indicates a response of the patient treated with the RNA nuclease agent. In some aspects, the one or more inflammatory-related molecules is an inflammatory-related gene. In some embodiments, the inflammatory-related gene is one or more of CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, PLA2G7, NUPR1, ZC3H12A, TPST1, CDK19, CCN3, IL5, IL1RAP, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and/or CXCL1. In some embodiments, the inflammatory-related gene is one or more of CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, ISG15, PLA2G7, NUPR1, ZC3H12A, TPST1, CDK19, CCN3, IL5, IL1RAP, CXCL10 TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, IL-18, F11R, ADAM8, NLRP12, NDST1, IL1R2, MAPK13, MMP9, NOD2, and/or CXCL1. In some embodiments, the inflammatory-related gene is one or more of NUPR1, ZC3H12A, TPST1, CDK19, CCN3, IL5, IL1RAP, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and/or CXCL1.

Further provided herein are methods for monitoring a response of a patient with Sjogren’s disease treated with an RNA nuclease agent (e.g., RSLV-132), the method comprising: determining the expression level and/or activity of one or more inflammatory-related molecules (e.g., an inflammatory-related gene), in a first sample from the patient obtained prior to treatment with the RNA nuclease agent; determining the expression level and/or activity of the one or more inflammatory-related molecules (e.g., an inflammatory-related gene) in a second sample from the patient obtained after treatment with the RNA nuclease agent; comparing the expression level and/or activity of the one or more inflammatory-related molecules (e.g., an inflammatory-related gene) in the second sample with the expression level and/or activity of the one or more inflammatory-related molecules (e.g., an inflammatory-related gene) in the first sample, wherein an increase in the expression level and/or activity of the one or more inflammatory-related molecules (e.g., an inflammatory-related gene) in the second sample relative to the expression level and/or activity of the one or more inflammatory-related molecules (e.g., an inflammatory-related gene) in the first sample indicates a response of the patient treated with the RNA nuclease agent. In some aspects, the one or more inflammatory-related molecules is an inflammatory-related gene. In some embodiments, the inflammatory-related gene is one or more of CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, PLA2G7, NUPR1, ZC3H12A, TPST1, CDK19, CCN3, IL5, IL1RAP, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and/or CXCL1. In some embodiments, the inflammatory-related gene is one or more of CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, ISG15, PLA2G7, NUPR1, ZC3H12A, TPST1, CDK19, CCN3, IL5, IL1RAP, CXCL10, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and/or CXCL1. In some embodiments, the inflammatory-related gene is one or more of CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, and/or PLA2G7.

### Determination of Inflammatory-Related Molecules

The presence of or an amount or expression level of one or more inflammatory-related molecules (e.g., inflammatory-related genes, inflammatory-related proteins, pro-inflammatory molecules, such as a pro-inflammatory gene or pro-inflammatory protein) described herein in a sample can be detected or determined by a number of methodologies and techniques, which are known in the art and understood by the skilled artisan, including, but not limited to, immunohistochemistry (IHC), immunofluorescence (IF), Western blot analysis, immunoprecipitation, molecular binding assays, enzyme-linked immunosorbent assay (ELISA), enzyme-linked immunofiltration assay (ELIFA), flow cytometry, MassARRAY, proteomics, quantitative blood based assays (e.g., Serum ELISA), biochemical enzymatic activity assays, in situ hybridization, fluorescence in situ hybridization (FISH), Southern analysis, Northern analysis, whole genome sequencing, polymerase chain reaction (PCR) including quantitative real time PCR (qRT-PCR) and other amplification type detection methods, such as, for example, branched DNA, SISBA, TMA and the like, RNA-Seq, microarray analysis, gene expression profiling, and/or serial analysis of gene expression (SAGE), as well as any one of the wide variety of assays that can be performed by protein, gene, and/or tissue array analysis. Typical protocols for evaluating the status of genes and gene products are found, for example in Ausubel et al., eds., 1 995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used. Diagnostic antibodies that bind the inflammatory-related molecules of the disclosure are available from a variety of commercial sources, such as BD Biosciences, ebiosciences, BioLegend, Abcam, and the like.

### Nucleic Acid Inflammatory-Related Molecule Techniques

In some embodiments, the expression level of an inflammatory-related molecule (e.g., an inflammatory-related gene), described herein, may be a nucleic acid expression level. In some embodiments, the nucleic acid expression level is determined using qPCR, rtPCR, RNA-Seq, multiplex qPCR or RT-qPCR, microarray analysis, gene expression profiling, SAGE, MassARRAY technique, or in situ hybridization (e.g., FISH). In some embodiments, the expression level of an inflammatory-related molecule (e.g., inflammatory-related gene) is determined in cells from a patient with Sjogren’s disease. In some embodiments, the expression level of a an inflammatory-related molecule (e.g., inflammatory-related gene) described herein is determined in blood cells from a patient with Sjogren’s disease.

Methods for the evaluation of mRNAs in cells are known in the art and include, for example, hybridization assays using complementary DNA probes (such as in situ hybridization using labeled riboprobes specific for the one or more genes, Northern blot and related techniques) and various nucleic acid amplification assays (such as RT-PCR using complementary primers specific for one or more of the genes, and other amplification type detection methods, such as, for example, branched DNA, SISBA, TMA and the like). In addition, such methods can include one or more steps that allow one to determine the levels of target mRNA in a biological sample (e.g., by simultaneously examining the levels a comparative control mRNA sequence of a “housekeeping” gene such as an actin family member).

The nucleic acid sequences of the inflammatory-related molecules (e.g., inflammatory-related genes, pro-inflammatory genes) of the disclosure are known in the art. In some embodiments, the inflammatory-related molecules (e.g., inflammatory-related genes) of the disclosure include one or more of CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, PLA2G7, NUPR1, ZC3H12A, TPST1, CDK19, CCN3, IL5, IL1RAP, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and/or CXCL1. In some embodiments, the inflammatory-related molecules (e.g., inflammatory-related genes) of the disclosure include one or more of CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, ISG15, PLA2G7, NUPR1, ZC3H12A, TPST1, CDK19, CCN3, IL5, IL1RAP, CXCL10, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, IL-18, F11R, ADAM8, NLRP12, NDST1, IL1R2, MAPK13, MMP9, NOD2, and/or CXCL1.

In some embodiments, the sequence of the amplified target cDNA can be determined. Methods include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes whose expression correlates with increased or reduced clinical benefit of treatment comprising an RNA nuclease agent (e.g., RSLV-132), may be arrayed on a solid support. Hybridization of a labeled probe with a particular array member indicates that the sample from which the probe was derived expresses that gene.

Inclusion of any of the diagnostic methods described herein as part of any method directed to methods for identifying patients likely to benefit from treatment as described herein (e.g., selection of a therapeutic treatment or intervention) or to the development of treatments (e.g., enrollment patients in clinical trials) provides an advantage over those methods that do not include the diagnostic methods, in that a patient population whose members are predicted to need and/or not need, benefit, or respond to treatment may be identified.

Accordingly, in some embodiments, the inflammatory-related molecule (e.g., inflammatory-related gene or pro-inflammatory molecules (e.g., pro-inflammatory gene)) suitable for use in the present disclosure is a diagnostic inflammatory-related molecule (e.g., inflammatory-related gene or pro-inflammatory molecules (e.g., pro-inflammatory gene)). In some embodiments, the inflammatory-related molecule (e.g., inflammatory-related gene or pro-inflammatory molecule (e.g., pro-inflammatory gene)) is a monitoring inflammatory-related molecule (e.g., inflammatory-related gene or pro-inflammatory molecules (e.g., pro-inflammatory gene)). In some embodiments, the inflammatory-related molecule (e.g., inflammatory-related gene or pro-inflammatory molecules (e.g., pro-inflammatory gene)) is a predictive inflammatory-related molecule (e.g., inflammatory-related gene or pro-inflammatory molecules (e.g., pro-inflammatory gene)).

Inflammatory-related molecules (e.g., inflammatory-related gene or pro-inflammatory molecules (e.g., pro-inflammatory gene)) can be a substance or a biological event whose detection indicates a particular physiological state (e.g., a diseased state). For example, the presence of an inflammatory-related gene in the serum of a patient may indicate disease (e.g., Sjogren’s syndrome). Inflammatory-related molecules (e.g., inflammatory-related genes or pro-inflammatory molecules (e.g., pro-inflammatory genes)) measured in patients before treatment can be used to identify suitable patients for inclusion in a clinical trial. Inflammatory-related molecule (e.g., inflammatory-related gene or pro-inflammatory molecule (e.g., pro-inflammatory gene)) changes after treatment may predict or identify safety problems related to a candidate drug, or reveal pharmacologic activity expected to predict an eventual benefit of treatment. Inflammatory-related molecules (e.g., inflammatory-related gene or pro-inflammatory molecules (e.g., pro-inflammatory gene)) may reduce uncertainty in drug development and evaluation by providing quantifiable predictions about drug performance, and they can contribute to dose selection. Composite inflammatory-related molecules (e.g., inflammatory-related gene or pro-inflammatory molecules (e.g., pro-inflammatory gene)) include several individual inflammatory-related molecules (e.g., inflammatory-related gene or pro-inflammatory molecules (e.g., pro-inflammatory gene)) in a stated algorithm which reaches a single interpretive readout when a single inflammatory-related molecules (e.g., inflammatory-related gene or pro-inflammatory molecules (e.g., pro-inflammatory gene)) fails to provide all the relevant information required for assessment.

A surrogate end point is an inflammatory-related molecules (e.g., inflammatory-related gene or pro-inflammatory molecules (e.g., pro-inflammatory gene)) that is intended to substitute for a clinical end point and is expected, based on epidemiologic, therapeutic, pathophysiologic, or other scientific evidence, to predict clinical benefit.

### Protein Inflammatory-Related Molecule Techniques

In some embodiments, the amount of an inflammatory-related molecule (e.g., an inflammatory-related protein) is measured by determining the protein expression level of the inflammatory-related molecules (e.g., inflammatory-related protein or pro-inflammatory molecule (e.g., pro-inflammatory protein)). There are a number of techniques that measure or determine protein expression levels known in the art and described herein that may be used in the methods provided by the disclosure. For example, in some embodiments, a protein expression level of a the inflammatory-related molecule (e.g., inflammatory-related protein or pro-inflammatory molecules (e.g., pro-inflammatory protein)) is determined using a method selected from the group consisting of flow cytometry (e.g., fluorescence-activated cell sorting (FACS™)), Western blot, enzyme-linked immunosorbent assay (ELISA), immunoprecipitation, immunohistochemistry (IHC), immunofluorescence, radioimmunoassay, dot blotting, immunodetection methods, HPLC, surface plasmon resonance, optical spectroscopy, mass spectrometry, and HPLC.

In some embodiments, a sample is contacted with an antibody that specifically binds to an inflammatory-related molecule (e.g., inflammatory-related protein or pro-inflammatory molecules (e.g., pro-inflammatory protein)) described herein under conditions permissive for binding of the inflammatory-related molecules (e.g., inflammatory-related protein or pro-inflammatory molecules (e.g., pro-inflammatory protein)), and the presence of a complex formed by the antibody and the inflammatory-related molecules (e.g., inflammatory-related protein or pro-inflammatory molecules (e.g., pro-inflammatory protein)) is detected. In some embodiments, a sample is contacted with a combination of antibodies that specifically bind to a combination of inflammatory-related molecules (e.g., inflammatory-related protein or pro-inflammatory molecules (e.g., pro-inflammatory protein)) described herein. In some embodiments, the protein expression level of the inflammatory-related molecules (e.g., inflammatory-related protein or pro-inflammatory molecules (e.g., pro-inflammatory protein)) is determined in cells from a patient with Sjogren’s′ disease. In some embodiments, the protein expression level of the inflammatory-related molecules (e.g., inflammatory-related protein or pro-inflammatory molecules (e.g., pro-inflammatory protein)) is determined in blood cells from a patient with Sjogren’s disease.

In some embodiments, the amount of an inflammatory-related molecule (e.g., inflammatory-related protein or pro-inflammatory molecule (e.g., pro-inflammatory protein)) in a sample is determined using a diagnostic antibody that binds the inflammatory-related molecule (e.g., inflammatory-related protein or pro-inflammatory molecule (e.g., pro-inflammatory protein)). In some embodiments, the anti-inflammatory-related molecule diagnostic antibody specifically binds the inflammatory-related molecule (e.g., inflammatory-related protein or pro-inflammatory molecules (e.g., pro-inflammatory protein)). In some embodiments, the diagnostic antibody is a non-human antibody. In some embodiments, the diagnostic antibody is a rat, mouse, or rabbit antibody. In some embodiments, the diagnostic antibody is a monoclonal antibody. In some embodiments, the diagnostic antibody is directly labeled. In other embodiments, the diagnostic antibody is indirectly labeled.

### Methods and Uses of Long Non-Coding RNA Molecules

Provided herein are diagnostic and therapeutic methods and uses of the long non-coding RNA (lncRNA) molecules described herein. Further provided herein are methods of identifying subjects having Sjogren’s syndrome that have achieved a response to treatment with an RNA nuclease agent as described herein by detecting the presence of or determining the amount or expression level of one or more lncRNA molecules (e.g., amount or expression level) in a sample obtained from the subject, wherein the presence of or the amount or expression level of the one or more IncRNA molecules indicates the subject achieved a response to the RNA nuclease agent. In some embodiments, a decrease in expression of one or more IncRNA molecules of the disclosure indicates the subject has achieved a response to the RNA nuclease agent (e.g., RSLV-132).

In some embodiments, the expression level of the one or more lncRNA molecule(s) in the sample from a patient with Sjogren’s syndrome is compared to the expression level of the one or more lncRNA molecule(s) in a control. In some embodiments, a control is a sample from a healthy subject. In some embodiments, a control is a sample taken from the patient prior to treatment. In some embodiments, a control is a sample take from the patient prior to disease onset. In some embodiments, a control is a sample taken from a subject that did not respond to treatment.

### Long Non-Coding RNA Molecules

In some aspects, the disclosure provides methods for detecting the presence of or determining an amount or expression level of one or more long non-coding RNA (lncRNA) molecules in a sample from a subject (e.g., a sample from a subject with Sjogren’s syndrome). In some aspects, the disclosure provides methods for treating a subject having Sjogren’s syndrome a with an RNase nuclease agent (e.g., RSLV-132), wherein treatment results in a decrease in the amount or expression level of one or more lncRNA molecules.

As used herein, the term “long non-coding RNA molecule” or “lncRNA” refers to an RNA molecule that exceeds 200 nucleotides in length and is not translated into protein. In some embodiments, the lncRNA molecules of the disclosure regulate the transcription of genes involved in inflammation and/or regulate the transcription of pro-inflammatory genes. In some embodiments, the pro-inflammatory genes are pro-inflammatory genes related to autoimmune disease. In some embodiments, the pro-inflammatory genes are pro-inflammatory genes related immune system function.

For example, in some embodiments, PXN-AS1, AC116366.2, HCG27, AL034417.2, ZMIZ1-AS1, AL513348.1, AC243829.2, LINC01227, LINC01091, AC080011.1, AL591368.1, AC109495.1, KLF3-AS1, and LINC01448 regulate the transcription of genes involved in inflammation. In some embodiments, PXN-AS1, AC116366.2, HCG27, AL034417.2, ZMIZ1-AS1, AL513348.1, AC243829.2, LINC01227, LINC01091, AC080011.1, AL591368.1, AC109495.1, KLF3-AS1, and LINC01448 regulate the transcription of pro-inflammatory genes. In some embodiments, PXN-AS1, AC116366.2, HCG27, AL034417.2, ZMIZ1-AS1, AL513348.1, AC243829.2, LINC01227, LINC01091, AC080011.1, AL591368.1, AC109495.1, KLF3-AS1, and LINC01448 regulate the transcription of pro-inflammatory genes related to autoimmune diseases and/or immune system function.

In some embodiments, one or more IncRNA molecules of the disclosure are selected from PXN-AS1, AP002852.1, AL009176.1, MKX-AS1, AC109464.1, AL590560.2, AC020908.3, AL731533.3, AL353743.2, AC007405.1, DPRXP2, ST20-AS1, AC020916.1, AC027601.1, AL358334.2, AL358394.2, AL022238.2, AC116366.2, AP003031.1, CYP4F30P, AL390071.1, AC008532.1, CPHL1P, AC008870.4, AC008763.1, AL355388.2, AC110926.2, AL133415.1, AL591503.1, DLGAP1-AS1, CHL1-AS2, AC010326.4, AC051649.1, HCG27, AL034417.2, AP001160.1, AC009435.1, LINC01499, AL161621.1, AP001469.1, AC021491.2, AC010168.1, SLC44A3-AS1, AC011445.2, ZMIZ1-AS1, AC027018.1, AL591379.1, AC061992.1, AL513348.1, AC092617.1, AC015853.3, AC138655.1, AC108134.3, LINC01515, AC243829.2, AC020911.1, AC016168.4, AL590705.1, FAM3D-AS1, AC078881.1, AC004466.2, AC018866.1, AC010531.3, AJ011931.2, TUSC8, PTOV1-AS1, RPS4XP2, AC021148.2, AC009041.2, AC093525.6, LINC01516, AL670729.3, C18ORF65, AC098818.2, FAM230C, LINC01988, AC068831.1, CYP51A1-AS1, LINC01227, LINC00895, LINC01519, AC114730.3, MIPEPP3, ITPRIP-AS1, AC138207.5, LINC01091, AC012645.3, AC082651.1, DLGAP1-AS3, AC080011.1, AC010148.1, AC078962.1, AL132822.1, LINC02552, AC098484.2, AC090587.1, LINC00173, PIK3CD-AS1, AL021393.1, AL450322.2, AL591368.1, AC010894.4, AC067930.8, AC024267.3, AC104984.1, AC109495.1, AC129926.1, KLF3-AS1, AL512306.3, LINC01448, AC080013.6, and DACT3-AS1.

In some embodiments, one or more lncRNA molecules of the disclosure are selected from PXN-AS1, AC116366.2, HCG27, AL034417.2, ZMIZ1-AS1, AL513348.1, AC243829.2, LINC01227, LINC01091, AC080011.1, AL591368.1, AC109495.1, KLF3-AS1, and LINC01448.

In some embodiments, an IncRNA molecule of the disclosure is PXN-AS1. In some embodiments, an IncRNA molecule of the disclosure is AP002852.1. In some embodiments, an IncRNA molecule of the disclosure is AL009176.1. In some embodiments, an IncRNA molecule of the disclosure is MKX-AS1. In some embodiments, an IncRNA molecule of the disclosure is AC109464.1. In some embodiments, an IncRNA molecule of the disclosure is AL590560.2. In some embodiments, an IncRNA molecule of the disclosure is AC020908.3. In some embodiments, an IncRNA molecule of the disclosure is AL731533.3. In some embodiments, an IncRNA molecule of the disclosure is AL353743.2. In some embodiments, an IncRNA molecule of the disclosure is AC007405.1. In some embodiments, an IncRNA molecule of the disclosure is DPRXP2. In some embodiments, an IncRNA molecule of the disclosure is ST20-AS1. In some embodiments, an IncRNA molecule of the disclosure is AC020916.1. In some embodiments, an IncRNA molecule of the disclosure is AC027601.1. In some embodiments, an IncRNA molecule of the disclosure is AL358334.2. In some embodiments, an IncRNA molecule of the disclosure is AL358394.2. In some embodiments, an IncRNA molecule of the disclosure is AL022238.2. In some embodiments, an IncRNA molecule of the disclosure is AC116366.2. In some embodiments, an IncRNA molecule of the disclosure is AP003031.1. In some embodiments, an IncRNA molecule of the disclosure is CYP4F30P. In some embodiments, an IncRNA molecule of the disclosure is AL390071.1. In some embodiments, an IncRNA molecule of the disclosure is AC008532.1. In some embodiments, an IncRNA molecule of the disclosure is CPHL1P. In some embodiments, an IncRNA molecule of the disclosure is AC008870.4. In some embodiments, an IncRNA molecule of the disclosure is AC008763.1. In some embodiments, an IncRNA molecule of the disclosure is AL355388.2. In some embodiments, an IncRNA molecule of the disclosure is AC110926.2. In some embodiments, an IncRNA molecule of the disclosure is AL133415.1. In some embodiments, an IncRNA molecule of the disclosure is AL591503.1. In some embodiments, an IncRNA molecule of the disclosure is DLGAP1-AS1. In some embodiments, an IncRNA molecule of the disclosure is CHL1-AS2. In some embodiments, an IncRNA molecule of the disclosure is AC010326.4. In some embodiments, an IncRNA molecule of the disclosure is AC051649.1. In some embodiments, an IncRNA molecule of the disclosure is HCG27. In some embodiments, an IncRNA molecule of the disclosure is AL034417.2. In some embodiments, an IncRNA molecule of the disclosure is AP001160.1. In some embodiments, an IncRNA molecule of the disclosure is AC009435.1. In some embodiments, an IncRNA molecule of the disclosure is LINC01499. In some embodiments, an IncRNA molecule of the disclosure is AL161621.1. In some embodiments, an IncRNA molecule of the disclosure is AP001469.1. In some embodiments, an IncRNA molecule of the disclosure is AC021491.2. In some embodiments, an IncRNA molecule of the disclosure is AC010168.1. In some embodiments, an IncRNA molecule of the disclosure is SLC44A3-AS1. In some embodiments, an IncRNA molecule of the disclosure is AC011445.2. In some embodiments, an IncRNA molecule of the disclosure is ZMIZ1-AS1. In some embodiments, an IncRNA molecule of the disclosure is AC027018.1. In some embodiments, an IncRNA molecule of the disclosure is AL591379.1. In some embodiments, an IncRNA molecule of the disclosure is AC061992.1. In some embodiments, an IncRNA molecule of the disclosure is AL513348.1. In some embodiments, an IncRNA molecule of the disclosure is AC092617.1. In some embodiments, an IncRNA molecule of the disclosure is AC015853.3. In some embodiments, an IncRNA molecule of the disclosure is AC138655.1. In some embodiments, an IncRNA molecule of the disclosure is AC108134.3. In some embodiments, an IncRNA molecule of the disclosure is LINC01515. In some embodiments, an IncRNA molecule of the disclosure is AC243829.2. In some embodiments, an IncRNA molecule of the disclosure is AC020911.1. In some embodiments, an IncRNA molecule of the disclosure is AC016168.4. In some embodiments, an IncRNA molecule of the disclosure is AL590705.1. In some embodiments, an IncRNA molecule of the disclosure is FAM3D-AS1. In some embodiments, an IncRNA molecule of the disclosure is AC078881.1. In some embodiments, an IncRNA molecule of the disclosure is AC004466.2. In some embodiments, an IncRNA molecule of the disclosure is AC018866.1. In some embodiments, an IncRNA molecule of the disclosure is AC010531.3. In some embodiments, an IncRNA molecule of the disclosure is AJ011931.2. In some embodiments, an IncRNA molecule of the disclosure is TUSC8. In some embodiments, an IncRNA molecule of the disclosure is PTOV1-AS1. In some embodiments, an IncRNA molecule of the disclosure is RPS4XP2. In some embodiments, an IncRNA molecule of the disclosure is AC021148.2. In some embodiments, an IncRNA molecule of the disclosure is AC009041.2. In some embodiments, an IncRNA molecule of the disclosure is AC093525.6, LINC01516. In some embodiments, an IncRNA molecule of the disclosure is AL670729.3. In some embodiments, an IncRNA molecule of the disclosure is C18ORF65. In some embodiments, an IncRNA molecule of the disclosure is AC098818.2. In some embodiments, an IncRNA molecule of the disclosure is FAM230C. In some embodiments, an IncRNA molecule of the disclosure is LINC01988. In some embodiments, an IncRNA molecule of the disclosure is AC068831.1. In some embodiments, an IncRNA molecule of the disclosure is CYP51A1-AS1. In some embodiments, an IncRNA molecule of the disclosure is LINC01227. In some embodiments, an IncRNA molecule of the disclosure is LINC00895. In some embodiments, an IncRNA molecule of the disclosure is LINC01519. In some embodiments, an IncRNA molecule of the disclosure is AC114730.3. In some embodiments, an IncRNA molecule of the disclosure is MIPEPP3. In some embodiments, an IncRNA molecule of the disclosure is ITPRIP-AS1. In some embodiments, an IncRNA molecule of the disclosure is AC138207.5. In some embodiments, an IncRNA molecule of the disclosure is LINC01091. In some embodiments, an IncRNA molecule of the disclosure is AC012645.3. In some embodiments, an IncRNA molecule of the disclosure is AC082651.1. In some embodiments, an IncRNA molecule of the disclosure is DLGAP1-AS3. In some embodiments, an IncRNA molecule of the disclosure is AC080011.1. In some embodiments, an IncRNA molecule of the disclosure is AC010148.1. In some embodiments, an IncRNA molecule of the disclosure is AC078962.1. In some embodiments, an IncRNA molecule of the disclosure is AL132822.1. In some embodiments, an IncRNA molecule of the disclosure is LINC02552. In some embodiments, an IncRNA molecule of the disclosure is AC098484.2. In some embodiments, an IncRNA molecule of the disclosure is AC090587.1. In some embodiments, an IncRNA molecule of the disclosure is LINC00173. In some embodiments, an IncRNA molecule of the disclosure is PIK3CD-AS1. In some embodiments, an IncRNA molecule of the disclosure is AL021393.1. In some embodiments, an IncRNA molecule of the disclosure is AL450322.2. In some embodiments, an IncRNA molecule of the disclosure is AL591368.1. In some embodiments, an IncRNA molecule of the disclosure is AC010894.4. In some embodiments, an IncRNA molecule of the disclosure is AC067930.8. In some embodiments, an IncRNA molecule of the disclosure is AC024267.3. In some embodiments, an IncRNA molecule of the disclosure is AC104984.1. In some embodiments, an IncRNA molecule of the disclosure is AC109495.1. In some embodiments, an IncRNA molecule of the disclosure is AC129926.1. In some embodiments, an IncRNA molecule of the disclosure is KLF3-AS1. In some embodiments, an IncRNA molecule of the disclosure is AL512306.3. In some embodiments, an IncRNA molecule of the disclosure is LINC01448. In some embodiments, an IncRNA molecule of the disclosure is AC080013.6. In some embodiments, an IncRNA molecule of the disclosure is DACT3-AS1.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a decrease in expression of one or more IncRNA molecules. In some embodiments, one or more IncRNA molecules are selected from PXN-AS1, AP002852.1, AL009176.1, MKX-AS1, AC109464.1, AL590560.2, AC020908.3, AL731533.3, AL353743.2, AC007405.1, DPRXP2, ST20-AS1, AC020916.1, AC027601.1, AL358334.2, AL358394.2, AL022238.2, AC116366.2, AP003031.1, CYP4F30P, AL390071.1, AC008532.1, CPHL1P, AC008870.4, AC008763.1, AL355388.2, AC110926.2, AL133415.1, AL591503.1, DLGAP1-AS1, CHL1-AS2, AC010326.4, AC051649.1, HCG27, AL034417.2, AP001160.1, AC009435.1, LINC01499, AL161621.1, AP001469.1, AC021491.2, AC010168.1, SLC44A3-AS1, AC011445.2, ZMIZ1-AS1, AC027018.1, AL591379.1, AC061992.1, AL513348.1, AC092617.1, AC015853.3, AC138655.1, AC108134.3, LINC01515, AC243829.2, AC020911.1, AC016168.4, AL590705.1, FAM3D-AS1, AC078881.1, AC004466.2, AC018866.1, AC010531.3, AJ011931.2, TUSC8, PTOV1-AS1, RPS4XP2, AC021148.2, AC009041.2, AC093525.6, LINC01516, AL670729.3, C18ORF65, AC098818.2, FAM230C, LINC01988, AC068831.1, CYP51A1-AS1, LINC01227, LINC00895, LINC01519, AC114730.3, MIPEPP3, ITPRIP-AS1, AC138207.5, LINC01091, AC012645.3, AC082651.1, DLGAP1-AS3, AC080011.1, AC010148.1, AC078962.1, AL132822.1, LINC02552, AC098484.2, AC090587.1, LINC00173, PIK3CD-AS1, AL021393.1, AL450322.2, AL591368.1, AC010894.4, AC067930.8, AC024267.3, AC104984.1, AC109495.1, AC129926.1, KLF3-AS1, AL512306.3, LINC01448, AC080013.6, and DACT3-AS1. In some embodiments, treatment results a decrease in expression of one or more IncRNA molecules of the disclosure and an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a decrease in expression of one or more IncRNA molecules. In some embodiments, one or more IncRNA molecules are selected from PXN-AS1, AC116366.2, HCG27, AL034417.2, ZMIZ1-AS1, AL513348.1, AC243829.2, LINC01227, LINC01091, AC080011.1, AL591368.1, AC109495.1, KLF3-AS1, and LINC01448. In some embodiments, treatment results a decrease in expression of one or more IncRNA molecules of the disclosure and an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a decrease in expression of one or more IncRNA molecules that regulate the transcription of genes involved in inflammation. In some embodiments, treatment results a decrease in expression of one or more IncRNA molecules that regulate the transcription of genes involved in inflammation and an improvement in fatigue. In some embodiments, one or more IncRNA molecules that regulate the transcription of genes involved in inflammation are selected from PXN-AS1, AC116366.2, HCG27, AL034417.2, ZMIZ1-AS1, AL513348.1, AC243829.2, LINC01227, LINC01091, AC080011.1, AL591368.1, AC109495.1, KLF3-AS1, and LINC01448.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a decrease in expression of PXN-AS1. In some embodiments, treatment results in decrease in expression of PXN-AS1 and an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a decrease in expression of AC116366.2. In some embodiments, treatment results in a decrease in expression of AC116366.2 and an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a decrease in expression of HCG27. In some embodiments, treatment results in a decrease in expression of HCG27 and an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a decrease in expression of AL034417.2. In some embodiments, treatment results in a decrease in expression of AL034417.2 and an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a decrease in expression of ZMIZ1-AS1. In some embodiments, treatment results in a decrease in expression of ZMIZ1-AS1 and an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a decrease in expression of AL513348.1. In some embodiments, treatment results a decrease in expression of AL513348.1 and in an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a decrease in expression of AC243829.2. In some embodiments, treatment results in a decrease in expression of AC243829.2 and an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a decrease in expression of LINC01227. In some embodiments, treatment results in a decrease in expression of LINC01227 and an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a decrease in expression of LINC01091. In some embodiments, treatment results in a decrease in expression of LINC01091 and an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a decrease in expression of AC080011.1. In some embodiments, treatment results in a decrease in expression of AC080011.1 and an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a decrease in expression of AL591368.1. In some embodiments, treatment results in a decrease in expression of AL591368.1 and an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a decrease in expression of AC109495.1. In some embodiments, treatment results in a decrease in expression of AC109495.1 and an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a decrease in expression of KLF3-AS1. In some embodiments, treatment results in a decrease in expression of KLF3-AS1 and an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a decrease in expression of LINC01448. In some embodiments, treatment results in a decrease in expression of LINC01448 and an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a decrease of at least 10%, 20%, 30%, 40%, 50%, 60%, 70%, 80%, 90%, or 100% of the expression of one or more of the IncRNA molecules of the disclosure. In some embodiments, treatment results in a decrease of at least 10%, 20%, 30%, 40%, 50%, 60%, 70%, 80%, 90%, or 100% of the expression of one or more IncRNA molecules of the disclosure and an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a decrease of at least 10%, 20%, 30%, 40%, 50%, 60%, 70%, 80%, 90%, or 100% of the expression of one or more IncRNA molecules selected from PXN-AS1, AP002852.1, AL009176.1, MKX-AS1, AC109464.1, AL590560.2, AC020908.3, AL731533.3, AL353743.2, AC007405.1, DPRXP2, ST20-AS1, AC020916.1, AC027601.1, AL358334.2, AL358394.2, AL022238.2, AC116366.2, AP003031.1, CYP4F30P, AL390071.1, AC008532.1, CPHL1P, AC008870.4, AC008763.1, AL355388.2, AC110926.2, AL133415.1, AL591503.1, DLGAP1-AS1, CHL1-AS2, AC010326.4, AC051649.1, HCG27, AL034417.2, AP001160.1, AC009435.1, LINC01499, AL161621.1, AP001469.1, AC021491.2, AC010168.1, SLC44A3-AS1, AC011445.2, ZMIZ1-AS1, AC027018.1, AL591379.1, AC061992.1, AL513348.1, AC092617.1, AC015853.3, AC138655.1, AC108134.3, LINC01515, AC243829.2, AC020911.1, AC016168.4, AL590705.1, FAM3D-AS1, AC078881.1, AC004466.2, AC018866.1, AC010531.3, AJ011931.2, TUSC8, PTOV1-AS1, RPS4XP2, AC021148.2, AC009041.2, AC093525.6, LINC01516, AL670729.3, C18ORF65, AC098818.2, FAM230C, LINC01988, AC068831.1, CYP51A1-AS1, LINC01227, LINC00895, LINC01519, AC114730.3, MIPEPP3, ITPRIP-AS1, AC138207.5, LINC01091, AC012645.3, AC082651.1, DLGAP1-AS3, AC080011.1, AC010148.1, AC078962.1, AL132822.1, LINC02552, AC098484.2, AC090587.1, LINC00173, PIK3CD-AS1, AL021393.1, AL450322.2, AL591368.1, AC010894.4, AC067930.8, AC024267.3, AC104984.1, AC109495.1, AC129926.1, KLF3-AS1, AL512306.3, LINC01448, AC080013.6, and DACT3-AS1. In some embodiments, treatment results in a decrease of at least 10%, 20%, 30%, 40%, 50%, 60%, 70%, 80%, 90%, or 100% of the expression of one or more IncRNA molecules of the disclosure and an improvement in fatigue.

In some embodiments, the disclosure provides methods for treating Sjogren’s syndrome in a patient in need thereof, the method comprising administering an effective amount of an RNA nuclease agent (e.g., RSLV-132) to the patient, wherein treatment results in a decrease of the expression of at least 10%, 20%, 30%, 40%, 50%, 60%, 70%, 80%, 90%, or 100% of one or more IncRNA molecules selected from PXN-AS1, AC116366.2, HCG27, AL034417.2, ZMIZ1-AS1, AL513348.1, AC243829.2, LINC01227, LINC01091, AC080011.1, AL591368.1, AC109495.1, KLF3-AS1, and LINC01448. In some embodiments, treatment results in a decrease of at least 10%, 20%, 30%, 40%, 50%, 60%, 70%, 80%, 90%, or 100% of the expression of one or more IncRNA molecules of the disclosure and an improvement in fatigue.

In some embodiments, treatment results a decrease of at least 10%, 20%, 30%, 40%, 50%, 60%, 70%, 80%, 90%, or 100% of the expression of one or more IncRNA molecules that regulate the transcription of genes involved in inflammation. In some embodiments, treatment results a decrease of at least 10%, 20%, 30%, 40%, 50%, 60%, 70%, 80%, 90%, or 100% of the expression of one or more IncRNA molecules that regulate the transcription of genes involved in inflammation and an improvement in fatigue.

### Diagnostic Methods

The disclosure provides methods related to detecting and/or quantifying one or more long non-coding RNA molecules (lncRNA) described herein, in one or more samples, wherein the detection and/or quantification of the one or more IncRNA molecules individually or in combination, will indicate a likelihood that a patient with Sjogren’s syndrome is responsive to treatment with an RNA nuclease agent (e.g., RSLV-132). In some embodiments, the detection and/or quantification of one or more IncRNA molecule(s) in a sample from a patient with Sjogren’s syndrome is compared to a control. In some embodiments, a control is a sample from a healthy subject. In some embodiments, a control is a sample taken from the patient prior to treatment. In some embodiments, a control is a sample take from the patient prior to disease onset. In some embodiments, a control is a sample taken from a subject that did not respond to treatment.

Provided herein are methods for monitoring a response of a patient with Sjogren’s syndrome treated with an RNA nuclease agent (e.g., RSLV-132), the method comprising: determining the expression level and/or activity of one or more IncRNA molecules of the disclosure, in a first sample from the patient obtained prior to treatment with the RNA nuclease agent; determining the expression level of the one or more IncRNA molecules in a second sample from the patient obtained after treatment with the RNA nuclease agent; comparing the expression level of the one or more IncRNA molecules in the second sample with the expression level of the one or more IncRNA molecules in the first sample, wherein a change of the expression level of the one or more IncRNA molecules in the second sample relative to the expression level of the one or more IncRNA molecules in the first sample indicates a response of the patient treated with the RNA nuclease agent. In some embodiments, one or more IncRNA molecules are selected from PXN-AS1, AP002852.1, AL009176.1, MKX-AS1, AC109464.1, AL590560.2, AC020908.3, AL731533.3, AL353743.2, AC007405.1, DPRXP2, ST20-AS1, AC020916.1, AC027601.1, AL358334.2, AL358394.2, AL022238.2, AC116366.2, AP003031.1, CYP4F30P, AL390071.1, AC008532.1, CPHL1P, AC008870.4, AC008763.1, AL355388.2, AC110926.2, AL133415.1, AL591503.1, DLGAP1-AS1, CHL1-AS2, AC010326.4, AC051649.1, HCG27, AL034417.2, AP001160.1, AC009435.1, LINC01499, AL161621.1, AP001469.1, AC021491.2, AC010168.1, SLC44A3-AS1, AC011445.2, ZMIZ1-AS1, AC027018.1, AL591379.1, AC061992.1, AL513348.1, AC092617.1, AC015853.3, AC138655.1, AC108134.3, LINC01515, AC243829.2, AC020911.1, AC016168.4, AL590705.1, FAM3D-AS1, AC078881.1, AC004466.2, AC018866.1, AC010531.3, AJ011931.2, TUSC8, PTOV1-AS1, RPS4XP2, AC021148.2, AC009041.2, AC093525.6, LINC01516, AL670729.3, C18ORF65, AC098818.2, FAM230C, LINC01988, AC068831.1, CYP51A1-AS1, LINC01227, LINC00895, LINC01519, AC114730.3, MIPEPP3, ITPRIP-AS1, AC138207.5, LINC01091, AC012645.3, AC082651.1, DLGAP1-AS3, AC080011.1, AC010148.1, AC078962.1, AL132822.1, LINC02552, AC098484.2, AC090587.1, LINC00173, PIK3CD-AS1, AL021393.1, AL450322.2, AL591368.1, AC010894.4, AC067930.8, AC024267.3, AC104984.1, AC109495.1, AC129926.1, KLF3-AS1, AL512306.3, LINC01448, AC080013.6, and DACT3-AS1. In some embodiments, one or more IncRNA molecules are selected from PXN-AS1, AC116366.2, HCG27, AL034417.2, ZMIZ1-AS1, AL513348.1, AC243829.2, LINC01227, LINC01091, AC080011.1, AL591368.1, AC109495.1, KLF3-AS1, and LINC01448. In some embodiments, one or more IncRNA molecules are selected from one or more IncRNA molecules that regulate the transcription of genes involved in inflammation.

Further provided herein are methods for monitoring a response of a patient with Sjogren’s syndrome treated with an RNA nuclease agent (e.g., RSLV-132), the method comprising: determining the expression level of one or more long non-coding RNA (lncRNA) molecules, in a first sample from the patient obtained prior to treatment with the RNA nuclease agent; determining the expression level of the one or more IncRNA molecules in a second sample from the patient obtained after treatment with the RNA nuclease agent; comparing the expression level of the one or more IncRNA molecules in the second sample with the expression level and/or activity of the one or more IncRNA molecules in the first sample, wherein a decrease in the expression level of the one or more IncRNA molecules in the second sample relative to the expression level of the one or more IncRNA molecules in the first sample indicates a response of the patient treated with the RNA nuclease agent. In some embodiments, one or more IncRNA molecules are selected from PXN-AS1, AP002852.1, AL009176.1, MKX-AS1, AC109464.1, AL590560.2, AC020908.3, AL731533.3, AL353743.2, AC007405.1, DPRXP2, ST20-AS1, AC020916.1, AC027601.1, AL358334.2, AL358394.2, AL022238.2, AC116366.2, AP003031.1, CYP4F30P, AL390071.1, AC008532.1, CPHL1P, AC008870.4, AC008763.1, AL355388.2, AC110926.2, AL133415.1, AL591503.1, DLGAP1-AS1, CHL1-AS2, AC010326.4, AC051649.1, HCG27, AL034417.2, AP001160.1, AC009435.1, LINC01499, AL161621.1, AP001469.1, AC021491.2, AC010168.1, SLC44A3-AS1, AC011445.2, ZMIZ1-AS1, AC027018.1, AL591379.1, AC061992.1, AL513348.1, AC092617.1, AC015853.3, AC138655.1, AC108134.3, LINC01515, AC243829.2, AC020911.1, AC016168.4, AL590705.1, FAM3D-AS1, AC078881.1, AC004466.2, AC018866.1, AC010531.3, AJ011931.2, TUSC8, PTOV1-AS1, RPS4XP2, AC021148.2, AC009041.2, AC093525.6, LINC01516, AL670729.3, C18ORF65, AC098818.2, FAM230C, LINC01988, AC068831.1, CYP51A1-AS1, LINC01227, LINC00895, LINC01519, AC114730.3, MIPEPP3, ITPRIP-AS1, AC138207.5, LINC01091, AC012645.3, AC082651.1, DLGAP1-AS3, AC080011.1, AC010148.1, AC078962.1, AL132822.1, LINC02552, AC098484.2, AC090587.1, LINC00173, PIK3CD-AS1, AL021393.1, AL450322.2, AL591368.1, AC010894.4, AC067930.8, A024267.3, AC104984.1, AC109495.1, AC129926.1, KLF3-AS1, AL512306.3, LINC01448, AC080013.6, and DACT3-AS1. In some embodiments, one or more IncRNA molecules are selected from PXN-AS1, AC116366.2, HCG27, AL034417.2, ZMIZ1-AS1, AL513348.1, AC243829.2, LINC01227, LINC01091, AC080011.1, AL591368.1, AC109495.1, KLF3-AS1, and LINC01448. In some embodiments, one or more IncRNA molecules are selected from one or more IncRNA molecules that regulate the transcription of genes involved in inflammation

### Determination of Long Non-Coding RNA Molecules

The presence of or an amount or expression level of one or more long non-coding RNA (lncRNA) molecules of the disclosure in a sample can be detected or determined by a number of methodologies and techniques, which are known in the art and understood by the skilled artisan, including, but not limited to, in situ hybridization, fluorescence in situ hybridization (FISH), Northern analysis, nuclease protection assays (NPA), reverse transcription-polymerase chain reaction (RT-PCT), whole genome sequencing, polymerase chain reaction (PCR) including quantitative real time PCR (qRT-PCR) and other amplification type detection methods, such as, for example, branched DNA, SISBA, TMA and the like, RNA-Seq, microarray analysis, tiling microarray, oligo microarray, massively parallel signature sequencing (MPSS), sequence tag based methods, Cap analysis of gene expression (CAGE, CAGE-seq), parallel analysis of RNA ends (PARE)/genome-wide mapping of uncapped and cleaved transcripts (GMUCT)/degradome-seq, Transcript isoform sequencing (TIF-seq), chromatin immunoprecipitation microarray (ChIP-ChIP), ChIP sequencing (ChIP-seq), RIP-chip, Global run-on sequencing (GRO-seq), precision nuclear run-on sequencing (PRO-seq), native elongating transcript sequencing (NET-seq), BRIC-eq/BrU-Seq/BrUChase-Seq, gene expression profiling, and/or serial analysis of gene expression (SAGE), nascent RNA sequencing, as well as any one of the wide variety of assays that can be performed by RNA, nucleic acid, gene, sequencing and/or array analysis. Typical protocols for evaluating the status of genes and gene products are found, for example in Ausubel et al., eds., 1 995, Current Protocols In Molecular Biology, Units 2 (Northern Blotting), 4 (Southern Blotting), 15 (Immunoblotting) and 18 (PCR Analysis).

### Nucleic Acid Techniques

In some embodiments, the expression level of a long non-coding RNA (IncRNA) molecule described herein, may be a nucleic acid expression level. In some embodiments, the nucleic acid expression level is determined using qPCR, rtPCR, RNA-Seq, multiplex qPCR or RT-qPCR, microarray analysis, MassARRAY technique, in situ hybridization, fluorescence in situ hybridization (FISH), Northern analysis, nuclease protection assays (NPA), reverse transcription-polymerase chain reaction (RT-PCT), whole genome sequencing, whole transcriptome sequencing, polymerase chain reaction (PCR) including quantitative real time PCR (qRT-PCR) and other amplification type detection methods, such as, for example, branched DNA, SISBA, TMA and the like, tiling microarray, oligo microarray, massively parallel signature sequencing (MPSS), sequence tag based methods, Cap analysis of gene expression (CAGE, CAGE-seq), parallel analysis of RNA ends (PARE)/genome-wide mapping of uncapped and cleaved transcripts (GMUCT)/degradome-seq, Transcript isoform sequencing (TIF-seq), chromatin immunoprecipitation microarray (ChIP-ChIP), ChIP sequencing (ChIP-seq), RIP-chipGlobal run-on sequencing (GRO-seq), precision nuclear run-on sequencing (PRO-seq), native elongating transcript sequencing (NET-seq), BRIC-eq/BrU-Seq/BrUChase-Seq, gene expression profiling, and/or serial analysis of gene expression (SAGE), nascent RNA sequencing. In some embodiments, the expression level of a IncRNA molecule is determined in cells from a patient with Sjogren’s syndrome. In some embodiments, the expression level of a IncRNA molecule described herein is determined in blood cells from a patient with Sjogren’s syndrome. In some embodiments, the expression level of a IncRNA molecule described herein is determined in whole blood from a patient with Sjogren’s syndrome.

Methods for the evaluation of IncRNA molecules in cells are known in the art and include, for example, hybridization assays (such as in situ hybridization using labeled riboprobes, FISH, Northern blot and related techniques) and various nucleic acid amplification assays (such as RT-PCR using complementary primers specific for one or more of the molecules), qPCR, rtPCR, RNA-Seq, multiplex qPCR or RT-qPCR, microarray analysis, MassARRAY technique, nuclease protection assays (NPA), reverse transcription-polymerase chain reaction (RT-PCT), whole genome sequencing, whole transcriptome sequencing, polymerase chain reaction (PCR) including quantitative real time PCR (qRT-PCR) and other amplification type detection methods, such as, for example, branched DNA, SISBA, TMA and the like, tiling microarray, oligo microarray, massively parallel signature sequencing (MPSS), sequence tag based methods, Cap analysis of gene expression (CAGE, CAGE-seq), parallel analysis of RNA ends (PARE)/genome-wide mapping of uncapped and cleaved transcripts (GMUCT)/degradome-seq, Transcript isoform sequencing (TIF-seq), chromatin immunoprecipitation microarray (ChIP-ChIP), ChIP sequencing (ChIP-seq), RIP-chipGlobal run-on sequencing (GRO-seq), precision nuclear run-on sequencing (PRO-seq), native elongating transcript sequencing (NET-seq), BRIC-eq/BrU-Seq/BrUChase-Seq, gene expression profiling, and/or serial analysis of gene expression (SAGE), nascent RNA sequencing.

In addition, such methods can include one or more steps that allow one to determine the levels of one or more IncRNA molecules in a biological sample (e.g., by simultaneously examining the levels a comparative control IncRNA molecules).

The nucleic acid sequences of the IncRNA molecules of the disclosure are known in the art. In some embodiments, one or more IncRNA molecules are selected from PXN-AS1, AP002852.1, AL009176.1, MKX-AS1, AC109464.1, AL590560.2, AC020908.3, AL731533.3, AL353743.2, AC007405.1, DPRXP2, ST20-AS1, AC020916.1, AC027601.1, AL358334.2, AL358394.2, AL022238.2, AC116366.2, AP003031.1, CYP4F30P, AL390071.1, AC008532.1, CPHL1P, AC008870.4, AC008763.1, AL355388.2, AC110926.2, AL133415.1, AL591503.1, DLGAP1-AS1, CHL1-AS2, AC010326.4, AC051649.1, HCG27, AL034417.2, AP001160.1, AC009435.1, LINC01499, AL161621.1, AP001469.1, AC021491.2, AC010168.1, SLC44A3-AS1, AC011445.2, ZMIZ1-AS1, AC027018.1, AL591379.1, AC061992.1, AL513348.1, AC092617.1, AC015853.3, AC138655.1, AC108134.3, LINC01515, AC243829.2, AC020911.1, AC016168.4, AL590705.1, FAM3D-AS1, AC078881.1, AC004466.2, AC018866.1, AC010531.3, AJ011931.2, TUSC8, PTOV1-AS1, RPS4XP2, AC021148.2, AC009041.2, AC093525.6, LINC01516, AL670729.3, C18ORF65, AC098818.2, FAM230C, LINC01988, AC068831.1, CYP51A1-AS1, LINC01227, LINC00895, LINC01519, AC114730.3, MIPEPP3, ITPRIP-AS1, AC138207.5, LINC01091, AC012645.3, AC082651.1, DLGAP1-AS3, AC080011.1, AC010148.1, AC078962.1, AL132822.1, LINC02552, AC098484.2, AC090587.1, LINC00173, PIK3CD-AS1, AL021393.1, AL450322.2, AL591368.1, AC010894.4, AC067930.8, AC024267.3, AC104984.1, AC109495.1, AC129926.1, KLF3-AS1, AL512306.3, LINC01448, AC080013.6, and DACT3-AS1. In some embodiments, one or more IncRNA molecules are selected from PXN-AS1, AC116366.2, HCG27, AL034417.2, ZMIZ1-AS1, AL513348.1, AC243829.2, LINC01227, LINC01091, AC080011.1, AL591368.1, AC109495.1, KLF3-AS1, and LINC01448. In some embodiments, one or more IncRNA molecules are selected from one or more IncRNA molecules that regulate the transcription of genes involved in inflammation. In some embodiments, one or more IncRNA molecules are selected from one or more IncRNA molecules that regulate the transcription of pro-inflammatory genes. In some embodiments, the pro-inflammatory genes are related to autoimmune disease and/or immune system function.

In some embodiments, a IncRNA molecule of the disclosure is PXN-AS1. In some embodiments, a IncRNA molecule of the disclosure is AC116366.2. In some embodiments, a IncRNA molecule of the disclosure is HCG27. In some embodiments, a IncRNA molecule of the disclosure is AL034417.2. In some embodiments, a IncRNA molecule of the disclosure is ZMIZ1-AS1. In some embodiments, a IncRNA molecule of the disclosure is AL513348.1. In some embodiments, a IncRNA molecule of the disclosure is AC243829.2. In some embodiments, a IncRNA molecule of the disclosure is LINC01227. In some embodiments, a IncRNA molecule of the disclosure is LINC01091. In some embodiments, a IncRNA molecule of the disclosure is AC080011.1. In some embodiments, a IncRNA molecule of the disclosure is AL591368.1. In some embodiments, a IncRNA molecule of the disclosure is AC109495.1. In some embodiments, a IncRNA molecule of the disclosure is KLF3-AS1. In some embodiments, a IncRNA molecule of the disclosure is LINC01448.

In some embodiments, the sequence of the amplified target IncRNA molecule can be determined. Methods include protocols which examine or detect IncRNA molecules, such as target IncRNA molecules, in a tissue or cell sample by microarray technologies. Using nucleic acid microarrays, test and control IncRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of IncRNA molecules whose expression or decrease in expression correlates with increased or reduced clinical benefit of treatment comprising an RNA nuclease agent (e.g., RSLV-132), may be arrayed on a solid support. Hybridization of a labeled probe with a particular array member indicates that the sample from which the probe was derived expresses that IncRNA molecule.

Inclusion of any of the diagnostic methods described herein as part of any method directed to methods for identifying patients likely to benefit from treatment as described herein (e.g., selection of a therapeutic treatment or intervention) or to the development of treatments (e.g., enrollment patients in clinical trials) provides an advantage over those methods that do not include the diagnostic methods, in that a patient population whose members are predicted to need and/or not need, benefit, or respond to treatment may be identified.

Accordingly, in some embodiments, the IncRNA molecule suitable for use in the present disclosure is a diagnostic IncRNA molecule. In some embodiments, the IncRNA molecule is a monitoring IncRNA molecule. In some embodiments, the IncRNA molecule is a predictive IncRNA molecule.

Long non-coding RNA (lncRNA) molecules can be a substance or a biological event whose detection indicates a particular physiological state (e.g., a diseased state). For example, the presence, absence, or decrease in expression of a IncRNA molecule in a sample from a patient may indicate disease (e.g., Sjogren’s syndrome). IncRNA molecules measured in patients before treatment can be used to identify suitable patients for inclusion in a clinical trial. lncRNA molecule changes after treatment may predict or identify safety problems related to a candidate drug, or reveal pharmacologic activity expected to predict an eventual benefit of treatment. ncRNA molecules may reduce uncertainty in drug development and evaluation by providing quantifiable predictions about drug performance, and they can contribute to dose selection. Composite lncRNA molecules include several individual lncRNA molecules in a stated algorithm which reaches a single interpretive readout when a single lncRNA molecule fails to provide all the relevant information required for assessment.

A surrogate end-point is an lncRNA molecule that is intended to substitute for a clinical end point and is expected, based on epidemiologic, therapeutic, pathophysiologic, or other scientific evidence, to predict clinical benefit.

### Kits

The present disclosure provides kits comprising an RNase-containing nuclease fusion protein of the disclosure, including an RNase-Fc fusion protein and instructions for use. The kits may comprise, in a suitable container, an RNase-Fc fusion protein disclosed herein, one or more controls, and various buffers, reagents, enzymes and other standard ingredients well known in the art. In some embodiments, the kit includes an injectable solution comprising an RNase-Fc fusion protein and one or more pharmaceutically acceptable carriers and/or diluents. In some embodiments, the injectable solution is formulated for intravenous administration. In some embodiment, the kit includes instructions for use.

The container can include at least one vial, well, test tube, flask, bottle, syringe, or other container means, into which an RNase-Fc fusion protein may be placed, and in some instances, suitably aliquoted. Where an additional component is provided, the kit can contain additional containers into which this component may be placed. The kits can also include a means for containing the RNase-Fc fusion protein and any other reagent containers in close confinement for commercial sale. Such containers may include injection or blow-molded plastic containers into which the desired vials are retained. Containers and/or kits can include labeling with instructions for use and/or warnings.

### Other Embodiments

The disclosure relates to the following embodiments. Throughout this section, the term embodiment is abbreviated as ‘E’ followed by an ordinal. For example, E1 is equivalent to Embodiment 1.
E1. A method for treating Sjogren’s disease by reducing fatigue in a human patient in need thereof, the method comprising administering an effective amount of an RNase-Fc fusion protein to the patient, thereby treating Sjogren’s disease by reducing fatigue in the patient.
E2. The method of embodiment 1, wherein the RNase-Fc fusion protein comprises a human pancreatic RNase 1.
E3. The method of embodiment 2, wherein the human pancreatic RNase 1 comprises the amino acid sequence as set forth in SEQ ID NO: 2.
E4. The method of embodiment 1, wherein the RNase-Fc fusion protein comprises a wild-type human IgG1 Fc domain or a human IgG1 Fc domain comprising one or more mutations.
E5. The method of embodiment 4, wherein the Fc domain comprising one or more mutations has decreased binding to Fcγ receptors on human cells.
E6. The method of embodiment 1, wherein the RNase-Fc fusion protein has a reduced effector function optionally selected from the group consisting of opsonization, phagocytosis, complement dependent cytotoxicity, and antibody-dependent cellular cytotoxicity.
E7. The method of embodiment 4, wherein the human IgG1 Fc domain comprises a P238S mutation and a P331S mutation according to EU numbering.
E8. The method of embodiment 4, wherein the human IgG1 Fc domain comprises a hinge domain, a CH2 domain and a CH3 domain.
E9. The method of embodiment 4, wherein the human IgG1 Fc domain comprises a substitution of one or more of three hinge region cysteine residues with serine.
E10. The method of embodiment 9, wherein the Fc domain comprises an SCC mutation (residues 220, 226, and 229), numbering according to the EU index.
E11. The method of embodiment 1, wherein the human IgG1 Fc domain comprises the amino acid sequence as set forth in SEQ ID NO: 22.
E12. The method of embodiment 1, wherein the RNase-Fc fusion protein comprises the amino acid sequence as set forth in SEQ ID NO: 50.
E13. The method of embodiment 1, wherein the RNase-Fc fusion protein is administered to the patient at a dose of about 5-10 mg/kg.
E14. The method of embodiment 1, wherein the RNase-Fc fusion protein is administered to the patient at a dose of about 10 mg/kg.
E15. The method of embodiment 1, wherein the RNase-Fc fusion protein is administered to the patient at a dose of about 5 mg/kg.
E16. The method of embodiment 1, wherein the RNase-Fc fusion protein is administered to the patient by intravenous injection.
E17. The method of embodiment 1, wherein the RNase-Fc fusion protein is administered to the patient at a dose of about 5-10 mg/kg every two weeks.
E18. The method of embodiment 1, wherein the RNase-Fc fusion protein is administered to the patient at a dose of about 5-10 mg/kg every two weeks for three months.
E19. The method of embodiment 1, wherein the RNase-Fc fusion protein is administered to the patient in six biweekly infusions over three months.
E20. The method of embodiment 1, wherein the RNase-Fc fusion protein is administered to the patient every week for three weeks, and then one administration every two weeks to achieve or maintain a therapeutic effect.
E21. The method of embodiment 1, wherein treatment reduces fatigue in the patient by at least a one point in an EULAR SS Patient Reported Index (ESSPRI) score relative to an ESSPRI score prior to treatment.
E22. The method of embodiment 1, wherein treatment reduces the ESSPRI score by at least one point relative to the ESSPRI score prior to treatment.
E23. The method of embodiment 21, wherein fatigue is reduced to a score of between 4.5 and 5.5 on an ESSPRI scale of 1 to 10.
E24. The method of embodiment 21, wherein the patient is administered an effective dose of the RNase-Fc every two weeks.
E25. The method of embodiment 1, wherein treatment improves fatigue in the patient by at least one point in a Functional Assessment of Chronic Illness Therapy (FACIT) fatigue scale relative to a FACIT score prior to treatment.
E26. The method of embodiment 25, wherein treatment improves fatigue in the patient by at least two points in a FACIT fatigue scale.
E27. The method of embodiment 1, wherein treatment increases the FACIT fatigue score by at least one point relative to the FACIT fatigue score prior to treatment.
E28. The method of embodiment 27, wherein treatment increases the FACIT fatigue score by at least two points relative to the FACIT fatigue score prior to treatment.
E29. The method of embodiment 1, wherein treatment reduces fatigue in the patient by at least one point in a Profile of Fatigue (ProF) score relative to a ProF score prior to treatment.
E30. The method of embodiment 1, wherein treatment reduces fatigue in the patient by at least one point in a mental component of Profile of Fatigue (ProF) score relative to a mental component PROF score prior to treatment.
E31. The method of embodiment 1, wherein treatment reduces fatigue in the patient by at least one point in a somatic component of Profile of Fatigue (ProF) score relative to a somatic component PROF score prior to treatment.
E32. The method of embodiment 1, wherein treatment improves cognitive function in the patient as measured by the Digit Symbol Substitution Test (DSST) test relative to a DSST test score prior to treatment.
E33. The method of embodiment 1, wherein treatment increases the number of matches completed in 90 seconds by the patient on a Digit Symbol Substitution Test (DSST) test.
E34. The method of embodiment 1, wherein treatment reduces the time to complete the DSST test by the patient.
E36. A method for treating Sjogren’s disease by improving cognitive effects in a human patient in need thereof, the method comprising administering an effective amount of an RNase-Fc fusion protein to the patient, thereby treating Sjogren’s disease by improving cognitive effects in the patient.
E37. The method of embodiment 36, wherein cognitive effects in the patient are improved by at least one point in a mental component of ProF relative to a mental component of ProF prior to treatment.
E38. A method for treating Sjogren’s disease by reducing fatigue in a human patient in need thereof, the method comprising administering an effective amount of an RNase-Fc fusion protein comprising the amino acid sequence as set forth in SEQ ID NO: 50 to the patient, thereby treating Sjogren’s disease by reducing fatigue in the patient.
E39. A method for treating Sjogren’s disease by reducing fatigue in a human patient in need thereof, comprising administering an effective amount of a pharmaceutical composition to the patient, wherein the composition comprises: an RNase-Fc fusion protein comprising the amino acid sequence as set forth in SEQ ID NO: 50; and one or more pharmaceutically acceptable carriers and/or diluents, thereby treating Sjogren’s disease by reducing fatigue in the patient.
E40. The method of embodiment 35 or 36, wherein the RNase-Fc fusion protein comprises a human pancreatic RNase 1.
E41. The method of embodiment 40, wherein the human pancreatic RNase 1 comprises the amino acid sequence as set forth in SEQ ID NO: 2.
E42. The method of any one of embodiments 35-41, wherein the RNase-Fc fusion protein comprises a wild-type human IgG1 Fc domain or a human IgG1 Fc domain comprising one or more mutations.
E43. The method of embodiment 42, wherein the Fc domain comprising one or more mutations has decreased binding to Fcγ receptors on human cells.
E44. The method of any one of embodiments 35-39, wherein the RNase-Fc fusion protein has a reduced effector function optionally selected from the group consisting of opsonization, phagocytosis, complement dependent cytotoxicity, and antibody-dependent cellular cytotoxicity.
E45. The method of embodiment 42, wherein the human IgG1 Fc domain comprises a P238S mutation and a P331S mutation according to EU numbering.
E46. The method of embodiment 42, wherein the human IgG1 Fc domain comprises a hinge domain, a CH2 domain and a CH3 domain.
E47. The method of embodiment 42, wherein the human IgG1 Fc domain comprises a substitution of one or more of three hinge region cysteine residues with serine.
E48. The method of embodiment 47, wherein the Fc domain comprises an SCC mutation (residues 220, 226, and 229), numbering according to the EU index.
E49. The method of embodiment 35 or 36, wherein the human IgG1 Fc domain comprises the amino acid sequence as set forth in SEQ ID NO: 22.
E50. The method of embodiment 35 or 36, wherein the RNase-Fc fusion protein comprises the amino acid sequence as set forth in SEQ ID NO: 50.
E51. The method of any one of embodiments 36-39, wherein the RNase-Fc fusion protein is administered to the patient at a dose of about 5-10 mg/kg.
E52. The method of any one of embodiments 35-39, wherein the RNase-Fc fusion protein is administered to the patient at a dose of about 10 mg/kg.
E53. The method of any one of embodiments 35-39, wherein the RNase-Fc fusion protein is administered to the patient at a dose of about 5 mg/kg.
E54. The method of any one of embodiments 36-39, wherein the RNase-Fc fusion protein is administered to the patient by intravenous injection.
E55. The method of any one of embodiments 35-39, wherein the RNase-Fc fusion protein is administered to the patient at a dose of about 5-10 mg/kg every two weeks.
E56. The method of any one of embodiments 35-39, wherein the RNase-Fc fusion protein is administered to the patient at a dose of about 5-10 mg/kg every two weeks for three months.
E57. The method of any one of embodiments 35-39, wherein the RNase-Fc fusion protein is administered to the patient in six biweekly infusions over three months.
E58. The method of any one of embodiments 35-39, wherein the RNase-Fc fusion protein is administered to the patient every week for three weeks, and then one administration every two weeks.
E59. The method of any one of embodiments 35-39, wherein treatment reduces fatigue in the patient by at least a one point in an EULAR SS Patient Reported Index (ESSPRI) score relative to an ESSPRI score prior to treatment.
E60. The method of any one of embodiments 35-39, wherein treatment reduces the ESSPRI score by at least one point relative to the ESSPRI score prior to treatment.
E61. The method of embodiment 59, wherein fatigue is reduced to a score of between 4.5 and 5.5 on an ESSPRI scale of 1 to 10.
E62. The method of embodiment 59, wherein the patient is administered an effective dose of the RNase-Fc every two weeks.
E63. The method of any one of embodiments 35-39, wherein treatment improves fatigue in the patient by at least one point in a Functional Assessment of Chronic Illness Therapy (FACIT) fatigue scale relative to a FACIT score prior to treatment.
E64. The method of embodiment 63, wherein treatment improves fatigue in the patient by at least two points in a FACIT fatigue scale.
E65. The method of any one of embodiments 35-39, wherein treatment increases the FACIT fatigue score by at least one point relative to the FACIT fatigue score prior to treatment.
E66. The method of embodiment 65, wherein treatment increases the FACIT fatigue score by at least two points relative to the FACIT fatigue score prior to treatment.
E67. The method of any one of embodiments 35-39, wherein treatment reduces fatigue in the patient by at least one point in a Profile of Fatigue (ProF) score relative to a ProF score prior to treatment.
E68. The method of any one of embodiments 35-39, wherein treatment reduces fatigue in the patient by at least one point in a mental component of Profile of Fatigue (ProF) score relative to a mental component ProF score prior to treatment.
E69. The method of any one of embodiments 35-39, wherein treatment reduces fatigue in the patient by at least one point in a somatic component of Profile of Fatigue (ProF) score relative to a somatic component ProF score prior to treatment.
E70. The method of any one of embodiments 35-39, wherein treatment improves cognitive function in the patient as measured by the Digit Symbol Substitution Test (DSST) test relative to a DSST test score prior to treatment.
E71. The method of any one of embodiments 35-39, wherein treatment increases the number of matches completed in 90 seconds by the patient on a Digit Symbol Substitution Test (DSST) test.
E72. The method of any one of embodiments 35-39, wherein treatment reduces the time to complete the DSST test by the patient.
E73. A kit comprising a container comprising an injectable solution and instructions for use in treating Sjogren’s disease by reducing fatigue in a human patient in need thereof, comprising:
   an effective amount of an RNase-Fc fusion protein as set forth in SEQ ID NO: 50; and
   one or more pharmaceutically acceptable carriers and/or diluents;
   wherein the injectable solution is formulated for intravenous administration.
E74. An RNase-Fc fusion protein for use in method for treating Sjogren’s disease by reducing fatigue in a human patient in need thereof, the treatment comprising administering an effective amount of an RNase-Fc fusion protein to the patient.
E75. Use of an RNase-Fc fusion protein for use in the manufacture of a medicament for treating Sjogren’s disease by reducing fatigue in a human patient in need thereof.
E76. An RNase-Fc fusion protein for use in a method for treating Sjogren’s disease by reducing fatigue in a human patient in need thereof, the treatment comprising administering by intravenous injection a dose of an RNase-Fc fusion protein of about 5-10 mg/kg to the patient.
E77. Use of an RNase-Fc fusion protein for use in the manufacture of a medicament for treating Sjogren’s disease by reducing fatigue in a human patient in need thereof, the use comprising administering by intravenous injection a dose of an RNase-Fc fusion protein of about 5-10 mg/kg to the patient.
E78. An RNase-Fc fusion protein for use in a method for treating Sjogren’s disease by improving cognitive effects in a human patient in need thereof, the treatment comprising administering an effective amount of an RNase-Fc fusion protein to the patient.
E79. Use of an RNase-Fc fusion protein for use in the manufacture of a medicament for treating Sjogren’s disease by improving cognitive effects in a human patient in need thereof, the use comprising administering an effective amount of an RNase-Fc fusion protein to the patient.
E80. An RNase-Fc fusion protein for use in a method for treating Sjogren’s disease by reducing fatigue in a human patient in need thereof, the treatment comprising administering an effective amount of an RNase-Fc fusion protein comprising the amino acid sequence as set forth in SEQ ID NO: 50 to the patient.
E81. Use of an RNase-Fc fusion protein for use in the manufacture of a medicament for treating Sjogren’s disease by reducing fatigue in a human patient in need thereof, the use comprising administering an effective amount of an RNase-Fc fusion protein comprising the amino acid sequence as set forth in SEQ ID NO: 50 to the patient.
E82. An RNase-Fc fusion protein for use in a method for treating Sjogren’s disease by reducing fatigue in a human patient in need thereof, the treatment comprising administering an effective amount of a pharmaceutical composition to the patient, wherein the composition comprises: an RNase-Fc fusion protein comprising the amino acid sequence as set forth in SEQ ID NO: 50; and one or more pharmaceutically acceptable carriers and/or diluents.
E83. An RNase-Fc fusion protein for use in the manufacture of a medicament for treating Sjogren’s disease by reducing fatigue in a human patient in need thereof, the use comprising administering an effective amount of a pharmaceutical composition to the patient, wherein the composition comprises: an RNase-Fc fusion protein comprising the amino acid sequence as set forth in SEQ ID NO: 50; and one or more pharmaceutically acceptable carriers and/or diluents.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T.E. Creighton, Proteins: Structures and Molecular Properties (W.H. Freeman and Company, 1993); A.L. Lehninger, Biochemistry (Worth Publishers, Inc., current addition); Sambrook, et al, Molecular Cloning: A Laboratory Manual (2nd Edition, 1989); Methods In Enzymology (S. Colowick and N. Kaplan eds., Academic Press, Inc.); Remington’s Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1

### Generation of RNase-Fc Fusion Protein Encoding Expression Vectors

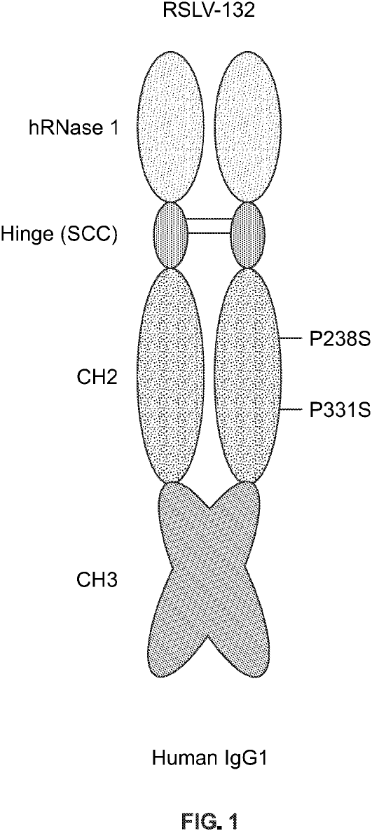

Various embodiments of the RNase-containing nuclease fusion proteins of the disclosure are presented in the Sequence Table (Table 1). An exemplary RNase-Fc fusion protein, RSLV-132, was constructed and has the configuration shown in FIG. 1. Specifically, starting from the amino acid sequence of the RNase-Fc fusion proteins, polynucleotides encoding the RNase-Fc fusion proteins were directly synthesized using codon optimization by Genescript (Genescript, Piscatawy, N.J.) to allow for optimal expression in mammalian cells. The process of optimization involved, e.g., avoiding regions of very high (>80%) or very low (<30%) GC content when possible, and avoiding cis-acting sequence motifs, such as internal TATA-boxes, chi-sites and ribosomal entry sites, AT-rich or GC-rich sequence stretches, RNA instability motifs, repeat sequences and RNA secondary structures, and cryptic splice donor and acceptor sites in higher eukaryotes. DNAs encoding the RNase-Fc fusion proteins are cloned into the pcDNA3.1+ mammalian expression vector. RSLV-132 is an RNase-Fc fusion protein that has the following configuration and was generated (FIG. 1).

RSLV-132 is a homodimer comprising two polypeptides each having the amino acid sequence set forth as SEQ ID NO: 50. Each polypeptide of the homodimer has the configuration RNase-Fc, wherein a wild-type human RNase 1 domain (SEQ ID NO: 2) is operably coupled without a linker to the N-terminus of a human IgG1 Fc domain comprising SCC hinge and CH2 mutations P238S and P331S (SEQ ID NO:22).

### Example 2

### Transient Expression of and Stable Mammalian Cell Lines Expressing RNase-Fc Fusion Proteins

For transient expression, expression vectors from Example 1 containing the RNase-Fc fusion protein inserts were transiently trasfected using FreeStyle™ MAX Reagent into Chinese Hamster Ovary (CHO) cells, e.g., CHO-S cells (e.g., FreeStyle™ CHO-S cells, Invitrogen), using the manufacturer recommended transfection protocol. CHO-S cells were maintained in FreeStyle™ CHO Expression Medium containing 2 mM L-Glutamine and penicillin-streptomycin.

Stable CHO-S cell lines expressing the RNase-Fc fusion proteins were generated using routine methods known in the art. For example, CHO-S cells can be infected with a virus (e.g., retrovirus, lentivirus) comprising the nucleic acid sequences of an RNase-Fc fusion protein, as well as the nucleic acid sequences encoding a marker (e.g., GFP, surface markers selectable by magnetic beads) that is selected for using, e.g., flow cytometry or magnetic bead separation (e.g., MACSelect™ system). Alternatively, CHO-S cells can be transfected using any transfection method known in the art, such as electroporation (Lonza) or the FreeStyle™ MAX Reagent as mentioned above, with a vector comprising the nucleic acid sequences of the RNase-Fc fusion proteins and a selectable marker, followed by selection using, e.g., flow cytometry. The selectable marker can be incorporated into the same vector as that encoding the RNase-Fc fusion proteins or a separate vector.

RNase-Fc fusion proteins were purified from culture supernatant by capturing the molecules using a column packed with Protein-A sepharose beads, followed by washes in column wash buffer (e.g., 90 mM Tris, 150 mM NaCl, 0.05% sodium azide) and releasing the molecules from the column using a suitable elution buffer (e.g., 0.1 M citrate buffer, pH 3.0). The eluted material was further concentrated by buffer exchange through serial spins in PBS using Centricon concentrators, followed by filtration through 0.2 µm filter devices. The concentration of the RNase-Fc fusion proteins was determined using standard spectrophotometric methods (e.g., Bradford, BCA, Lowry, Biuret assays).

### Example 3

### Study Design and Patient Characteristics

A multi-center, double-blind placebo-controlled study to evaluate the impact of eight intravenous infusions of RSLV-132 in patients with primary Sjogren’s syndrome (pSS) was performed. Study participants were ages 18-85 and diagnosed with primary Sjogrens syndrome, according to the 2002 American-European Consensus Group (AEGC). Specifically, the study was conducted in a subset of Sjogren’s patients who had elevated levels of anti-Ro 52/60 autoantibodies and a pattern of elevated interferon-stimulated gene expression in blood cells, e.g., a positive interferon signature at screening. A positive interferon signature was defined as expression of HERC5, CMPK2, and EPSTI1 two standard deviations above healthy volunteers. Study subjects were required to have concomitant medications kept stable for the 30 days prior to the baseline visit. The use of hydroxychloroquine, within 30 days of baseline; belimumab, abatacept, or TNF inhibitors within 90 days of baseline; or cyclophosphamide or rituximab within 180 days of baseline were prohibited. Patients were further required to not have had past head and neck radiation, lymphoma, graft versus host disease, or IgG4-related disease. Potential subjects were screened to assess their eligibility to enter the study within 60 days prior to study entry (i.e., prior to baseline visit). Thirty-two subjects were screened for entry into the study, two subjects not meeting the autoantibody criteria were excluded. The remaining thirty subjects were randomized into the study. Two subjects in the RSLV-132 arm withdrew consent prior to receiving study treatment at the baseline visit. One subject in the placebo group withdrew consent after receiving at least one study treatment. Twenty subjects in the RSLV-132 group and seven subjects in the placebo group completed the study. The modified intent-to-treat analysis set consisted of twenty-eight subjects who received at least one infusion of study drug. Data from eight subjects in the placebo group and twenty subjects in the RSLV-132 group was analyzed in this randomized, double-blind, placebo-controlled phase 2 study (clinicaltrials.gov: NCT03247686). Baseline evaluations were performed on Day 1 of the study. Following the baseline evaluation, patients received their first infusion of RSLV-132 or placebo.

Each of the subjects was randomized 3:1 (active:placebo) and received eight infusions of 10 mg/kg of RSLV-132 or placebo at baseline, weekly for two weeks (three doses), and then once every two weeks for the next 10 weeks (i.e., intravenous infusions were administered on days 1 (baseline), 8, 15, 29, 43, 57, 71, and 85) of the study. Patient reported outcomes as measured by EESPRI, FACIT, and Profile of Fatigue (PROF) were used to assess the active versus control groups comparing baseline and day 99 of the study. The patient reported outcomes were measured on days 1, 29, 57, 85, and 99 (or the end of treatment) prior to receiving the dose for that day. The efficacy endpoints were measured at day 99, and safety follow-ups were conducted at days 141, 176, and 211.

RSLV-132 was presented at a concentration of 9.5 mg/mL in a single-use vial containing 5.3 mL of preservative-free sterile solution including a buffer for dilution for intravenous infusion. 0.9% sodium chloride solution was used as placebo for infusion.

The study was conducted in accordance with the principles of the Declaration of Helsinki and the International Conference on the Harmonisation Guidelines for Good Clinical Practice. Ethics committee and institutional review board approval were obtained and all patients provided written informed consent.

### Example 4

### Evaluation of Patient Characteristics at Baseline.

To assess differences between treatment groups (i.e., patients treated with placebo as compared to patients treated with RSLV-132) and establish baseline levels for analysis, demographics and disease characteristics were obtained prior to treatment. Analysis of patient characteristics at baseline included analysis of Complement C3 and Complement C4 levels, IgG levels (mg/dL), ESR levels, ESSDAI score, ESSPRI score, FACIT score, and profile of fatigue (ProF),

Complement C3 and Complement C4 (C3/C4) measurement is used to assess activation of the immune system. Measurement of C3/C4 in the blood is used as a readout for immune activity. A blood test measures the specific complement protein (C3 or C4) and reports as milligrams per deciliter. Low levels of C3/C4 in the blood may indicate disease or autoimmunity.

Immunoglobulin G (IgG) constitutes about eighty percent of serum immunoglobulins. Measurement of IgG levels from a blood sample may indicate disease states. The amount of IgG in blood is typically reported as milligrams per deciliter.

The Erythrocyte Sedimentation Rate (ESR) is used as a screen of inflammation in the body. Typically, red blood cells settle more rapidly in some disease states due to increases in plasma fibrinogen, immunoglobulins, and other acute-phase reaction proteins. Changes in red blood cell shape or numbers may also affect ESR. Anticoagulated whole blood is allowed to stand in a narrow vertical tube and the red blood cells under the influence of gravity settle out of the plasma. The rate at which they settle is measured as the number of millimeters of clear plasma present at the top of the column after one hour (mm/hr).

The European League Against Rheumatism (EULAR) Sjogren’s Syndrome (SS) Disease Activity Index (ESSDAI) was developed as a homogenous evaluation of systemic activity (Seror et al., Ann Rheum Dis. 2010; 69(6):1103-1109). ESSDAI includes 12 domains (ie, organ systems: cutaneous, respiratory, renal, articular, muscular, peripheral nervous system. Central nervous system, hematological, glandular, constitutional lymphadenopathic, biological). Each domain is divided into 3-4 levels of activity. The definition of each activity level is provided by a detailed description of what should be considered in that domain. Possible scores range between 0 -123 and about eighty percent of patients have a score ≤13.

Baseline demographics, disease characteristics and biochemical data were similar between the treatment groups (Table 2). Specifically, the study population had mild to moderate disease as determined by ESSDAI scores and high disease activity (symptom burden) by ESSPRI scores. Study subjects also reported profound fatigue on the FACIT-F and ProF instruments. ESSDAI and ESSPRI scores in the placebo group were modestly higher than those in the RSLV-132 group. Complement 3, Complement 4, ESR, and IgG measurements were comparable to healthy values and similar between the two groups Table 2.

TABLE 2

Study subject demographics and mean clinical characteristics at baseline

| | Placebo (n=8) | RSLV-132 (n=20) |
|---|---|---|
| Age | 59.6 ± 8.8 | 56.5 ± 12.9 |
| Gender | | |
| Female | 100% | 100% |
| Male | 0% | 0% |
| Race | | |
| White | 87.5% | 95% |
| Asian | 12.5% | 5% |
| Ethnicity | | |
| Not Hispanic or latino | 100% | 95% |
| Hispanic or latino | 0% | 5% |
| Height (cm) | 165.13 ± 4.97 | 163.22 ± 8.05 |
| Weight (kg) | 81.4 ± 22.71 | 70.66 ± 13.95 |
| BMI (kg/m²) | 29.79 ± 8.20 | 26.52 ± 4.56 |
| Complement C3 | 125.3 ± 33.1 | 134.1 ± 24.0 |
| Complement C4 | 19.0 ± 6.2 | 19.6 ± 8.2 |
| IgG (mg/dL) | 1686 ± 563 | 1683 ± 810 |
| ESR | 23.3 ± 12.1 | 33.2 ± 33.9 |
| ESSDAI | 5.4 ± 4.1 | 5.0 ± 4.6 |
| ESSDAI ≤ 4 | 4/8 (50%) | 12/20 (60%) |
| ESSDAI ≥ 5 | 4/8 (50%) | 8/20 (40%) |
| ESSPRI | 6.42 ± 2.48 | 5.97 ± 1.57 |
| FACIT | 23.9 ± 11.41 | 29.6 ± 12.09 |
| Profile of fatigue | 4.0 ± 1.9 | 3.5 ± 1.2 |
| Immunomodulatory concomitant medications | | |
| Prednisone | 2 (25%) | 1 (5%) |

### Example 5

### Patients Treated With RSLV-132 Experienced a Clinically Meaningful Improvement in ESSPRI Score and an Improvement in ESSPRI Fatigue.

pSS is an autoimmune disorder characterized by the lymphatic infiltration of salivary and lacrimal glands with subsequent inflammation, damage and loss of function of the glands causing dry eyes and dry mouth. Clinical features of pSS can be separated into two groups (1) benign symptoms such as dryness, pain and fatigue that can be disabling and affect most patients; and (2) systemic manifestations that can be severe and affect 20-40% of patients (Seror et al. Ann Rheum Dis 2011; 70:968-972).

The EULAR SS Patient Reported Index (ESSPRI) was developed to assess patient’s symptoms in primary Sjogren’s syndrome and has been validated and accepted by the FDA. ESSPRI assesses dryness, pain, and fatigue in patients, and each symptom is assessed on a 0-10 numerical scale. A decrease of at least one point in the ESSPRI score is clinically meaningful.

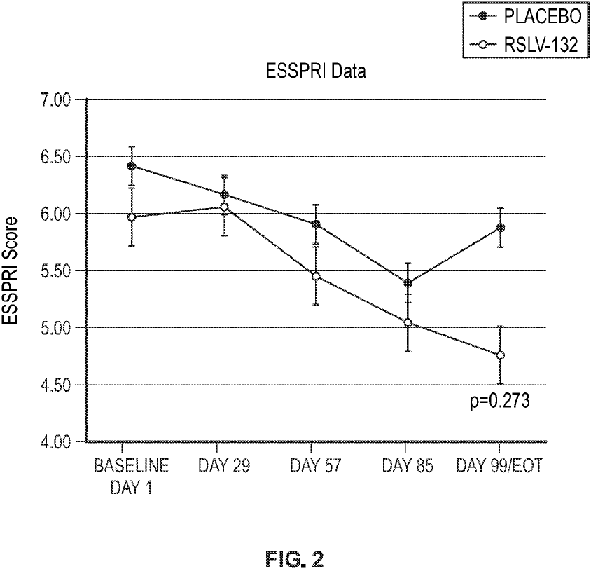

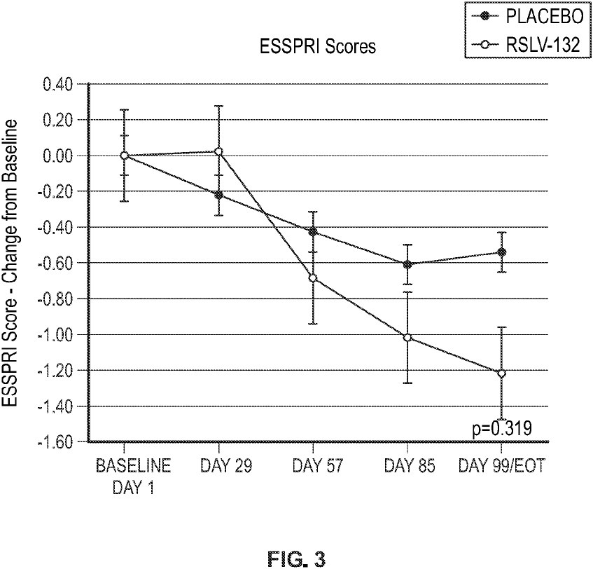

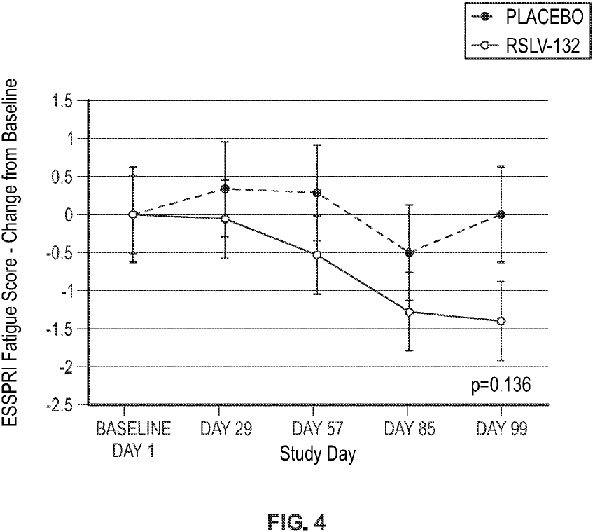

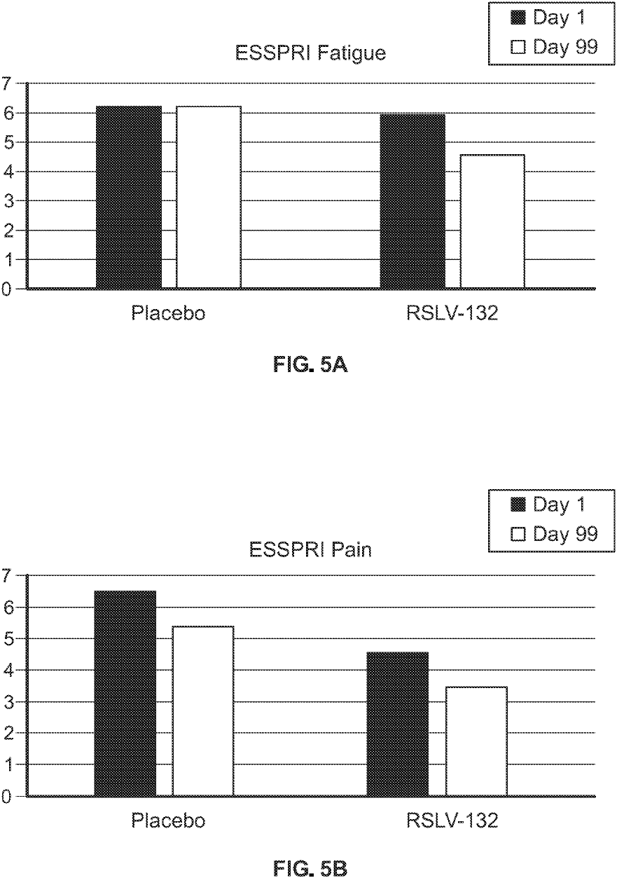

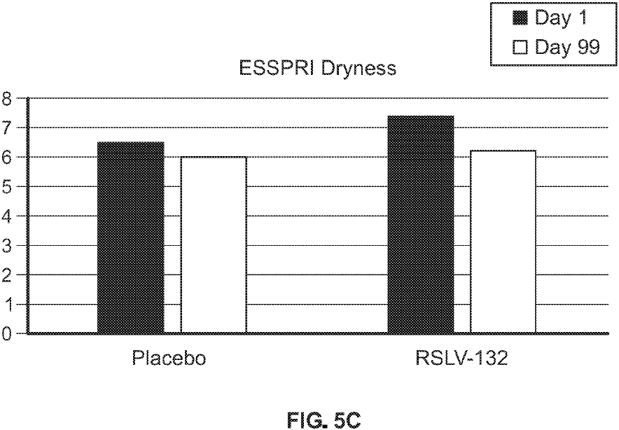

RSLV-132 treated patients experienced a greater than 1 point decrease in ESSPRI score over the course of the study (FIG. 2). Specifically, the ESSPRI score of patients receiving RSLV-132 decreased from approximately 6 at baseline to approximately 4.5 at day 99 (FIG. 2) and the change from baseline for patients was -1.20 (FIG. 3 and Table 3). This improvement in ESSPRI score was clinically meaningful. In contrast, as shown in FIG. 3 and Table 3, patients treated with placebo had a change from baseline of -0.54. Furthermore, patients treated with RSLV-132 experienced an improvement in ESSPRI fatigue of approximately -1.4 from baseline to day 99 of the study compared to 0 in the placebo group (FIG. 4). When the three components of the ESSPRI score were assessed individually, patients treated with RSLV-132 experienced a decrease in Sjogren’s related fatigue as compared to control patients treated with placebo (FIGS. 5A-5C). Subject level data revealed that 12.5% of subjects in the placebo group and 60% or RSLV-132 treated subjects had a minimal clinically important improvement (MCII) in ESSPRI (≥ 1 point decrease) by day 99 (Table 3). These data provide evidence that RSLV-132 treatment improves symptoms in patients with pSS and that there is a clinically meaningful reduction in fatigue.

TABLE 3

Clinical Efficacy Measures at Day 99

| | Mean day 99 score (change from BL) | | |
|---|---|---|---|
| | Placebo | RSLV-132 | P (RSLV-132 vs. placebo at D99) |
| ESSDAI | 2.9 (-2.50) | 5.0 (0.00) | 0.28* |
| ESSPRI | 5.88 (-0.54) | 4.75 (-1.22) | 0.27* |
| ESSPRI Fatigue | 6.30 (0.00) | 4.60 (-1.40) | 0.19* |
| FACIT | 25.00 (1.13) | 35.50 (5.90) | 0.05* |
| Pro-F | 3.98 (-0.02) | 2.54 (-1.04) | 0.07* |
| Somatic fatigue | 4.17 (0.00) | 2.87 (-0.80) | 0.13* |
| Mental fatigue | 3.75 (0.06) | 2.13 (-1.53) | 0.04* |
| DSST (change from BL; time to complete, seconds) | +2.8 | -16.4 | 0.02* |
| ESSDAI Responders (≥ 3 point decrease) | ⅜ (37.5%) | 4/20 (20%) | 0.75** |
| ESSPRI Responders (≥ 1 point decrease) | ⅛ (12.5%) | 12/20 (60%) | 0.06** |
| FACIT Responders (≥ 6 point increase) | 2/8 (25%) | 9/20 (45%) | 0.002** |
| Profile of Fatigue | | | |
| Somatic fatigue response (≥ 1 point decrease) | 2/8 (25%) | 10/20 (50%) | 0.37** |
| Mental fatigue response (≥ 1 point decrease) | 2/8 (25%) | 11/20 (55%) | 0.19** |

* The mean values for change from baseline for each group were analyzed using separate 1-way Analysis of Variance* (ANOVA) models for each visit, each testing the null hypothesis (Ho) that the true mean difference between treatment groups was zero. Unadjusted alpha = 0.05.
**To determine the significance of the difference in the rate of response to treatment in placebo versus RSLV-132 treated subjects, a Fisher’s Exact Test** was used for testing the null hypothesis for each clinical instrument.

### Example 6

### Patients Treated With RSLV-132 Experienced a Clinically Meaningful Improvement in FACIT Fatigue Score

The Functional Assessment of Chronic Illness Test (FACIT) fatigue scale is used to measure fatigue in chronic illness and is widely used in patients with Sjorgren’s syndrome. The FACIT fatigue (FACIT-F) questionnaire includes 13 questions about fatigue that are measured on a 4-point Likert scale. Total scores range from 0-52 and higher scores represent less fatigue (Chandran et al., Ann Rheum Dis 2007; 66:936-939).

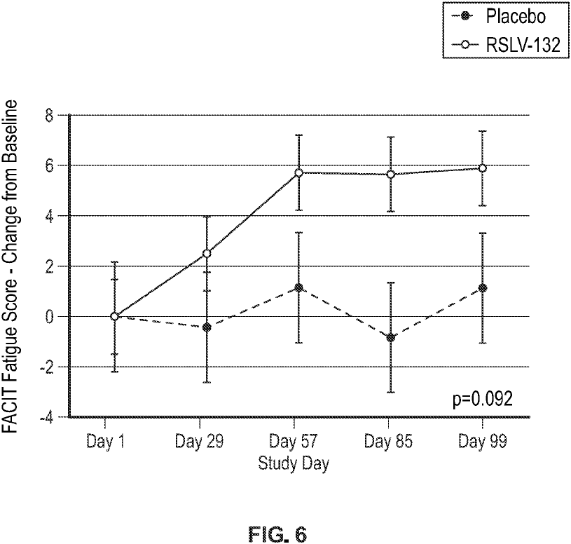

As depicted in FIG. 6, patients treated with RSLV-132 experienced a clinically meaningful improvement in FACIT fatigue score. In particular, the FACIT score for patients treated with RSLV-132 increased by approximately six points between baseline and day 57 of the study. In contrast, at day 57 of the study, the FACIT score for patients treated with placebo increased by approximately one point. At day 99 of the study, the FACIT score for patients treated with RSLV-132 increased by approximately six points (mean 5.9 increase) from baseline. In contrast, at day 99 of the study, the FACIT score for patients treated with placebo increased by approximately one point (mean increase 1.13) from baseline. Subject level data revealed that 25% of placebo subjects and 45% of the RSLV-132 treated subjects had a minimal clinically important improvement (MCII) in FACIT score (≥6 point increase) by day 99 (Table 3). These data provide evidence that RSLV-132 treatment improves fatigue in patients with pSS.

### Example 7

### Patients Treated With RSLV-132 Experienced an Improvement in PROF

The Profile of Fatigue (ProF) is used to measure fatigue associated with chronic illness and has been used to assess fatigue in patients with Sjogren’s syndrome. ProF consists of 16 items divided into two domains: (1) somatic fatigue, and (2) mental fatigue. The ProF is scored from 0 to 7 with a high score representing more fatigue. The scoring can be presented as a profile or as a calculated total score (Strombeck et al., Scand J Rheumatol 2005; 34:455-459).

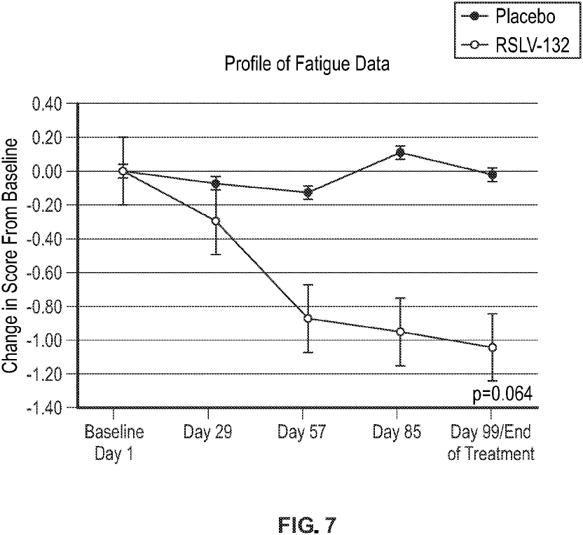

RSLV-132 treated patients experienced an improvement in ProF over the duration of the study. Specifically, the ProF score for patients treated with RSLV-132 decreased by more than 1 point (mean reduction of 1.04 points) from baseline to day 99 of the study (FIG. 7). In contrast, patients treated with placebo did not experience a decrease in ProF, with a mean reduction of 0.02 points (FIG. 7).

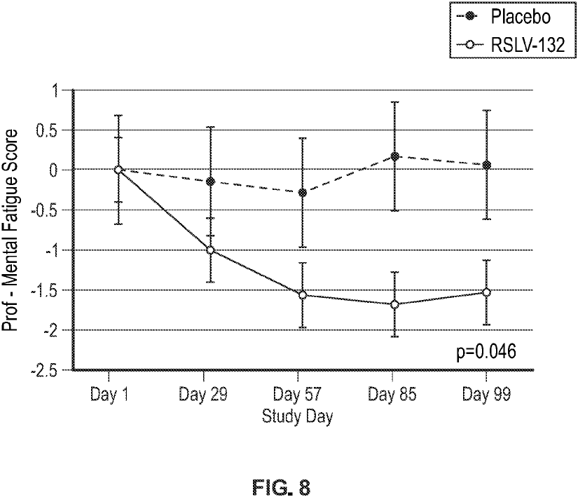

Notably, patients treated with RSLV-132 experienced a clinically meaningful improvement in the mental component of ProF over the course of the study as the mental score decreased by approximately 1.5 points (mean decrease of 1.53 points in the mental fatigue component) from baseline to day 99 of the study (FIG. 8). In contrast, patients treated with placebo experienced a mean decrease in ProF score of 0.06 points (FIG. 8). As shown in Table 3, mental fatigue response (≥1 point decrease) was observed in 25% of placebo patients and 55% of RSLV-132 treated patients at day 99. These data provide evidence that the active group (patients treated with RSLV-132) experienced a clinically meaningful improvement in the mental component of ProF (≥1 point decrease).

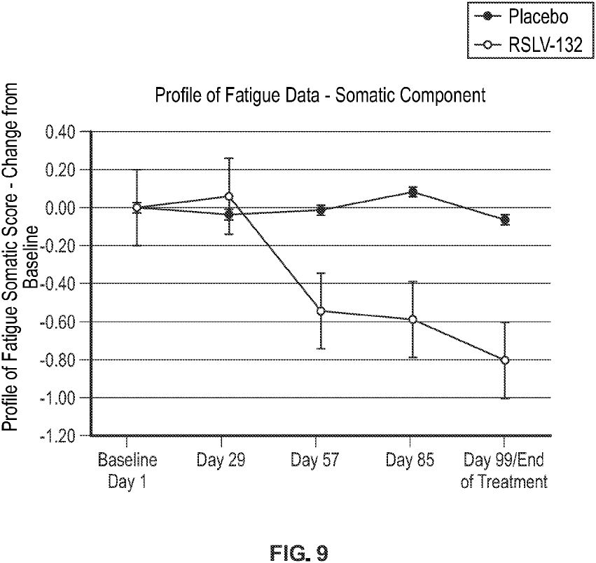

Patients treated with RSLV-132 also experienced an improvement in the somatic component of PROF as the somatic score decreased by approximately 0.8 points from baseline over the course of the study (FIG. 9). As shown in Table 3, somatic fatigue response (≥1 point decrease) was observed in 25% of placebo patients and 50% of RSLV-132 treated patients at day 99. Patients treated with placebo did not experience a decrease in either the mental or somatic component of PROF. These data provide evidence that RSLV-132 treatment improves fatigue in patients with pSS.

### Example 8

### Patients Treated With RSLV-132 Experienced a Statistically Significant Improvement in DSST

The Digit Symbol Substitution Test (DSST) was used to measure cognitive function (e.g., attention and focus) in Sjogren’s syndrome patients. DSST is a highly validated, sensitive instrument that is widely used in clinical studies involving CNS drugs as a readout on executive function. The DSST is a time limited paper-and-pencil cognitive test. The test requires a patient to match symbols to numbers according to a key at the top of the paper. The patient copies the symbol into spaces below a row of numbers and the number of correct symbols within the allowed time is calculated.

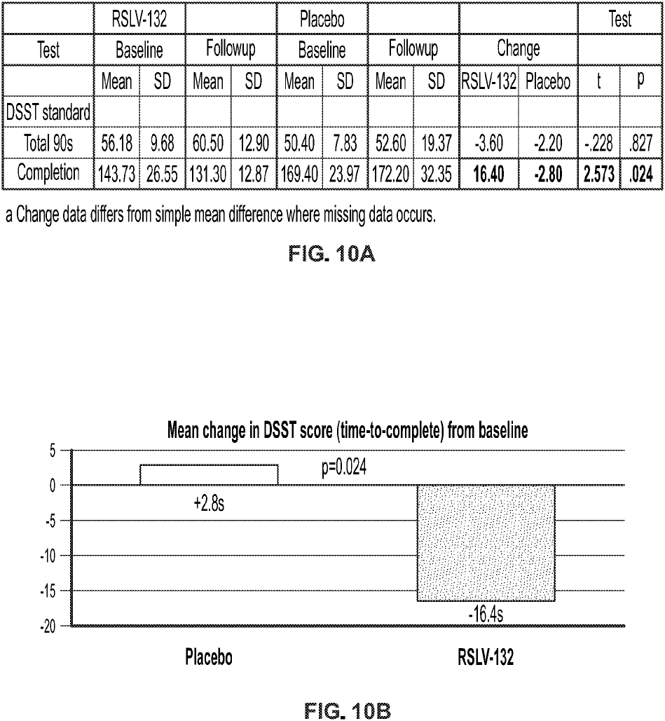

A subset of 16 patients were administered the digit symbol substitution test (DSST). The results of DSST neuropsychological testing support the above-described fatigue results. Patients were administered the DSST at baseline and follow-up (day 99). The total number of symbols matched to numbers completed in 90 seconds was measured as well as the time to complete the test in seconds. An increase in the number of matches completed in the allotted time demonstrates improvement. A decrease in the time to complete the test also demonstrates an improvement. Notably, patients treated with RSLV-132 demonstrated a statistically significant improvement in the time to complete the test between baseline and follow-up with a change of 16.40 as compared to a change of -2.80 for patients treated with placebo (FIG. 10A). As shown in FIG. 10B, RSLV-132 patients completed the task 16.40 seconds faster (loss of 16.4 seconds) than baseline whereas placebo patients were 2.80 seconds slower (added 2.80 seconds) than their original time at baseline. Patients treated with RSLV-132 also demonstrated an improvement in the number of matches completed in 90 seconds between baseline and follow-up (FIG. 10A). An improvement in the DSST test supports a finding of reduced fatigue in patients with Sjogren’s syndrome as a reduction in fatigue corresponds with improved cognitive ability.

### Example 9

### Safety and Tolerability of RSLV-132 Treatment

To assess the overall safety and tolerability of treatment with RSLV-132, adverse events were measured throughout the study. Adverse events were monitored out to 211 days following the final treatment. The incidence of treatment-emergent adverse events, serious adverse events, and drug-related adverse events were comparable between the RSLV-132 and placebo treatment groups (Table 4). No deaths occurred during the study. Fatigue was the most common adverse event (AE) in the study. Most of the fatigue AE’s were reported early in the study. There were no serious infections, or infusion reactions observed in either treatment group during the study. No patients discontinued study drug due to an adverse event AE. One patient in the RSLV-132 group experienced a serious adverse event and was hospitalized for parotitis 88 days after the last dose of study drug. This adverse event did not appear to be correlated with RSLV-132 treatment.

TABLE 4

Treatment emergent adverse events (TEAE) (safety analysis set)

| | Placebo (N=8) | RSLV-132 (N=20) |
|---|---|---|
| At least one TEAE | 8 (100%) | 20 (100%) |
| At least one drug-related TEAE | 5 (62.5%) | 13 (65%) |
| At least one serious adverse event | 0 | 1 (5%)* |
| At least one drug-related serious TEAE | 0 | 0 |
| Infections | 6 (75%) | 16 (80%) |
| Deaths | 0 | 0 |
| Most common AE’s | | |
| Fatigue | 1 (12.5%) | 6 (30%) |
| URTI | 2 (25%) | 5 (25%) |
| Arthralgia | 0 | 5 (25%) |
| Viral URI | 1 (13%) | 4 (20%) |
| Conjunctivitis | 1 (13%) | 3 (15%) |
| Headache | 1 (13%) | 3 (15%) |
| LRTI | 3 (38%) | 1 (5%) |
| Sjogren’s syndrome | 2 (25%) | 0 |

*hospitalized for parotitis 88 days after last dose of study drug; unrelated to study drug

### Example 10

### Statistical Analysis

To analyze the ESSDAI, ESSPRI, FACIT-F, ProF, and DSST data, the mean values for change from baseline for each group were analyzed using separate 1-way Analysis of Variance* (ANOVA) models for each visit, each testing the null hypothesis (Ho) that the true mean difference between treatment groups was zero, with an unadjusted significance level of alpha = 0.05. To analyze the rate of response to treatment in placebo versus RSLV-132 treated subjects, a Fisher’s Exact Test was used for testing the null hypothesis for each clinical instrument. For analysis of gene expression data, DESeq2 estimates of the fold change in IFN-inducible gene expression between experimental conditions using a Negative Binomial generalized linear regression model (GLM) with a logarithmic link function were used. Fold changes and p-values are derived by carrying out Wald tests on the resulting likelihood functions. In the implementation of the Wald test used by DESeq2, the maximum likelihood estimate for the fold change is divided by its standard error to obtain a test statistic which is then compared to a standard Normal distribution.

### Example 11

### RSLV-132 Treated Patients Exhibit Increased Expression of Selected IFN-Inducible Genes

Previously, it was demonstrated that RSLV-132 treated patients experiencing a clinical response achieved a broad reduction in inflammatory-related gene expression, which was not observed in in patients that did not achieve a clinical response. Furthermore, an increase in other genes was also observed in patients achieving a clinical response to RSLV-132 (PCT/US2020/012258). In the present disclosure, additional analysis surprisingly revealed that patients achieving a clinical response to RSLV-132 experienced an increase in expression of selected interferon-inducible genes.

To evaluate a change in the inflammatory state of patients treated with RSLV-132, the expression of groups of interferon-inducible genes contained in three modules (M1.2, M3.4, and M5.12 was examined. The modules have been described by Chiche et al., Arthritis and Rheumatology, Vol 66, No. 6, 2014, 1583-1595. Each of the three modules contain genes that are under the regulation of interferon-α and are used as surrogates for the interferon pathway. The modules provide a readout on interferon activity.

RNA sequencing of whole blood samples from 28 patients (20 treated with RSLV-132 and 8 treated with placebo) was performed at Q2 Solutions | EA Genomics in Morrisville, NC. Whole blood was collected from each patient in PAXGene® collection tubes on days 1 at baseline prior to treatment and day 99 prior to treatment. RNA was extracted and quantitated by spectrophotometry using Thermo-Fisher’s NanoDrop 8000 and integrity assessed using the RNA 6000 Nano Assay on a Bioanalyzer 2100. Fifty base-pair stranded and paired-end sequencing libraries were generated using the Illumina TruSeq Stranded Total RNA protocol with RiboZero Magnetic Gold depletion of rRNA. Libraries were sequenced on an Illumina HiSeq 2500 to a target depth of 50 million reads. Prior to gene mapping, adapter trimming, homopolymer filtering and low quality read filtering were performed. Pre-processed reads were mapped to hg38 assembly of the human genome using kallisto 0.45.0 with the use of Gencode release 31 of human reference gene annotations. For analysis of the interferon-inducible genes, reads were mapped to Gencode release 33 (GRCh38) transcript sequences using Salmon Version 1.1.0 to obtain transcript level quantification estimates. These transcript level estimates were aggregated to gene-level counts using tximport version 1.14.0. Differential gene expression analysis was carried out using Bioconductor DESeq2 version 1.26.0.

For analysis of gene expression data, DESeq2 estimates of fold change between experimental conditions using a Negative Binomial GLM with a logarithmic link function were used. Fold changes and p-values are derived by carrying out Wald tests on the resulting likelihood functions. In the implementation of the Wald test used by DESeq2, the maximum likelihood estimate for the fold change is divided by its standard error to obtain a test statistic which is then compared to a standard Normal distribution.

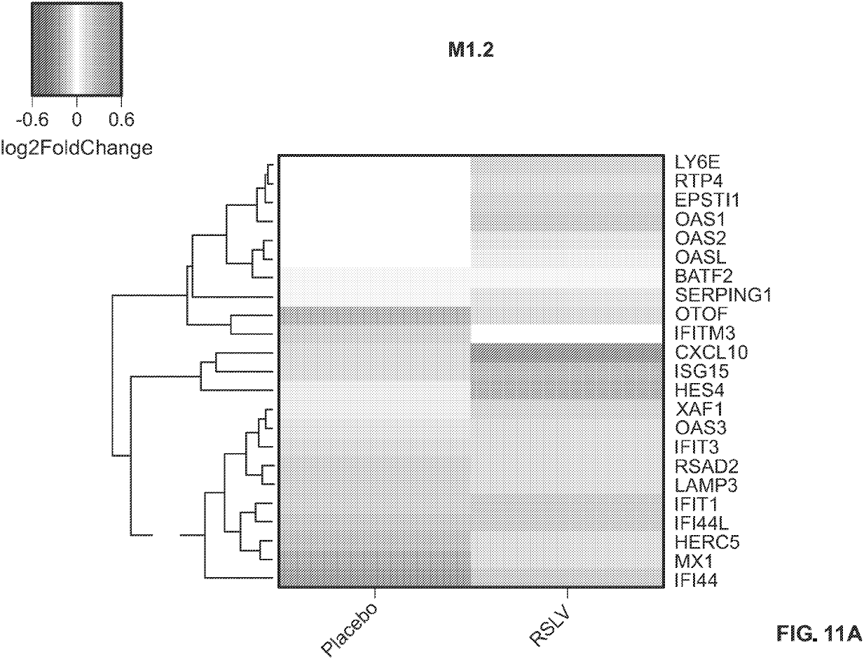

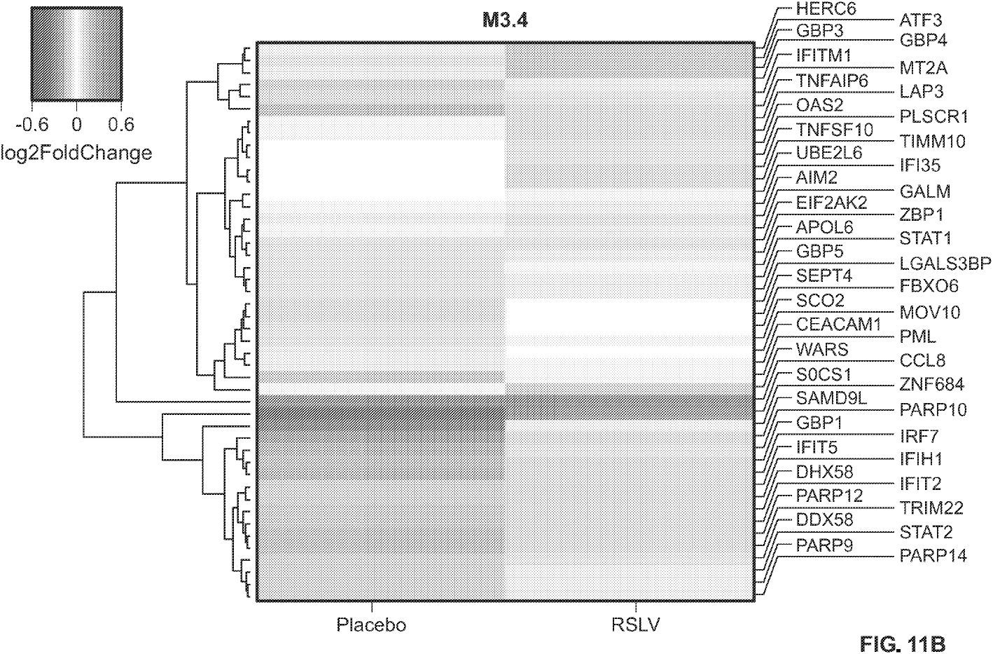

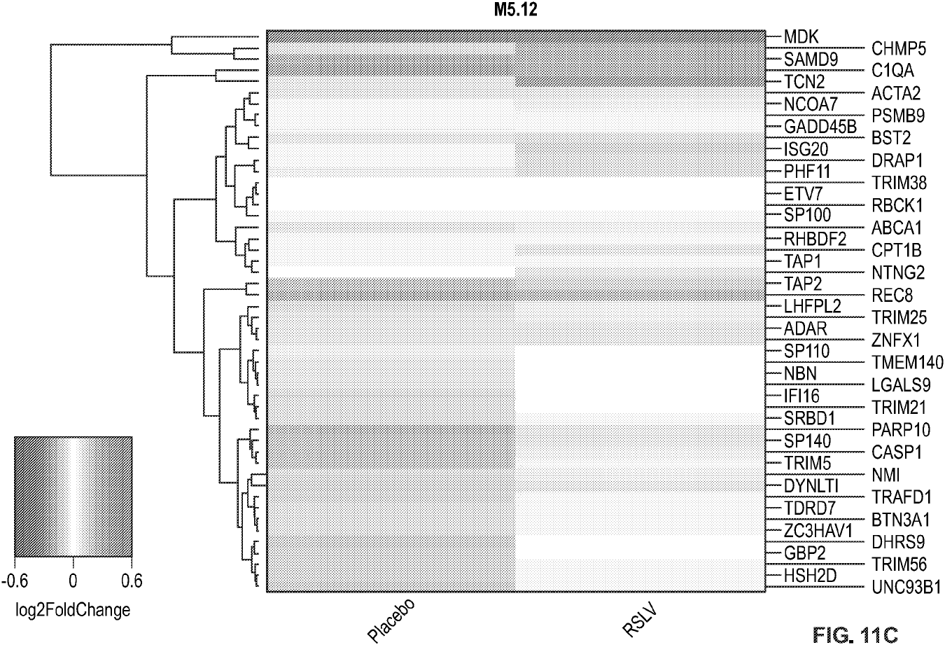

The mean expression of interferon-inducible genes within the three modules (M1.2, M3.4, M5.12) revealed increased expression of selected IFN-inducible genes in RSLV-132 treated patients as compared to placebo treated patients. In module M1.2, the mean log2 fold change between days 1 and 99 was -0.03 in the placebo group and 0.13 in the RSLV-132 group (FIG. 11A). For module M3.4, the mean log2 fold change between days 1 and 99 was -0.03 in the placebo group and 0.08 in the RSLV-132 group (FIG. 11B). For module M5.12, the mean log2 fold change between days 1 and 99 was -0.06 in the placebo group and 0.03 in the RSLV-132 group (FIG. 11C).

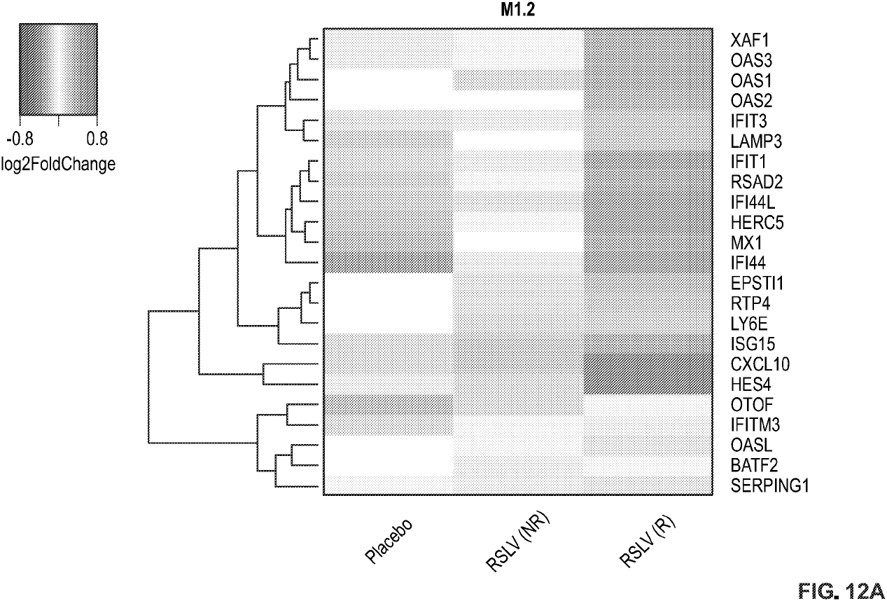

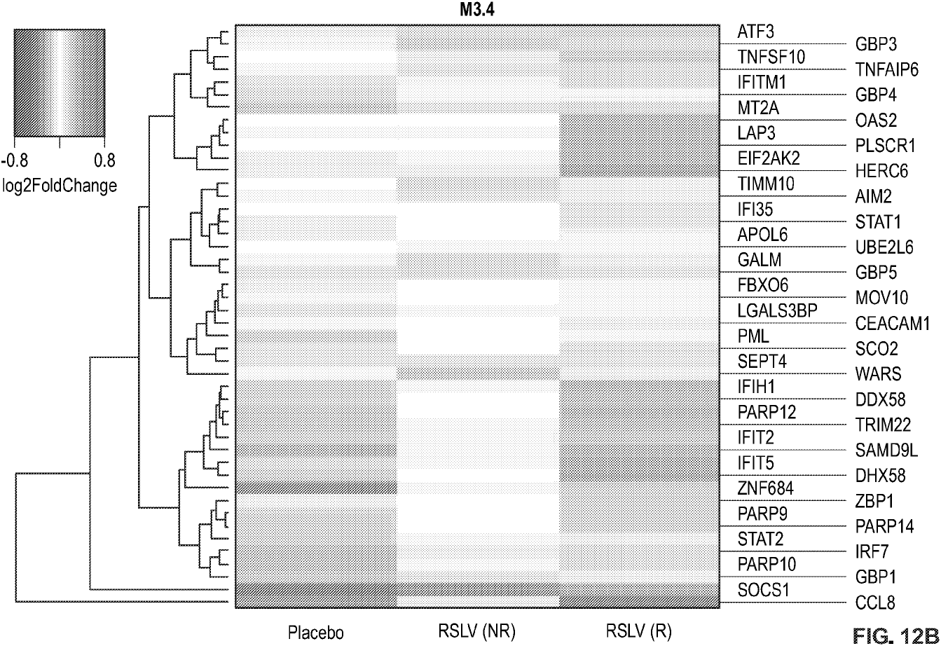

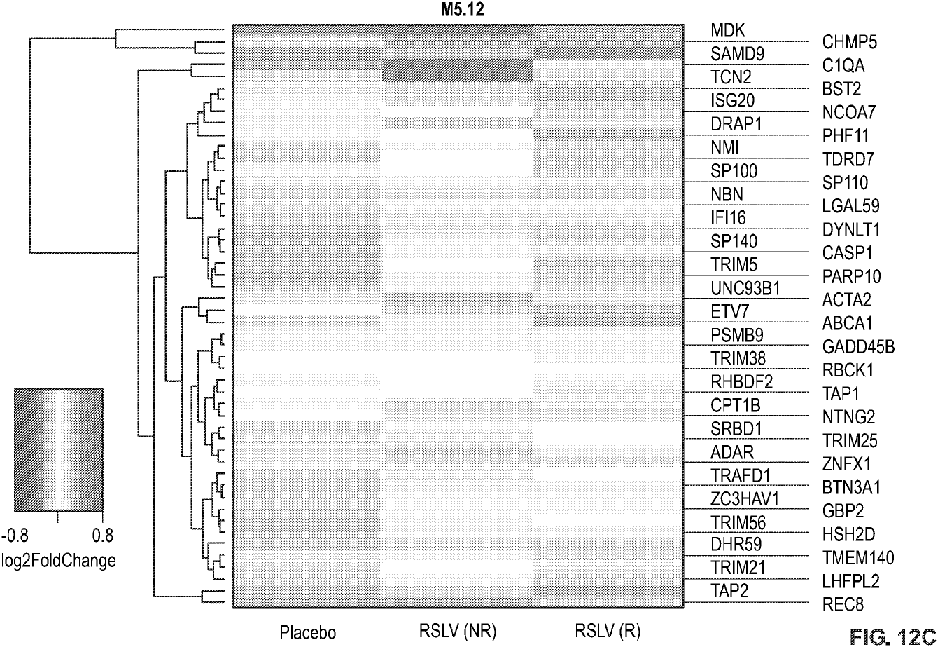

Comparing the subgroup of RSLV-132 treated subjects who experienced a clinical response (defined as subjects who achieved a minimal clinically important improvement (MCII) in two of the three instruments, ESSPRI, FACIT-F, or ProF) to those RSLV-132 subjects who did not experience a clinical response (non-responders), revealed a larger difference in RSLV-132 stimulated IFN-inducible gene upregulation (10 of the 20 subjects treated with RSLV-132 achieved a clinical response and 2 of the 8 placebo treated patients has a clinical response). In module M1.2, the mean log2 fold change between days 1 and 99 for non-responders was 0.07 and in the responder group 0.25 (FIG. 12A). For module M3.4, the mean log2 fold change for the non-responder subgroup was 0.04 and 0.16 for the responder subgroup (FIG. 12B). For module M5.12, the mean log2 fold change between for the non-responder subgroup was 0.02 and for the responder subgroup 0.04 (FIG. 12C).

Analysis of the correlation of changes in gene expression in these IFN-inducible genes and performance on three patient-reported outcome measures (ESSPRI, ProF, and FACIT-F) were conducted by analyzing the changes between day 1 and day 99 of the study. A strong, statistically significant correlation was observed between the mental fatigue scores and the expression of IFN-inducible genes contained in the M1.2 module among patients in the RSLV-132 treated group (P = 0.014).

All of the patients in the study presented with an interferon signature at baseline (day 1). Unexpectedly, several of the patients that achieved a clinical response to RSLV-132 experienced an increase in the expression of selected interferon-inducible genes. These data provide evidence that improvement in fatigue in RSLV-132 treated patients correlated with an increase in the expression of selected interferon-inducible genes.

### Example 12

### Statistical Analysis

To analyze the correlation between the expression patterns of the individual genes contained in the three modules of IFN-inducible genes and the scores from the patient-reported instruments (mental fatigue, somatic fatigue, and ESSPRI), module scores for each patient were calculated as the mean normalized gene expression across all genes in each module. The normalization method used was the median of ratios method, as used by DESeq2. Pearson’s correlation coefficients were calculated to assess the correlations between changes in the mean normalized gene expression for each module and changes in the mean scores for mental fatigue, somatic fatigue, and ESSPRI.

### Example 13

### Pharmacokinetics and Immunogenicity

RSLV-132 protein concentrations in the serum were measured using a validated electrochemiluminescence-based assay. In addition, RSLV-132 catalytic RNase activity was measured using an RNase enzyme assay. It was observed that the two measurements were highly correlated. The median RSLV-132 serum concentration, as determined by enzyme-linked immunosorbent assay in the serum at steady state, was 4 µg/ml (1.3-11.0 µg/ml). The RNase catalytic enzyme activity measurement of serum drug levels was 4.6 µg/ml (2.8-12 µg/ml) at steady state on day 57 (data not shown). Serum was assayed at multiple time points for anti-RSLV-132 antibodies using an assay validated in accordance with US Food and Drug Administration guidance. None of the subjects in the study were positive for anti-RSLV-132 antibodies (data not shown).

### Example 14

### RSLV-132 Responders Exhibit a Decrease in Expression of Select Inflammatory Related Genes

To further evaluate a change in the inflammatory state of patients treated with RSLV-132, the expression of select inflammatory-related genes were examined. RNA sequencing and analysis was performed as described in Example 11.

Patients were selected by treatment (placebo treated or RSLV-132 treated) and RSLV-132 treated patients were further divided as responders (R) or non-responders (NR). Responders were defined as subjects who experienced a minimal clinically important improvement (MCII) in two of three instruments (FACIT-F, ProF, or ESSPRI instrument).

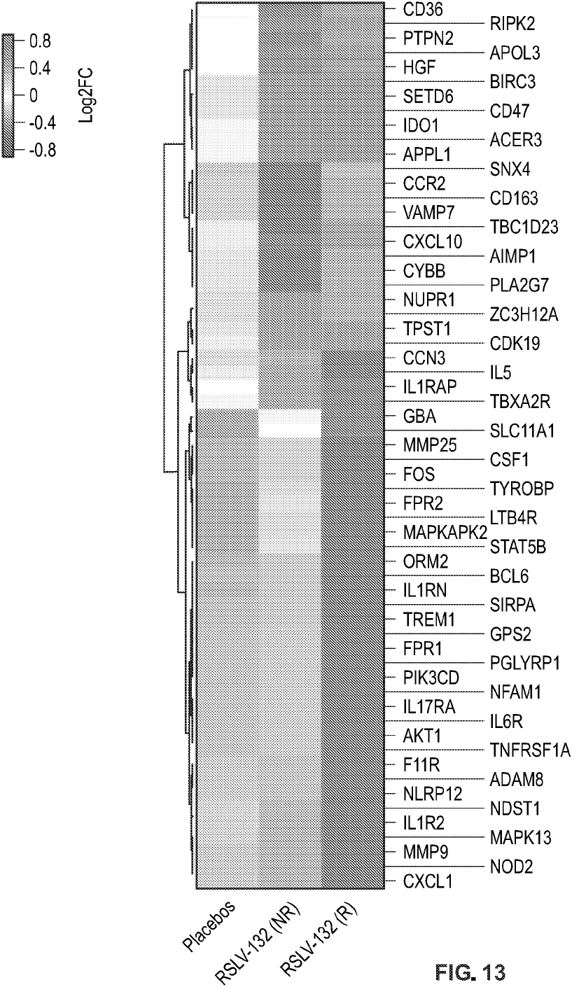

Mean values for changes (log2-fold changes) in expression of the inflammation-related genes were assessed by comparing treatment days 1 and 99 for the placebo group and non-responders (NR) and responders (R) in the RSLV-132 treatment group. A general decrease in expression of inflammatory related genes was observed in patients that responded to treatment with RSLV-132 as compared to non-responders and placebo treated patients (FIG. 13). The expression level of inflammatory related genes in placebo treated patients and non-responders was similar.

These data provide evidence that there is a change in the inflammatory state in patients that respond to treatment with RSLV-132 and that an improvement in fatigue as assessed by FACIT-F, ProF, or ESSPRI instrument correlated with a general decrease in expression of inflammatory related genes.

### Example 15

### RSLV-132 Responders Exhibit a Decrease in Expression of Select IncRNA Molecules

To further analyze the inflammatory state of patients treated with RSLV-132 the expression of select long non-coding RNAs (IncRNAs) that regulate expression of genes involved in inflammation was examined. RNA sequencing of IncRNAs and analysis was performed as described in Example 11.

Patients were selected by treatment (placebo treated or RSLV-132 treated) and RSLV-132 treated patients were further divided as responders (R) or non-responders (NR). Responders were defined as subjects who experienced a minimal clinically important improvement (MCII) in two of three instruments (FACIT-F, ProF, or ESSPRI instrument).

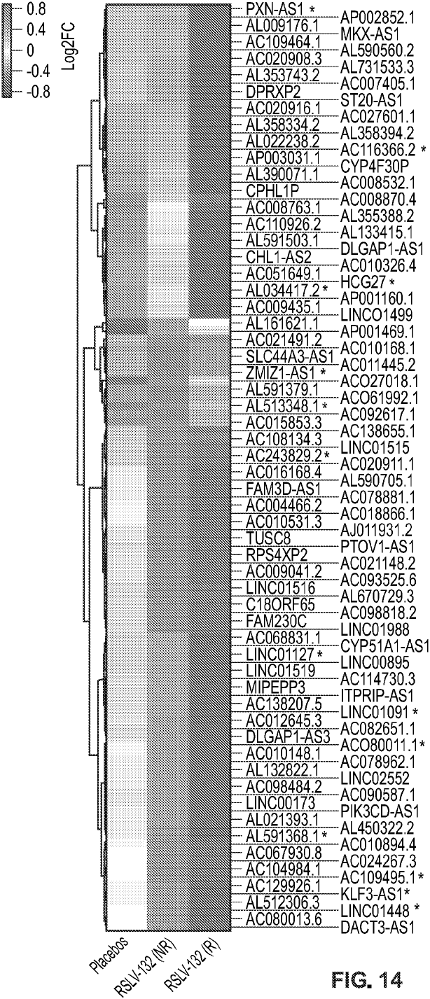

An overall decrease in expression of IncRNAs that regulate transcription of genes involved in inflammation was observed in patients that responded to treatment with RSLV-132. The mean values for change in expression in IncRNA molecules showed a strong negative correlation with an improvement in FACIT-F score, using log2-fold changes comparing treatment days 1 and 99 for the placebo group and non-responders (NR) and responders (R) in the RSLV-132 treatment group (FIG. 14). Astrisks indicate IncRNA molecules which have been identified in genome-wide association studies (GWAS) as being associated with autoimmune diseases and immune system function (FIG. 14). For example, the expression of each of the following IncRNAs was decreased in patients that responded to treatment with RSLV-132: PXN-AS1, AC116366.2, HCG27, AL034417.2, ZMIZ1-AS1, AL513348.1, AC243829.2, LINC01127, LINC01091, AC080011.1, AL591368.1, AC109495.1, KLF3-AS1, and LINC01448. Each of these IncRNAs was previously identified in GWAS studies as being associated with autoimmune diseases and immune system function.

These data provide evidence that there is a change in the inflammatory state in patients that respond to treatment with RSLV-132 and that an improvement in fatigue as assessed by FACIT-F, ProF, or ESSPRI instrument correlated with an overall decrease in expression of IncRNAs.

TABLE 1

Sequence Table

| SEQ ID NO. | Description | Sequence |
|---|---|---|
| 1 | Precursor human RNase1 | MALEKSLVRLLLLVLILLVLGWVQPSLGKESRAKKFQRQHMDSDS SPSSSSTYCNQMMRRRNMTQGRCKPVNTFVHEPLVDVQNVCFQEK VTCKNGQGNCYKSNSSMHITDCRLTNGSRYPNCAYRTSPKERHII VACEGSPYVPVHFDASVEDST |
| 2 | Mature human RNase1 | KESRAKKFQRQHMDSDSSPSSSSTYCNQMMRRRNMTQGRCKPVNT FVHEPLVDVQNVCFQEKVTCKNGQGNCYKSNSSMHITDCRLTNGS RYPNCAYRTSPKERHIIVACEGSPYVPVHFDASVEDST |
| 3 | hRNase-G88D-3′ | KESRAKKFQRQHMDSDSSPSSSSTYCNQMMRRRNMTQGRCKPVNT FVHEPLVDVQNVCFQEKVTCKNGQGNCYKSNSSMHITDCRLTNDS RYPNCAYRTSPKERHIIVACEGSPYVPVHFDASVEDST |
| 4 | Mature human RNase1 N34S/N76S/N 88S | KESRAKKFQRQHMDSDSSPSSSSTYCNQMMRRRSMTQGRCKPVNT FVHEPLVDVQNVCFQEKVTCKNGQGNCYKSSSSMHITDCRLTSGS RYPNCAYRTSPKERHIIVACEGSPYVPVHFDASVEDST |
| 5 | Precursor human DNase1 | MRGMKLLGALLATAATLLQGAVSLKIAAFNIQTFGETKMSNATLVS YIVQILSRYDIALVQEVRDSHLTAVGKLLDNLNQDAPDTYHYVVS EPLGRNSYKERYLFVYRPDQVSAVDSYYYDDGCEPCGNDTFNREP AIVRFFSRFTEVREFAIVPLHAAPGDAVAEIDALYDVYLDVQEKW GLEDVMLMGDFNAGCSYVRPSQWSSIRLWTSPTFQWLIPDSADTT ATPTHCAYDRIVVAGMLLRGAVVPDSALPFNFQAAYGLSDQLAQA ISDHYPVEVMLK |
| 6 | Mature wild type Human DNase1 UniProt P24855 | LKIAAFNIQTFGETKMSNATLVSYIVQILSRYDIALVQEVRDSHL TAVGKLLDNLNQDAPDTYHYVVSEPLGRNSYKERYLFVYRPDQVS AVDSYYYDDGCEPCGNDTFNREPAIVRFFSRFTEVREFAIVPLHA APGDAVAEIDALYDVYLDVQEKWGLEDVMLMGDFNAGCSYVRPSQ WSSIRLWTSPTFQWLIPDSADTTATPTHCAYDRIVVAGMLLRGAV VPDSALPFNFQAAYGLSDQLAQAISDHYPVEVMLK |
| 7 | hDNase1-3′-G105R; A114F | lkiaafniqtfgetkmsnatlvsyivqilsrydialvqevrdshl tavgklldnlnqdapdtyhyvvseplgrnsykerylfvyrpdqvs avdsyyyddgcepcrndtfnrepfivrffsrftevrefaivplha apgdavaeidalydvyldvqekwgledvmlmgdfnagcsyvrpsq wssirlwtsptfqwlipdsadttatpthcaydrivvagmllrgav vpdsalpfnfqaayglsdqlaqaisdhypvevmlk |
| 8 | hDNase1-3′ A114F | lkiaafniqtfgetkmsnatlvsyivqilsrydialvqevrdshl tavgklldnlnqdapdtyhyvvseplgrnsykerylfvyrpdqvs avdsyyyddgcepcgndtfnrepfivrffsrftevrefaivplha apgdavaeidalydvyldvqekwgledvmlmgdfnagcsyvrpsq wssirlwtsptfqwlipdsadttatpthcaydrivvagmllrgav vpdsalpfnfqaayglsdqlaqaisdhypvevmlk |
| 9 | hDNase1-5′-G105R | lkiaafniqtfgetkmsnatlvsyivqilsrydialvqevrdshl tavgklldnlnqdapdtyhyvvseplgrnsykerylfvyrpdqvs avdsyyyddgcepcrndtfnrepaivrffsrftevrefaivplha apgdavaeidalydvyldvqekwgledvmlmgdfnagcsyvrpsq wssirlwtsptfqwlipdsadttatpthcaydrivvagmllrgav vpdsalpfnfqaayglsdqlaqaisdhypvevmlk |
| 10 | human DNase1+VK3L P | metpaqllfllllwlpdttglkiaafniqtfgetkmsnatlvsyi vqilsrydialvqevrdshltavgklldnlnqdapdtyhyvvsep lgrnsykerylfvyrpdqvsavdsyyyddgcepcgndtfnrepai vrffsrftevrefaivplhaapgdavaeidalydvyldvqekwgl edvmlmgdfnagcsyvrpsqwssirlwtsptfqwlipdsadttat pthcaydrivvagmllrgavvpdsalpfnfqaayglsdqlaqais dhypvevmlk |
| 11 | Mature human DNase1 N18S/N106S/A114F | LKIAAFNIQTFGETKMSSATLVSYIVQILSRYDIALVQEVRDSHL TAVGKLLDNLNQDAPDTYHYVVSEPLGRNSYKERYLFVYRPDQVS AVDSYYYDDGCEPCGSDTFNREPFIVRFFSRFTEVREFAIVPLHA APGDAVAEIDALYDVYLDVQEKWGLEDVMLMGDFNAGCSYVRPSQ WSSIRLWTSPTFQWLIPDSADTTATPTHCAYDRIVVAGMLLRGAV VPDSALPFNFQAAYGLSDQLAQAISDHYPVEVMLK |
| 12 | Mature human DNase1 E13R/N74K/A 114F/ T205K | LKIAAFNIQTFGRTKMSNATLVSYIVQILSRYDIALVQEVRDSHL TAVGKLLDNLNQDAPDTYHYVVSEPLGRKSYKERYLFVYRPDQVS AVDSYYYDDGCEPCGNDTFNREPFIVRFFSRFTEVREFAIVPLHA APGDAVAEIDALYDVYLDVQEKWGLEDVMLMGDFNAGCSYVRPSQ WSSIRLWTSPTFQWLIPDSADTTAKPTHCAYDRIVVAGMLLRGAV VPDSALPFNFQAAYGLSDQLAQAISDHYPVEVMLK |
| 13 | Mature human DNase1 E13R/N74K/A 114F/T205K/N18S/N106S | LKIAAFNIQTFGRTKMSSATLVSYIVQILSRYDIALVQEVRDSHL TAVGKLLDNLNQDAPDTYHYVVSEPLGRKSYKERYLFVYRPDQVS AVDSYYYDDGCEPCGSDTFNREPFIVRFFSRFTEVREFAIVPLHA APGDAVAEIDALYDVYLDVQEKWGLEDVMLMGDFNAGCSYVRPSQ WSSIRLWTSPTFQWLIPDSADTTAKPTHCAYDRIVVAGMLLRGAV VPDSALPFNFQAAYGLSDQLAQAISDHYPVEVMLK |
| 14 | DNase1L3 | msrelapllllllsihsalamricsfnvrsfgeskqedknamdvi vkvikrcdiilvmeikdsnnricpilmeklnrnsrrgitynyvis srlgrntykeqyaflykeklvsvkrsyhyhdyqdgdadvfsrepf vvwfqsphtavkdfviiplhttpetsvkeidelvevytdvkhrwk aenfifmgdfnagcsyvpkkawknirlrtdprfvwligdqedttv kkstncaydrivlrgqeivssvvpksnsvfdfqkayklteeeald vsdhfpvefklqssraftnskksvtlrkktkskrs |
| 15 | hDNase 1L3 (mature) UniProt Q13609 | mricsfnvrsfgeskqedknamdvivkvikrcdiilvmeikdsnn ricpilmeklnrnsrrgitynyvissrlgrntykeqyaflykekl vsvkrsyhyhdyqdgdadvfsrepfvvwfqsphtavkdfviiplh ttpetsvkeidelvevytdvkhrwkaenfifmgdfnagcsyvpkk awknirlrtdprfvwligdqedttvkkstncaydrivlrgqeivs svvpksnsvfdfqkayklteeealdvsdhfpvefklqssraftns kksvtlrkktkskrs |
| 16 | hTREX1 | mgpgarrqgrivqgrpemcfcppptplpplriltlgthtptpcss pgsaagtyptmgsqalppgpmqtliffdmeatglpfsqpkvtelc llavhrcalespptsqgppptvpppprvvdklslcvapgkacspa aseitglstavlaahgrqcfddnlanlllaflrrqpqpwclvahn gdrydfpllqaelamlgltsaldgafcvdsitalkalerasspse hgprksyslgsiytrlygqsppdshtaegdvlallsicqwrpqal lrwvdaharpfgtirpmygvtasartkprpsavtttahlattrnt spslgesrgtkdlppvkdpgalsregllaplgllailtlavatly glslatpge |
| 17 | hTREX1 (C-terminal 72 aa truncated) | mgpgarrqgrivqgrpemcfcppptplpplriltlgthtptpcss pgsaagtyptmgsqalppgpmqtliffdmeatglpfsqpkvtelc llavhrcalespptsqgppptvpppprvvdklslcvapgkacspa aseitglstavlaahgrqcfddnlanlllaflrrqpqpwclvahn gdrydfpllqaelamlgltsaldgafcvdsitalkalerasspse hgprksyslgsiytrlygqsppdshtaegdvlallsicqwrpqal lrwvdaharpfgtirpmygvtasartk |
| 18 | Human DNase2 alpha (NP_001366. 1) | MIPLLLAALLCVPAGALTCYGDSGQPVDWFVVYKLPALRGSGEAA QRGLQYKYLDESSGGWRDGRALINSPEGAVGRSLQPLYRSNTSQL AFLLYNDQPPQPSKAQDSSMRGHTKGVLLLDHDGGFWLVHSVPNF PPPASSAAYSWPHSACTYGQTLLCVSFPFAQFSKMGKQLTYTYPW VYNYQLEGIFAQEFPDLENVVKGHHVSQEPWNSSITLTSQAGAVF QSFAKFSKFGDDLYSGWLAAALGTNLQVQFWHKTVGILPSNCSDI WQVLNVNQIAFPGPAGPSFNSTEDHSKWCVSPKGPWTCVGDMNRN QGEEQRGGGTLCAQLPALWKAFQPLVKNYQPCNGMARKPSRAYKI |
| 19 | human DNase2 beta | MKQKMMARLLRTSFALLFLGLFGVLGAATISCRNEEGKAVDWFTF YKLPKRQNKESGETGLEYLYLDSTTRSWRKSEQLMNDTKSVLGRT LQQLYEAYASKSNNTAYLIYNDGVPKPVNYSRKYGHTKGLLLWNR VQGFWLIHSIPQFPPIPEEGYDYPPTGRRNGQSGICITFKYNQYE AIDSQLLVCNPNVYSCSIPATFHQELIHMPQLCTRASSSEIPGRL LTTLQSAQGQKFLHFAKSDSFLDDIFAAWMAQRLKTHLLTETWQR KRQELPSNCSLPYHVYNIKATKLSRHSYFSSYQDHAKWCISQKGT KNRWTCIGDLNRSPHQAFRSGGFICTQNWQIYQAFQGLVLYYESC K |
| 20 | Human IgG1 Fc domain (wild-type) | EPKSCDKTHTCPPCPAPELLGGPSVFLFPPKPKDTLMISRTPEVT CVVVDVSHEDPEVKFNWYVDGVEVHNAKTKPREEQYNSTYRVVSV LTVLHQDWLNGKEYKCKVSNKALPAPIEKTISKAKGQPREPQVYT LPPSRDELTKNQVSLTCLVKGFYPSDIAVEWESNGQPENNYKTTP PVLDSDGSFFLYSKLTVDKSRWQQGNVFSCSVMHEALHNHYTQKS LSLSPGK |
| 21 | Fc region N83S | EPKSCDKTHTCPPCPAPELLGGPSVFLFPPKPKDTLMISRTPEVT CVVVDVSHEDPEVKFNWYVDGVEVHNAKTKPREEQYSSTYRVVSV LTVLHQDWLNGKEYKCKVSNKALPAPIEKTISKAKGQPREPQVYT LPPSRDELTKNQVSLTCLVKGFYPSDIAVEWESNGQPENNYKTTP PVLDSDGSFFLYSKLTVDKSRWQQGNVFSCSVMHEALHNHYTQKS LSLSPGK |
| 22 | IgG1 Fc domain with SCC, P238S, and P331S | EPKSSDKTHTCPPCPAPELLGGSSVFLFPPKPKDTLMISRTPEVT CVVVDVSHEDPEVKFNWYVDGVEVHNAKTKPREEQYNSTYRVVSV LTVLHQDWLNGKEYKCKVSNKALPASIEKTISKAKGQPREPQVYT LPPSRDELTKNQVSLTCLVKGFYPSDIAVEWESNGQPENNYKTTP PVLDSDGSFFLYSKLTVDKSRWQQGNVFSCSVMHEALHNHYTQKS LSLSPGK |
| 23 | Fc region with P238S, P331S mutations | EPKSCDKTHTCPPCPAPELLGGSSVFLFPPKPKDTIMISRTPEVT CVVVDVSHEDPEVKFNWYVDGVEVHNAKTKPREEQYNSTYRVVSV LTVLHQDWLNGKEYKCKVSNKALPASIEKTISKAKGQPREPQVYT LPPSRDELTKNQVSLTCLVKGFYPSDIAVEWESNGQPENNYKTTP PVLDSDGSFFLYSKLTVDKSRWQQGNVFSCSVMHEALHNHYTQKS LSLSPGK |
| 24 | IgG1 Fc (SCC hinge) | EPKSSDKTHTCPPCPAPELLGGPSVFLFPPKPKDTLMISRTPEVT CVVVDVSHEDPEVKFNWYVDGVEVHNAKTKPREEQYNSTYRVVSV LTVLHQDWLNGKEYKCKVSNKALPAPIEKTISKAKGQPREPQVYT LPPSRDELTKNQVSLTCLVKGFYPSDIAVEWESNGQPENNYKTTP PVLDSDGSFFLYSKLTVDKSRWQQGNVFSCSVMHEALHNHYTQKS LSLSPGK |
| 25 | IgG1 Fc domain (SSS hinge) | EPKSSDKTHTSPPSPAPELLGGPSVFLFPPKPKDTLMISRTPEVT CVVVDVSHEDPEVKFNWYVDGVEVHNAKTKPREEQYNSTYRVVSV LTVLHQDWLNGKEYKCKVSNKALPAPIEKTISKAKGQPREPQVYT LPPSRDELTKNQVSLTCLVKGFYPSDIAVEWESNGQPENNYKTTP PVLDSDGSFFLYSKLTVDKSRWQQGNVFSCSVMHEALHNHYTQKS LSLSPGK |
| 26 | IgG1 Fc domain with P238S (SCC hinge) | EPKSSDKTHTCPPCPAPELLGGSSVFLFPPKPKDTLMISRTPEVT CVVVDVSHEDPEVKFNWYVDGVEVHNAKTKPREEQYNSTYRVVSV LTVLHQDWLNGKEYKCKVSNKALPAPIEKTISKAKGQPREPQVYT LPPSRDELTKNQVSLTCLVKGFYPSDIAVEWESNGQPENNYKTTP PVLDSDGSFFLYSKLTVDKSRWQQGNVFSCSVMHEALHNHYTQKS LSLSPGK |
| 27 | IgG1 Fc domain with P331S | EPKSSDKTHTCPPCPAPELLGGPSVFLFPPKPKDTLMISRTPEVT CVVVDVSHEDPEVKFNWYVDGVEVHNAKTKPREEQYNSTYRVVSV LTVLHQDWLNGKEYKCKVSNKALPASIEKTISKAKGQPREPQVYT LPPSRDELTKNQVSLTCLVKGFYPSDIAVEWESNGQPENNYKTTP PVLDSDGSFFLYSKLTVDKSRWQQGNVFSCSVMHEALHNHYTQKS LSLSPGK |
| 28 | IgG1 Fc domain with SSS, P238S, and P331S | EPKSSDKTHTSPPSPAPELLGGSSVFLFPPKPKDTLMISRTPEVT CVVVDVSHEDPEVKFNWYVDGVEVHNAKTKPREEQYNSTYRVVSV LTVLHQDWLNGKEYKCKVSNKALPASIEKTISKAKGQPREPQVYT LPPSRDELTKNQVSLTCLVKGFYPSDIAVEWESNGQPENNYKTTP PVLDSDGSFFLYSKLTVDKSRWQQGNVFSCSVMHEALHNHYTQKS LSLSPGK |
| 29 | Human IgG4 | ASTKGPSVFPLAPCSRSTSESTAALGCLVKDYFPEPVTVSWNSGA LTSGVHTFPAVLQSSGLYSLSSVVTVPSSSLGTKTYTCNVDHKPS NTKVDKRVESKYGPPCPSCPAPEFLGGPSVFLFPPKPKDTLMISR TPEVTCVVVDVSQEDPEVQFNWYVDGVEVHNAKTKPREEQFNSTY RVVSVLTVLHQDWLNGKEYKCKVSNKGLPSSIEKTISKAKGQPRE PQVYTLPPSQEEMTKNQVSLTCLVKGFYPSDIAVEWESNGQPENN YKTTPPVLDSDGSFFLYSRLTVDKSRWQEGNVFSCSVMHEALHNH YTQKSLSLSLGK |
| 30 | Human IgG4 Fc domain | PAPEFLGGPSVFLFPPKPKDTLMISRTPEVTCVVVDVSQEDPEVQ FNWYVDGVEVHNAKTKPREEQFNSTYRVVSVLTVLHQDWLNGKEY KCKVSNKGLPSSIEKTISKAKGQPREPQVYTLPPSQEEMTKNQVS LTCLVKGFYPSDIAVEWESNGQPENNYKTTPPVLDSDGSFFLYSR LTVDKSRWQEGNVFSCSVMHEALHNHYTQKSLSLSLGK |
| 31 | Human IgG4 Hinge + Fc domain | ESKYGPPCPSCPAPEFLGGPSVFLFPPKPKDTLMISRTPEVTCVV VDVSQEDPEVQFNWYVDGVEVHNAKTKPREEQFNSTYRVVSVLTV LHQDWLNGKEYKCKVSNKGLPSSIEKTISKAKGQPREPQVYTLPP SQEEMTKNQVSLTCLVKGFYPSDIAVEWESNGQPENNYKTTPPVL DSDGSFFLYSRLTVDKSRWQEGNVFSCSVMHEALHNHYTQKSLSL SLGK |
| 32 | (Gly4Ser) 4 | GGGGSGGGGSGGGGSGGGGS |
| 33 | (Gly4Ser)3 | GGGGSGGGGSGGGGS |
| 34 | (Gly4Ser) 5 | GGGGSGGGGSGGGGSGGGGSGGGGS |
| 35 | (Gly4Ser) 2 | GGGGSGGGGS |
| 36 | (Gly4Ser) 1 | GGGGS |
| 37 | NLG linker | VDGASSPVNVSSPSVQDI |
| 38 | Linker | LEA (EAAAK) ₄ALEA (EAAAK) ₄ |
| 39 | linker | LEA (EAAAK) ₄ALEA (EAAAK) ₄ALE |
| 40 | Linker | GGSG |
| 41 | Linker | GSAT |
| 42 | Leader Sequence | MDWTWRILFLVAAATGTHA |
| 43 | VK3LP leader sequence | METPAQLLFLLLLWLPDTTG |
| 44 | RSLV-124 hVK3LP-hRNase (WT) -hIgG1 WT | metpaqllfllllwlpdttgkesrakkfqrqhmdsdsspssssty cnqmmrrrnmtqgrckpvntfvheplvdvqnvcfqekvtckngqg ncyksnssmhitdcrltngsrypncayrtspkerhiivacegspy vpvhfdasvedstlepkssdkthtcppcpapellggpsvflfppk pkdtlmisrtpevtcvvvdvshedpevkfnwyvdgvevhnaktkp reeqynstyrvvsvltvlhqdwlngkeykckvsnkalpapiekti skakgqprepqvytlppsrdeltknqvsltclvkgfypsdiavew esngqpennykttppvldsdgsfflyskltvdksrwqqgnvfscs vmhealhnhytqkslslspgk |
| 45 | RSLV125: huVK3LP-wthRNase-SSS-mthIgG1 P238S P331S | metpaqllfllllwlpdttgkesrakkfqrqhmdsdsspssssty cnqmmrrrnmtqgrckpvntfvheplvdvqnvcfqekvtckngqg ncyksnssmhitdcrltngsrypncayrtspkerhiivacegspy vpvhfdasvedstlepkssdkthtsppspapellggssvflfppk pkdtlmisrtpevtcvvvdvshedpevkfnwyvdgvevhnaktkp reeqynstyrvvsvltvlhqdwlngkeykckvsnkalpasiekti skakgqprepqvytlppsrdeltknqvsltclvkgfypsdiavew esngqpennykttppvldsdgsfflyskltvdksrwqqgnvfscs vmhealhnhytqkslslspgk |
| 46 | RSLV125-2: huVK3LP-wthRNase-SCC-mthIgG1 P238S P331S | metpaqllfllllwlpdttgkesrakkfqrqhmdsdsspssssty cnqmmrrrnmtqgrckpvntfvheplvdvqnvcfqekvtckngqg ncyksnssmhitdcrltngsrypncayrtspkerhiivacegspy vpvhfdasvedstlepkssdkthtcppcpapellggssvflfppk pkdtlmisrtpevtcvvvdvshedpevkfnwyvdgvevhnaktkp reeqynstyrvvsvltvlhqdwlngkeykckvsnkalpasiekti skakgqprepqvytlppsrdeltknqvsltclvkgfypsdiavew esngqpennykttppvldsdgsfflyskltvdksrwqqgnvfscs vmhealhnhytqkslslspgk |
| 47 | RSLV126: huVK3LP-WThRNase-(g4s) 4-SSS-mthIgG1-P238S-P331S | metpaqllfllllwlpdttgkesrakkfqrqhmdsdsspssssty cnqmmrrrnmtqgrckpvntfvheplvdvqnvcfqekvtckngqg ncyksnssmhitdcrltngsrypncayrtspkerhiivacegspy vpvhfdasvedstggggsggggsggggsggggslepkssdkthts ppspapellggssvflfppkpkdtlmisrtpevtcvvvdvshedp evkfnwyvdgvevhnaktkpreeqynstyrvvsvltvlhqdwlng keykckvsnkalpasiektiskakgqprepqvytlppsrdeltkn qvsltclvkgfypsdiavewesngqpennykttppvldsdgsffl yskltvdksrwqqgnvfscsvmhealhnhytqkslslspgk |
| 48 | RSLV126-2: huVK3LP-WThRNase-(g4s) 4-SCC-mthIgG1-P238S-P331S | metpaqllfllllwlpdttgkesrakkfqrqhmdsdsspssssty cnqmmrrrnmtqgrckpvntfvheplvdvqnvcfqekvtckngqg ncyksnssmhitdcrltngsrypncayrtspkerhiivacegspy vpvhfdasvedstggggsggggsggggsggggslepkssdkthtc ppcpapellggssvflfppkpkdtlmisrtpevtcvvvdvshedp evkfnwyvdgvevhnaktkpreeqynstyrvvsvltvlhqdwlng keykckvsnkalpasiektiskakgqprepqvytlppsrdeltkn qvsltclvkgfypsdiavewesngqpennykttppvldsdgsffl yskltvdksrwqqgnvfscsvmhealhnhytqkslslspgk |
| 49 | RSLV132: huVK3LP-wthRNase-SCC-mthIgG1 P238S P331S | metpaqllfllllwlpdttgkesrakkfqrqhmdsdsspssssty cnqmmrrrnmtqgrckpvntfvheplvdvqnvcfqekvtckngqg ncyksnssmhitdcrltngsrypncayrtspkerhiivacegspy vpvhfdasvedstlepkssdkthtcppcpapellggssvflfppk pkdtlmisrtpevtcvvvdvshedpevkfnwyvdgvevhnaktkp reeqynstyrvvsvltvlhqdwlngkeykckvsnkalpasiekti skakgqprepqvytlppsrdeltknqvsltclvkgfypsdiavew esngqpennykttppvldsdgsfflyskltvdksrwqqgnvfscs vmhealhnhytqkslslspgk |
| 50 | RSLV132: wthRNase-SCC-mthIgG1 P238S P331S | kesrakkfqrqhmdsdsspsssstycnqmmrrrnmtqgrckpvnt fvheplvdvqnvcfqekvtckngqgncyksnssmhitdcrltngs rypncayrtspkerhiivacegspyvpvhfdasvedstlepkssd kthtcppcpapellggssvflfppkpkdtlmisrtpevtcvvvdv shedpevkfnwyvdgvevhnaktkpreeqynstyrvvsvltvlhq dwlngkeykckvsnkalpasiektiskakgqprepqvytlppsrd eltknqvsltclvkgfypsdiavewesngqpennykttppvldsd gsfflyskltvdksrwqqgnvfscsvmhealhnhytqkslslspg k |
| 51 | RSLV127: huVK3LP-hDNase1 105/114-(g4s)4-SSS-mthIgG1-P238S-P331S-NLG-RNase | metpaqllfllllwlpdttglkiaafniqtfgetkmsnatlvsyi vqilsrydialvqevrdshltavgklldnlnqdapdtyhyvvsep lgrnsykerylfvyrpdqvsavdsyyyddgcepcrndtfnrepfi vrffsrftevrefaivplhaapgdavaeidalydvyldvqekwgl edvmlmgdfnagcsyvrpsqwssirlwtsptfqwlipdsadttat pthcaydrivvagmllrgavvpdsalpfnfqaayglsdqlaqais dhypvevmlkggggsggggsggggsggggslepkssdkthtspps papellggssvflfppkpkdtlmisrtpevtcvvvdvshedpevk fnwyvdgvevhnaktkpreeqynstyrvvsvltvlhqdwlngkey kckvsnkalpasiektiskakgqprepqvytlppsrdeltknqvs ltclvkgfypsdiavewesngqpennykttppvldsdgsfflysk ltvdksrwqqgnvfscsvmhealhnhytqkslslspgkvdgassp vnvsspsvqdikesrakkfqrqhmdsdsspsssstycnqmmrrrn mtqgrckpvntfvheplvdvqnvcfqekvtckngqgncyksnssm hitdcrltngsrypncayrtspkerhiivacegspyvpvhfdasv edst |
| 52 | RSLV127-2: huVK3LP-hDNase1 105/114-(g4s) 4-SCC-mthIgG1-P238S- | metpaqllfllllwlpdttglkiaafniqtfgetkmsnatlvsyi vqilsrydialvqevrdshltavgklldnlnqdapdtyhyvvsep lgrnsykerylfvyrpdqvsavdsyyyddgcepcrndtfnrepfi vrffsrftevrefaivplhaapgdavaeidalydvyldvqekwgl edvmlmgdfnagcsyvrpsqwssirlwtsptfqwlipdsadttat pthcaydrivvagmllrgavvpdsalpfnfqaayglsdqlaqais dhypvevmlkggggsggggsggggsggggslepkssdkthtcppc papellggssvflfppkpkdtlmisrtpevtcvvvdvshedpevk |
| | P331S-NLG-RNase | fnwyvdgvevhnaktkpreeqynstyrvvsvltvlhqdwlngkey kckvsnkalpasiektiskakgqprepqvytlppsrdeltknqvs ltclvkgfypsdiavewesngqpennykttppvldsdgsfflysk ltvdksrwqqgnvfscsvmhealhnhytqkslslspgkvdgassp vnvsspsvqdikesrakkfqrqhmdsdsspsssstycnqmmrrrn mtqgrckpvntfvheplvdvqnvcfqekvtckngqgncyksnssm hitdcrltngsrypncayrtspkerhiivacegspyvpvhfdasv edst |
| 53 | RSLV128: huVK3LP-hRNase WT-(g4s)4-SSS-mthIgG1-P238S-P331S-NLG-hDNase 105/114 | metpaqllfllllwlpdttgkesrakkfqrqhmdsdsspssssty cnqmmrrrnmtqgrckpvntfvheplvdvqnvcfqekvtckngqg ncyksnssmhitdcrltngsrypncayrtspkerhiivacegspy vpvhfdasvedstggggsggggsggggsggggslepkssdkthts ppspapellggssvflfppkpkdtlmisrtpevtcvvvdvshedp evkfnwyvdgvevhnaktkpreeqynstyrvvsvltvlhqdwlng keykckvsnkalpasiektiskakgqprepqvytlppsrdeltkn qvsltclvkgfypsdiavewesngqpennykttppvldsdgsffl yskltvdksrwqqgnvfscsvmhealhnhytqkslslspgkvdga sspvnvsspsvqdilkiaafniqtfgetkmsnatlvsyivqilsr ydialvqevrdshltavgklldnlnqdapdtyhyvvseplgrnsy kerylfvyrpdqvsavdsyyyddgcepcrndtfnrepfivrffsr ftevrefaivplhaapgdavaeidalydvyldvqekwgledvmlm gdfnagcsyvrpsqwssirlwtsptfqwlipdsadttatpthcay drivvagmllrgavvpdsalpfnfqaayglsdqlaqaisdhypve vmlk |
| 54 | RSLV128-2: huVK3LP-hRNase WT-(g4s) 4-SCC-mthIgG1-P238S-P331S-NLG-hDNase 105/114 | metpaqllfllllwlpdttgkesrakkfqrqhmdsdsspssssty cnqmmrrrnmtqgrckpvntfvheplvdvqnvcfqekvtckngqg ncyksnssmhitdcrltngsrypncayrtspkerhiivacegspy vpvhfdasvedstggggsggggsggggsggggslepkssdkthtc ppcpapellggssvflfppkpkdtlmisrtpevtcvvvdvshedp evkfnwyvdgvevhnaktkpreeqynstyrvvsvltvlhqdwlng keykckvsnkalpasiektiskakgqprepqvytlppsrdeltkn qvsltclvkgfypsdiavewesngqpennykttppvldsdgsffl yskltvdksrwqqgnvfscsvmhealhnhytqkslslspgkvdga sspvnvsspsvqdilkiaafniqtfgetkmsnatlvsyivqilsr ydialvqevrdshltavgklldnlnqdapdtyhyvvseplgrnsy kerylfvyrpdqvsavdsyyyddgcepcrndtfnrepfivrffsr ftevrefaivplhaapgdavaeidalydvyldvqekwgledvmlm gdfnagcsyvrpsqwssirlwtsptfqwlipdsadttatpthcay drivvagmllrgavvpdsalpfnfqaayglsdqlaqaisdhypve vmlk |
| 55 | RSLV129: huVK3LP-hRNAseWT-SSS-mthIgG1-P238S-P331S-NLG-hDNAse 105/114 | metpaqllfllllwlpdttgkesrakkfqrqhmdsdsspssssty cnqmmrrrnmtqgrckpvntfvheplvdvqnvcfqekvtckngqg ncyksnssmhitdcrltngsrypncayrtspkerhiivacegspy vpvhfdasvedstlepkssdkthtsppspapellggssvflfppk pkdtlmisrtpevtcvvvdvshedpevkfnwyvdgvevhnaktkp reeqynstyrvvsvltvlhqdwlngkeykckvsnkalpasiekti skakgqprepqvytlppsrdeltknqvsltclvkgfypsdiavew esngqpennykttppvldsdgsfflyskltvdksrwqqgnvfscs vmhealhnhytqkslslspgkvdgasspvnvsspsvqdilkiaaf niqtfgetkmsnatlvsyivqilsrydialvqevrdshltavgkl ldnlnqdapdtyhyvvseplgrnsykerylfvyrpdqvsavdsyy yddgcepcrndtfnrepfivrffsrftevrefaivplhaapgdav aeidalydvyldvqekwgledvmlmgdfnagcsyvrpsqwssirl wtsptfqwlipdsadttatpthcaydrivvagmllrgavvpdsal pfnfqaayglsdqlaqaisdhypvevmlk |
| 56 | RSLV129-2: huVK3LP-hRNAseWT-SCC-mthIgG1-P238S-P331S-NLG-hDNAse 105/114 | metpaqllfllllwlpdttgkesrakkfqrqhmdsdsspssssty cnqmmrrrnmtqgrckpvntfvheplvdvqnvcfqekvtckngqg ncyksnssmhitdcrltngsrypncayrtspkerhiivacegspy vpvhfdasvedstlepkssdkthtcppcpapellggssvflfppk pkdtlmisrtpevtcvvvdvshedpevkfnwyvdgvevhnaktkp reeqynstyrvvsvltvlhqdwlngkeykckvsnkalpasiekti skakgqprepqvytlppsrdeltknqvsltclvkgfypsdiavew esngqpennykttppvldsdgsfflyskltvdksrwqqgnvfscs vmhealhnhytqkslslspgkvdgasspvnvsspsvqdilkiaaf niqtfgetkmsnatlvsyivqilsrydialvqevrdshltavgkl ldnlnqdapdtyhyvvseplgrnsykerylfvyrpdqvsavdsyy yddgcepcrndtfnrepfivrffsrftevrefaivplhaapgdav aeidalydvyldvqekwgledvmlmgdfnagcsyvrpsqwssirl wtsptfqwlipdsadttatpthcaydrivvagmllrgavvpdsal pfnfqaayglsdqlaqaisdhypvevmlk |
| 57 | RSLV133: huVK3LP-hRNAseWT-SCC-mthIgG1-P238S-P331S-NLG-hDNAse 105/114 | metpaqllfllllwlpdttgkesrakkfqrqhmdsdsspssssty cnqmmrrrnmtqgrckpvntfvheplvdvqnvcfqekvtckngqg ncyksnssmhitdcrltngsrypncayrtspkerhiivacegspy vpvhfdasvedstlepkssdkthtcppcpapellggssvflfppk pkdtlmisrtpevtcvvvdvshedpevkfnwyvdgvevhnaktkp reeqynstyrvvsvltvlhqdwlngkeykckvsnkalpasiekti skakgqprepqvytlppsrdeltknqvsltclvkgfypsdiavew esngqpennykttppvldsdgsfflyskltvdksrwqqgnvfscs vmhealhnhytqkslslspgkvdgasspvnvsspsvqdilkiaaf niqtfgetkmsnatlvsyivqilsrydialvqevrdshltavgkl ldnlnqdapdtyhyvvseplgrnsykerylfvyrpdqvsavdsyy yddgcepcrndtfnrepfivrffsrftevrefaivplhaapgdav aeidalydvyldvqekwgledvmlmgdfnagcsyvrpsqwssirl wtsptfqwlipdsadttatpthcaydrivvagmllrgavvpdsal pfnfqaayglsdqlaqaisdhypvevmlk |
| 58 | RSLV133: hRNAseWT-SCC-mthIgG1-P238S-P331S-NLG-hDNAse 105/114 | kesrakkfqrqhmdsdsspsssstycnqmmrrrnmtqgrckpvnt fvheplvdvqnvcfqekvtckngqgncyksnssmhitdcrltngs rypncayrtspkerhiivacegspyvpvhfdasvedstlepkssd kthtcppcpapellggssvflfppkpkdtlmisrtpevtcvvvdv shedpevkfnwyvdgvevhnaktkpreeqynstyrvvsvltvlhq dwlngkeykckvsnkalpasiektiskakgqprepqvytlppsrd eltknqvsltclvkgfypsdiavewesngqpennykttppvldsd gsfflyskltvdksrwqqgnvfscsvmhealhnhytqkslslspg kvdgasspvnvsspsvqdilkiaafniqtfgetkmsnatlvsyiv qilsrydialvqevrdshltavgklldnlnqdapdtyhyvvsepl grnsykerylfvyrpdqvsavdsyyyddgcepcrndtfnrepfiv rffsrftevrefaivplhaapgdavaeidalydvyldvqekwgle dvmlmgdfnagcsyvrpsqwssirlwtsptfqwlipdsadttatp thcaydrivvagmllrgavvpdsalpfnfqaayglsdqlaqaisd hypvevmlk |
| 59 | O-linked glycosylati | CXXGG-T/S-C |
| | on consensus | |
| 60 | O-linked glycosylati on consensus | NST-E/D-A |
| 61 | O-linked glycosylati on consensus | NITQS |
| 62 | O-linked glycosylati on consensus | QSTQS |
| 63 | O-linked glycosylati on consensus | D/EFT-R/K-V |
| 64 | O-linked glycosylati on consensus | C-E/D-SN |
| 65 | O-linked glycosylati on consensus | GGSC-K/R |

We claim:

1. A method of treating Sjogren’s syndrome in a patient in need thereof, the method comprising administering an effective amount of an RNA nuclease agent to the patient, wherein treatment results in an increase in one or more interferon-inducible genes, wherein the one or more interferon-inducible genes is selected from the group consisting of LY6E, RTP4, EPSTI1, OAS1, OAS2, OASL, BATF2, SERPING1, OTOF, IFITM3, ISG15, HES4, XAF1, OAS3, IFIT3, RSAD2, LAMP3, IFIT1, IFI44L, HERC5, MX1, and IFI44.

2. The method of claim 1, wherein the interferon-inducible gene is OAS1.

3. The method of claim 1, wherein the interferon-inducible gene is ISG15.

4. The method of claim 1, wherein the interferon-inducible gene is HES4.

5. The method of claim 1, wherein the interferon-inducible gene is IFIT1.

6. The method of claim 1, wherein the interferon-inducible gene is IFI44L.

7. The method of claim 1, wherein the interferon-inducible gene is IFI44.

8. The method of any one of claims 1-7, wherein treatment results in an improvement in fatigue.

9. The method of any one of claims 1-8, wherein the RNA nuclease agent is an RNase-Fc.

10. The method of any one of claims 1-8, wherein the RNA nuclease agent is RSLV-132.

11. A method of treating Sjogren’s syndrome in a patient in need thereof, the method comprising administering an effective amount of an RNA nuclease agent to the patient, wherein treatment results in an increase in one or more interferon-inducible genes, wherein the one or more interferon-inducible genes is selected from the group consisting of HERC6, ATF3, GBP3. GBP4, IFITM1, MT2A, TNFAIP6, LAP3, OAS2, PLSCR1, TNFSF10, TIMM10, UBE2L6, IFI35, AIM2, GALM, EIF2AK2, ZBP1, APOL6, STAT1, GBP5, LGALS3BP, SEPT4, FBXO6, SCO2, MOV10, CEACAM1, PML, WARS, CCL8, SOCS1, ZNF684, SAMD9L, PARP10, GBP1, IRF7, IFIT5, IFIH1, DHX58, IFIT2, PARP12, TRIM22, DDX58, STAT2, PARP9, and PARP14.

12. The method of claim 11, wherein the interferon-inducible gene is HERC6.

13. The method of claim 11, wherein the interferon-inducible gene is ATF3.

14. The method of claim 11, wherein the interferon-inducible gene is GBP3.

15. The method of claim 11, wherein the interferon-inducible gene is TNFSF10.

16. The method of claim 11, wherein the interferon-inducible gene is TIMMIO.

17. The method of claim 11, wherein the interferon-inducible gene is CCL8.

18. The method of claim 11, wherein the interferon-inducible gene is SOCS1.

19. The method of claim 11, wherein the interferon-inducible gene is SAMD9L.

20. The method of claim 11, wherein the interferon-inducible gene is DHX58.

21. The method of any one of claims 11-20, wherein treatment results in an improvement in fatigue.

22. The method of any one of claims 11-21, wherein the RNA nuclease agent is an RNase-Fc.

23. The method of any one of claims 11-21, wherein the RNA nuclease agent is RSLV-132.

24. A method of treating Sjogren’s syndrome in a patient in need thereof, the method comprising administering an effective amount of an RNA nuclease agent to the patient, wherein treatment results in an increase in one or more interferon-inducible genes, wherein the one or more interferon-inducible genes is selected from the group consisting of MDK, CHMP5, SAMD9, C1QA, TCN2, ACTA2, NCOA7, PSMB9, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, SP140, CASP1, TRIM5, NMI, DYNLT1, TRAFD1, TDRD7, BTN3A1, ZC3HAV1, DHRS9, GBP2, TRIM56, HSH2D, and UNC93B1.

25. The method of claim 24, wherein the interferon-inducible gene is MDK.

26. The method of claim 24, wherein the interferon-inducible gene is CHMP5.

27. The method of claim 24, wherein the interferon-inducible gene is SAMD9.

28. The method of claim 24, wherein the interferon inducible gene is BST2.

29. The method of claim 24, wherein the interferon-inducible gene is ISG20.

30. The method of claim 24, wherein the interferon-inducible gene is DRAP1.

31. The method of claim 24, wherein the interferon-inducible gene is PHF11.

32. The method of any one of claims 24-31, wherein treatment results in an improvement in fatigue.

33. The method of any one of claims 24-32, wherein the RNA nuclease agent is an RNase-Fc.

34. The method of any one of claims 24-32, wherein the RNA nuclease agent is RSLV-132.

35. Use of an RNA nuclease agent in the manufacture of a medicament for the treatment of Sjogren’s syndrome in a patient, the use comprising administering an effective amount of an RNA nuclease agent to the patient, wherein treatment results in an increase in one or more interferon-inducible genes, wherein the one or more interferon-inducible genes is selected from the group consisting of LY6E, RTP4, EPSTI1, OAS1, OAS2, OASL, BATF2, SERPING1, OTOF, IFITM3, CXCL10, ISG15, HES4, XAF1, OAS3, IFIT3, RSAD2, LAMP3, IFIT1, IFI44L, HERC5, MX1, and IFI44.

36. Use of an RNA nuclease agent in the manufacture of a medicament for the treatment of Sjogren’s syndrome in a patient, the use comprising administering an effective amount of an RNA nuclease agent to the patient, wherein treatment results in an increase in one or more interferon-inducible genes, wherein the one or more interferon-inducible genes is selected from the group consisting of HERC6, ATF3, GBP3. GBP4. IFITM1, MT2A, TNFAIP6, LAP3, OAS2, PLSCR1, TNFSF10, TIMM10, UBE2L6, IFI35, AIM2, GALM, EIF2AK2, ZBP1, APOL6, STAT1, GBP5, LGALS3BP, SEPT4, FBXO6, SCO2, MOV10, CEACAM1, PML, WARS, CCL8, SOCS1, ZNF684, SAMD9L, PARP10, GBP1, IRF7, IFIT5, IFIH1, DHX58, IFIT2, PARP12, TRIM22, DDX58, STAT2, PARP9, and PARP14.

37. Use of an RNA nuclease agent in the manufacture of a medicament for the treatment of Sjogren’s syndrome in a patient, the use comprising administering an effective amount of an RNA nuclease agent to the patient, wherein treatment results in an increase in one or more interferon-inducible genes, wherein the one or more interferon-inducible genes is selected from the group consisting of MDK, CHMP5, SAMD9, C1QA, TCN2, ACTA2, NCOA7, PSMB9, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, SP140, CASP1, TRIM5, NMI, DYNLT1, TRAFD1, TDRD7, BTN3A1, ZC3HAV1, DHRS9, GBP2, TRIM56, HSH2D, and UNC93B1.

38. The use of any one of claims 35-37, wherein treatment results in an improvement in fatigue.

39. The use of any one of claims 35-38, wherein the RNA nuclease agent is an RNase-Fc.

40. The use of any one of claims 35-38, wherein the RNA nuclease agent is RSLV-132.

41. An RNA nuclease agent for use in a method of treating Sjogren’s syndrome in a patient, the use comprising administering an effective amount of an RNA nuclease agent to the patient, wherein treatment results in an increase in one or more interferon-inducible genes, wherein the one or more interferon-inducible genes is selected from the group consisting of LY6E, RTP4, EPSTI1, OAS1, OAS2, OASL, BATF2, SERPING1, OTOF, IFITM3, CXCL10, ISG15, HES4, XAF1, OAS3, IFIT3, RSAD2, LAMP3, IFIT1, IFI44L, HERC5, MX1, and IFI44.

42. An RNA nuclease agent for use in a method of treating Sjogren’s syndrome in a patient, the use comprising administering an effective amount of an RNA nuclease agent to the patient, wherein treatment results in an increase in one or more interferon-inducible genes, wherein the one or more interferon-inducible genes is selected from the group consisting of HERC6, ATF3, GBP3. GBP4. IFITM1, MT2A, TNFAIP6, LAP3, OAS2, PLSCR1, TNFSF10, TIMM10, UBE2L6, IFI35, AIM2, GALM, EIF2AK2, ZBP1, APOL6, STAT1, GBP5, LGALS3BP, SEPT4, FBXO6, SCO2, MOV10, CEACAM1, PML, WARS, CCL8, SOCS1, ZNF684, SAMD9L, PARP10, GBP1, IRF7, IFIT5, IFIH1, DHX58, IFIT2, PARP12, TRIM22, DDX58, STAT2, PARP9, and PARP14.

43. An RNA nuclease agent for use in a method of treating Sjogren’s syndrome in a patient, the use comprising administering an effective amount of an RNA nuclease agent to the patient, wherein treatment results in an increase in one or more interferon-inducible genes, wherein the one or more interferon-inducible genes is selected from the group consisting of MDK, CHMP5, SAMD9, C1QA, TCN2, ACTA2, NCOA7, PSMB9, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, SP140, CASP1, TRIM5, NMI, DYNLT1, TRAFD1, TDRD7, BTN3A1, ZC3HAV1, DHRS9, GBP2, TRIM56, HSH2D, and UNC93B1.

44. An RNA nuclease agent for use in a method of treating Sjogren’s syndrome in a patient, the use comprising administering an effective amount of an RNA nuclease agent to the patient, wherein treatment results in an increase in one or more interferon-inducible genes, wherein the one or more interferon-inducible genes is selected from the group consisting of MDK, CHMP5, SAMD9, C1QA, TCN2, ACTA2, NCOA7, PSMB9, GADD45B, BST2, ISG20, DRAP1, PHF11, TRIM38, ETV7, RBCK1, SP100, ABCA1, IL-18, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, SP140, CASP1, ISG15, TRIM5, NMI, DYNLT1, TRAFD1, TDRD7, BTN3A1, ZC3HAV1, DHRS9, GBP2, CXCL10, TRIM56, HSH2D, and UNC93B1.

45. The RNA nuclease agent of any one of claims 41-44, wherein treatment results in an improvement in fatigue.

46. A method of identifying a patient having Sjogren’s syndrome as a candidate for treatment with an RNA nuclease agent, comprising
(a) determining an interferon-inducible gene expression profile in a sample obtained from the patient; and
(b) comparing the interferon-inducible gene expression profile determined in step (a) with an interferon-inducible gene expression profile in a sample obtained from a suitable control subject,
wherein the interferon-inducible genes include one or more of the following interferon-inducible genes: MDK, CHMP5, SAMD9, C1QA, TCN2, ACTA2, NCOA7, PSMB9, GADD45B, BST2, ISG20, DRAP1, PHF11, TRIM38, ETV7, RBCK1, SP100, ABCA1, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, SP140, CASP1, TRIM5, NMI, DYNLT1, TRAFD1, TDRD7, BTN3A1, ZC3HAV1, DHRS9, GBP2, TRIM56, HSH2D, and UNC93B1; and
wherein the interferon-inducible gene expression profile indicates the patient is a candidate for treatment with an RNA nuclease agent.

47. A method of identifying a patient having Sjogren’s syndrome as a candidate for treatment with an RNA nuclease agent, comprising
(a) determining an interferon-inducible gene expression profile in a sample obtained from the patient; and
(b) comparing the interferon-inducible gene expression profile determined in step (a) with an interferon-inducible gene expression profile in a sample obtained from a suitable control subject,
wherein the interferon-inducible genes include one or more of the following interferon-inducible genes: MDK, CHMP5, SAMD9, C1QA, TCN2, ACTA2, NCOA7, PSMB9, GADD45B, BST2, ISG20, DRAP1, PHF11, TRIM38, ETV7, RBCK1, SP100, ABCA1, IL-18, RHBDF2, CPT1B, TAP1, NTNG2, TAP2, REC8, LHFPL2, TRIM25, ADAR, ZNFX1, SP110, TMEM140, NBN, LGALS9, IFI16, TRIM21, SRBD1, PARP10, SP140, CASP1, ISG15, TRIM5, NMI, DYNLT1, TRAFD1, TDRD7, BTN3A1, ZC3HAV1, DHRS9, GBP2, CXCL10, TRIM56, HSH2D, and UNC93B 1; and
wherein the interferon-inducible gene expression profile indicates the patient is a candidate for treatment with an RNA nuclease agent.

48. The RNA nuclease agent of any one of claims 41-47, wherein the RNA nuclease agent is an RNase-Fc.

49. The RNA nuclease agent of any one of claims 41-47, wherein the RNA nuclease agent is RSLV-132.

50. A method of treating Sjogren’s syndrome in a patient in need thereof, the method comprising administering an effective amount of an RNA nuclease agent to the patient, wherein treatment results in an increase in one or more interferon-inducible genes, wherein the one or more interferon-inducible genes is selected from one or more module M1.2 interferon inducible genes.

51. A method of treating Sjogren’s syndrome in a patient in need thereof, the method comprising administering an effective amount of an RNA nuclease agent to the patient, wherein treatment results in an increase in one or more interferon-inducible genes, wherein the one or more interferon-inducible genes is selected from one or more module M3.4 interferon inducible genes.

52. A method of treating Sjogren’s syndrome in a patient in need thereof, the method comprising administering an effective amount of an RNA nuclease agent to the patient, wherein treatment results in an increase in one or more interferon-inducible genes, wherein the one or more interferon-inducible genes is selected from one or more module M5.12 interferon inducible genes.

53. The method of any one of claims 50-52, wherein treatment results in an improvement in fatigue.

54. The method of any one of claims 50-53, wherein the RNA nuclease agent is an RNase-Fc.

55. The method of any one of claims 50-53, wherein the RNA nuclease agent is RSLV-132.

56. Use of an RNA nuclease agent in the manufacture of a medicament for the treatment of Sjogren’s syndrome in a patient, the use comprising administering an effective amount of an RNA nuclease agent to the patient, wherein treatment results in an increase in one or more interferon-inducible genes, wherein the one or more interferon-inducible genes is selected from one or more module M1.2 interferon inducible genes.

57. Use of an RNA nuclease agent in the manufacture of a medicament for the treatment of Sjogren’s syndrome in a patient, the use comprising administering an effective amount of an RNA nuclease agent to the patient, wherein treatment results in an increase in one or more interferon-inducible genes, wherein the one or more interferon-inducible genes is selected from one or more module M3.4 interferon inducible genes.

58. Use of an RNA nuclease agent in the manufacture of a medicament for the treatment of Sjogren’s syndrome in a patient, the use comprising administering an effective amount of an RNA nuclease agent to the patient, wherein treatment results in an increase in one or more interferon-inducible genes, wherein the one or more interferon-inducible genes is selected from one or more module M5.12 interferon inducible genes.

59. The use of any one of claims 56-58, wherein treatment results in an improvement in fatigue.

60. The use of any one of claims 56-59, wherein the RNA nuclease agent is an RNase-Fc.

61. The use of any one of claims 56-59, wherein the RNA nuclease agent is RSLV-132.

62. An RNA nuclease agent for use in a method of treating Sjogren’s syndrome in a patient, the use comprising administering an effective amount of an RNA nuclease agent to the patient, wherein treatment results in an increase in one or more interferon-inducible genes, wherein the one or more interferon-inducible genes is selected from one or more module M1.2 interferon inducible genes.

63. An RNA nuclease agent for use in a method of treating Sjogren’s syndrome in a patient, the use comprising administering an effective amount of an RNA nuclease agent to the patient, wherein treatment results in an increase in one or more interferon-inducible genes, wherein the one or more interferon-inducible genes is selected from one or more module M3.4 interferon inducible genes.

64. An RNA nuclease agent for use in a method of treating Sjogren’s syndrome in a patient, the use comprising administering an effective amount of an RNA nuclease agent to the patient, wherein treatment results in an increase in one or more interferon-inducible genes, wherein the one or more interferon-inducible genes is selected from one or more module M5.12 interferon inducible genes.

65. An RNA nuclease agent of any one of claims 62-64, wherein treatment results in an improvement in fatigue.

66. An RNA nuclease agent of any one of claims 62-65, wherein the RNA nuclease agent is an RNase-Fc.

67. An RNA nuclease agent of any one of claims 62-65, wherein the RNA nuclease agent is RSLV-132.

68. A method of treating Sjogren’s syndrome in a patient in need thereof, the method comprising administering an effective amount of an RNA nuclease agent to the patient, wherein treatment results in a decrease in one or more long non-coding RNA (lncRNA) molecules.

69. The method of claim 68, wherein the one or more lncRNA molecules is one or more lncRNA molecules that mediates the expression of one or more pro-inflammatory genes.

70. The method of claim 69, wherein the one or more pro-inflammatory genes is a pro-inflammatory gene related to autoimmune disease and/or immune system function.

71. The method of claim 68, wherein the one or more lncRNA molecules is selected from the group consisting of PXN-AS1, AP002852.1, AL009176.1, MKX-AS1, AC109464.1, AL590560.2, AC020908.3, AL731533.3, AL353743.2, AC007405.1, DPRXP2, ST20-AS1, AC020916.1, AC027601.1, AL358334.2, AL358394.2, AL022238.2, AC116366.2, AP003031.1, CYP4F30P, AL390071.1, AC008532.1, CPHL1P, AC008870.4, AC008763.1, AL355388.2, AC110926.2, AL133415.1, AL591503.1, DLGAP1-AS1, CHL1-AS2, AC010326.4, AC051649.1, HCG27, AL034417.2, AP001160.1, AC009435.1, LINC01499, AL161621.1, AP001469.1, AC021491.2, AC010168.1, SLC44A3-AS1, AC011445.2, ZMIZ1-AS1, AC027018.1, AL591379.1, AC061992.1, AL513348.1, AC092617.1, AC015853.3, AC138655.1, AC108134.3, LINC01515, AC243829.2, AC020911.1, AC016168.4, AL590705.1, FAM3D-AS1, AC078881.1, AC004466.2, AC018866.1, AC010531.3, AJ011931.2, TUSC8, PTOV1-AS1, RPS4XP2, AC021148.2, AC009041.2, AC093525.6, LINC01516, AL670729.3, C18ORF65, AC098818.2, FAM230C, LINC01988, AC068831.1, CYP51A1-AS1, LINC01227, LINC00895, LINC01519, AC114730.3, MIPEPP3, ITPRIP-AS1, AC138207.5, LINC01091, AC012645.3, AC082651.1, DLGAP1-AS3, AC080011.1, AC010148.1, AC078962.1, AL132822.1, LINC02552, AC098484.2, AC090587.1, LINC00173, PIK3CD-AS1, AL021393.1, AL450322.2, AL591368.1, AC010894.4, AC067930.8, AC024267.3, AC104984.1, AC109495.1, AC129926.1, KLF3-AS1, AL512306.3, LINC01448, AC080013.6, and DACT3-AS1.

72. The method of claim 68, wherein the one or more lncRNA molecules is selected from the group consisting of PXN-AS1, AC116366.2, HCG27, AL034417.2, ZMIZ1-AS1, AL513348.1, AC243829.2, LINC01227, LINC01091, AC080011.1, AL591368.1, AC109495.1, KLF3-AS1, and LINC01448.

73. The method of any one of claims 68-72, wherein treatment results in an improvement in fatigue.

74. The method of any one of claims 68-73, wherein the RNA nuclease agent is an RNase-Fc.

75. The method of any one of claims 68-73, wherein the RNA nuclease agent is RSLV-132.

76. Use of an RNA nuclease agent in the manufacture of a medicament for the treatment of Sjogren’s syndrome in a patient, the use comprising administering an effective amount of an RNA nuclease agent to the patient, wherein treatment results in a decrease in one or more lncRNA molecules.

77. The use of claim 76, wherein the one or more lncRNA molecules is an lncRNA molecule that regulates the transcription of a gene involved in inflammation.

78. The use of claim 76, wherein the one or more lncRNA molecules is an lncRNA molecule that regulates the transcription of a pro-inflammatory gene.

79. The use of claim 76, wherein the one or more lncRNA molecules is selected from the group consisting of PXN-AS1, AP002852.1, AL009176.1, MKX-AS1, AC109464.1, AL590560.2, AC020908.3, AL731533.3, AL353743.2, AC007405.1, DPRXP2, ST20-AS1, AC020916.1, AC027601.1, AL358334.2, AL358394.2, AL022238.2, AC116366.2, AP003031.1, CYP4F30P, AL390071.1, AC008532.1, CPHL1P, AC008870.4, AC008763.1, AL355388.2, AC110926.2, AL133415.1, AL591503.1, DLGAP1-AS1, CHL1-AS2, AC010326.4, AC051649.1, HCG27, AL034417.2, AP001160.1, AC009435.1, LINC01499, AL161621.1, AP001469.1, AC021491.2, AC010168.1, SLC44A3-AS1, AC011445.2, ZMIZ1-AS1, AC027018.1, AL591379.1, AC061992.1, AL513348.1, AC092617.1, AC015853.3, AC138655.1, AC108134.3, LINC01515, AC243829.2, AC020911.1, AC016168.4, AL590705.1, FAM3D-AS1, AC078881.1, AC004466.2, AC018866.1, AC010531.3, AJ011931.2, TUSC8, PTOV1-AS1, RPS4XP2, AC021148.2, AC009041.2, AC093525.6, LINC01516, AL670729.3, C18ORF65, AC098818.2, FAM230C, LINC01988, AC068831.1, CYP51A1-AS1, LINC01227, LINC00895, LINC01519, AC114730.3, MIPEPP3, ITPRIP-AS1, AC138207.5, LINC01091, AC012645.3, AC082651.1, DLGAP1-AS3, AC080011.1, AC010148.1, AC078962.1, AL132822.1, LINC02552, AC098484.2, AC090587.1, LINC00173, PIK3CD-AS1, AL021393.1, AL450322.2, AL591368.1, AC010894.4, AC067930.8, AC024267.3, AC104984.1, AC109495.1, AC129926.1, KLF3-AS1, AL512306.3, LINC01448, AC080013.6, and DACT3-AS1.

80. The use of claim 76, wherein the one or more lncRNA molecules is selected from the group consisting of PXN-AS1, AC116366.2, HCG27, AL034417.2, ZMIZ1-AS1, AL513348.1, AC243829.2, LINC01227, LINC01091, AC080011.1, AL591368.1, AC109495.1, KLF3-AS1, and LINC01448.

81. The use of any one of claims 76-80, wherein treatment results in an improvement in fatigue.

82. The use of any one of claims 76-81, wherein the RNA nuclease agent is an RNase-Fc.

83. The use of any one of claims 76-81, wherein the RNA nuclease agent is RSLV-132.

84. An RNA nuclease agent for use in a method of treating Sjogren’s syndrome in a patient, the use comprising administering an effective amount of an RNA nuclease agent to the patient, wherein treatment results in a decrease in one or more lncRNA molecules.

85. An RNA nuclease agent of claim 84, wherein the one or more lncRNA molecules is an lncRNA molecule that regulates the transcription of a gene involved in inflammation.

86. An RNA nuclease agent of claim 84, wherein the one or more lncRNA molecules is an lncRNA molecule that regulates the transcription of a pro-inflammatory gene.

87. An RNA nuclease agent of claim 84, wherein the one or more lncRNA molecules is selected from the group consisting of PXN-AS1, AP002852.1, AL009176.1, NMX-AS1, AC109464.1, AL590560.2, AC020908.3, AL731533.3, AL353743.2, AC007405.1, DPRXP2, ST20-AS1, AC020916.1, AC027601.1, AL358334.2, AL358394.2, AL022238.2, AC116366.2, AP003031.1, CYP4F30P, AL390071.1, AC008532.1, CPHL1P, AC008870.4, AC008763.1, AL355388.2, AC110926.2, AL133415.1, AL591503.1, DLGAP1-AS1, CHL1-AS2, AC010326.4, AC051649.1, HCG27, AL034417.2, AP001160.1, AC009435.1, LINC01499, AL161621.1, AP001469.1, AC021491.2, AC010168.1, SLC44A3-AS1, AC011445.2, ZMIZ1-AS1, AC027018.1, AL591379.1, AC061992.1, AL513348.1, AC092617.1, AC015853.3, AC138655.1, AC108134.3, LINC01515, AC243829.2, AC020911.1, AC016168.4, AL590705.1, FAM3D-AS1, AC078881.1, AC004466.2, AC018866.1, AC010531.3, AJ011931.2, TUSC8, PTOV1-AS1, RPS4XP2, AC021148.2, AC009041.2, AC093525.6, LINC01516, AL670729.3, C18ORF65, AC098818.2, FAM230C, LINC01988, AC068831.1, CYP51A1-AS1, LINC01227, LINC00895, LINC01519, AC114730.3, MIPEPP3, ITPRIP-AS1, AC138207.5, LINC01091, AC012645.3, AC082651.1, DLGAP1-AS3, AC080011.1, AC010148.1, AC078962.1, AL132822.1, LINC02552, AC098484.2, AC090587.1, LINC00173, PIK3CD-AS1, AL021393.1, AL450322.2, AL591368.1, AC010894.4, AC067930.8, AC024267.3, AC104984.1, AC109495.1, AC129926.1, KLF3-AS1, AL512306.3, LINC01448, AC080013.6, and DACT3-AS1.

88. An RNA nuclease agent of claim 84, wherein the one or more lncRNA molecules is selected from the group consisting of PXN-AS1, AC116366.2, HCG27, AL034417.2, ZMIZ1-AS1, AL513348.1, AC243829.2, LINC01227, LINC01091, AC080011.1, AL591368.1, AC109495.1, KLF3-AS1, and LINC01448.

89. The RNA nuclease agent of any one of claims 84-88, wherein treatment results in an improvement in fatigue.

90. The RNA nuclease agent of any one of claims 84-89, wherein the RNA nuclease agent is an RNase-Fc.

91. The RNA nuclease agent of any one of claims 84-89, wherein the RNA nuclease agent is RSLV-132.

92. A method of treating Sjogren’s syndrome in a patient in need thereof, the method comprising administering an effective amount of an RNA nuclease agent to the patient, wherein treatment results in a change in expression of one or more inflammatory-related genes, wherein the one or more inflammatory-related gene(s) is selected from the group consisting of CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, PLA2G7, NUPR1, ZC3H12A, TPST1, CDK19, CCN3, IL5, ILIRAP, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and CXCL1..

93. A method of treating Sjogren’s syndrome in a patient in need thereof, the method comprising administering an effective amount of an RNA nuclease agent to the patient, wherein treatment results in a decrease in expression of one or more inflammatory-related genes.

94. The method of claim 93, wherein the one or more inflammatory-related gene(s) is selected from the group consisting of NUPR1, ZC3H12A, TPST1, CDK19, CCN3, IL5, IL1RAP, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and/or CXCL1.

95. A method of treating Sjogren’s syndrome in a patient in need thereof, the method comprising administering an effective amount of an RNA nuclease agent to the patient, wherein treatment results in an increase in expression of one or more inflammatory-related genes.

96. The method of claim 95, wherein the one or more inflammatory-related gene(s) is selected from the group consisting of CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, and/or PLA2G7.

97. The method of any one of claims 92-96, wherein treatment results in an improvement in fatigue.

98. The method of any one of claims 92-97, wherein the RNA nuclease agent is an RNase-Fc.

99. The method of any one of claims 92-97, wherein the RNA nuclease agent is RSLV-132.

100. Use of an RNA nuclease agent in the manufacture of a medicament for the treatment of Sjogren’s syndrome in a patient, the use comprising administering an effective amount of an RNA nuclease agent to the patient, wherein treatment results in a change in expression of one or more inflammatory-related genes.

101. The use of claim 100, wherein the one or more inflammatory-related gene(s) is selected from the group consisting of CD36, RIPK2, PTPN2, APOL3, HGF, BIRC3, SETD6, CD47, IDO1, ACER3, APPL1, SNX4, CCR2, CD163, VAMP7, TBC1D23, CXCL10, AIMP1, CYBB, PLA2G7, NUPR1, ZC3H12A, TPST1, CDK19, CCN3, IL5, IL1RAP, TBXA2R, GBA, SLC11A1, MMP25, CSF1, FOS, TYROBP, FPR2, LTBR4, MAPKAPK2, STAT5B, ORM2, BCL6, IL1RN, SIRPA, TREM1, GPS2, FPR1, PGLYRP1, PIK3CD, NFAM1, IL17RA, IL6R, AKT1, TNFRSF1A, F11R, ADAM8, NLRP12, NDST1, IL1R2, MAPK13, MMP9, NOD2, and CXCL1.

102. Use of an RNA nuclease agent in the manufacture of a medicament for the treatment of Sjogren’s syndrome in a patient, the use comprising administering an effective amount of an RNA nuclease agent to the patient, wherein treatment results in a decrease in expression of one or more inflammatory-related genes.


SEQUENCE LISTING (69 sequences)

SEQ ID NO: 1 - protein, 156 aa, Homo sapiens
  MISC_FEATURE (1)..(156): Precursor human RNase1
MALEKSLVRLLLLVLILLVLGWVQPSLGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 2 - protein, 128 aa, Homo sapiens
  MISC_FEATURE (1)..(128): Mature human RNase1
KESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 3 - protein, 128 aa, Artificial Sequence
  Synthetic: hRNase-G88D-3
KESRAKKFQRQHMDSDSSPSSSSTYCNQMMRRRNMTQGRCKPVNTFVHEPLVDVQNVCFQEKVTCKNGQGNCYKSNSSMHITDCRLTNDSRYPNCAYRTSPKERHIIVACEGSPYVPVHFDASVEDST

SEQ ID NO: 4 - protein, 128 aa, Artificial Sequence
  Synthetic: Mature human RNase1 N34S/N76S/N88S
KESRAKKFQRQHMDSDSSPSSSSTYCNQMMRRRSMTQGRCKPVNTFVHEPLVDVQNVCFQEKVTCKNGQGNCYKSSSSMHITDCRLTSGSRYPNCAYRTSPKERHIIVACEGSPYVPVHFDASVEDST

SEQ ID NO: 5 - protein, 282 aa, Homo sapiens [sequence omitted]

SEQ ID NO: 6 - protein, 260 aa, Homo sapiens
  MISC_FEATURE (1)..(260): Mature wild type Human DNase1
LKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 7 - protein, 260 aa, Artificial Sequence
  Synthetic: hDNase1-3-G105R;A114F
LKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 8 - protein, 260 aa, Artificial Sequence
  Synthetic: hDNase1-3A114F
LKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 9 - protein, 260 aa, Artificial Sequence
  Synthetic: hDNase1-5-G105R
LKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCRNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 10 - protein, 280 aa, Artificial Sequence
  Synthetic: human DNase1+VK3LP
METPAQLLFLLLLWLPDTTGLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 11 - protein, 260 aa, Artificial Sequence
  Synthetic: Mature human DNase1 N18S/N106S/A114F
LKIAAFNIQTFGETKMSSATLVSYIVQILSRYDIALVQEVRDSHLTAVGKLLDNLNQDAPDTYHYVVSEPLGRNSYKERYLFVYRPDQVSAVDSYYYDDGCEPCGSDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 12 - protein, 260 aa, Artificial Sequence
  Synthetic: Mature human DNase1 E13R/N74K/A114F/ T205K
LKIAAFNIQTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTAKPTHCAYDRIVVAGMLLRGAVVPDSALPFNFQAAYGLSDQLAQAISDHYPVEVMLK

SEQ ID NO: 13 - protein, 260 aa, Artificial Sequence
  Synthetic: Mature human DNase1 E13R/N74K/A114F/ T205K/N18S/N106S
LKIAAFNIQTFGRTKMSSATLVSYIVQILSRYDIALVQEVRDSHLTAVGKLLDNLNQDAPDTYHYVVSEPLGRKSYKERYLFVYRPDQVSAVDSYYYDDGCEPCGSDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTAKPTHCAYDRIVVAGMLLRGAVVPDSALPFNFQAAYGLSDQLAQAISDHYPVEVMLK

SEQ ID NO: 14 - protein, 305 aa, Homo sapiens
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 15 - protein, 285 aa, Homo sapiens
MRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 16 - protein, 369 aa, Homo sapiens
MGPGARRQGRIVQGRPEMCFCPPPTPLPPLRILTLGTHTPTPCSSPGSAAGTYPTMGSQALPPGPMQTLIFFDMEATGLPFSQPKVTELCLLAVHRCALESPPTSQGPPPTVPPPPRVVDKLSLCVAPGKACSPAASEITGLSTAVLAAHGRQCFDDNLANLLLAFLRRQPQPWCLVAHNGDRYDFPLLQAELAMLGLTSALDGAFCVDSITALKALERASSPSEHGPRKSYSLGSIYTRLYGQSPPDSHTAEGDVLALLSICQWRPQALLRWVDAHARPFGTIRPMYGVTASARTKPRPSAVTTTAHLATTRNTSPSLGESRGTKDLPPVKDPGALSREGLLAPLGLLAILTLAVATLYGLSLATPGE

SEQ ID NO: 17 - protein, 297 aa, Homo sapiens
MGPGARRQGRIVQGRPEMCFCPPPTPLPPLRILTLGTHTPTPCSSPGSAAGTYPTMGSQALPPGPMQTLIFFDMEATGLPFSQPKVTELCLLAVHRCALESPPTSQGPPPTVPPPPRVVDKLSLCVAPGKACSPAASEITGLSTAVLAAHGRQCFDDNLANLLLAFLRRQPQPWCLVAHNGDRYDFPLLQAELAMLGLTSALDGAFCVDSITALKALERASSPSEHGPRKSYSLGSIYTRLYGQSPPDSHTAEGDVLALLSICQWRPQALLRWVDAHARPFGTIRPMYGVTASARTK

SEQ ID NO: 18 - protein, 360 aa, Homo sapiens
  MISC_FEATURE (1)..(360): Human DNase2 alpha
MIPLLLAALLCVPAGALTCYGDSGQPVDWFVVYKLPALRGSGEAAQRGLQYKYLDESSGGWRDGRALINSPEGAVGRSLQPLYRSNTSQLAFLLYNDQPPQPSKAQDSSMRGHTKGVLLLDHDGGFWLVHSVPNFPPPASSAAYSWPHSACTYGQTLLCVSFPFAQFSKMGKQLTYTYPWVYNYQLEGIFAQEFPDLENVVKGHHVSQEPWNSSITLTSQAGAVFQSFAKFSKFGDDLYSGWLAAALGTNLQVQFWHKTVGILPSNCSDIWQVLNVNQIAFPGPAGPSFNSTEDHSKWCVSPKGPWTCVGDMNRNQGEEQRGGGTLCAQLPALWKAFQPLVKNYQPCNGMARKPSRAYKI

SEQ ID NO: 19 - protein, 361 aa, Homo sapiens
  MISC_FEATURE (1)..(361): human DNase2 beta
MKQKMMARLLRTSFALLFLGLFGVLGAATISCRNEEGKAVDWFTFYKLPKRQNKESGETGLEYLYLDSTTRSWRKSEQLMNDTKSVLGRTLQQLYEAYASKSNNTAYLIYNDGVPKPVNYSRKYGHTKGLLLWNRVQGFWLIHSIPQFPPIPEEGYDYPPTGRRNGQSGICITFKYNQYEAIDSQLLVCNPNVYSCSIPATFHQELIHMPQLCTRASSSEIPGRLLTTLQSAQGQKFLHFAKSDSFLDDIFAAWMAQRLKTHLLTETWQRKRQELPSNCSLPYHVYNIKAIKLSRHSYFSSYQDHAKWCISQKGTKNRWTCIGDLNRSPHQAFRSGGFICTQNWQIYQAFQGLVLYYESCK

SEQ ID NO: 20 - protein, 232 aa, Homo sapiens
  MISC_FEATURE (1)..(232): Human IgG1 Fc domain (wild-type)
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 232 aa, Artificial Sequence
  Synthetic: Fc region N83S
EPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 232 aa, Artificial Sequence
  Synthetic: IgG1 Fc domain with SCC, P238S, and P331S
EPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 232 aa, Artificial Sequence
  Synthetic: Fc region with P238S, P331S mutations
EPKSCDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 232 aa, Artificial Sequence
  Synthetic: IgG1 Fc (SCC hinge)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 232 aa, Artificial Sequence
  Synthetic: IgG1 Fc domain (SSS hinge)
EPKSSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 232 aa, Artificial Sequence
  Synthetic: IgG1 Fc domain with P238S (SCC hinge)
EPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 232 aa, Artificial Sequence
  Synthetic: IgG1 Fc domain with P331S
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 232 aa, Artificial Sequence
  Synthetic: IgG1 Fc domain with SSS, P238S, and P331S
EPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 327 aa, Homo sapiens
  MISC_FEATURE (1)..(327): Human IgG4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 218 aa, Homo sapiens
  MISC_FEATURE (1)..(218): Human IgG4 Fc domain
PAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 229 aa, Homo sapiens
  MISC_FEATURE (1)..(229): Human IgG4 Hinge + Fc domain
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 20 aa, Artificial Sequence
  Synthetic: (Gly4Ser)4
GGGGSGGGGSGGGGSGGGGS

SEQ ID NO: 33 - protein, 15 aa, Artificial Sequence
  Synthetic: (Gly4Ser)3
GGGGSGGGGSGGGGS

SEQ ID NO: 34 - protein, 25 aa, Artificial Sequence
  Synthetic: (Gly4Ser)5
GGGGSGGGGSGGGGSGGGGSGGGGS

SEQ ID NO: 35 - protein, 10 aa, Artificial Sequence
  Synthetic: (Gly4Ser)2
GGGGSGGGGS

SEQ ID NO: 36 - protein, 5 aa, Artificial Sequence
  Synthetic: (Gly4Ser)1
GGGGS

SEQ ID NO: 37 - protein, 18 aa, Artificial Sequence
  Synthetic: NLG linker
VDGASSPVNVSSPSVQDI

SEQ ID NO: 38 - protein, 47 aa, Artificial Sequence
  Synthetic: Linker
LEAEAAAKEAAAKEAAAKEAAAKALEAEAAAKEAAAKEAAAKEAAAK

SEQ ID NO: 39 - protein, 50 aa, Artificial Sequence
  Synthetic: linker
LEAEAAAKEAAAKEAAAKEAAAKALEAEAAAKEAAAKEAAAKEAAAKALE

SEQ ID NO: 40 - protein, 4 aa, Artificial Sequence
  Synthetic: Linker
GGSG

SEQ ID NO: 41 - protein, 4 aa, Artificial Sequence
  Synthetic: Linker
GSAT

SEQ ID NO: 42 - protein, 19 aa, Artificial Sequence
  Synthetic: Leader Sequence
MDWTWRILFLVAAATGTHA

SEQ ID NO: 43 - protein, 20 aa, Artificial Sequence
  Synthetic: VK3LP leader sequence
METPAQLLFLLLLWLPDTTG

SEQ ID NO: 44 - protein, 381 aa, Artificial Sequence
  Synthetic: RSLV-124 hVK3LP-hRNase(WT)-hIgG1 WT
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 381 aa, Artificial Sequence
  Synthetic: RSLV125: huVK3LP-wthRNase-SSS-mthIgG1 P238S P331S
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 381 aa, Artificial Sequence
  Synthetic: RSLV125-2: huVK3LP-wthRNase-SCC-mthIgG1 P238S P331S
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTLEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 401 aa, Artificial Sequence
  Synthetic: RSLV126: huVK3LP-WThRNase-(g4s)4-SSS-mthIgG1-P238S-P331S
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTGGGGSGGGGSGGGGSGGGGSLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 401 aa, Artificial Sequence
  Synthetic: RSLV126-2: huVK3LP-WThRNase-(g4s)4-SCC-mthIgG1-P238S-P331S
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTGGGGSGGGGSGGGGSGGGGSLEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 381 aa, Artificial Sequence; identical to SEQ ID NO: 46
  Synthetic: RSLV132: huVK3LP-wthRNase-SCC-mthIgG1 P238S P331S

SEQ ID NO: 50 - protein, 361 aa, Artificial Sequence
  Synthetic: RSLV132: wthRNase-SCC-mthIgG1 P238S P331S
KESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTLEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 679 aa, Artificial Sequence
  Synthetic: RSLV127: huVK3LP-hDNase1 105/114-(g4s)4-SSS-mthIgG1-P238S-P331S-NLG-RNase
METPAQLLFLLLLWLPDTTGLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGGGGSGGGGSGGGGSGGGGSLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDIKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 52 - protein, 679 aa, Artificial Sequence
  Synthetic: RSLV127-2: huVK3LP-hDNase1 105/114-(g4s)4-SCC-mthIgG1-P238S-P331S-NLG-RNase
METPAQLLFLLLLWLPDTTGLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGGGGSGGGGSGGGGSGGGGSLEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDIKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 53 - protein, 679 aa, Artificial Sequence
  Synthetic: RSLV128: huVK3LP-hRNase WT-(g4s)4-SSS-mthIgG1-P238S-P331S-NLG-hDNase 105/114
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTGGGGSGGGGSGGGGSGGGGSLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 54 - protein, 679 aa, Artificial Sequence
  Synthetic: RSLV128-2: huVK3LP-hRNase WT-(g4s)4-SCC-mthIgG1-P238S-P331S-NLG-hDNase 105/114
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTGGGGSGGGGSGGGGSGGGGSLEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 55 - protein, 659 aa, Artificial Sequence
  Synthetic: RSLV129: huVK3LP-hRNAseWT-SSS-mthIgG1-P238S-P331S-NLG-hDNAse 105/114
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 56 - protein, 659 aa, Artificial Sequence
  Synthetic: RSLV129-2: huVK3LP-hRNAseWT-SCC-mthIgG1-P238S-P331S-NLG-hDNAse 105/114
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTLEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 57 - protein, 659 aa, Artificial Sequence; identical to SEQ ID NO: 56
  Synthetic: RSLV133: huVK3LP-hRNAseWT-SCC-mthIgG1-P238S-P331S-NLG-hDNAse 105/114

SEQ ID NO: 58 - protein, 639 aa, Artificial Sequence
  Synthetic: RSLV133: hRNAseWT-SCC-mthIgG1-P238S-P331S-NLG-hDNAse 105/114
KESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTLEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 59 - protein, 7 aa, Artificial Sequence
  Synthetic: O-linked glycosylation consensus
  MISC_FEATURE (2)..(3): Xaa can be any naturally occurring amino acid
  MISC_FEATURE (6)..(6): X is E or D
CXXGGXC

SEQ ID NO: 60 - protein, 5 aa, Artificial Sequence
  Synthetic: O-linked glycosylation consensus
  VARIANT (4)..(4): X is E or D
NSTXA

SEQ ID NO: 61 - protein, 5 aa, Artificial Sequence
  Synthetic: O-linked glycosylation consensus
NITQS

SEQ ID NO: 62 - protein, 5 aa, Artificial Sequence
  Synthetic: O-linked glycosylation consensus
QSTQS

SEQ ID NO: 63 - protein, 5 aa, Artificial Sequence
  Synthetic: O-linked glycosylation consensus
  VARIANT (1)..(1): X is D or E
  VARIANT (4)..(4): X is R or K
XFTXV

SEQ ID NO: 64 - protein, 4 aa, Artificial Sequence
  Synthetic: O-linked glycosylation consensus
  VARIANT (2)..(2): X is E or D
CXSN

SEQ ID NO: 65 - protein, 5 aa, Artificial Sequence
  Synthetic: O-linked glycosylation consensus
  VARIANT (5)..(5): X is K or R
GGSCX

SEQ ID NO: 66 - protein, 25 aa, Artificial Sequence
  Synthetic: Linker
  MISC_FEATURE (6)..(25): "Gly Gly Gly Gly Ser" may or may not be present
GGGGSGGGGSGGGGSGGGGSGGGGS

SEQ ID NO: 67 - protein, 27 aa, Artificial Sequence
  Synthetic: Linker
  MISC_FEATURE (12)..(26): "Glu Ala Ala Ala Lys" may or may not be present
AEAAAKEAAAKEAAAKEAAAKEAAAKA

SEQ ID NO: 68 - protein, 4 aa, Artificial Sequence
  Synthetic: Linker
  MISC_FEATURE: "Gly Gly Ser Gly" may repeat indefinitely
GGSG

SEQ ID NO: 69 - protein, 30 aa, Artificial Sequence
  Synthetic: Linker
  MISC_FEATURE (7)..(30): "Gly Gly Ser Gly Gly Ser" may or may not be present
GGSGGSGGSGGSGGSGGSGGSGGSGGSGGS